(19) United States
(12) Patent Application Publication
(10) Pub. No.: US 2022/0331304 A1
(43) Pub. Date: Oct. 20, 2022

(54) COMPOUNDS FOR USE IN TREATING NEUROLOGICAL DISORDERS

(71) Applicant: Constellation Pharmaceuticals, Inc., Cambridge, MA (US)

(72) Inventors: Francois Brucelle, Cambridge, MA (US); Julian R. Levell, Arlington, MA (US); Jonathan E. Wilson, Arlington, MA (US)

(21) Appl. No.: 17/631,010

(22) PCT Filed: Jul. 29, 2020
(86) PCT No.: PCT/US2020/044014

§ 371 Date: Jan. 28, 2022

Related U.S. Application Data
(60) Provisional application No. 62/879,870, filed on Jul. 29, 2019.

(51) Int. Cl. A61K 31/4439; A61K 31/415; A61K 31/506; A61K 31/4164; A61K 31/50; A61K 31/501; A61K 31/495; A61K 31/451; A61K 31/4155; A61K 31/4427; A61K 31/4545; A61K 31/496; A61K 31/4045

(52) U.S. Cl. CPC A61K 31/4439; A61K 31/415; A61K 31/506; A61K 31/4164; A61K 31/50; A61K 31/501; A61K 31/495; A61K 31/451; A61K 31/4155; A61K 31/4427; A61K 31/4545; A61K 31/496; A61K 31/4045

(57) ABSTRACT

Provided are methods for treating neurological disorders using compounds of Formula (I), and pharmaceutically acceptable salts and compositions thereof.

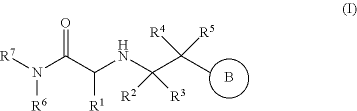

### RELATED APPLICATIONS

This application claims priority to U.S. Provisional Application No. 62/879,870, filed Jul. 29, 2019, the entire contents of which are incorporated herein by reference.

### BACKGROUND

Neurological disorders affect the central nervous system, the peripheral nervous system or the autonomic nervous system. The specific causes of neurological problems vary, but can include genetic disorders, congenital abnormalities or disorders, infections, lifestyle or environmental health problems including malnutrition, and brain injury, spinal cord injury, nerve injury and gluten sensitivity (with or without intestinal damage or digestive symptoms).

According to the World Health Organization (WHO), neurological disorders are one of the greatest threats to public health. In 2006 alone, the WHO estimated that neurological disorders and their direct consequences affect as many as one billion people worldwide. Additionally, according to the Federal Interagency Forum on Aging-Related Statistics, 35.8% of persons aged 85 years and older have moderate or severe memory impairment, with over 16% of US households containing an individual with brain impairment. Despite current research, the prevalence of neurological disorders. Thus, there is an ongoing need for new therapeutic agents that are effective against neurological disorders.

### SUMMARY

Provided herein are compounds that are useful for treating neurological disorders. Such compounds include those having the Formula I:

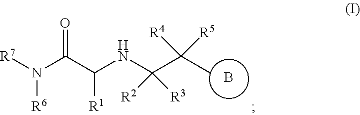

and pharmaceutically acceptable salts and compositions thereof, wherein B, R¹, R², R³, R⁴, R⁵, R⁶, and R⁷ are as described herein.

### DETAILED DESCRIPTION

### 1. General Description of Compounds

The compound(s) described in the methods herein include both the neutral form and a pharmaceutically acceptable salt thereof.

In a first embodiment, provided herein are methods of treating a neurological disorder comprising administering to a subject an effective amount of a compound of Formula I:

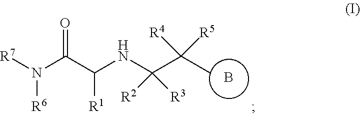

or a pharmaceutically acceptable salt thereof, wherein

Ring B is aryl, heterocyclyl, or heteroaryl each of which may be optionally substituted with 1 to 4 groups selected from R^{b};

R⁶ is a hydrogen or C₁₋₆alkyl;

R⁷ is aryl or heteroaryl, each of which is substituted with one group selected from R^{f}, and wherein said aryl and heteroaryl for R⁷ may also be optionally substituted with 1 to 4 groups selected from R^{a}; or R⁶ and R⁷ taken together with the nitrogen ring to which they are attached form a fused bicyclic heterocyclyl optionally substituted with 1 to 4 groups selected from R^{a};

R¹ is C₁₋₆alkyl, C₁₋₆haloalkyl, C₂₋₆alkenyl, —C₁₋₆alkylOR^{c}, —C₁₋₆alkylN(R^{d})₂, —C₁₋₆alkylC(O)OR^{d}, —C₁₋₆alkylOC₁₋₆alkylN(R^{d})₂, —C₁₋₆alkylSOR^{d}, —C₁₋₆alkylS(O)₂R^{d}, —C₁₋₆alkylSON(R^{d})₂, —C₁₋₆alkylSO₂N(R^{d})₂, —C₁₋₆alkylcycloalkyl, —C₁₋₆alkylheterocyclyl, —C₁₋₆alkylheteroaryl, —C₁₋₆alkylaryl, cycloalkyl, aryl, heteroaryl, or heterocyclyl, wherein each of said cycloalkyl, heterocyclyl, aryl, and heteroaryl alone and in connection with —C₁₋₆alkylcycloalkyl, —C₁₋₆alkylaryl, —C₁₋₆alkylheteroaryl, and —C₁₋₆alkylheterocyclyl are optionally substituted with 1 to 3 groups selected from R^{c};

each of R², R³, R⁴, and R⁵ are independently hydrogen or C₁₋₆alkyl, wherein said C₁₋₆alkyl is optionally substituted with 1 or 2 groups selected from halo, —C(O)OR^{d}, —OC₁₋₆alkylN(R^{d})₂, —C₁₋₆alkylN(R^{d})₂, —N(R^{d})₂, —NR^{d}C₁₋₆alkylOR^{d}, —SOR^{d}, —S(O)₂R^{d}, —SON(R^{d})₂, —SO₂N(R^{d})₂, C₃₋₁₀cycloalkyl, C₅₋₁₀heterocyclyl, C₅₋₁₀heteroaryl, and C₆₋₁₀aryl;

each of R^{a}, R^{b}, and R^{c} are each independently halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅, —Ocycloalkyl, —O—C₁₋₄alkylaryl, —C₁₋₆alkylcycloalkyl, —C₁₋₆alkylaryl, —C₁₋₆alkylheteroaryl, —C₁₋₆alkylheterocyclyl, cycloalkyl, heterocyclyl, heteroaryl, or aryl, wherein each of said cycloalkyl, heterocyclyl, aryl, and heteroaryl alone and in connection with —Ocycloalkyl, —C₁₋₆alkylcycloalkyl, —C₁₋₆alkylaryl, —C₁₋₆alkylheteroaryl, and —C₁₋₆alkylheterocyclyl are optionally substituted with 1 to 3 groups selected from halo, C₁₋₆alkyl, C₁₋₆haloalkyl, C₁₋₆alkoxy, C₁₋₆haloalkoxy, —N(R^{d})₂, —C(O)R^{d}, and —C₁₋₆alkylOR^{d};

each R^{d} is independently hydrogen, C₁₋₆haloalkyl, or C₁₋₆alkyl; and

each R^{f} is independently cycloalkyl, heterocyclyl, heteroaryl, or aryl, wherein each of said cycloalkyl, heterocyclyl, aryl, and heteroaryl are optionally substituted with 1 to 3 groups selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅, —Ocycloalkyl. Also provided, as part of a first embodiment, is the use of an effective amount of a compound of Formula I or a pharmaceutically acceptable salt thereof for treating a neurological disorder, wherein the variables Formula I are as described above in this paragraph. Also provided, as part of a first embodiment, is the use of a compound of Formula I or a pharmaceutically acceptable salt thereof for the manufacture of a medicament for treating a neurological disorder, wherein the variables Formula I are as described above in this paragraph. Further provided is a pharmaceutical composition comprising a compound of Formula I or a pharmaceutically acceptable salt thereof for treating a neurological disorder, wherein the variables Formula I are as described above in this paragraph.

### 2. Definitions

When used in connection to describe a chemical group that may have multiple points of attachment, a hyphen (-) designates the point of attachment of that group to the variable to which it is defined. For example, —N(R^{d})₂ and —NR^{d}C₁₋₆alkylOR^{d} mean that the point of attachment for this group occurs on the nitrogen atom.

The terms “halo” and “halogen” refer to an atom selected from fluorine (fluoro, —F), chlorine (chloro, —C₁), bromine (bromo, —Br), and iodine (iodo, —I).

The term “alkyl” when used alone or as part of a larger moiety, such as “haloalkyl”, “alkylC₅₋₁₀heterocyclyl”, and the like, means saturated straight-chain or branched monovalent hydrocarbon radical. Unless otherwise specified, an alkyl group typically has 1-6 carbon atoms, i.e., (C₁-C₆)alkyl.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., but are not limited to —OCHCF₂ or —OCF₃.

The term “oxo” refers to the diradical ═O

The term “aryl” refers to an aromatic carbocyclic single ring or two fused ring system containing 6 to 10 carbon atoms. Examples include phenyl, indanyl, tetrahydronaphthalene, and naphthyl.

The term “carbocyclyl” means a monocyclic, bicyclic (e.g., a bridged or spiro bicyclic ring), polycyclic (e.g., tricyclic), or fused hydrocarbon ring system that is completely saturated or that contains one or more units of unsaturation, but where there is no aromatic ring. Cycloalkyl is a completely saturated carbocycle. Monocyclic cycloalkyl groups include, without limitation, cyclopropyl, cyclobutyl, cyclopentyl, cyclohexyl, cycloheptyl, and cyclooctyl. Bridged bicyclic cycloalkyl groups include, without limitation, bicyclo[3.2.1]octane, bicyclo[2.2.1]heptane, bicyclo[3.1.0]hexane, bicyclo[1.1.1]pentane, and the like. Spiro bicyclic cycloalkyl groups include, e.g., spiro[3.6]decane, spiro[4.5]decane, and the like. Fused cycloalkyl rings include, e.g., decahydronaphthalene, octahydropentalene, and the like. It will be understood that when specified, optional substituents on a carbocyclyl (e.g., in the case of an optionally substituted cycloalkyl) may be present on any substitutable position and, include, e.g., the position at which the carbocyclyl group is attached.

The term “heteroaryl” used alone or as part of a larger moiety refers to a 5- to 12-membered aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 5- to 12-membered saturated or partially unsaturated heterocyclic ring containing 1 to 4 heteroatoms independently selected from N, O, and S. It can be mononcyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “spiro” refers to two rings that shares one ring atom (e.g., carbon).

The term “fused” refers to two rings that share two adjacent ring atoms with one another.

The term “bridged” refers to two rings that share three ring atoms with one another.

The disclosed compounds exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms.

“Racemate” or “racemic mixture” means a compound of equimolar quantities of two enantiomers, wherein such mixtures exhibit no optical activity, i.e., they do not rotate the plane of polarized light.

The compounds of the methods herein may be prepared as individual enantiomers by either enantio-specific synthesis or resolved from an enantiomerically enriched mixture. Conventional resolution techniques include forming the salt of a free base of each isomer of an enantiomeric pair using an optically active acid (followed by fractional crystallization and regeneration of the free base), forming the salt of the acid form of each enantiomer of an enantiomeric pair using an optically active amine (followed by fractional crystallization and regeneration of the free acid), forming an ester or amide of each of the enantiomers of an enantiomeric pair using an optically pure acid, amine or alcohol (followed by chromatographic separation and removal of the chiral auxiliary), or resolving an enantiomeric mixture of either a starting material or a final product using various well known chromatographic methods. Additionally, the compounds can be prepared as individual enantiomers by separating a racemic mixture using conventional chiral chromatography techniques.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomer is at least 60%, 70%, 80%, 90%, 99% or 99.9% by weight pure relative to all of the other stereoisomers. The stereoisomeric purity in this case is determined by dividing the total weight in the mixture of the stereoisomers encompassed by the name or structure by the total weight in the mixture of all of the stereoisomers.

When a disclosed compound is named or depicted by structure without indicating the stereochemistry, and the compound has one chiral center, it is to be understood that the name or structure encompasses one enantiomer of compound free from the corresponding optical isomer, a racemic mixture of the compound, or mixtures enriched in one enantiomer relative to its corresponding optical isomer.

When a disclosed compound is named or depicted by structure without indicating the stereochemistry and e.g., the compound has more than one chiral center (e.g., at least two chiral centers), it is to be understood that the name or structure encompasses one stereoisomer free of other stereoisomers, mixtures of stereoisomers, or mixtures of stereoisomers in which one or more stereoisomers is enriched relative to the other stereoisomer(s). For example, the name or structure may encompass one stereoisomer free of other diastereomers, mixtures of stereoisomers, or mixtures of stereoisomers in which one or more diastereomers is enriched relative to the other diastereomer(s).

Unless otherwise specified, when only some of the stereochemical centers in a disclosed compound are depicted or named by structure, the named or depicted configuration is enriched relative to the remaining configurations, for example, by a molar excess of at least 60%, 70%, 80%, 90%, 99% or 99.9%. For example, the structure:

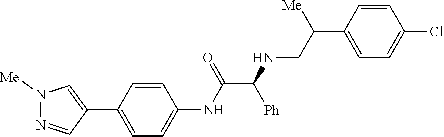 [SMILES: CC(CN[C@H](C(=O)Nc1ccc(-c2cnn(C)c2)cc1)c1ccccc1)c1ccc(Cl)cc1]

means that that the configuration about the chiral carbon where the stereochemistry is depicted is stereochemically enriched as S (e.g., by a molar excess of at least 60%, 70%, 80%, 90%, 99% or 99.9%) and that the stereochemistry at the other chiral center, to which the stereochemistry is not identified, may be R or S, or a mixture thereof.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a neurological disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “effective amount” or “therapeutically effective amount” refers to an amount of a compound described herein that will elicit a biological or medical response of a subject e.g., a dosage of between 0.01-100 mg/kg body weight/day.

### 3. Compounds

In a second embodiment of the methods described herein, the is compound of the Formula I:

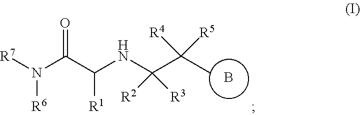

or a pharmaceutically acceptable salt thereof, wherein the variables are as described above; provided the compound is not N-[1,1′-biphenyl]-2-yl-2-[[2-(3,4-dimethoxyphenyl)ethyl]amino]-propanamide, or 2-[(2-phenylpropyl)amino]-N-[4-(1H-1,2,4-triazol-1-yl)phenyl]-propanamide, or a salt thereof.

In a third embodiment of the methods described herein, the compound of Formula I is of the Formula II or III:

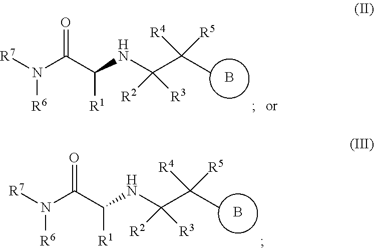

or a pharmaceutically acceptable salt thereof, wherein the remaining variables are as described for Formula I or the second embodiment.

In a fourth embodiment of the methods described herein, R⁶ in the compounds of Formula I, II, or III is hydrogen; and R⁷ is aryl or heteroaryl, each of which is substituted with one group selected from R^{f}, and wherein said aryl and heteroaryl for R⁷ may also be optionally substituted with 1 to 4 groups selected from R^{a}; or R⁶ and R⁷ taken together with the nitrogen ring to which they are attached form a fused bicyclic heterocyclyl optionally substituted with 1 to 4 groups selected from R^{a}, wherein the remaining variables are as described above for Formula I or the second embodiment. Alternatively, R⁶ in the compounds of Formula I, II, or III is hydrogen; and R⁷ is phenyl, pyridyl, pyrimidinyl, or quinolinyl, each of which is substituted with one group selected from R^{f}, and wherein said phenyl, pyridyl, pyrimidinyl, and quinolinyl for R⁷ may also be optionally substituted with 1 to 4 groups selected from R^{a}; or R⁶ and R⁷ taken together with the nitrogen ring to which they are attached form a 5,6- or 6,6-fused bicyclic heterocyclyl optionally substituted with 1 to 4 groups selected from R^{a}, wherein the remaining variables are as described above for Formula I or the second embodiment. In another alternative, R⁶ in the compounds of Formula I, II, or III is hydrogen; R⁷ is selected from phenyl, 2-pyridinyl, 3-pyridinyl, pyrimidin-5-yl, and quinolin-6-yl, each of which is substituted with one group from R^{f}, and wherein said phenyl, 2-pyridinyl, 3-pyridinyl, pyrimidin-5-yl, and quinolin-6-yl for R⁷ may also be optionally substituted with 1 to 4 groups selected from R^{a}; or R⁶ and R⁷ taken together with the nitrogen ring to which they are attached form indolin-1-yl or dihydroquinolin-1(2H)-yl, each of which may be optionally substituted with 1 to 4 groups selected from R^{a}, wherein the remaining variables are as described above for Formula I or the second embodiment.

In a fifth embodiment of the methods described herein, Ring B in the compounds of Formula I, II, or III is phenyl optionally substituted with 1 to 3 groups selected from R^{b}, wherein the remaining variables are as described above for Formula I or the second or fourth embodiment.

In a sixth embodiment of the methods described herein, R¹ in the compounds of Formula I, II, or III is phenyl optionally substituted with 1 to 3 groups selected from R^{c}, wherein the remaining variables are as described above for Formula I or the second, fourth, or fifth embodiment.

In a seventh embodiment of the methods described herein, R³ in the compounds of Formula I, II, or III is hydrogen, wherein the remaining variables are as described above for Formula I or the second, fourth, fifth, or sixth embodiment.

In an eighth embodiment of the methods described herein, R⁵ in the compounds of Formula I, II, or III is hydrogen, wherein the remaining variables are as described above for Formula I or the second, fourth, fifth, sixth, or seventh embodiment.

In a ninth embodiment of the methods described herein, R² in the compounds of Formula I, II, or III is hydrogen or C₁₋₄alkyl, wherein the remaining variables are as described above for Formula I or the second, fourth, fifth, sixth, seventh, or eighth embodiment. Alternatively, R² in the compounds of Formula I, II, or III is hydrogen or methyl, wherein the remaining variables are as described above for Formula I or the second, fourth, fifth, sixth, seventh, or eighth embodiment. In another alternative, R² in the compounds of Formula I, II, or III is hydrogen, wherein the remaining variables are as described above for Formula I or the second, fourth, fifth, sixth, seventh, or eighth embodiment.

In a tenth embodiment of the methods described herein, R⁴ in the compounds of Formula I, II, or III is hydrogen or C₁₋₄alkyl, wherein the remaining variables are as described above for Formula I or the second, fourth, fifth, sixth, seventh, eighth, or ninth embodiment. Alternatively, R⁴ in the compounds of Formula I, II, or III is hydrogen, methyl, or ethyl, wherein the remaining variables are as described above for Formula I or the second, fourth, fifth, sixth, seventh, eighth, or ninth embodiment. In another alternative, R⁴ in the compounds of Formula I, II, or III is hydrogen, wherein the remaining variables are as described above for Formula I or the second, fourth, fifth, sixth, seventh, eighth, or ninth embodiment.

In an eleventh embodiment of the methods described herein, the compound of Formula I is of the Formula IV or V:

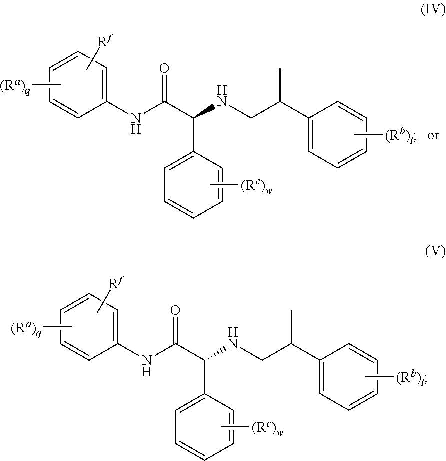

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. Alternatively, the compound of Formula I is of the Formula VI or VII:

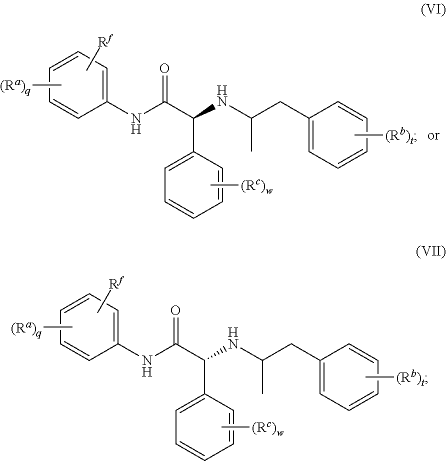

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. In another alternative, the compound of Formula I is of the Formula VIII or IX:

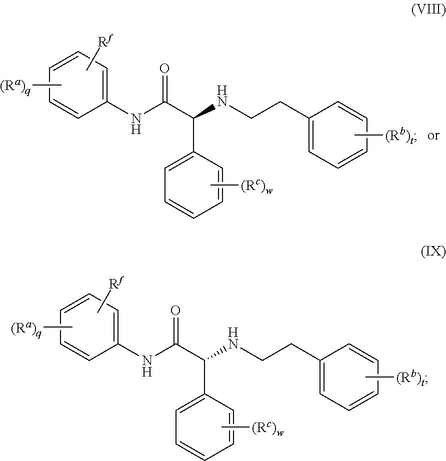

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment.

In an twelfth embodiment of the methods described herein, R^{c}, if present, in the compounds of Formula I, II, III, IV, V, VI, VII, VIII, or IX is C₁₋₆alkyl, C₁₋₆alkoxy, C₁₋₆haloalkoxy, or C₁₋₆haloalkyl, wherein the remaining variables are as described above for Formula I or the second, fourth, fifth, sixth, seventh, eighth, ninth, tenth, or eleventh embodiment.

In a thirteenth embodiment of the methods described herein, the compound of Formula I is of the Formula X or XI:

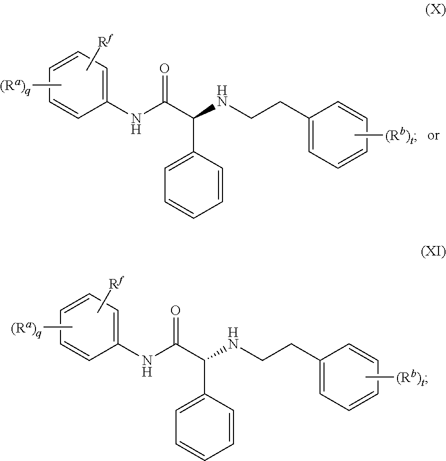

or a pharmaceutically acceptable salt thereof, wherein the remaining variables are as described above for Formula I or the second, fourth, fifth, sixth, seventh, eighth, ninth, tenth, eleventh, or twelfth embodiment.

In a fourteenth embodiment of the methods described herein, q in the compounds of Formula IV, V, VI, VII, VIII, or IX is 0 or 1, wherein the remaining variables are as described above for Formula I or the second, fourth, fifth, sixth, seventh, eighth, ninth, tenth, eleventh, twelfth, or thirteenth embodiment.

In a fifteenth embodiment of the methods described herein, R^{a} in the compounds of Formula I, II, III, IV, V, VI, VII, VIII, IX, X, and XI is C₁₋₄alkoxy or halo, wherein the remaining variables are as described above for Formula I or the second, fourth, fifth, sixth, seventh, eighth, ninth, tenth, eleventh, twelfth, thirteenth, or fourteenth embodiment.

In a sixteenth embodiment of the methods described herein, R^{f} in the compounds of Formula I, II, III, IV, V, VI, VII, VIII, IX, X, and XI is heteroaryl or heterocyclyl, each of which may be optionally substituted with 1 to 3 groups selected from selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅, —Ocycloalkyl, wherein the remaining variables are as described above for Formula I or the second, fourth, fifth, sixth, seventh, eighth, ninth, tenth, eleventh, twelfth, thirteenth, fourteenth, or fifteenth embodiment. Alternatively, R^{f} in the compounds of Formula I, II, III, IV, V, VI, VII, VIII, IX, X, and XI is pyrazolyl, imidazolyl, pyridazinyl, piperazinyl, or piperidinyl, each of which may be optionally substituted with 1 to 3 groups selected from selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅, —Ocycloalkyl, wherein the remaining variables are as described above for Formula I or the second, fourth, fifth, sixth, seventh, eighth, ninth, tenth, eleventh, twelfth, thirteenth, fourteenth, or fifteenth embodiment.

In a seventeenth embodiment of the methods described herein, R^{f} in the compounds of Formula I, II, III, IV, V, VI, VII, VIII, IX, X, and XI is pyrazolyl, imidazolyl, pyridazinyl, piperazinyl, or piperidinyl, each of which may be optionally substituted with 1 to 3 groups selected from selected from C₁₋₄alkyl and —C(O)R^{d}, wherein R^{d} is C₁₋₄alkyl, wherein the remaining variables are as described above for Formula I or the second, fourth, fifth, sixth, seventh, eighth, ninth, tenth, eleventh, twelfth, thirteenth, fourteenth, fifteenth, or sixteenth embodiment.

In an eighteenth embodiment of the methods described herein, R^{b} in the compounds of Formula I, II, III, IV, V, VI, VII, VIII, IX, X, and XI is halo, wherein the remaining variables are as described above for Formula I or the second, fourth, fifth, sixth, seventh, eighth, ninth, tenth, eleventh, twelfth, thirteenth, fourteenth, fifteenth, sixteenth, or seventeenth embodiment.

In a nineteenth embodiment of the methods described herein, the compound of Formula I is of the Formula XII or XIII:

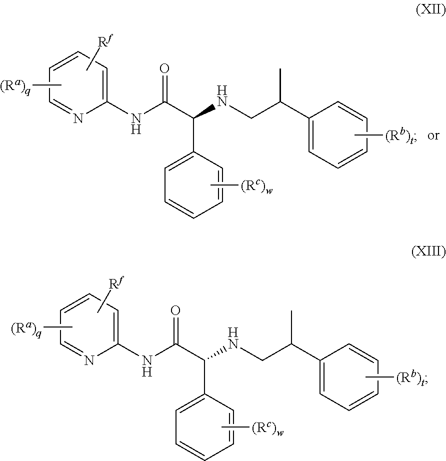

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. Alternatively, the compound of Formula I is of the Formula XIV or XV:

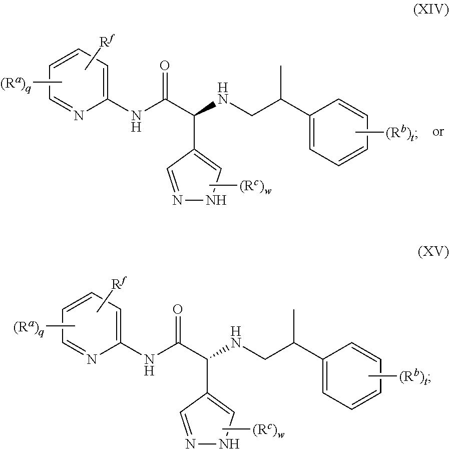

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. In another alternative, the compound of Formula I is of the Formula XVI or XVII:

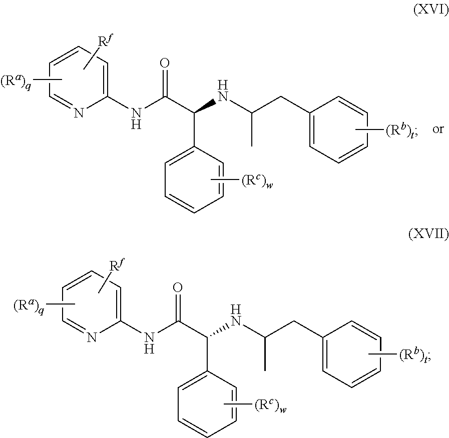

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. In another alternative, the compound of Formula I is of the Formula XVIII or XIX:

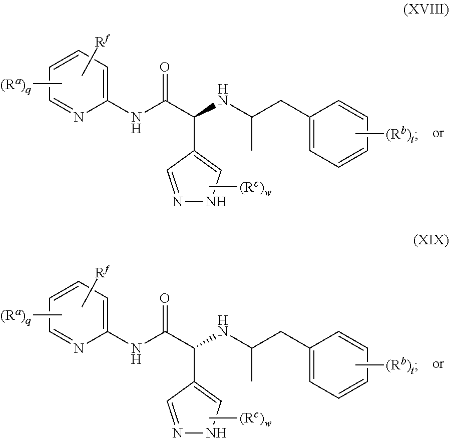

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. In another alternative, the compound of Formula I is of the Formula XX or XXI:

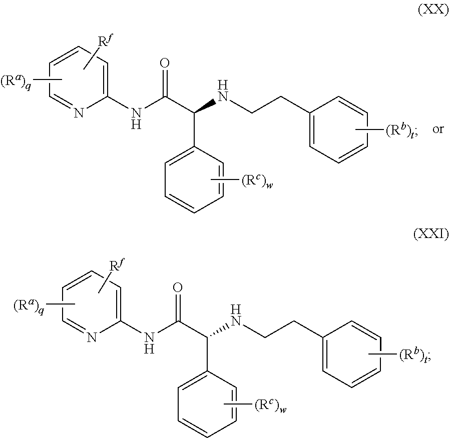

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment. In another alternative, the compound of Formula I is of the Formula XXII or XXIII:

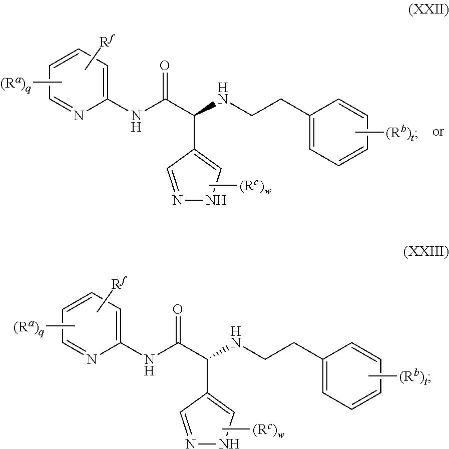

or a pharmaceutically acceptable salt thereof, wherein w, q, and t are each independently 0, 1, or 2, and wherein the remaining variables are as described above for Formula I or the second, fourth, fifth, sixth, seventh, eighth, ninth, or tenth embodiment.

In a twentieth embodiment of the methods described herein, R^{c}, if present, in the compounds of Formula XII, XIII, XIV, XV, XVI, XVII, XVIII, XIX, XX, XXI, XXII, and XXIII is independently C₁₋₆alkyl, halo, or CN, wherein the remaining variables are as described above for Formula I or the second, fourth, fifth, sixth, seventh, eighth, ninth, tenth or nineteenth embodiment. Alternatively, R^{c}, if present, in the compounds of Formula XII, XIII, XIV, XV, XVI, XVII, XVIII, XIX, XX, XXI, XXII, and XXIII is C₁₋₄alkyl, wherein the remaining variables are as described above for Formula I or the second, fourth, fifth, sixth, seventh, eighth, ninth, tenth, or nineteenth embodiment.

In a twenty-first embodiment of the methods described herein, w in the compounds of Formula XII, XIII, XIV, XV, XVI, XVII, XVIII, XIX, XX, XXI, XXII, and XXIII is 0 or 1, wherein the remaining variables are as described above for Formula I or the second, fourth, fifth, sixth, seventh, eighth, ninth, tenth, nineteenth, or twentieth embodiment.

In a twenty-second embodiment of the methods described herein, R^{b} in the compounds of Formula XII, XIII, XIV, XV, XVI, XVII, XVIII, XIX, XX, XXI, XXII, and XXIII is cyano, wherein the remaining variables are as described above for Formula I or the second, fourth, fifth, sixth, seventh, eighth, ninth, tenth, nineteenth, twentieth, or twenty-first embodiment.

In a twenty-third embodiment of the methods described herein, t in the compounds of Formula XII, XIII, XIV, XV, XVI, XVII, XVIII, XIX, XX, XXI, XXII, and XXIII is 1, wherein the remaining variables are as described above for Formula I or the second, fourth, fifth, sixth, seventh, eighth, ninth, tenth, nineteenth, twentieth, twenty-first, or twenty-second embodiment.

In a twenty-fourth embodiment of the methods described herein, q in the compounds of Formula XII, XIII, XIV, XV, XVI, XVII, XVIII, XIX, XX, XXI, XXII, and XXIII is 1, wherein the remaining variables are as described above for Formula I or the second, fourth, fifth, sixth, seventh, eighth, ninth, tenth, nineteenth, twentieth, twenty-first, twenty-second, or twenty third embodiment.

In a twenty-fifth embodiment of the methods described herein, R in the compounds of Formula XII, XIII, XIV, XV, XVI, XVII, XVIII, XIX, XX, XXI, XXII, and XXIII is cycloalkyl, phenyl, heteroaryl, or heterocyclyl, each of which may be optionally substituted with 1 to 3 groups selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅, —Ocycloalkyl, wherein the remaining variables are as described above for Formula I or the second, fourth, fifth, sixth, seventh, eighth, ninth, tenth, nineteenth, twentieth, twenty-first, twenty-second, twenty third, or twenty-fourth embodiment. Alternatively, R in the compounds of Formula XII, XIII, XIV, XV, XVI, XVII, XVIII, XIX, XX, XXI, XXII, and XXIII is pyrimidinyl, phenyl, cyclobutanyl, cyclopropyl, pyrazolyl, imidazolyl, azetidinyl, piperidinyl, pyrrolidinyl, piperazinyl, triazolopyrazinyl, triazolyl, imidazolidinyl, thiadiazolidinyl, morpholinyl, oxaazaspiroheptanyl, oxaazaspirooctanyl, dihydropyrimidinyl, oxadiazolyl, isoxazolyl, or dihydropyridazinyl, each of which may be optionally substituted with 1 to 3 groups selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅, —Ocycloalkyl, wherein the remaining variables are as described above for Formula I or the second, fourth, fifth, sixth, seventh, eighth, ninth, tenth, nineteenth, twentieth, twenty-first, twenty-second, twenty third, or twenty-fourth embodiment. In another alternative, R^{f} in the compounds of Formula XII, XIII, XIV, XV, XVI, XVII, XVIII, XIX, XX, XXI, XXII, and XXIII is pyrimidinyl, phenyl, pyrazolyl, imidazolyl, azetidinyl, piperidinyl, pyrrolidinyl, piperazinyl, triazolopyrazinyl, triazolyl, imidazolidinyl, thiadiazolidinyl, morpholinyl, oxaazaspiroheptanyl, oxaazaspirooctanyl, dihydropyrimidinyl, oxadiazolyl, isoxazolyl, or dihydropyridazinyl, each of which may be optionally substituted with 1 to 3 groups selected from halo, oxo, C₁₋₆alkyl, C₁₋₆alkoxy, C₁₋₆haloalkyl, —C₁₋₆alkylOR^{d}, —C(O)R^{d}, —C(O)N(R^{d})₂, —C₁₋₆alkylC(O)N(R^{d})₂, and —S(O)₂R^{d}, wherein the remaining variables are as described above for Formula I or the second, fourth, fifth, sixth, seventh, eighth, ninth, tenth, nineteenth, twentieth, twenty-first, twenty-second, twenty third, or twenty-fourth embodiment. In another alternative, R^{f} in the compounds of Formula XII, XIII, XIV, XV, XVI, XVII, XVIII, XIX, XX, XXI, XXII, and XXIII is pyrazolyl or triazolyl, each of which may be optionally substituted with C₁₋₃alkyl or —C(O)N(R^{d})₂, wherein the remaining variables are as described above for Formula I or the second, fourth, fifth, sixth, seventh, eighth, ninth, tenth, nineteenth, twentieth, twenty-first, twenty-second, twenty third, or twenty-fourth embodiment.

In a twenty-sixth embodiment of the methods described herein, R^{d} in the compounds of Formula XII, XIII, XIV, XV, XVI, XVII, XVIII, XIX, XX, XXI, XXII, and XXIII is hydrogen or C₁₋₃alkyl, wherein the remaining variables are as described above for Formula I or the second, fourth, fifth, sixth, seventh, eighth, ninth, tenth, nineteenth, twentieth, twenty-first, twenty-second, twenty third, twenty-fourth, or twenty-fifth embodiment. Alternatively, R^{d} in the compounds of Formula XII, XIII, XIV, XV, XVI, XVII, XVIII, XIX, XX, XXI, XXII, and XXIII is C₁₋₃alkyl, wherein the remaining variables are as described above for Formula I or the second, fourth, fifth, sixth, seventh, eighth, ninth, tenth, nineteenth, twentieth, twenty-first, twenty-second, twenty third, twenty-fourth, or twenty-fifth embodiment.

In a twenty-seventh embodiment of the methods described herein, the compound of Formula XX or XXI excludes a compound having the Formula:

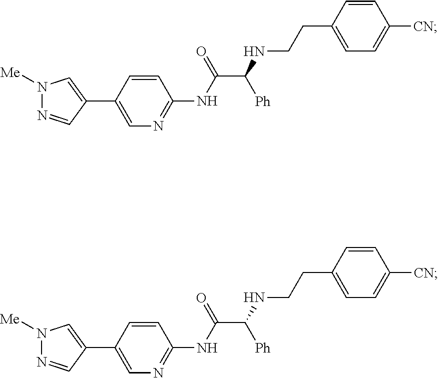 [SMILES: Cn1cc(-c2ccc(NC(=O)[C@@H](NCCc3ccc(C#N)cc3)c3ccccc3)nc2)cn1 ; Cn1cc(-c2ccc(NC(=O)[C@H](NCCc3ccc(C#N)cc3)c3ccccc3)nc2)cn1]

or a pharmaceutically acceptable salt thereof, wherein the remaining variables are as described above for Formula I or the second, fourth, fifth, sixth, seventh, eighth, ninth, tenth, nineteenth, twentieth, twenty-first, twenty-second, twenty third, twenty-fourth, twenty-fifth, or twenty-sixth embodiment.

In a twenty-eighth embodiment of the methods described herein, the compound of Formula I is selected from the following formula:

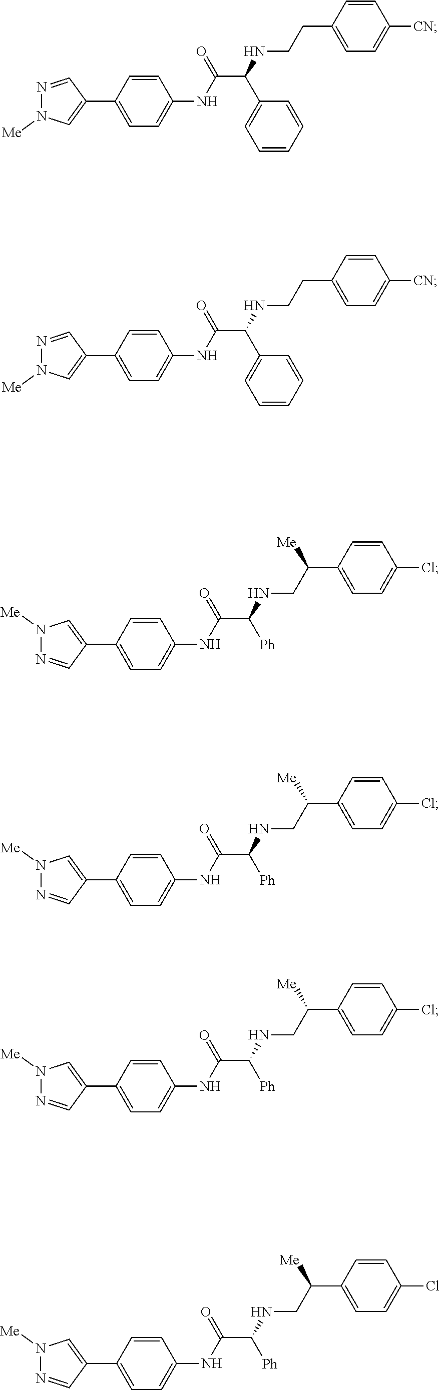 [SMILES: Cn1cc(-c2ccc(NC(=O)[C@@H](NCCc3ccc(C#N)cc3)c3ccccc3)cc2)cn1 ; Cn1cc(-c2ccc(NC(=O)[C@H](NCCc3ccc(C#N)cc3)c3ccccc3)cc2)cn1 ; C[C@H](CN[C@H](C(=O)Nc1ccc(-c2cnn(C)c2)cc1)c1ccccc1)c1ccc(Cl)cc1 ; C[C@@H](CN[C@H](C(=O)Nc1ccc(-c2cnn(C)c2)cc1)c1ccccc1)c1ccc(Cl)cc1 ; C[C@@H](CN[C@@H](C(=O)Nc1ccc(-c2cnn(C)c2)cc1)c1ccccc1)c1ccc(Cl)cc1 ; C[C@H](CN[C@@H](C(=O)Nc1ccc(-c2cnn(C)c2)cc1)c1ccccc1)c1ccc(Cl)cc1]

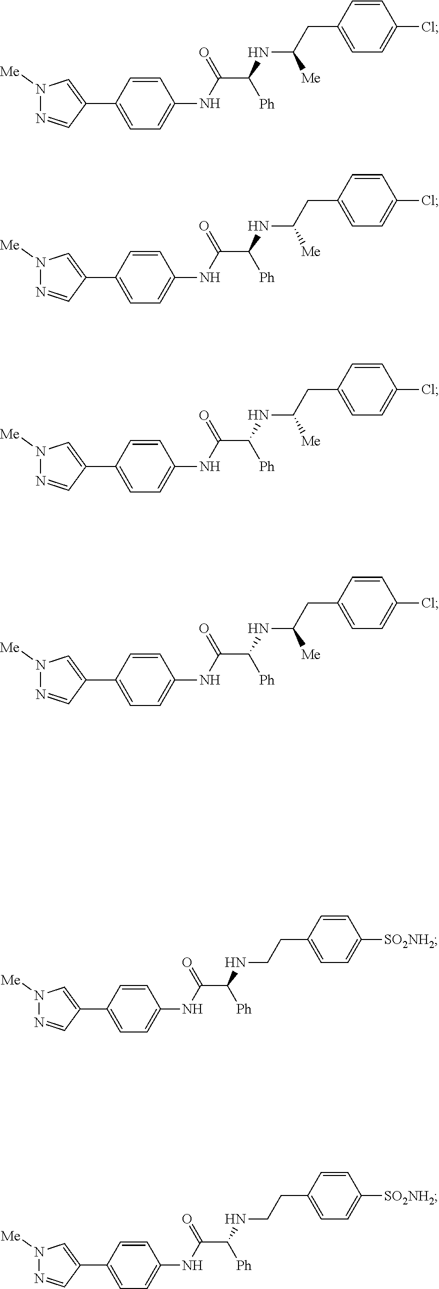 [SMILES: C[C@H](Cc1ccc(Cl)cc1)N[C@H](C(=O)Nc1ccc(-c2cnn(C)c2)cc1)c1ccccc1 ; C[C@@H](Cc1ccc(Cl)cc1)N[C@H](C(=O)Nc1ccc(-c2cnn(C)c2)cc1)c1ccccc1 ; C[C@@H](Cc1ccc(Cl)cc1)N[C@@H](C(=O)Nc1ccc(-c2cnn(C)c2)cc1)c1ccccc1 ; C[C@H](Cc1ccc(Cl)cc1)N[C@@H](C(=O)Nc1ccc(-c2cnn(C)c2)cc1)c1ccccc1 ; Cn1cc(-c2ccc(NC(=O)[C@@H](NCCc3ccc(S(N)(=O)=O)cc3)c3ccccc3)cc2)cn1 ; Cn1cc(-c2ccc(NC(=O)[C@H](NCCc3ccc(S(N)(=O)=O)cc3)c3ccccc3)cc2)cn1]

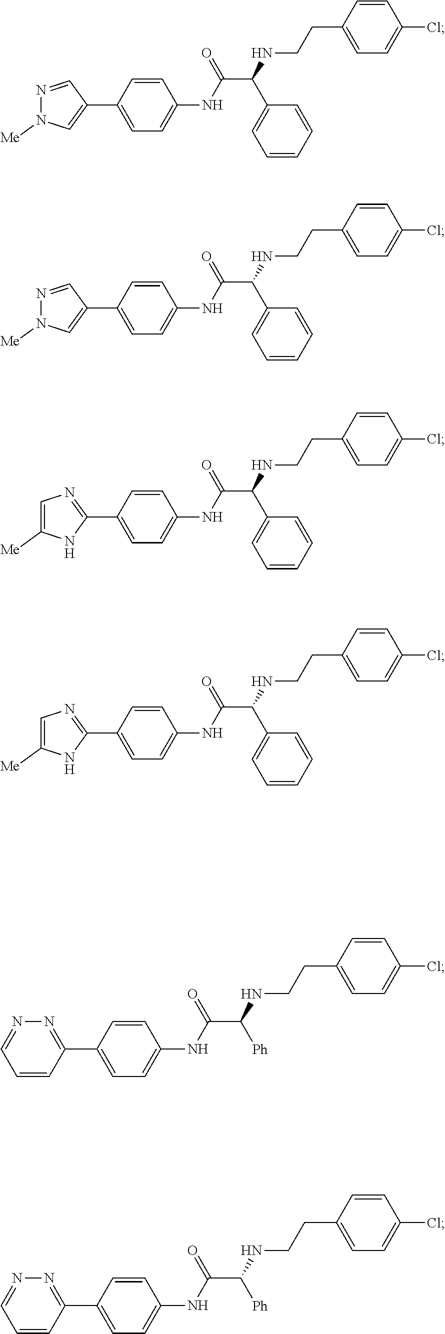 [SMILES: Cn1cc(-c2ccc(NC(=O)[C@@H](NCCc3ccc(Cl)cc3)c3ccccc3)cc2)cn1 ; Cn1cc(-c2ccc(NC(=O)[C@H](NCCc3ccc(Cl)cc3)c3ccccc3)cc2)cn1 ; Cc1cnc(-c2ccc(NC(=O)[C@@H](NCCc3ccc(Cl)cc3)c3ccccc3)cc2)[nH]1 ; Cc1cnc(-c2ccc(NC(=O)[C@H](NCCc3ccc(Cl)cc3)c3ccccc3)cc2)[nH]1 ; O=C(Nc1ccc(-c2cccnn2)cc1)[C@@H](NCCc1ccc(Cl)cc1)c1ccccc1 ; O=C(Nc1ccc(-c2cccnn2)cc1)[C@H](NCCc1ccc(Cl)cc1)c1ccccc1]

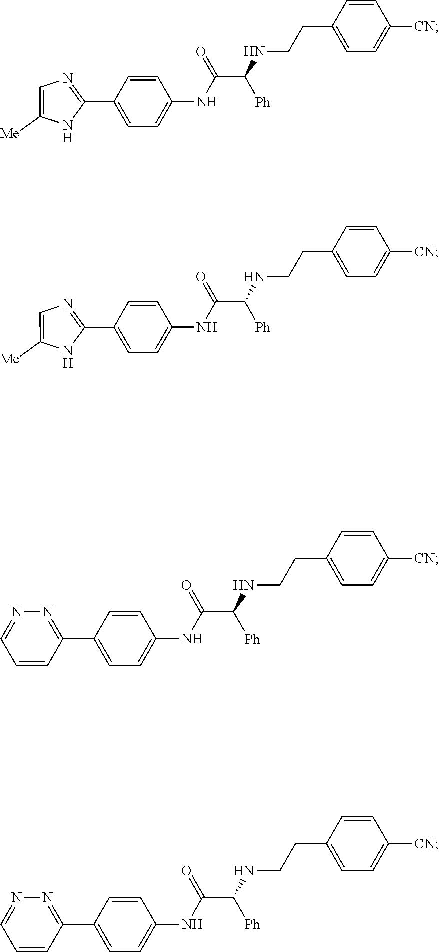 [SMILES: Cc1cnc(-c2ccc(NC(=O)[C@@H](NCCc3ccc(C#N)cc3)c3ccccc3)cc2)[nH]1 ; Cc1cnc(-c2ccc(NC(=O)[C@H](NCCc3ccc(C#N)cc3)c3ccccc3)cc2)[nH]1 ; N#Cc1ccc(CCN[C@H](C(=O)Nc2ccc(-c3cccnn3)cc2)c2ccccc2)cc1 ; N#Cc1ccc(CCN[C@@H](C(=O)Nc2ccc(-c3cccnn3)cc2)c2ccccc2)cc1]

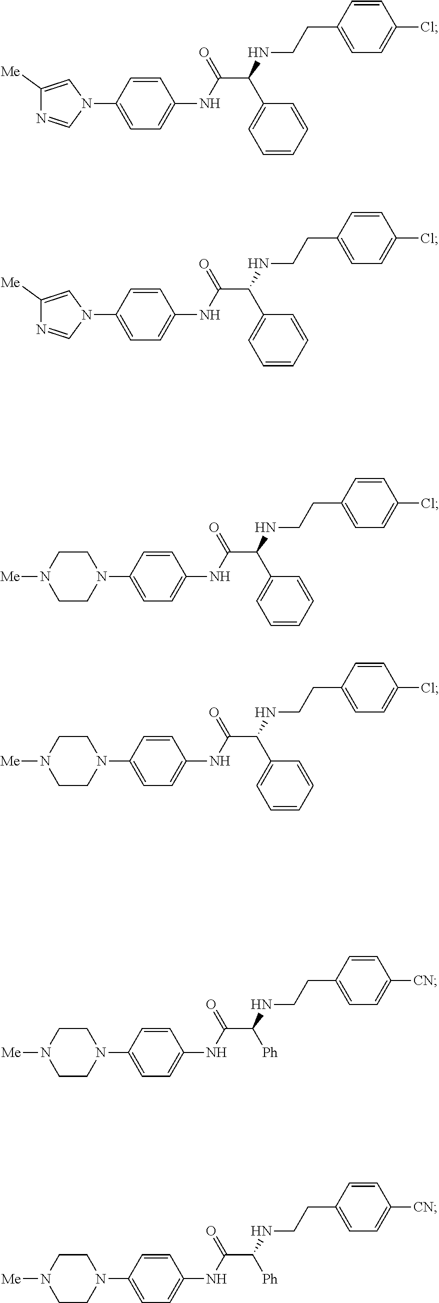 [SMILES: Cc1cn(-c2ccc(NC(=O)[C@@H](NCCc3ccc(Cl)cc3)c3ccccc3)cc2)cn1 ; Cc1cn(-c2ccc(NC(=O)[C@H](NCCc3ccc(Cl)cc3)c3ccccc3)cc2)cn1 ; CN1CCN(c2ccc(NC(=O)[C@@H](NCCc3ccc(Cl)cc3)c3ccccc3)cc2)CC1 ; CN1CCN(c2ccc(NC(=O)[C@H](NCCc3ccc(Cl)cc3)c3ccccc3)cc2)CC1 ; CN1CCN(c2ccc(NC(=O)[C@@H](NCCc3ccc(C#N)cc3)c3ccccc3)cc2)CC1 ; CN1CCN(c2ccc(NC(=O)[C@H](NCCc3ccc(C#N)cc3)c3ccccc3)cc2)CC1]

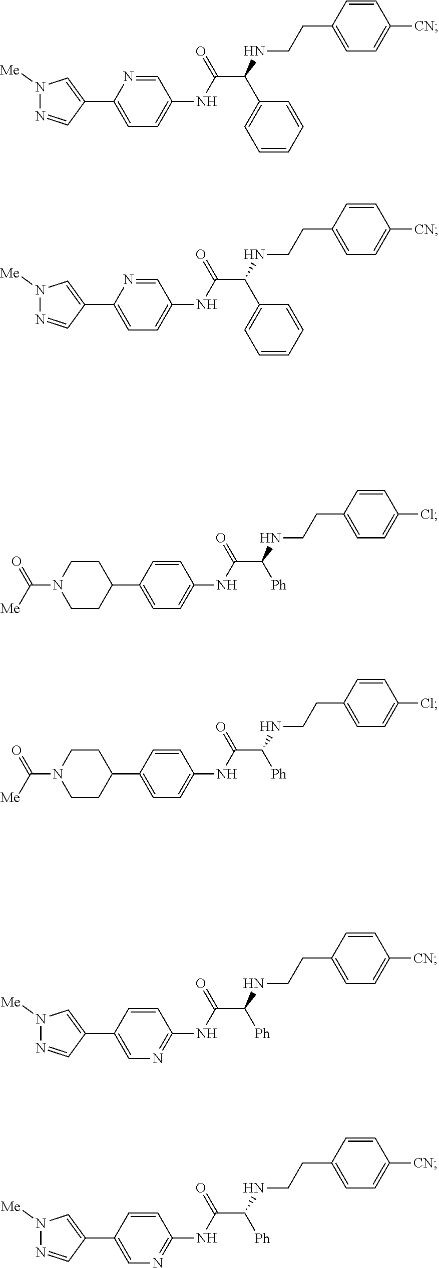 [SMILES: Cn1cc(-c2ccc(NC(=O)[C@@H](NCCc3ccc(C#N)cc3)c3ccccc3)cn2)cn1 ; Cn1cc(-c2ccc(NC(=O)[C@H](NCCc3ccc(C#N)cc3)c3ccccc3)cn2)cn1 ; CC(=O)N1CCC(c2ccc(NC(=O)[C@@H](NCCc3ccc(Cl)cc3)c3ccccc3)cc2)CC1 ; CC(=O)N1CCC(c2ccc(NC(=O)[C@H](NCCc3ccc(Cl)cc3)c3ccccc3)cc2)CC1 ; Cn1cc(-c2ccc(NC(=O)[C@@H](NCCc3ccc(C#N)cc3)c3ccccc3)nc2)cn1 ; Cn1cc(-c2ccc(NC(=O)[C@H](NCCc3ccc(C#N)cc3)c3ccccc3)nc2)cn1]

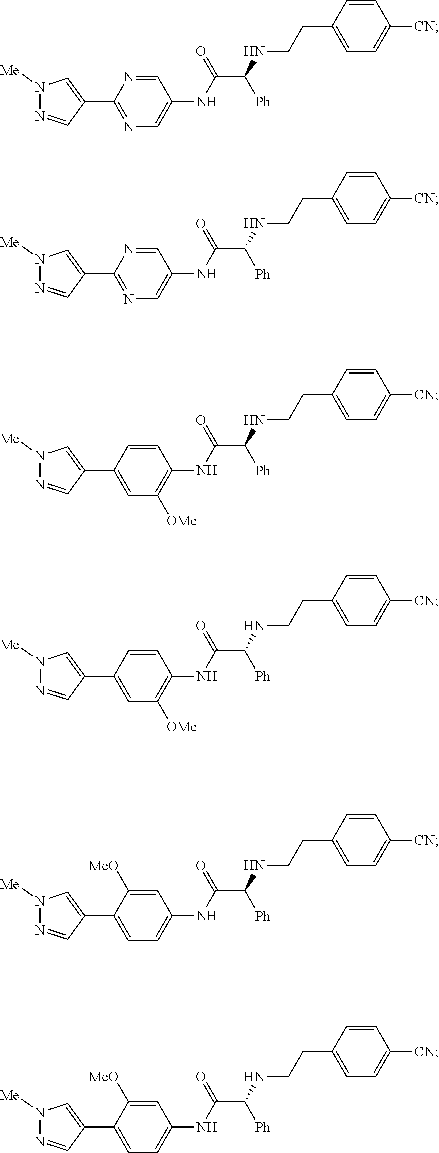 [SMILES: Cn1cc(-c2ncc(NC(=O)[C@@H](NCCc3ccc(C#N)cc3)c3ccccc3)cn2)cn1 ; Cn1cc(-c2ncc(NC(=O)[C@H](NCCc3ccc(C#N)cc3)c3ccccc3)cn2)cn1 ; COc1cc(-c2cnn(C)c2)ccc1NC(=O)[C@@H](NCCc1ccc(C#N)cc1)c1ccccc1 ; COc1cc(-c2cnn(C)c2)ccc1NC(=O)[C@H](NCCc1ccc(C#N)cc1)c1ccccc1 ; COc1cc(NC(=O)[C@@H](NCCc2ccc(C#N)cc2)c2ccccc2)ccc1-c1cnn(C)c1 ; COc1cc(NC(=O)[C@H](NCCc2ccc(C#N)cc2)c2ccccc2)ccc1-c1cnn(C)c1]

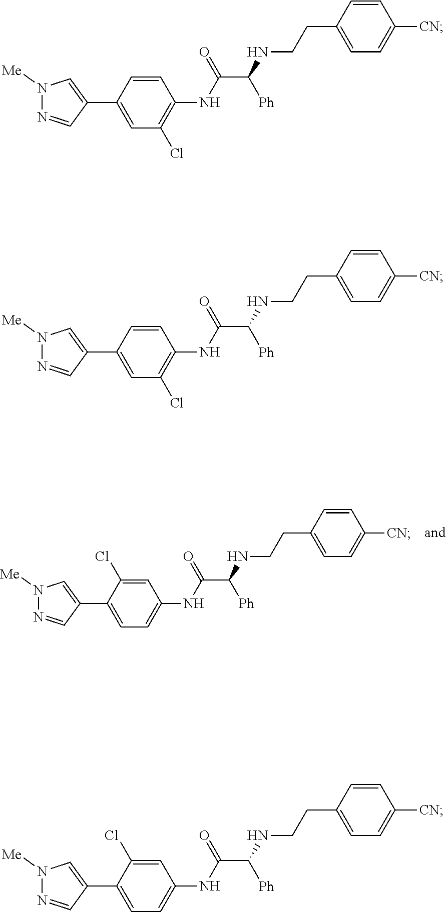 [SMILES: Cn1cc(-c2ccc(NC(=O)[C@@H](NCCc3ccc(C#N)cc3)c3ccccc3)c(Cl)c2)cn1 ; Cn1cc(-c2ccc(NC(=O)[C@H](NCCc3ccc(C#N)cc3)c3ccccc3)c(Cl)c2)cn1 ; Cn1cc(-c2ccc(NC(=O)[C@@H](NCCc3ccc(C#N)cc3)c3ccccc3)cc2Cl)cn1 ; Cn1cc(-c2ccc(NC(=O)[C@H](NCCc3ccc(C#N)cc3)c3ccccc3)cc2Cl)cn1]

or a pharmaceutically acceptable salt thereof of any of the foregoing.

In a twenty-ninth embodiment of the methods described herein, the compound of Formula I is selected from the following formula:

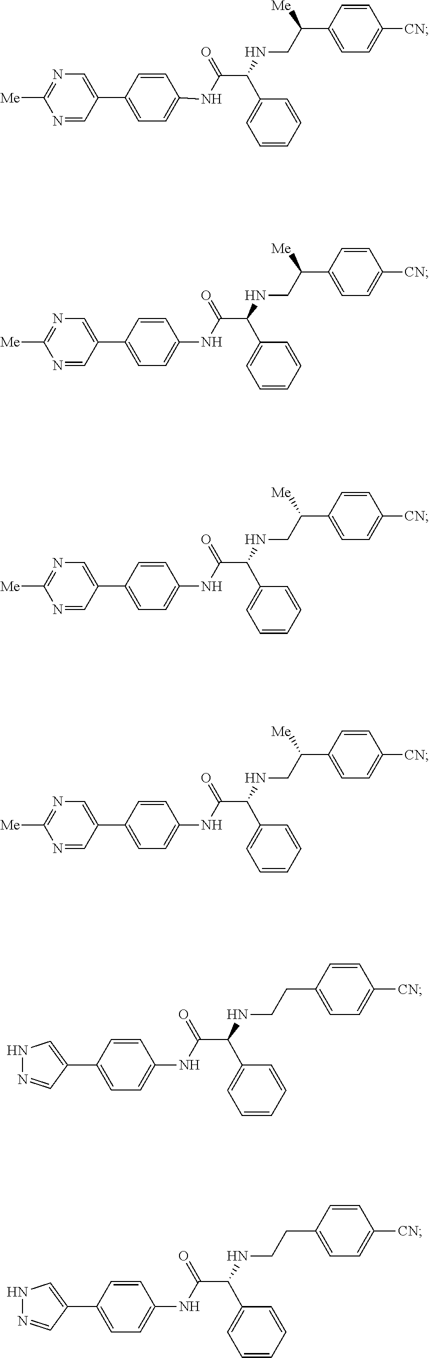 [SMILES: Cc1ncc(-c2ccc(NC(=O)[C@H](NC[C@@H](C)c3ccc(C#N)cc3)c3ccccc3)cc2)cn1 ; Cc1ncc(-c2ccc(NC(=O)[C@@H](NC[C@@H](C)c3ccc(C#N)cc3)c3ccccc3)cc2)cn1 ; Cc1ncc(-c2ccc(NC(=O)[C@H](NC[C@H](C)c3ccc(C#N)cc3)c3ccccc3)cc2)cn1 ; Cc1ncc(-c2ccc(NC(=O)[C@H](NC[C@H](C)c3ccc(C#N)cc3)c3ccccc3)cc2)cn1 ; N#Cc1ccc(CCN[C@H](C(=O)Nc2ccc(-c3cn[nH]c3)cc2)c2ccccc2)cc1 ; N#Cc1ccc(CCN[C@@H](C(=O)Nc2ccc(-c3cn[nH]c3)cc2)c2ccccc2)cc1]

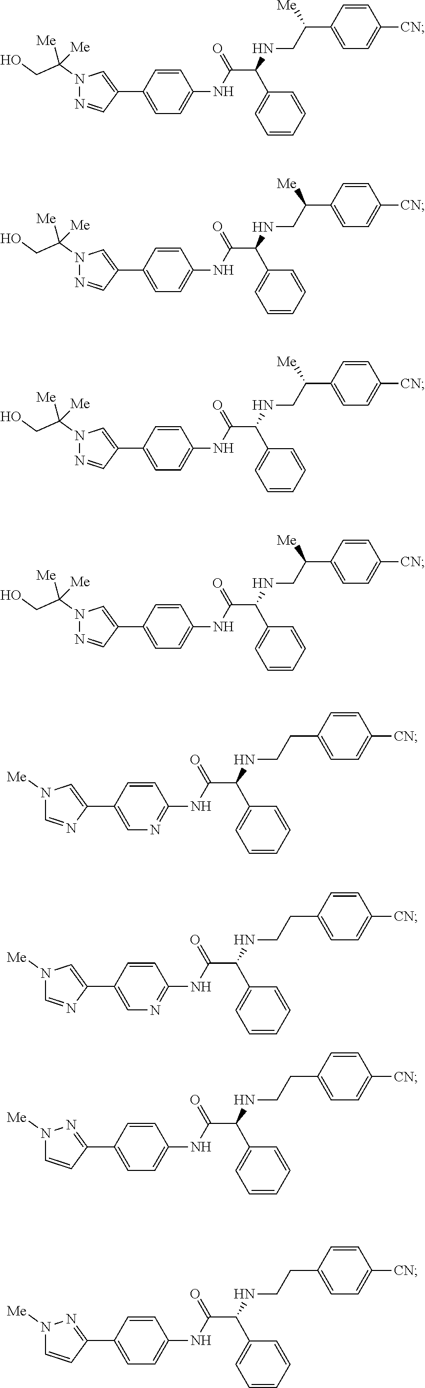

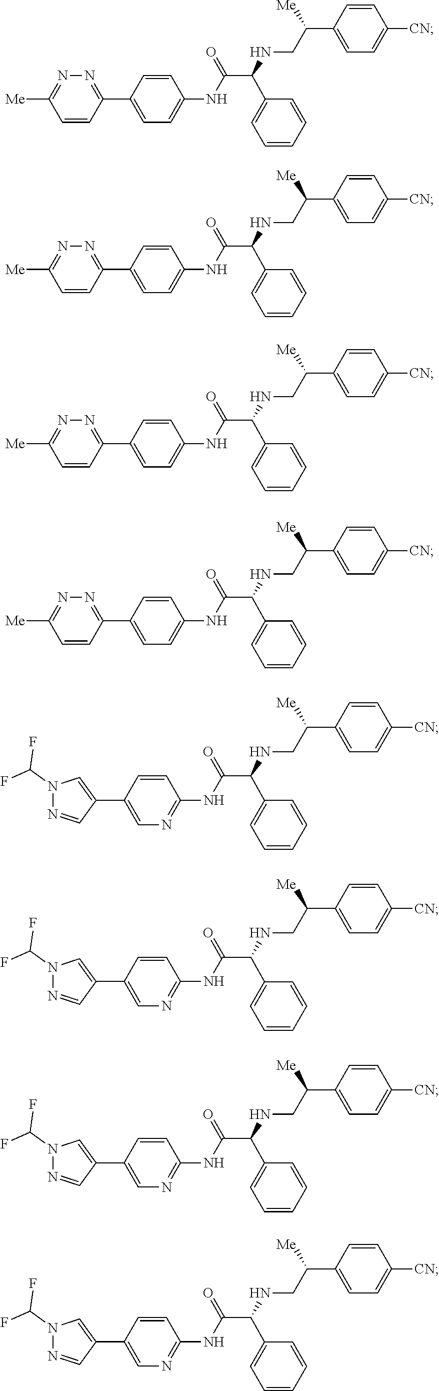

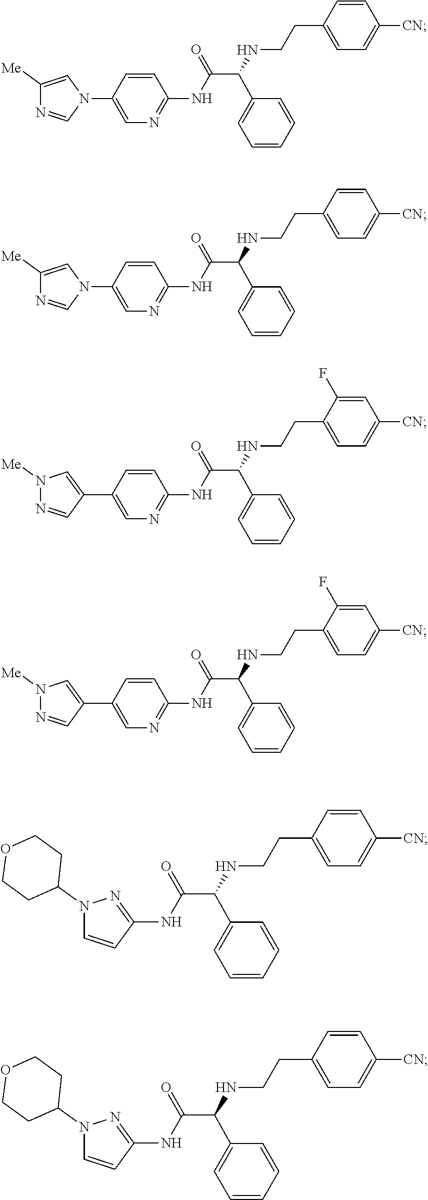 [SMILES: Cc1cn(-c2ccc(NC(=O)[C@H](NCCc3ccc(C#N)cc3)c3ccccc3)nc2)cn1 ; Cc1cn(-c2ccc(NC(=O)[C@@H](NCCc3ccc(C#N)cc3)c3ccccc3)nc2)cn1 ; Cn1cc(-c2ccc(NC(=O)[C@H](NCCc3ccc(C#N)cc3F)c3ccccc3)nc2)cn1 ; Cn1cc(-c2ccc(NC(=O)[C@@H](NCCc3ccc(C#N)cc3F)c3ccccc3)nc2)cn1 ; N#Cc1ccc(CCN[C@@H](C(=O)Nc2ccn(C3CCOCC3)n2)c2ccccc2)cc1 ; N#Cc1ccc(CCN[C@H](C(=O)Nc2ccn(C3CCOCC3)n2)c2ccccc2)cc1]

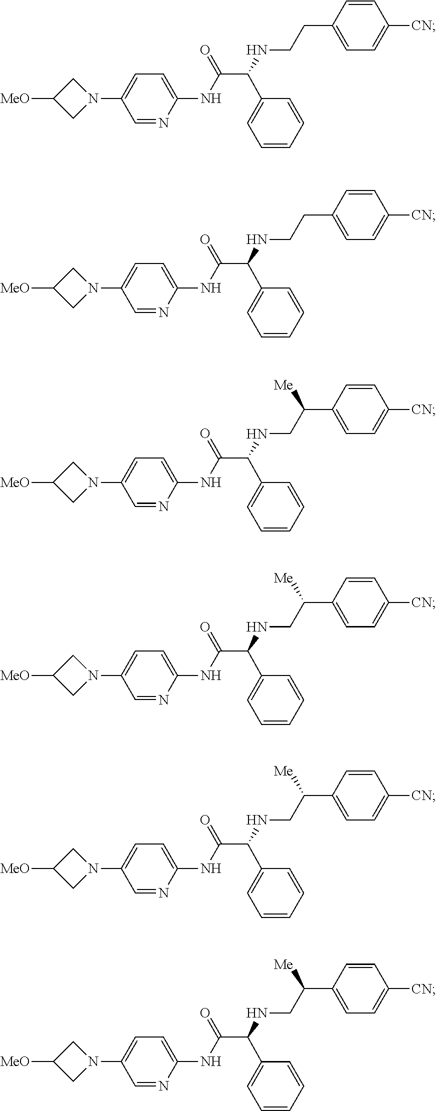 [SMILES: COC1CN(c2ccc(NC(=O)[C@H](NCCc3ccc(C#N)cc3)c3ccccc3)nc2)C1 ; COC1CN(c2ccc(NC(=O)[C@@H](NCCc3ccc(C#N)cc3)c3ccccc3)nc2)C1 ; COC1CN(c2ccc(NC(=O)[C@H](NC[C@@H](C)c3ccc(C#N)cc3)c3ccccc3)nc2)C1 ; COC1CN(c2ccc(NC(=O)[C@@H](NC[C@H](C)c3ccc(C#N)cc3)c3ccccc3)nc2)C1 ; COC1CN(c2ccc(NC(=O)[C@H](NC[C@H](C)c3ccc(C#N)cc3)c3ccccc3)nc2)C1 ; COC1CN(c2ccc(NC(=O)[C@@H](NC[C@@H](C)c3ccc(C#N)cc3)c3ccccc3)nc2)C1]

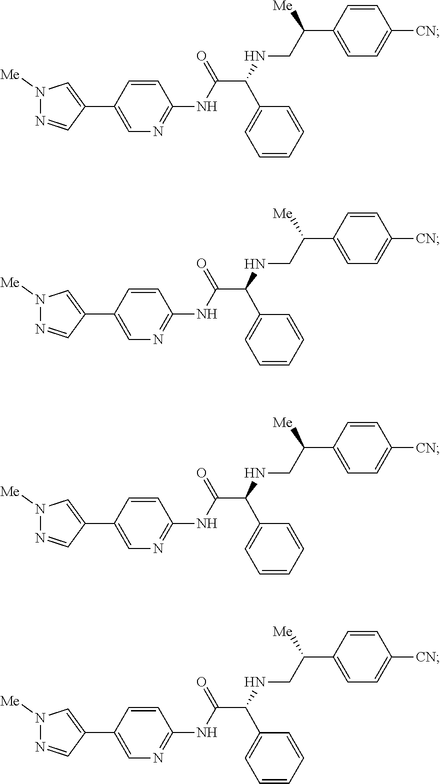 [SMILES: C[C@H](CN[C@@H](C(=O)Nc1ccc(-c2cnn(C)c2)cn1)c1ccccc1)c1ccc(C#N)cc1 ; C[C@@H](CN[C@H](C(=O)Nc1ccc(-c2cnn(C)c2)cn1)c1ccccc1)c1ccc(C#N)cc1 ; C[C@H](CN[C@H](C(=O)Nc1ccc(-c2cnn(C)c2)cn1)c1ccccc1)c1ccc(C#N)cc1 ; C[C@@H](CN[C@@H](C(=O)Nc1ccc(-c2cnn(C)c2)cn1)c1ccccc1)c1ccc(C#N)cc1]

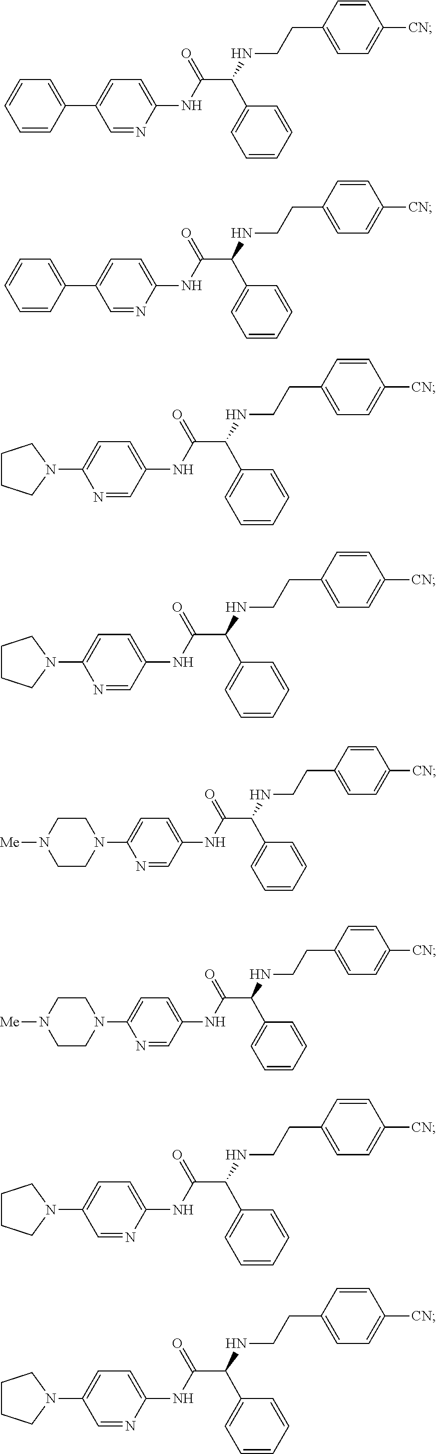

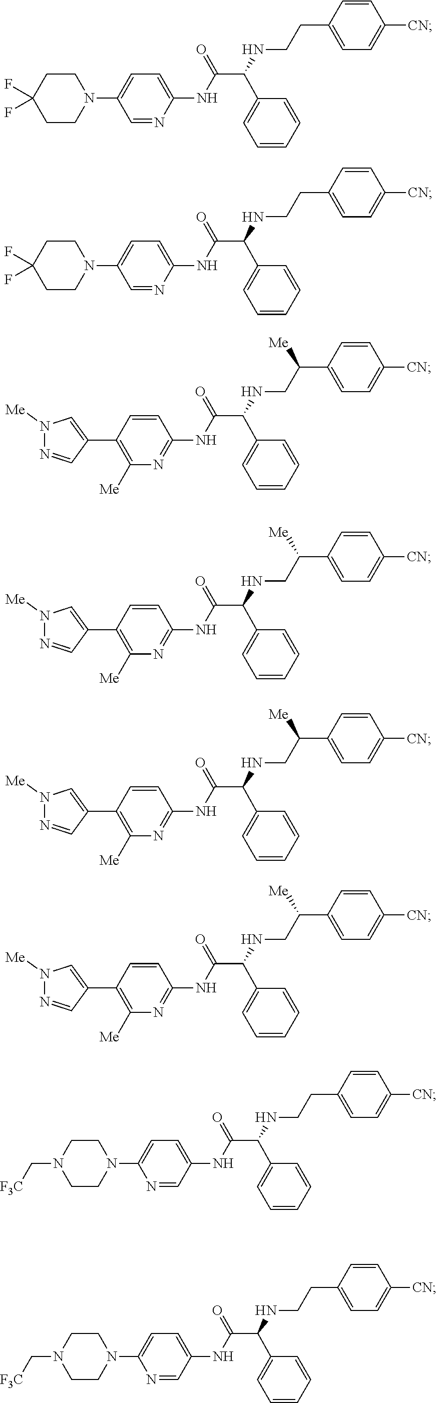

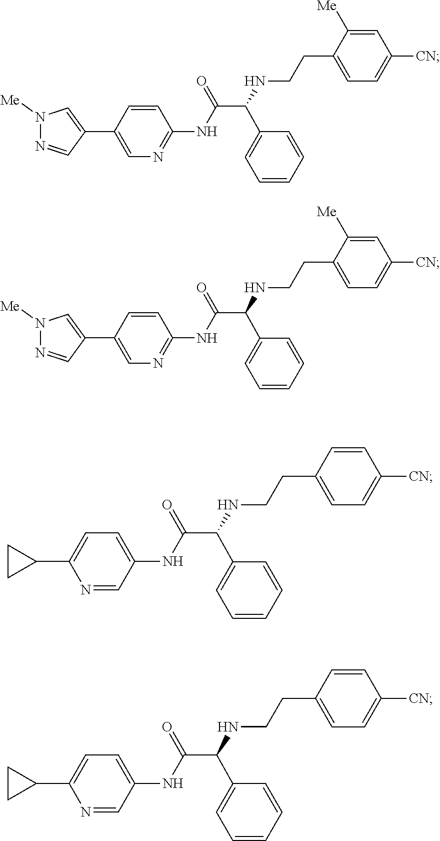 [SMILES: Cc1cc(C#N)ccc1CCN[C@@H](C(=O)Nc1ccc(-c2cnn(C)c2)cn1)c1ccccc1 ; Cc1cc(C#N)ccc1CCN[C@H](C(=O)Nc1ccc(-c2cnn(C)c2)cn1)c1ccccc1 ; N#Cc1ccc(CCN[C@@H](C(=O)Nc2ccc(C3CC3)nc2)c2ccccc2)cc1 ; N#Cc1ccc(CCN[C@H](C(=O)Nc2ccc(C3CC3)nc2)c2ccccc2)cc1]

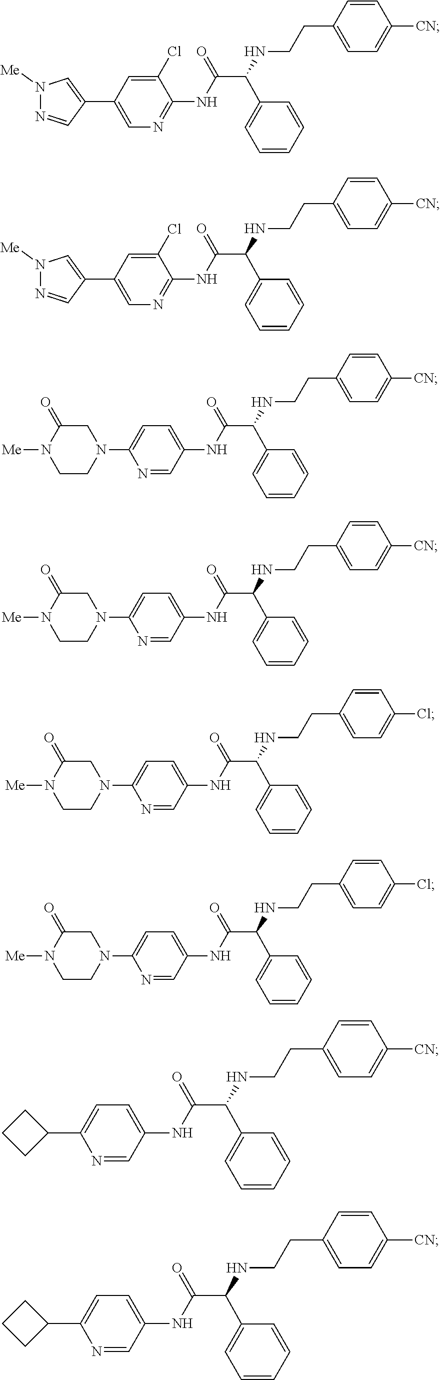

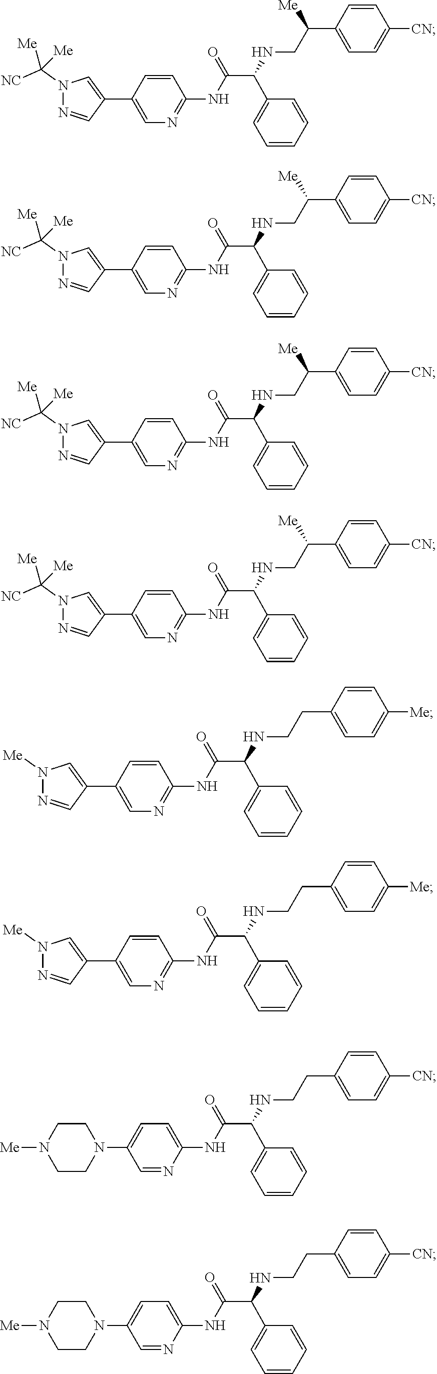

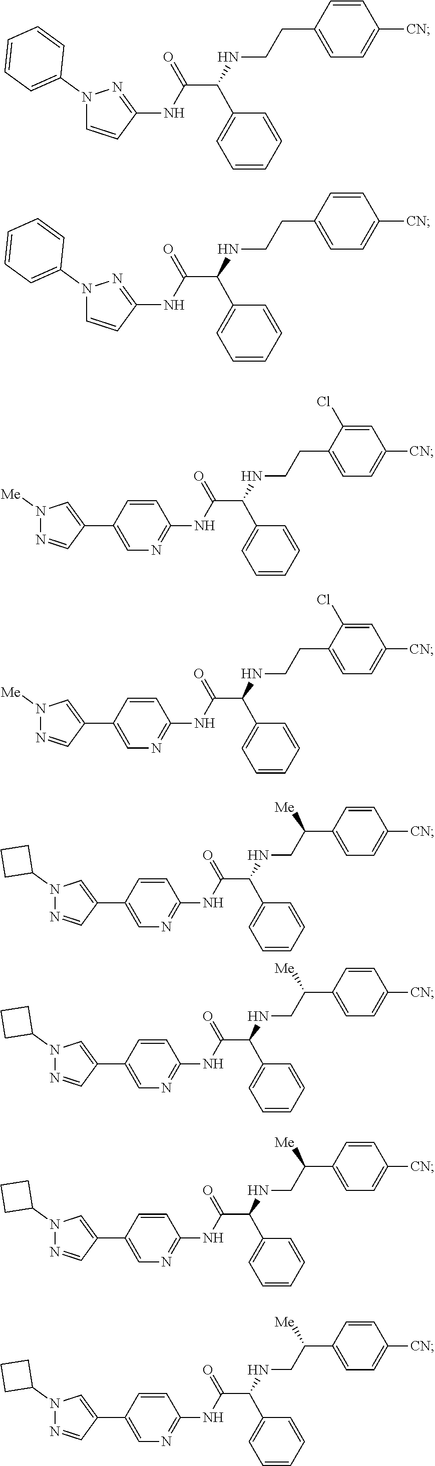

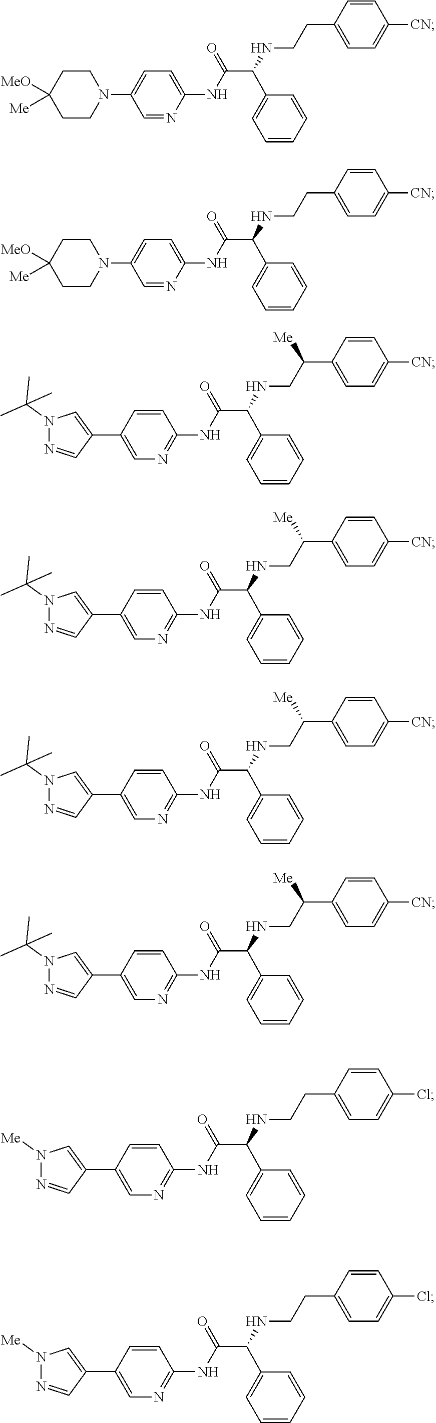

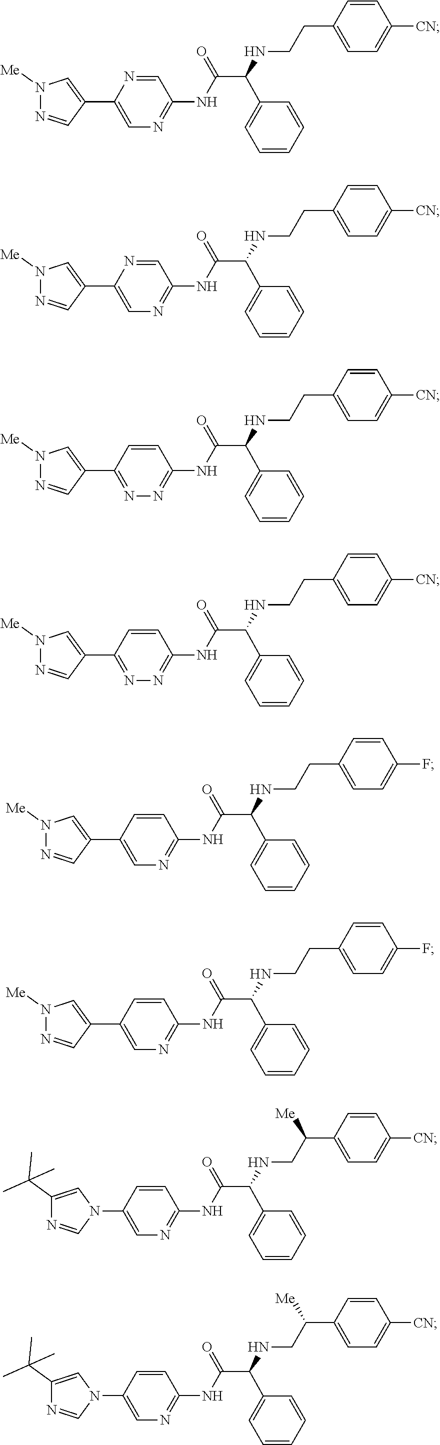

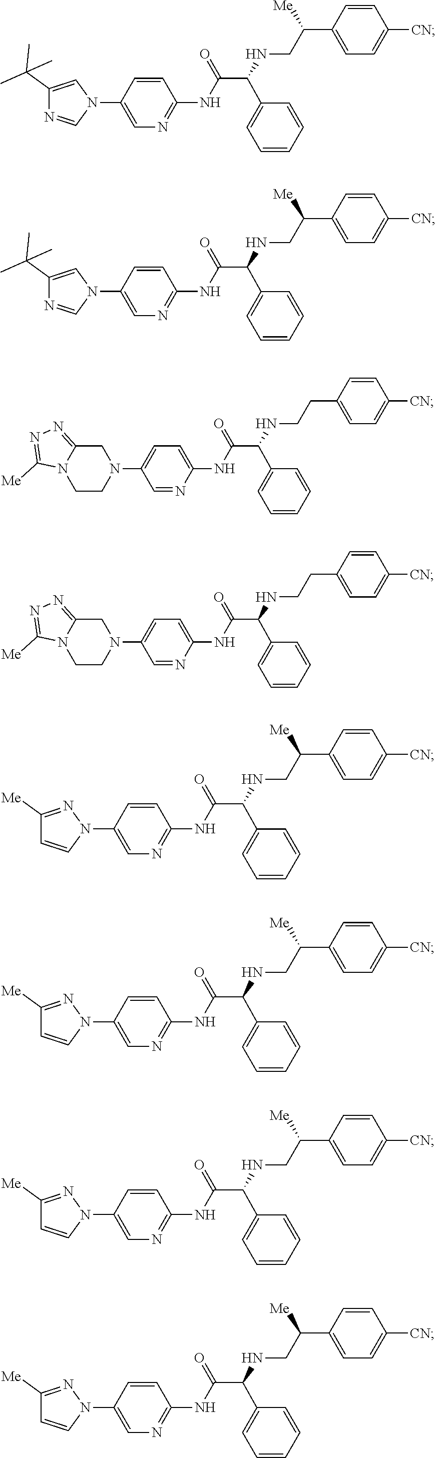

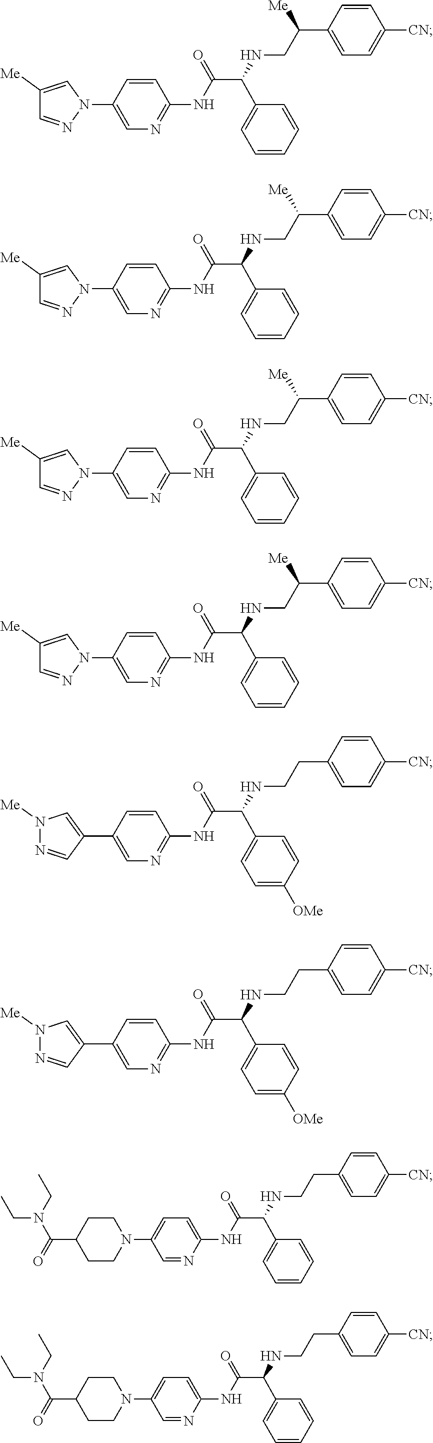

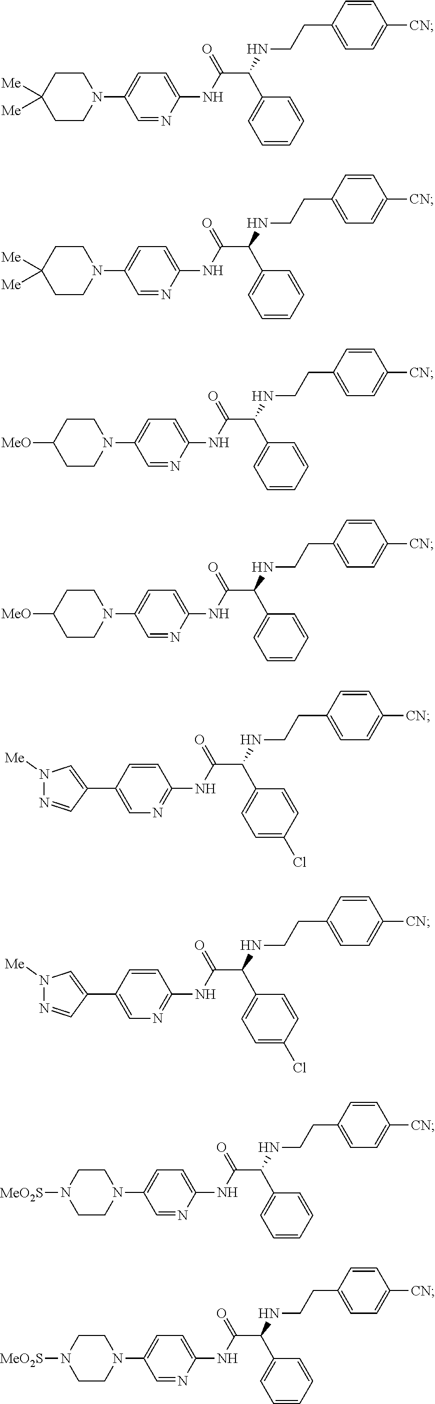

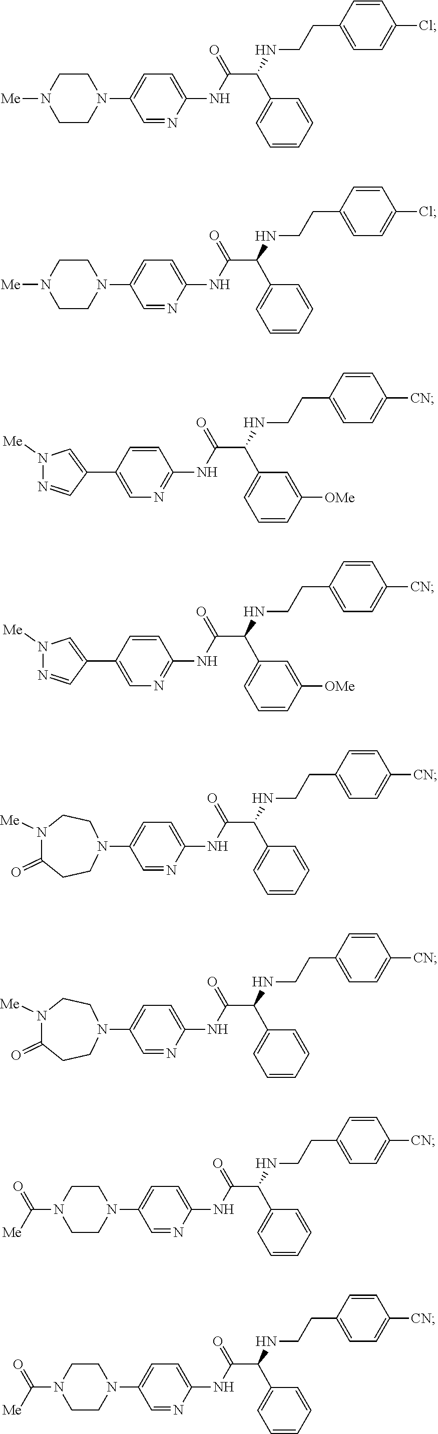

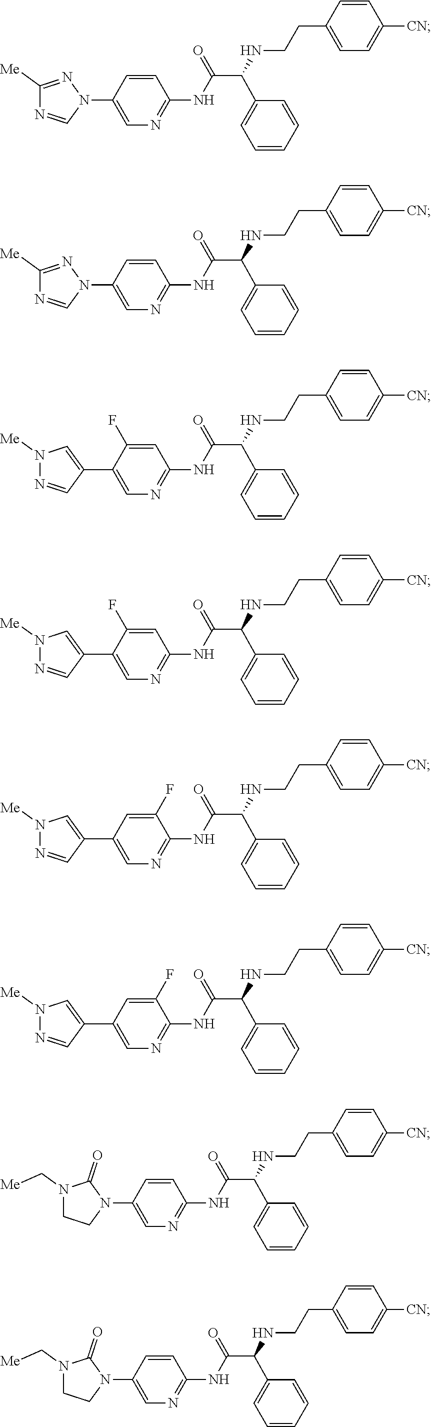

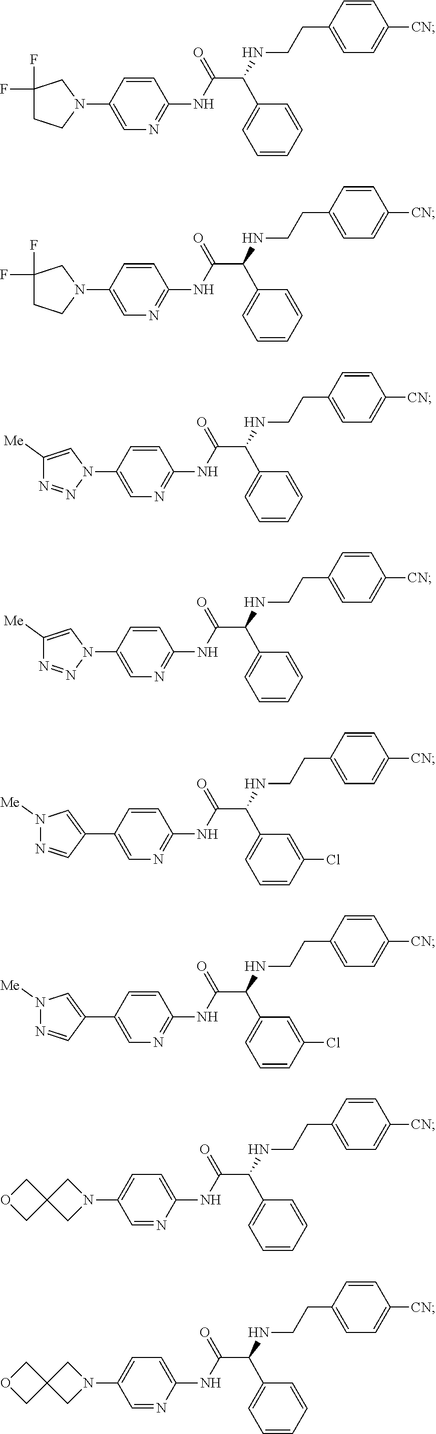

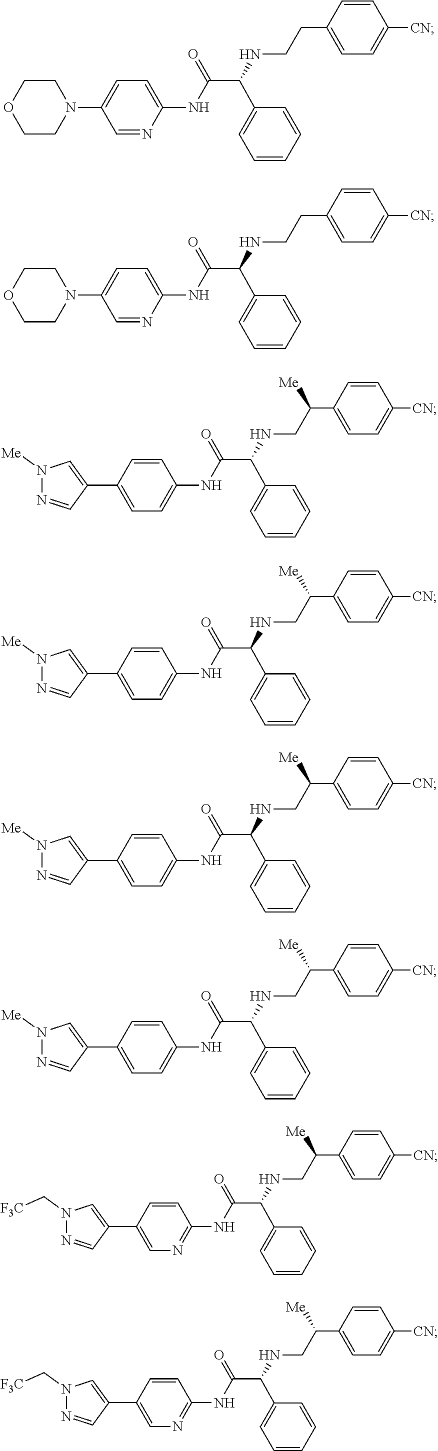

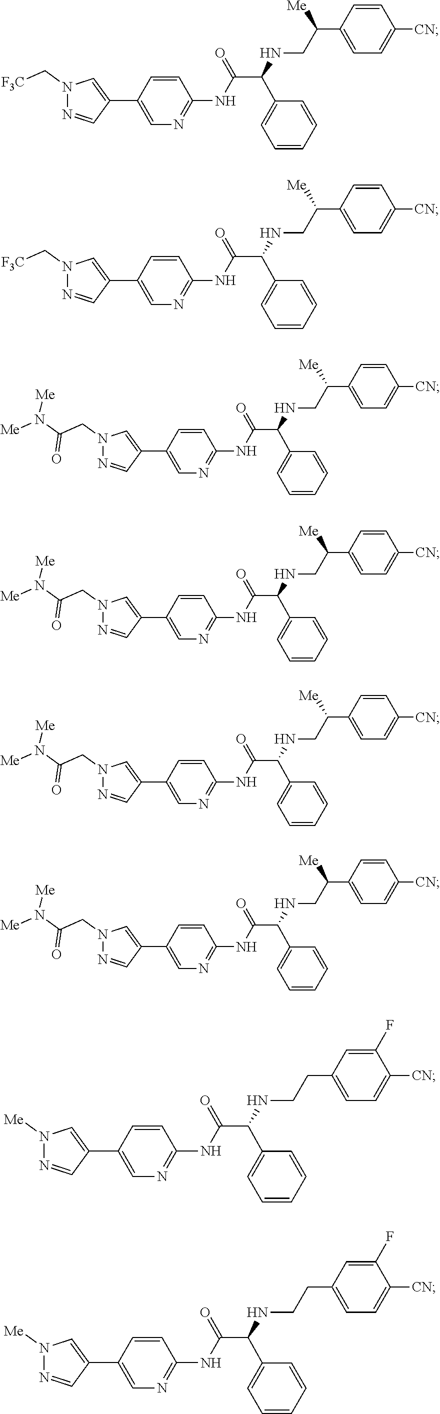

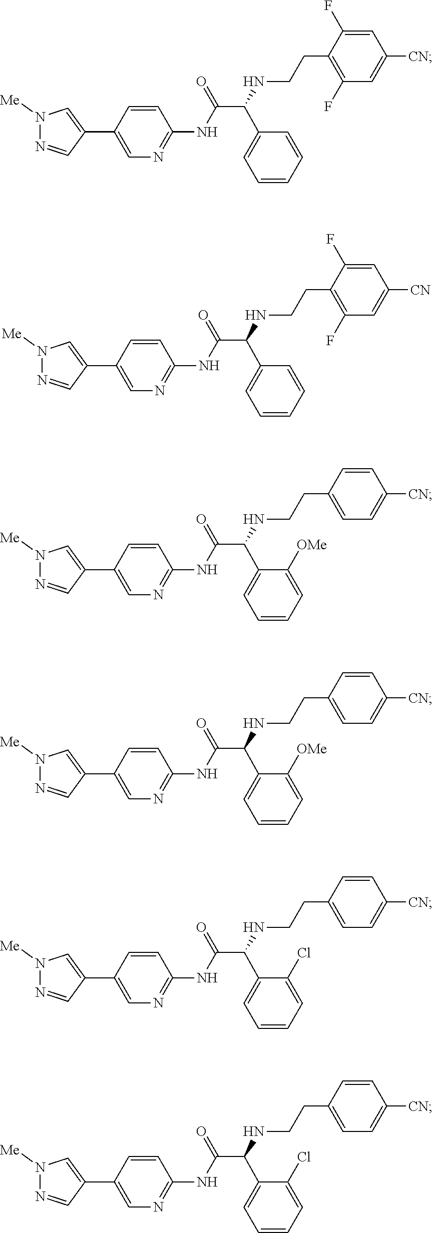 [SMILES: Cn1cc(-c2ccc(NC(=O)[C@H](NCCc3c(F)cc(C#N)cc3F)c3ccccc3)nc2)cn1 ; Cn1cc(-c2ccc(NC(=O)[C@@H](NCCc3c(F)cc(C#N)cc3F)c3ccccc3)nc2)cn1 ; COc1ccccc1[C@@H](NCCc1ccc(C#N)cc1)C(=O)Nc1ccc(-c2cnn(C)c2)cn1 ; COc1ccccc1[C@H](NCCc1ccc(C#N)cc1)C(=O)Nc1ccc(-c2cnn(C)c2)cn1 ; Cn1cc(-c2ccc(NC(=O)[C@H](NCCc3ccc(C#N)cc3)c3ccccc3Cl)nc2)cn1 ; Cn1cc(-c2ccc(NC(=O)[C@@H](NCCc3ccc(C#N)cc3)c3ccccc3Cl)nc2)cn1]

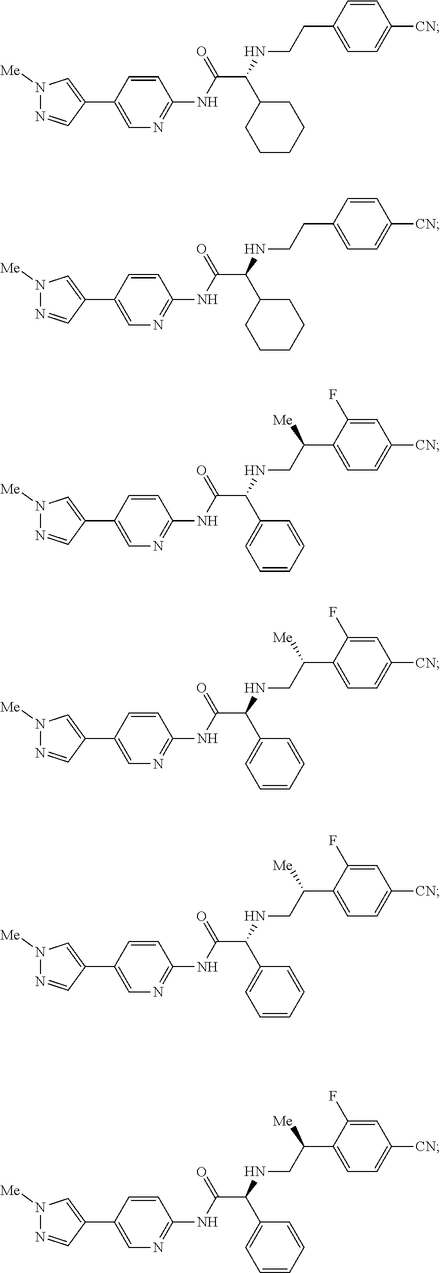

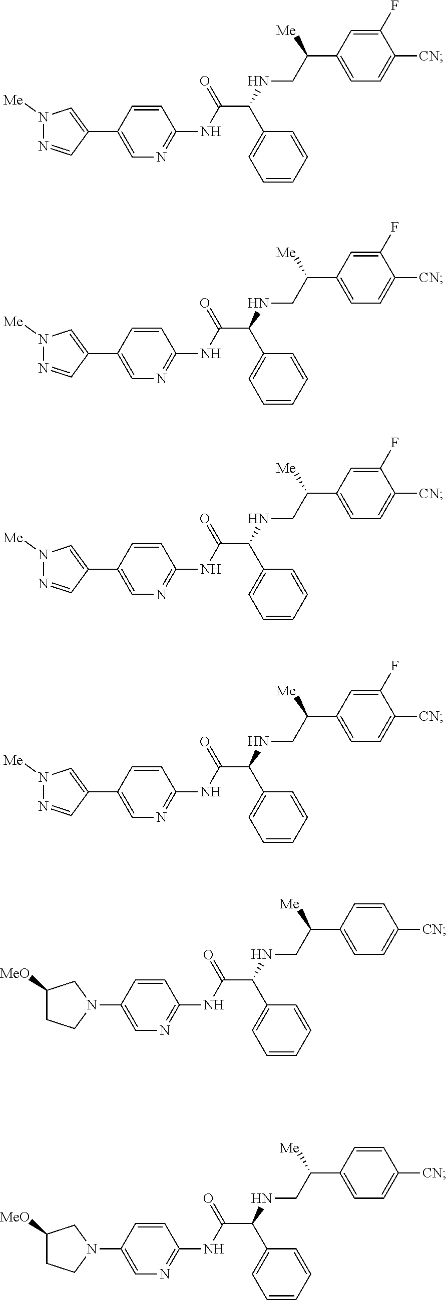

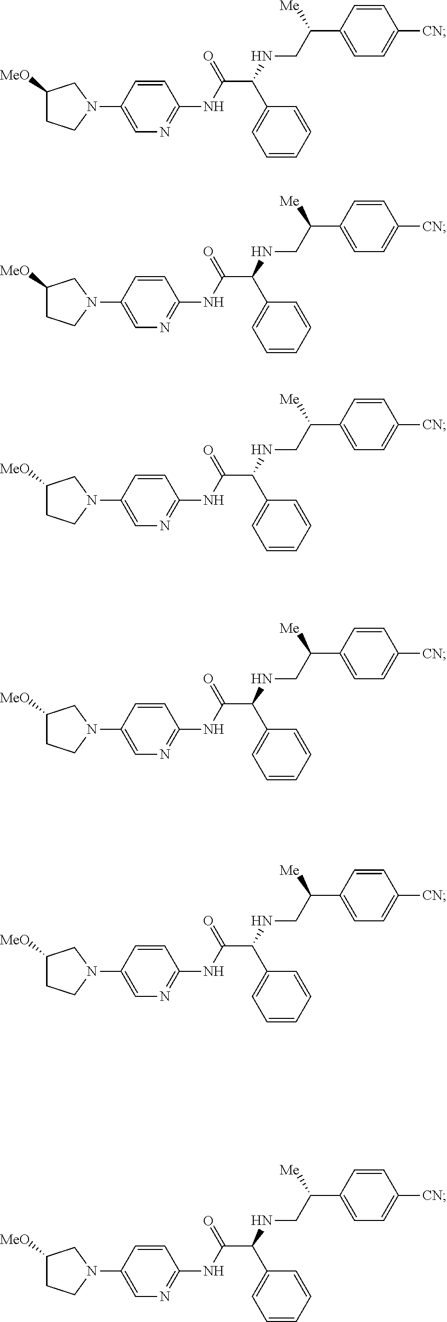

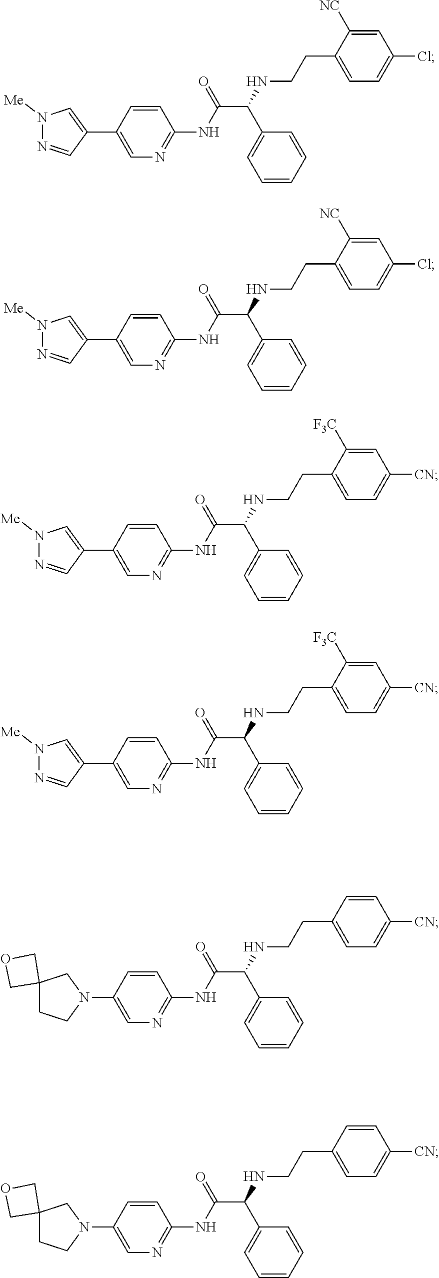 [SMILES: Cn1cc(-c2ccc(NC(=O)[C@H](NCCc3ccc(Cl)cc3C#N)c3ccccc3)nc2)cn1 ; Cn1cc(-c2ccc(NC(=O)[C@@H](NCCc3ccc(Cl)cc3C#N)c3ccccc3)nc2)cn1 ; Cn1cc(-c2ccc(NC(=O)[C@H](NCCc3ccc(C#N)cc3C(F)(F)F)c3ccccc3)nc2)cn1 ; Cn1cc(-c2ccc(NC(=O)[C@@H](NCCc3ccc(C#N)cc3C(F)(F)F)c3ccccc3)nc2)cn1 ; N#Cc1ccc(CCN[C@@H](C(=O)Nc2ccc(N3CCC4(COC4)C3)cn2)c2ccccc2)cc1 ; N#Cc1ccc(CCN[C@H](C(=O)Nc2ccc(N3CCC4(COC4)C3)cn2)c2ccccc2)cc1]

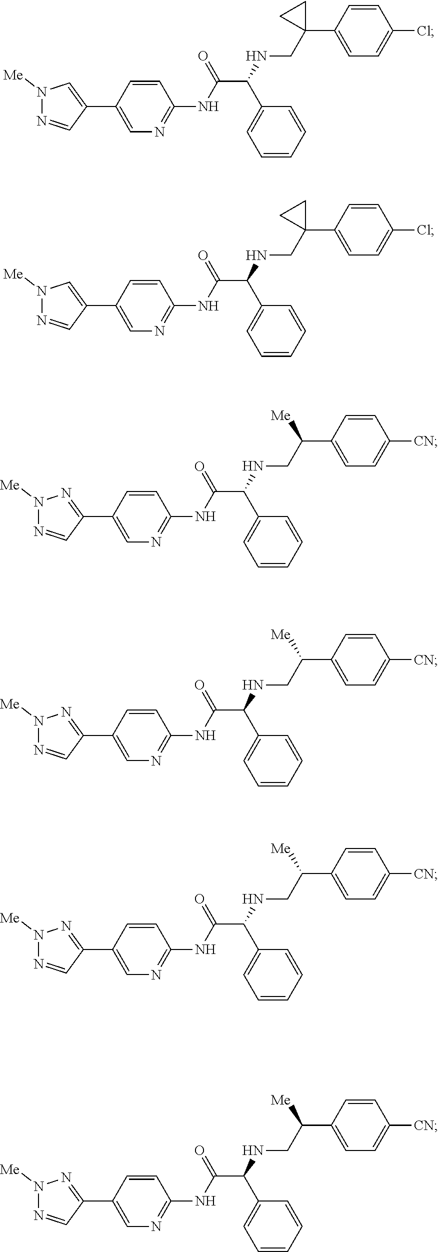

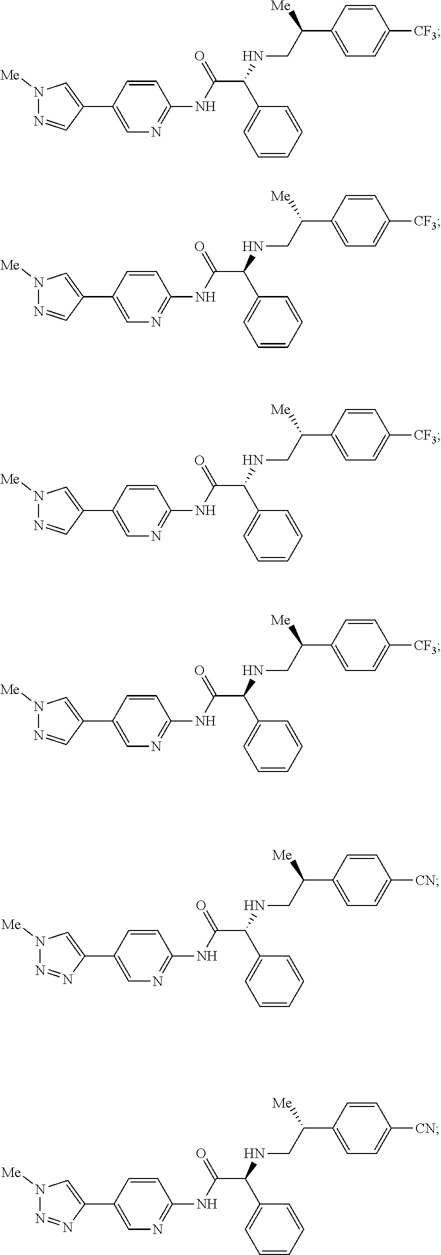

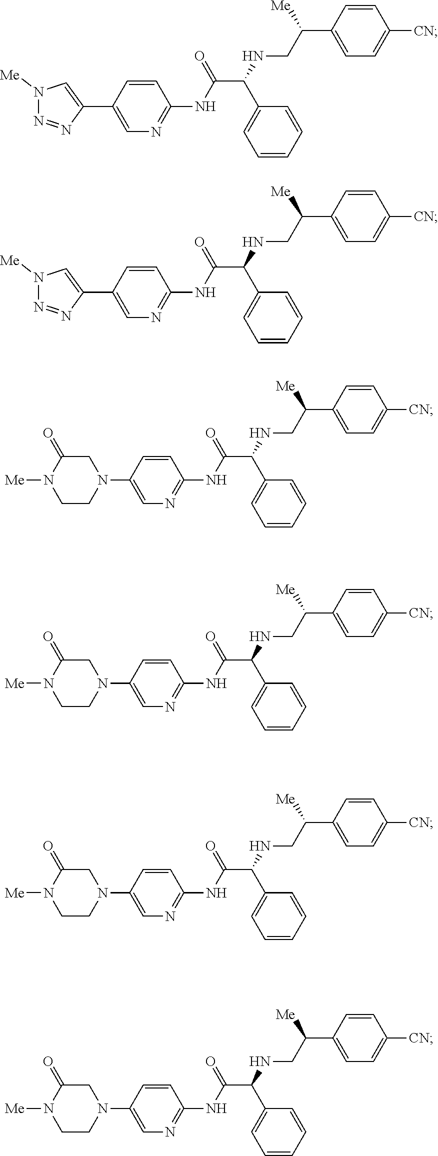

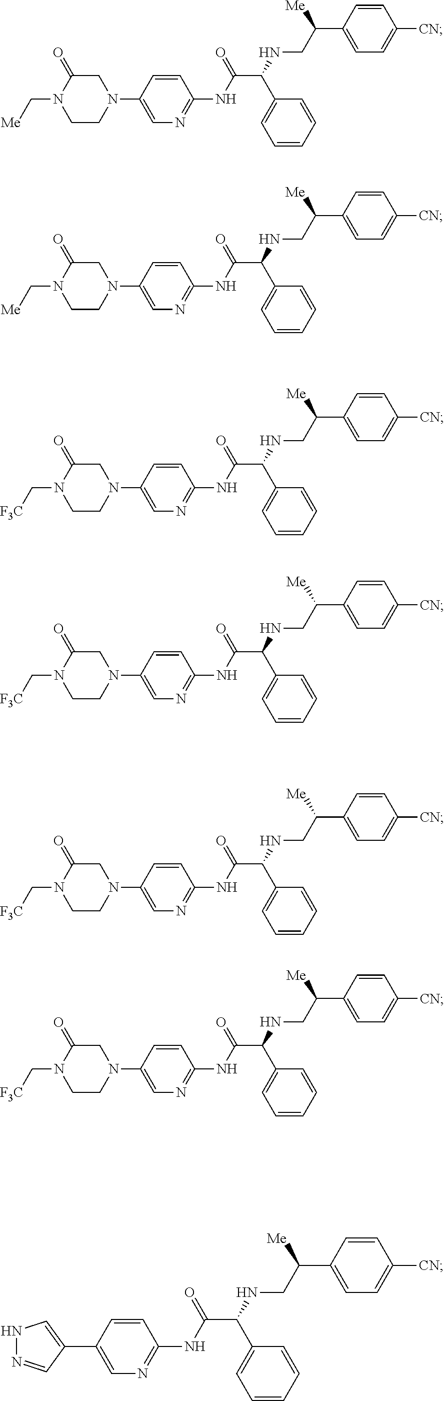

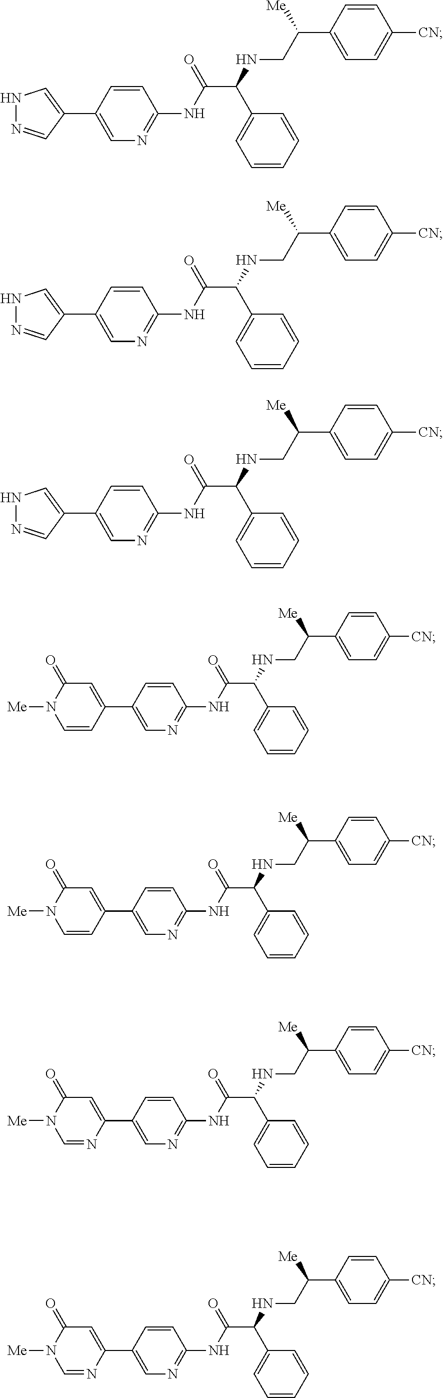

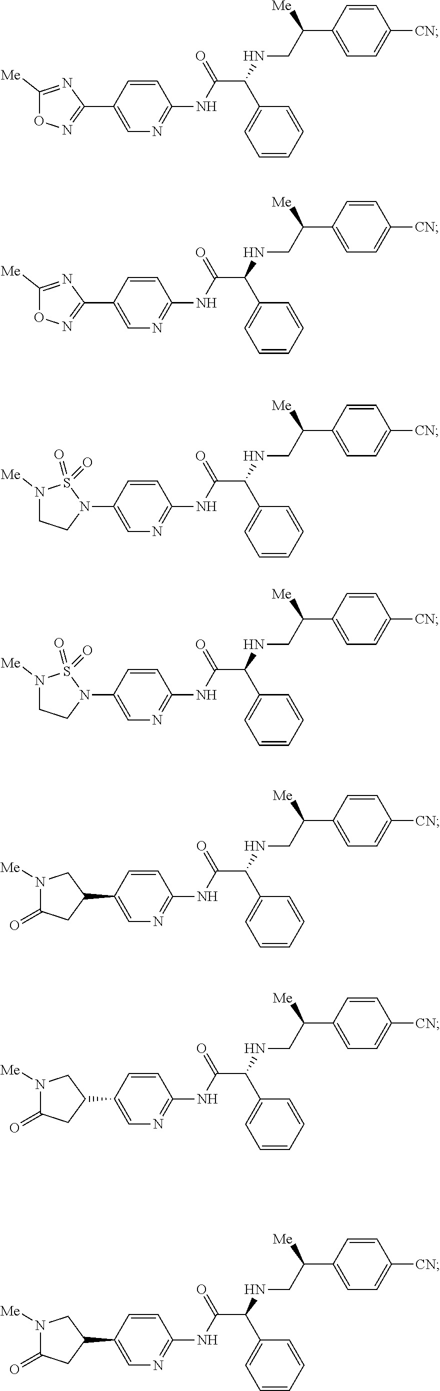

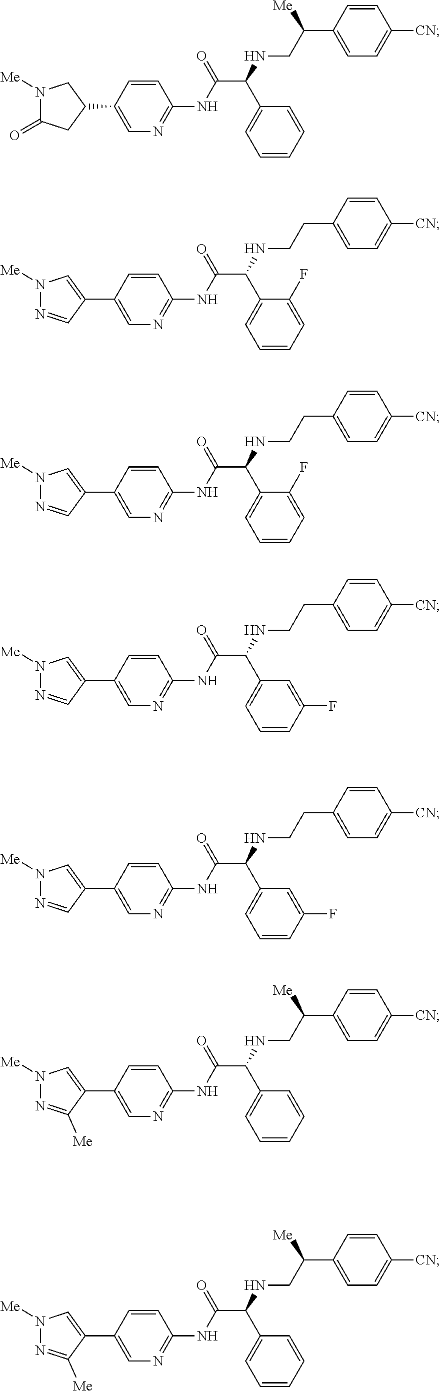

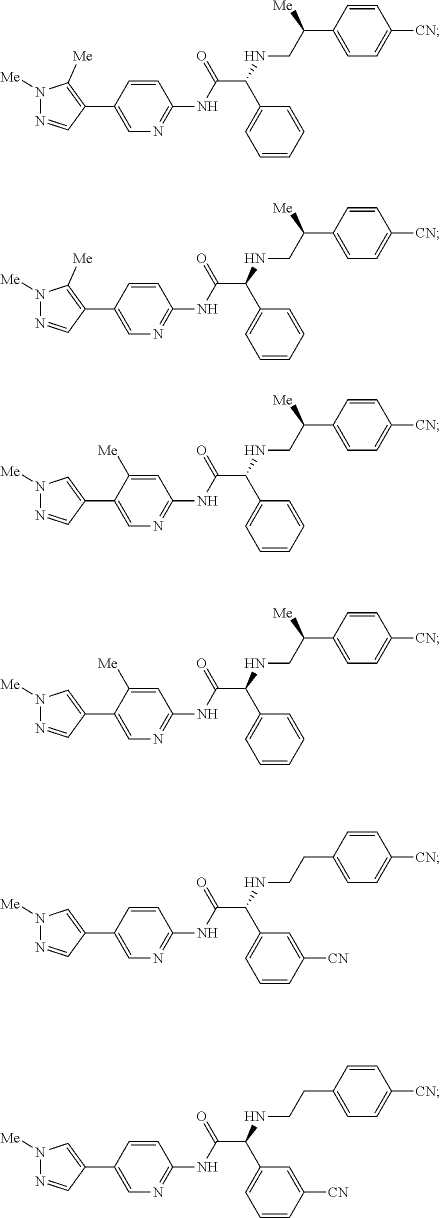

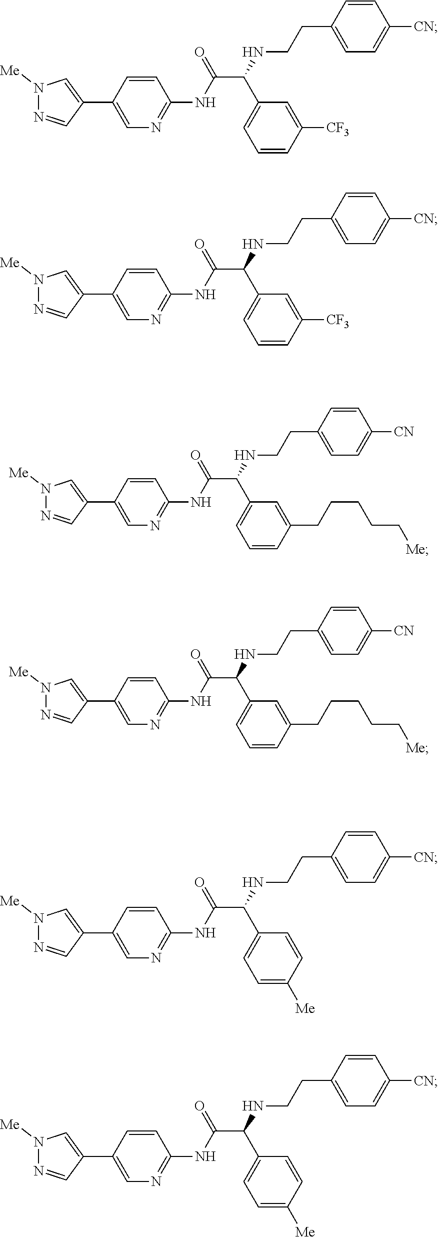 [SMILES: Cn1cc(-c2ccc(NC(=O)[C@H](NCCc3ccc(C#N)cc3)c3cccc(C(F)(F)F)c3)nc2)cn1 ; Cn1cc(-c2ccc(NC(=O)[C@@H](NCCc3ccc(C#N)cc3)c3cccc(C(F)(F)F)c3)nc2)cn1 ; CCCCCCc1cccc([C@@H](NCCc2ccc(C#N)cc2)C(=O)Nc2ccc(-c3cnn(C)c3)cn2)c1 ; CCCCCCc1cccc([C@H](NCCc2ccc(C#N)cc2)C(=O)Nc2ccc(-c3cnn(C)c3)cn2)c1 ; Cc1ccc([C@@H](NCCc2ccc(C#N)cc2)C(=O)Nc2ccc(-c3cnn(C)c3)cn2)cc1 ; Cc1ccc([C@H](NCCc2ccc(C#N)cc2)C(=O)Nc2ccc(-c3cnn(C)c3)cn2)cc1]

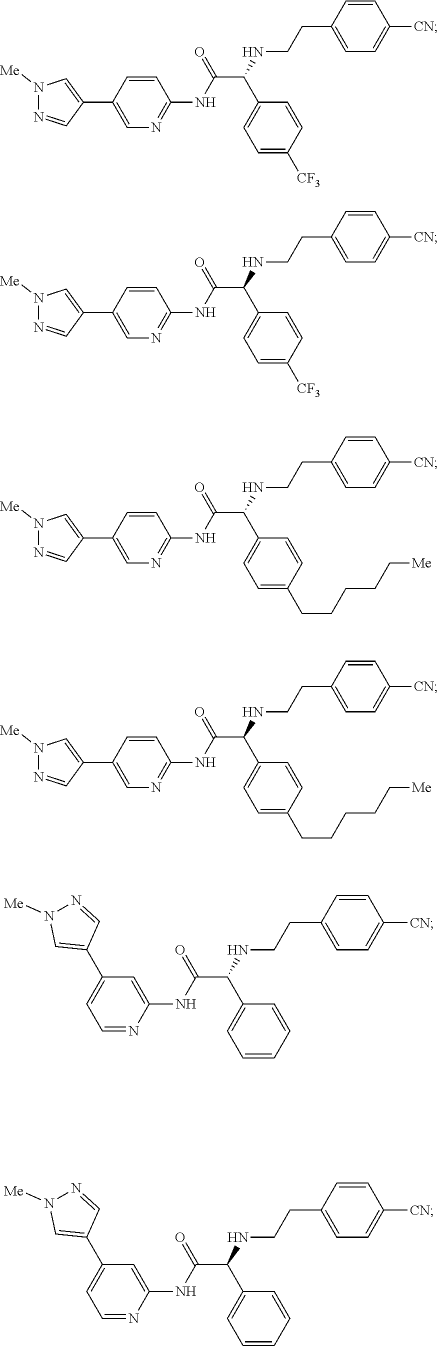 [SMILES: Cn1cc(-c2ccc(NC(=O)[C@H](NCCc3ccc(C#N)cc3)c3ccc(C(F)(F)F)cc3)nc2)cn1 ; Cn1cc(-c2ccc(NC(=O)[C@@H](NCCc3ccc(C#N)cc3)c3ccc(C(F)(F)F)cc3)nc2)cn1 ; CCCCCCc1ccc([C@@H](NCCc2ccc(C#N)cc2)C(=O)Nc2ccc(-c3cnn(C)c3)cn2)cc1 ; CCCCCCc1ccc([C@H](NCCc2ccc(C#N)cc2)C(=O)Nc2ccc(-c3cnn(C)c3)cn2)cc1 ; Cn1cc(-c2ccnc(NC(=O)[C@H](NCCc3ccc(C#N)cc3)c3ccccc3)c2)cn1 ; Cn1cc(-c2ccnc(NC(=O)[C@@H](NCCc3ccc(C#N)cc3)c3ccccc3)c2)cn1]

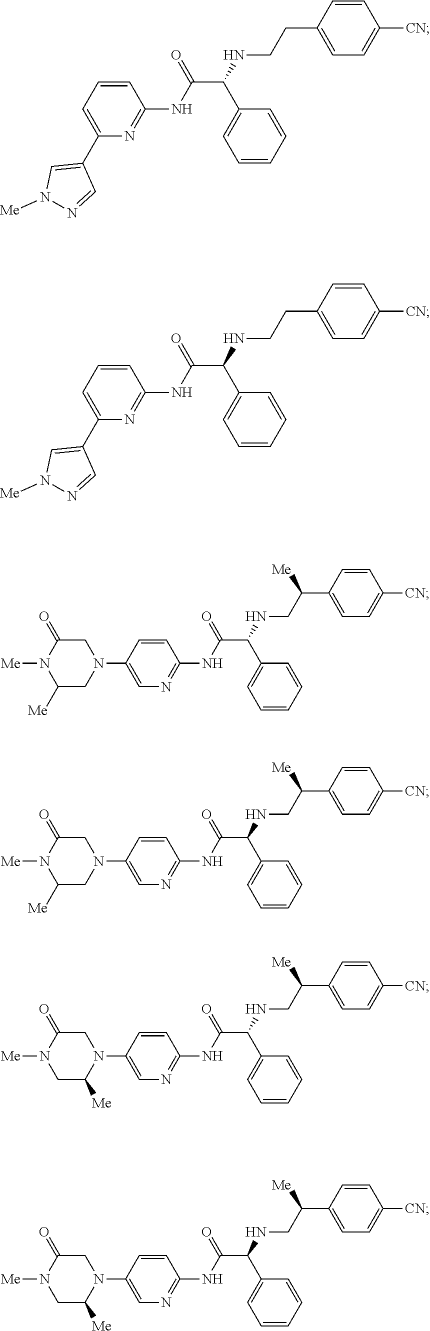

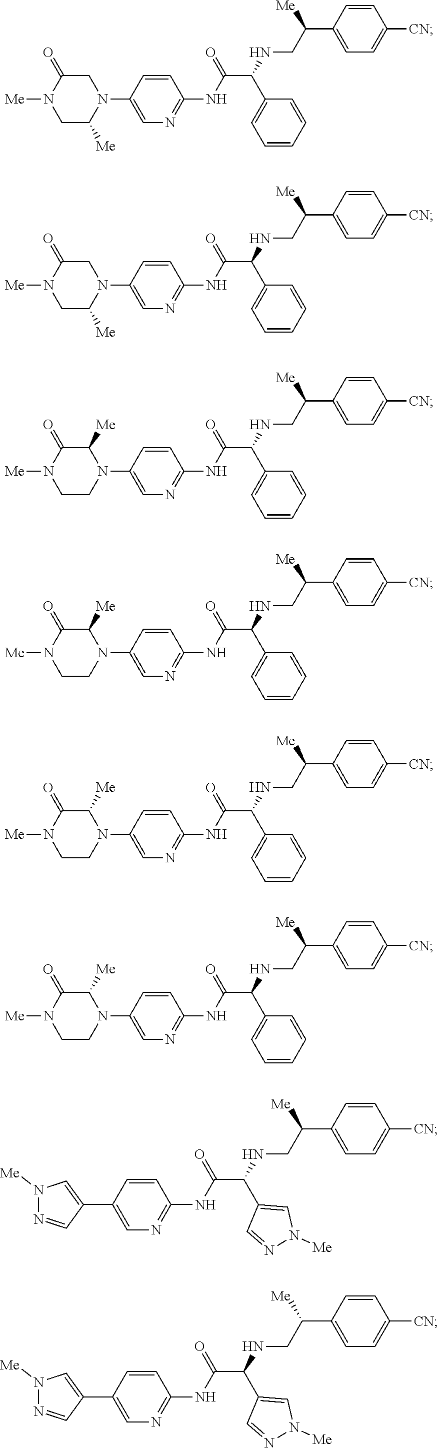

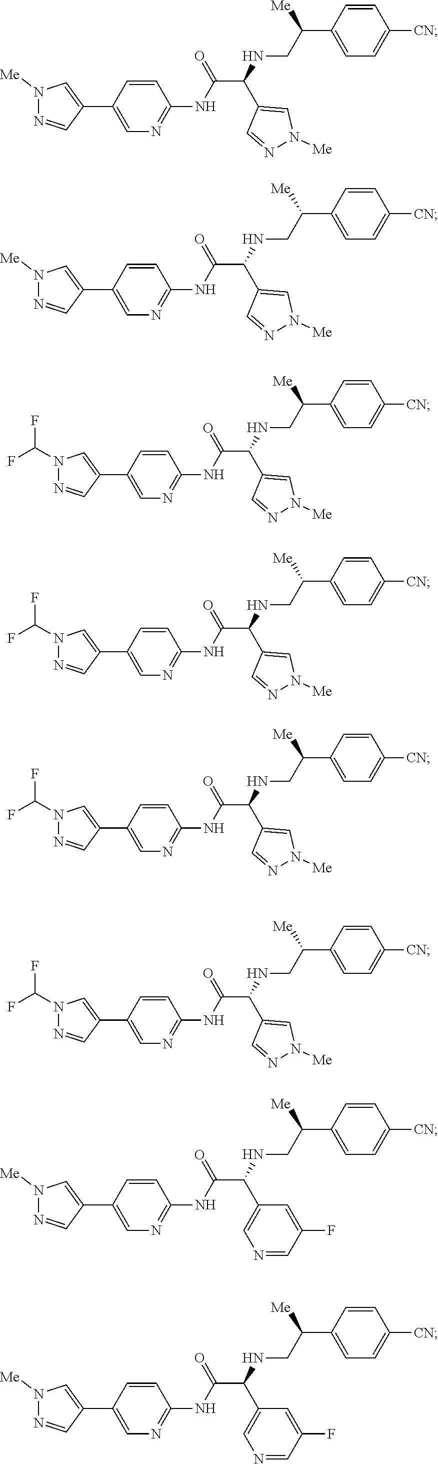

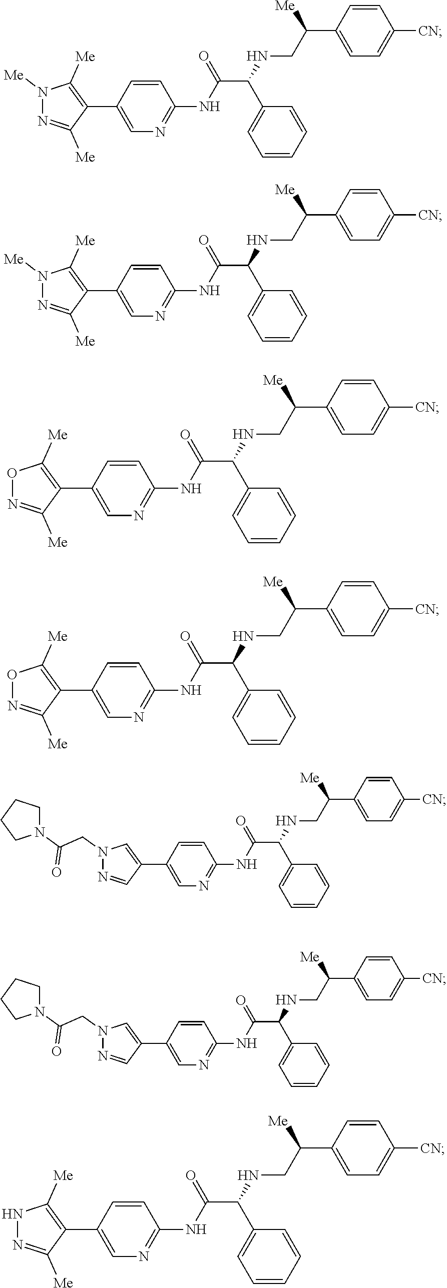

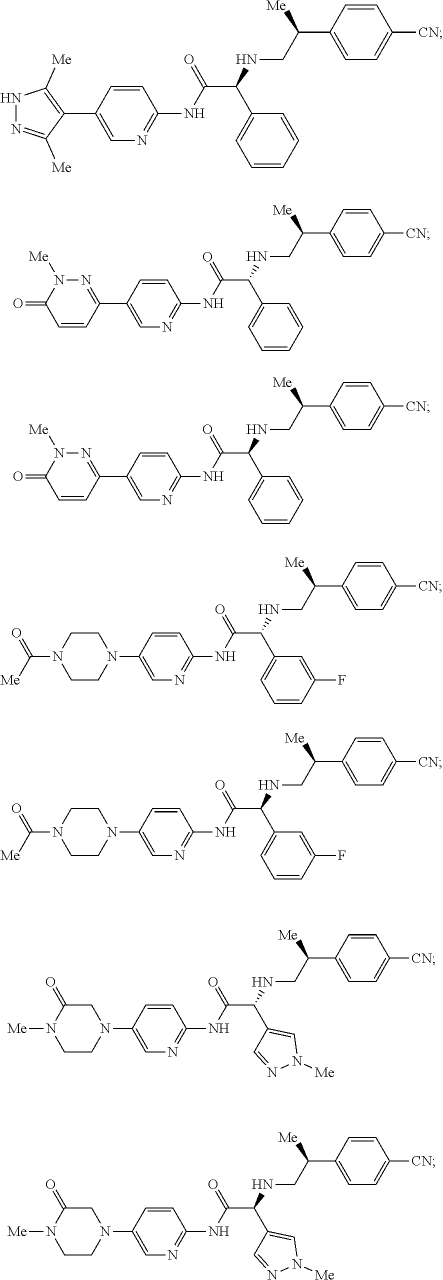

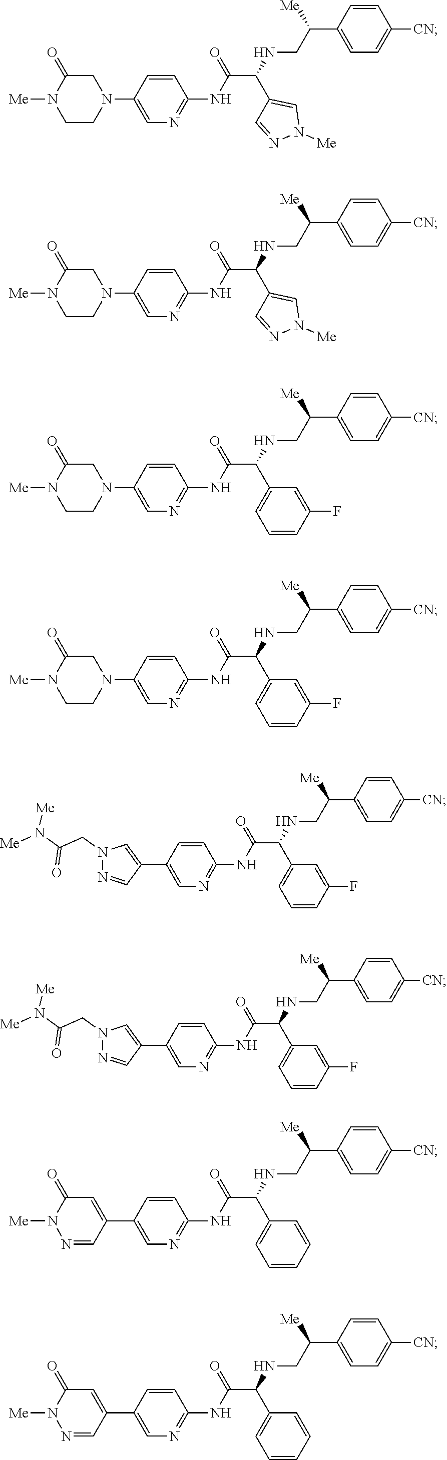

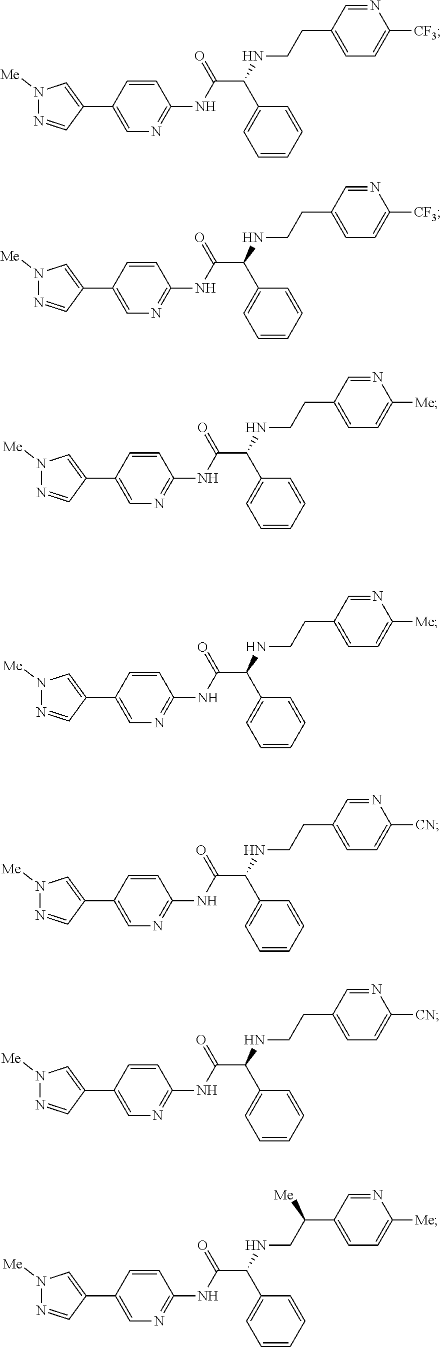

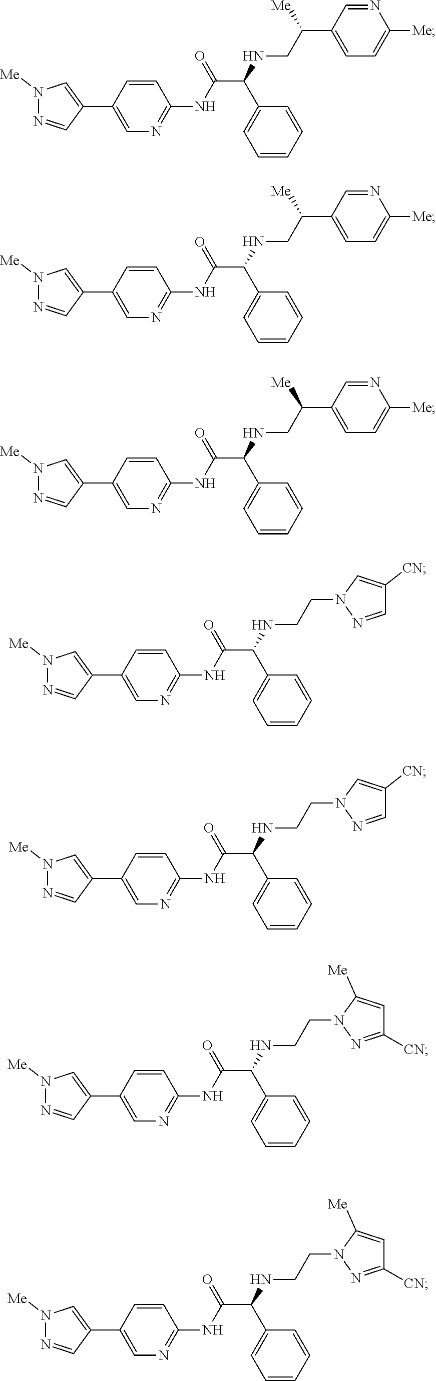

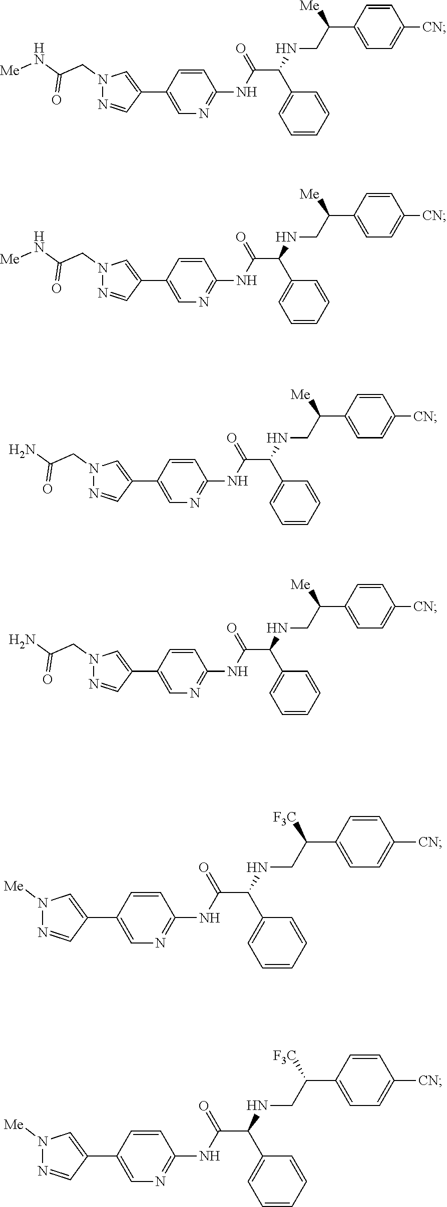

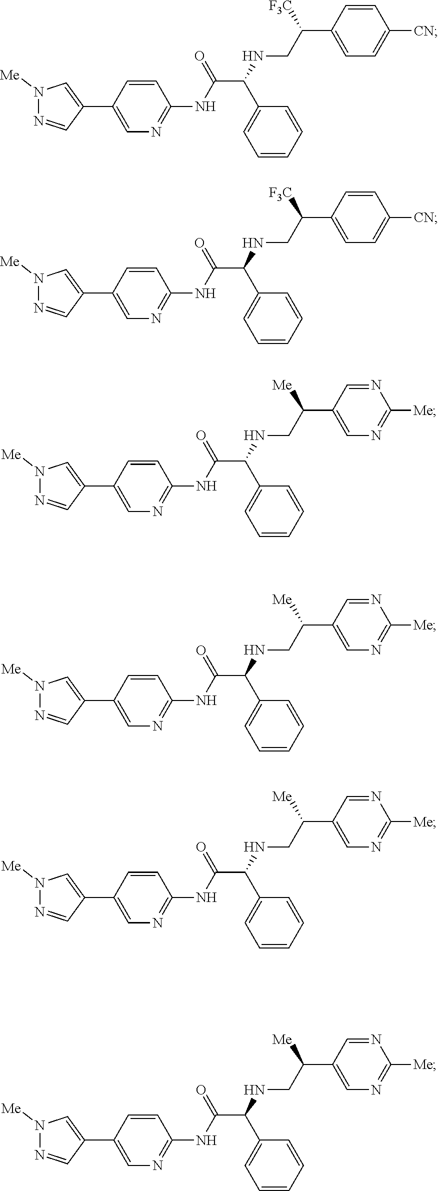

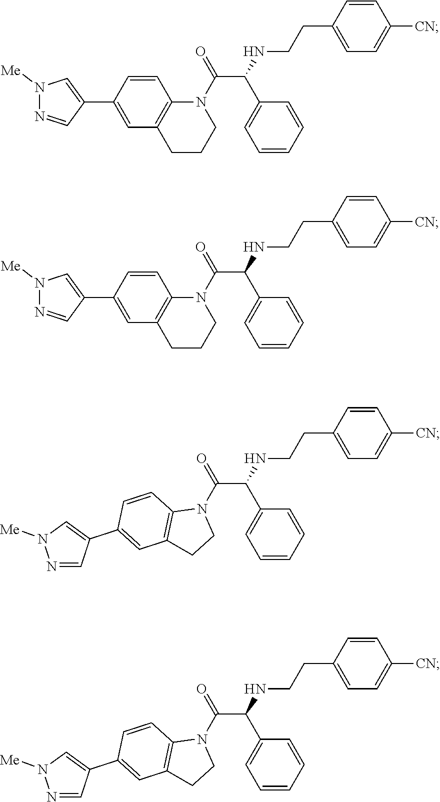 [SMILES: Cn1cc(-c2ccc3c(c2)CCCN3C(=O)[C@H](NCCc2ccc(C#N)cc2)c2ccccc2)cn1 ; Cn1cc(-c2ccc3c(c2)CCCN3C(=O)[C@@H](NCCc2ccc(C#N)cc2)c2ccccc2)cn1 ; Cn1cc(-c2ccc3c(c2)CCN3C(=O)[C@H](NCCc2ccc(C#N)cc2)c2ccccc2)cn1 ; Cn1cc(-c2ccc3c(c2)CCN3C(=O)[C@@H](NCCc2ccc(C#N)cc2)c2ccccc2)cn1]

or a pharmaceutically acceptable salt thereof of any of the foregoing.

Specific examples of compounds are provided in the EXEMPLIFICATION section and are included herein. Pharmaceutically acceptable salts as well as the neutral forms of these compounds are also included.

### 4. Uses, Formulation and Administration

Compounds and compositions described herein are useful for treating neurological disorders.

Examples of neurological disorders include: (i) chronic neurodegenerative diseases such as fronto-temporal lobar degeneration (frontotemporal dementia, FTD), FTD-GRN, familial and sporadic amyotrophic lateral sclerosis (FALS and ALS, respectively), familial and sporadic Parkinson's disease, Parkinson's disease dementia, Huntington's disease, familial and sporadic Alzheimer's disease, multiple sclerosis, muscular dystrophy, olivopontocerebellar atrophy, multiple system atrophy, Wilson's disease, progressive supranuclear palsy, diffuse Lewy body disease, corticodentatonigral degeneration, progressive familial myoclonic epilepsy, striatonigral degeneration, torsion dystonia, familial tremor, Down's Syndrome, Gilles de la Tourette syndrome, Hallervorden-Spatz disease, peripheral neuropathy, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapie, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease; pathologies arising with chronic alcohol or drug abuse including, for example, the degeneration of neurons in locus coeruleus and cerebellum, drug-induced movement disorders; pathologies arising with aging including degeneration of cerebellar neurons and cortical neurons leading to cognitive and motor impairments; and pathologies arising with chronic amphetamine abuse to including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor) and Wernicke-Korsakoff's related dementia.

Other neurological disorders include nerve injury or trauma associated with spinal cord injury. Neurological disorders of limbic and cortical systems include e.g., cerebral amyloidosis, Pick's atrophy, and Rett syndrome. In another aspect, neurological disorders include disorders of mood, such as affective disorders and anxiety; disorders of social behavior, such as character defects and personality disorders; disorders of learning, memory, and intelligence, such as mental retardation and dementia. Thus, in one aspect the disclosed compounds and compositions may be useful in treating schizophrenia, delirium, attention deficit hyperactivity disorder (ADHD), schizoaffective disorder, Alzheimer's disease, vascular dementia, Rubinstein-Taybi syndrome, depression, mania, attention deficit disorders, drug addiction, dementia, and dementia including BPSD manifestations.

Further neurological conditions include e.g., tauopathies, spinal and bulbar muscular atrophy, spinocerebellar ataxia type 3, pain (including e.g., acute and chronic pain, somatic pain, visceral pain, neuropathic pain, peripheral neuropathy, nociceptive pain, central pain syndrome, muscular or joint pain), and neuroinflammation.

In one aspect, the compounds and compositions described herein are useful in treating a neurological disorder selected from frontotemporal dementia, Alzheimer's disease, tauopathies, vascular dementia, Parkinson's disease, and dementia with Lewy bodies.

In certain aspects, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

In some aspects, the compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

Combination therapies using a therapeutically effective amount of a compound of Formula I, or a pharmaceutically acceptable salt thereof, and an effective amount of one or more additional pharmaceutically active agents are also included herein. Additional active agents that can be combined with a compound of Formula I, or a pharmaceutically acceptable salt thereof, include e.g., those which target the estrogen receptor (ER). These include, but are not limited to selective estrogen receptor degraders (SERDs), ER antagonists, selective estrogen receptor modulators (SERMs), and aromatase inhibitors (AIs). Examples of SERDs and ER antagonists include, but are not limited to, fulvestrant, RAD-1901 (elacestrant), GDC-0927 ((2S)-2-(4-{2-[3-(fluoromethyl)-1-azetidinyl]ethoxy}phenyl)-3-(3-hydroxyphenyl)-4-methyl-2H-chromen-6-ol), GDC-0810 (brilanestrant), AZD-9496 ((2E)-3-[3,5-difluoro-4-[(1R,3R)-2-(2-fluoro-2-methylpropyl)-2,3,4,9-tetrahydro-3-methyl-1H-pyrido[3,4-b]indol-1-yl]phenyl]-2-propenoic acid), OP-1250 (a prodrug of (S)-3-(4-hydroxyphenyl)-4-methyl-2-(4-(2-((R)-3-methylpyrrolidin-1-yl)ethoxy)phenyl)-2H-chromen-7-ol found in U.S. Pat. No. 9,018,244, the contents of which are incorporated herein by reference), (S)-3-(4-hydroxyphenyl)-4-methyl-2-(4-(2-((R)-3-methylpyrrolidin-1-yl)ethoxy)phenyl)-2H-chromen-7-ol, also found in U.S. Pat. No. 9,018,244, the contents of which are incorporated herein by reference), LSZ102 ((E)-3-(4-((2-(2-(1,1-difluoroethyl)-4-fluorophenyl)-6-hydroxybenzo[b]thiophen-3-yl)oxy)phenyl)acrylic acid), and H3B-6545 ((E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide). Examples of SERMs include, but are not limited to, tamoxifen, toremifene, raloxifene, bazedoxifene, ospemifene, and nafoxidene. Examples of AIs include, but are not limited to, anastrozole, letrozole, exemestane, vorozole, formestane and fadrozole. In one aspect, provided is a compound of Formula I, or a pharmaceutically acceptable salt thereof, and an additional therapeutic agent selected from fulvestrant, RAD-1901, GDC-0927, GDC-0810, AZD-9496, OP-1250, LSZ102, H3B-6545, tamoxifen, toremifene, raloxifene, bazedoxifene, ospemifene, nafoxidene, anastrozole, letrozole, exemestane, vorozole, formestane and fadrozole. In one aspect, the additional therapeutic agent is fulvestrant. The use of one or more of the combination therapies discussed above for treating a condition recited herein is also included within the scope of the present disclosure.

### EXEMPLIFICATION

Representative examples of the disclosed compounds are illustrated in the following non-limiting methods, schemes, and examples.

General starting materials used were obtained from commercial sources or prepared in other examples, unless otherwise noted.

The following abbreviations have the indicated meanings:

Ac=acetyl; ACN=acetonitrile; AcO acetate; BOC=t-butyloxycarbonyl; CBZ=carbobenzoxy; CDI=carbonyldiimidazole; DBU=1,8-Diazabicycloundec-7-ene; DCC=1,3-dicyclohexylcarbodiimide; DCE=1,2-dichloroethane; DI=de-ionized; DIAD=Diisopropyl azodicarboxylate; DIBAL=diisobutyl aluminum hydride; DIPA=diisopropylamine; DIPEA or DIEA=N,N-diisoproylethylamine, also known as Hunig's base; DMA=dimethylacetamide; DMAP=4-(dimethylamino)pyridine; DMF=dimethylformamide; DMP=Dess-Martin periodinane; DPPA=Diphenylphosphoryl azide; DPPP=1,3-bis(diphenylphosphino)propane; Dtbbpy=4,4′-di-/e/7-butyl-2,2′-dipyridyl; EDC or EDCI=1-(3-dimethylaminopropyl)-3-ethylcarbodiimide hydrochloride; EDTA=ethylenediaminetetraacetic acid, tetrasodium salt; EtOAc=ethyl acetate; FAB=fast atom bombardment; FMOC=9-fluorenylmethoxycarbonyl; HMPA=hexamethylphosphoramide; HATU=(9-(7-Azabenzotriazol-1-yl)-N, N, N, N-tetramethyluroniumhexafluorophosphate; HOAt=1-Hydroxy-7-azabenzotriazole or 3H-[1,2,3]triazolo[4,5-b]pyridin-3-ol; HOBt=1-hydroxybenzotriazole; HRMS=high resolution mass spectrometry; KHMDS=potassium hexamethyldisilazane; LC-MS=Liquid chromatography-mass spectrometry; LDA=lithium diisopropylamide; LiHMDS=lithium hexamethyldisilazane; MCPBA=meta-chloroperbenzoic acid; MMPP=magnesium monoperoxyphthlate hexahydrate; Ms=methanesulfonyl=mesyl; MsO=methanefulfonate=mesylate; MTBE=Methyl t-butyl ether; NBS=N-bromosuccinimide; NMM=4-methylmorpholine; NMP=N-methylpyrrolidinone; NMR=Nuclear magnetic resonance; PCC=pyridinium chlorochromate; PDC=pyridinium dichromate; Ph=phenyl; PPTS=pyridiniump-toluene sulfonate; pTSA=p-toluene sulfonic acid; r.t./RT=room temperature; rac.=racemic; T3P=2,4,6-Tripropyl-1,3,5,2,4,6-trioxatriphosphinane 2,4,6-trioxide; TEA=triethylamine; TFA=trifluoroacetic acid; TfO=trifluoromethanesulfonate=triflate; THE=tetrahydrofuran; TLC=thin layer chromatography; TMSC1=trimethylsilyl chloride.

Unless otherwise stated, the absolute configuration of each eluting stereoisomer in the following examples was not identified.

The progress of reactions was often monitored by TLC or LC-MS. The LC-MS was recorded using one of the following methods.

### Method-C3

| Mobile | (A ) | 2 mM Ammonium acetate + | | |
|---|---|---|---|---|
| Phase | | 0.1% Formic Acid in Water | | |
| | (B) | 0.1% Formic Acid in Acetonitrile | | |
| Column | | BEH C18 (50*2.1 mm) 1.7 um | | |
| Column Flow | | 0.55 ml/min | | |
| Gradient | | Time (min) | % A | % B |
| | | 0.01 | 98 | 2 |
| | | 0.30 | 98 | 2 |
| | | 0.60 | 50 | 50 |
| | | 1.10 | 25 | 75 |
| | | 2.00 | 0 | 100 |
| | | 2.70 | 0 | 100 |
| | | 2.71 | 98 | 2 |
| | | 3.00 | 98 | 2 |

### PDS Method-J

| Mobile | (A) | 5 mM Ammonium Acetate + 0.1% | | |
|---|---|---|---|---|
| Phase | | Formic Acid in Water | | |
| | (B) | 0.1% Formic Acid in Acetonitrile | | |
| Column | | BEH C18 (50*2.1 mm), 1.7 um or Equivalent | | |
| Column Flow | | 0.45 ml/min | | |
| Gradient | | Time (min) | % A | % B |
| | | 0.01 | 98 | 2 |
| | | 0.50 | 98 | 2 |
| | | 5.00 | 10 | 90 |
| | | 6.00 | 5 | 95 |
| | | 7.00 | 5 | 95 |
| | | 7.01 | 98 | 2 |
| | | 8.00 | 98 | 2 |

### Method-H

| Mobile | (A) | 5 mM Ammonium bicarbonate in water | | |
|---|---|---|---|---|
| Phase | (B) | Acetonitrile | | |
| Column | | X-Bridge C18 (50*4.6 mm), 3.5 um | | |
| Column Flow | | 1.0 ml/min | | |
| Gradient | | Time (min) | % A | % B |
| | | 0.01 | 95 | 5 |
| | | 5.00 | 10 | 90 |
| | | 5.80 | 5 | 95 |
| | | 7.20 | 5 | 95 |
| | | 7.21 | 95 | 5 |
| | | 10.00 | 95 | 5 |

### Method-F

| Mobile | (A) | 10 mM Ammonium Acetate in WATER | | |
|---|---|---|---|---|
| Phase | (B) | 100% Acetonitrile | | |
| Column | | X-Bridge C18 (150*4.6 mm), | | |
| | | 5 um or Equivalent | | |
| Column Flow | | 1.0 ml/min | | |
| Gradient | | Time (min) | % A | % B |
| | | 0.01 | 90 | 10 |
| | | 5.00 | 10 | 90 |
| | | 7.00 | 0 | 100 |
| | | 11.00 | 0 | 100 |
| | | 11.01 | 90 | 10 |
| | | 12.00 | 90 | 10 |

### Method-G

| Mobile Phase | (A) | 10 mM Ammonium Acetate in Water | | |
|---|---|---|---|---|
| | (B) | 100% Acetonitrile | | |
| Column | | X-Bridge C18 (150*4.6 mm), | | |
| | | 5 um or Equivalent | | |
| Column Flow | | 1.0 ml/min | | |
| Gradient | | Time (min) | % A | % B |
| | | 0.01 | 100 | 0 |
| | | 7.00 | 50 | 50 |
| | | 9.00 | 0 | 100 |
| | | 11.00 | 0 | 100 |
| | | 11.01 | 100 | 0 |
| | | 12.00 | 100 | 0 |

NMR was recorded at room temperature unless noted otherwise on Varian Inova 400 or 500 MHz spectrometers with the solvent peak used as the reference or on Bruker 300 or 400 MHz spectrometers with the TMS peak used as internal reference.

The compounds described herein may be prepared using the following methods and schemes. Unless specified otherwise, all starting materials used are commercially available.

### Method 1

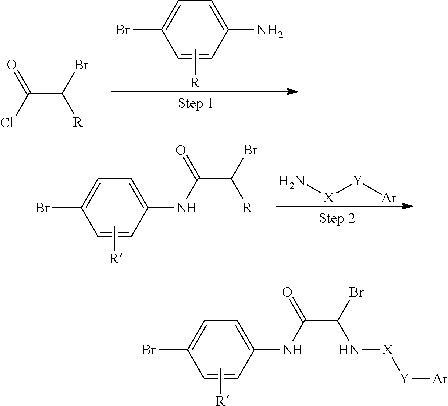

Method 1 is a 2-step protocol, consisting of an acylation reaction with a 2-bromoacylchloride and a subsequent alkylation reaction with a substituted ethylamine, for the preparation of N-(haloaryl)-2-(arylethylamino)-2-substituted acetamides or N-(haloheteroaryl)-2-(arylethylamino)-2-substituted acetamides, that is useful for the synthesis of intermediates en route to the compounds described herein.

### Method 2

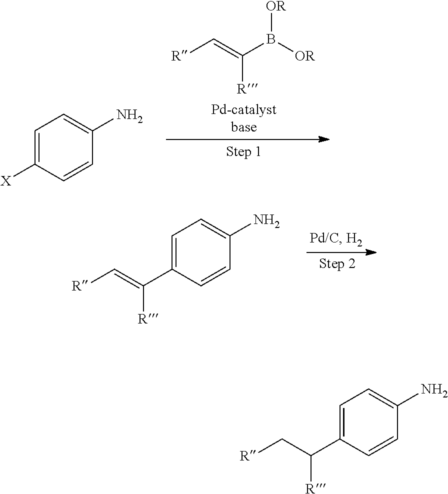

Method 2 is a 2-step protocol, which consists of a Suzuki cross-coupling reaction and a palladium-catalyzed hydrogenation reaction, for the preparation of methyl 4-alkylanilines starting from a haloaniline and an alkenylboronic ester that is useful for the synthesis of intermediates en route to the compounds described herein.

### Method 3

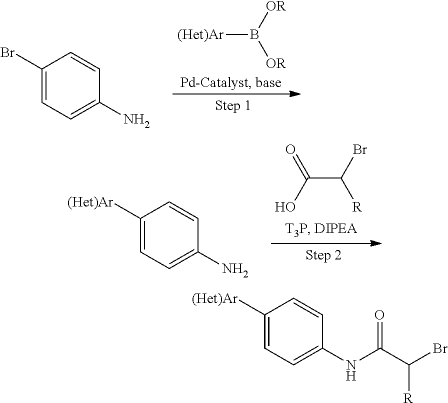

Method 3 is a 2-step protocol, which consists of a Suzuki cross-coupling reaction and an amide coupling, for the preparation of 2-bromo-N-(4-heteroaryl)-2-substituted acetamides starting from a haloaniline and an heteroarylboronic ester that is useful for the synthesis of intermediates en route to the compounds described herein.

### Methods 4, 5 and 6

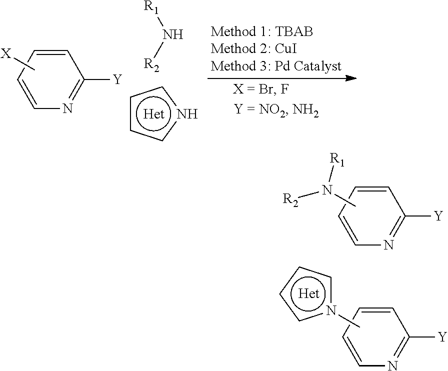

Methods 4, 5, and 6 are protocols for the coupling of substituted nitropyridines or aminopyridines with aliphatic and heteroaromatic amines for the preparation of substituted pyridines that are useful for the synthesis of intermediates en route to the compounds described herein.

### Method 7

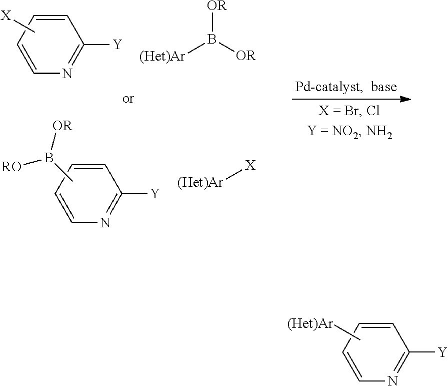

Method 7 is a protocol for the preparation of substituted pyridines, a Suzuki cross-coupling reaction of pyridine boronic acids and esters with aryl- and heteroaryl halides or a suzuki cross coupling reaction of halopyridines with aryl- or heteroaryl boronic acids and esters, that is useful for the synthesis of intermediates en route to the compounds described herein.

### Method 8

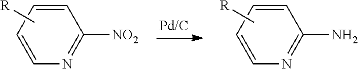

Method 8 is a protocol for the preparation of substituted 2-amino pyridines from 2-nitro pyridines via a palladium-catalyzed hydrogenation reaction that is useful for the synthesis of intermediates en route to the compounds described herein.

### Method 9

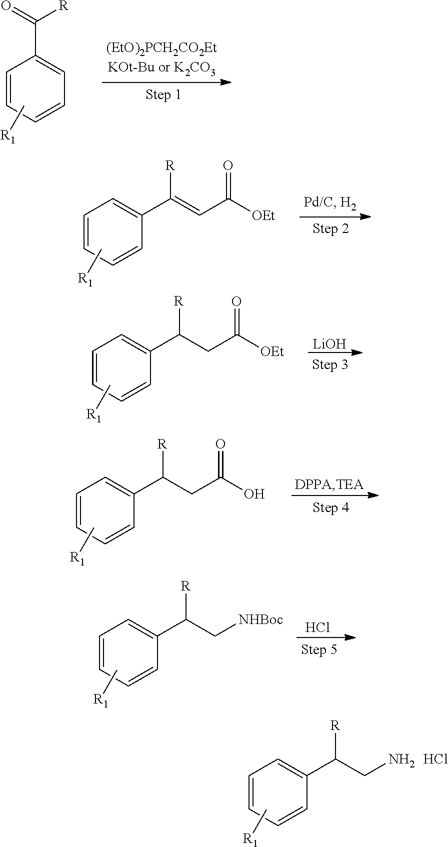

Method 9 is a 5 step-protocol for the preparation of substituted 2-arylethylamines and 2-heteroarylethylamines employing substituted benzaldehydes or ketones that is useful for the synthesis of intermediates en route to the compounds described herein.

### Method 10

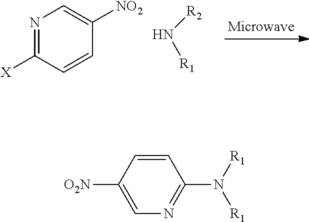

Method 10 is a protocol for the preparation of 2-substituted nitro pyridines from 2-halonitro pyridines and amines that is useful for the synthesis of intermediates en route to the compounds described herein.

### Method 11

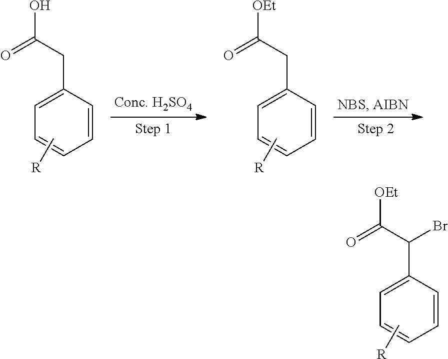

Method 11 is a 2 step-protocol for the preparation of substituted ethyl 2-bromo-2-phenylacetates from substituted phenyl acetic acid derivatives that is useful for the synthesis of intermediates en route to the compounds described herein.

### Method 12

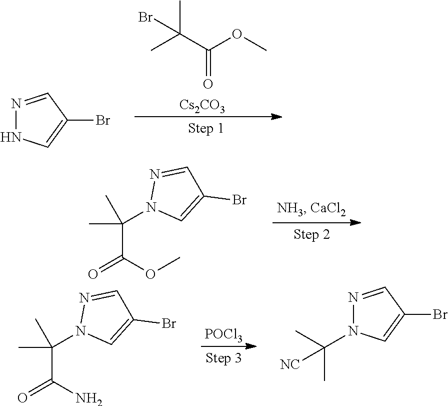

Method 12 is a 3 step-protocol for the synthesis of methyl 2-(4-bromo-1H-pyrazol-1-yl)-2-methylpropanenitrile from 4-bromo-1H-pyrazole that is useful for the synthesis of intermediates en route to the compounds described herein.

### Method 13

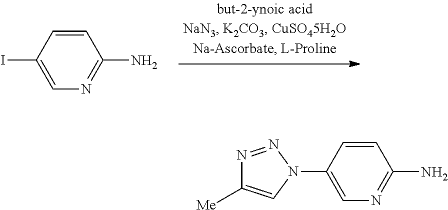 [SMILES: Nc1ccc(I)cn1 ; Cc1cn(-c2ccc(N)nc2)nn1]

Method 13 is a protocol for the preparation of 5-(4-methyl-1H-1,2,3-triazol-1-yl)pyridin-2-amine from 5-iodopyridin-2-amine that is useful for the synthesis of intermediates en route to the compounds described herein.

### Method 14

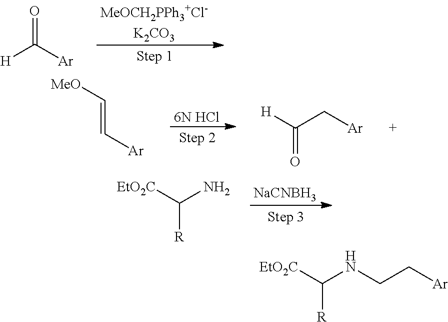

Method 14 is a 3-step protocol, used for the preparation of substituted ethyl phenethylamino-2-phenylacetates starting from substituted benzaldehydes that is useful for the synthesis of intermediates en route to the compounds described herein.

### Method 15

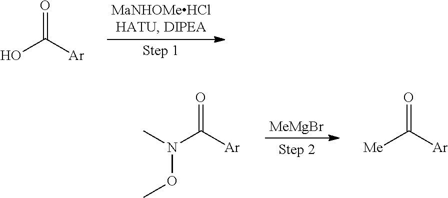

Method 15 is a 2-step protocol, used for the preparation of substituted acetophenones starting from substituted benzoic acids that is useful for the synthesis of intermediates en route to the compounds described herein.

### Method 16

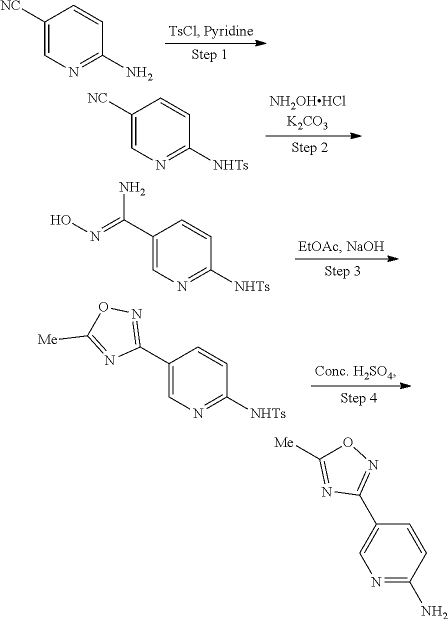

Method 16 is a 4-step protocol, used for the preparation of 5-(5-methyl-1,2,4-oxadiazol-3-yl)pyridin-2-amine starting from substituted 6-aminonicotinonitrile that is useful for the synthesis of intermediates en route to the compounds described herein.

### Method 17

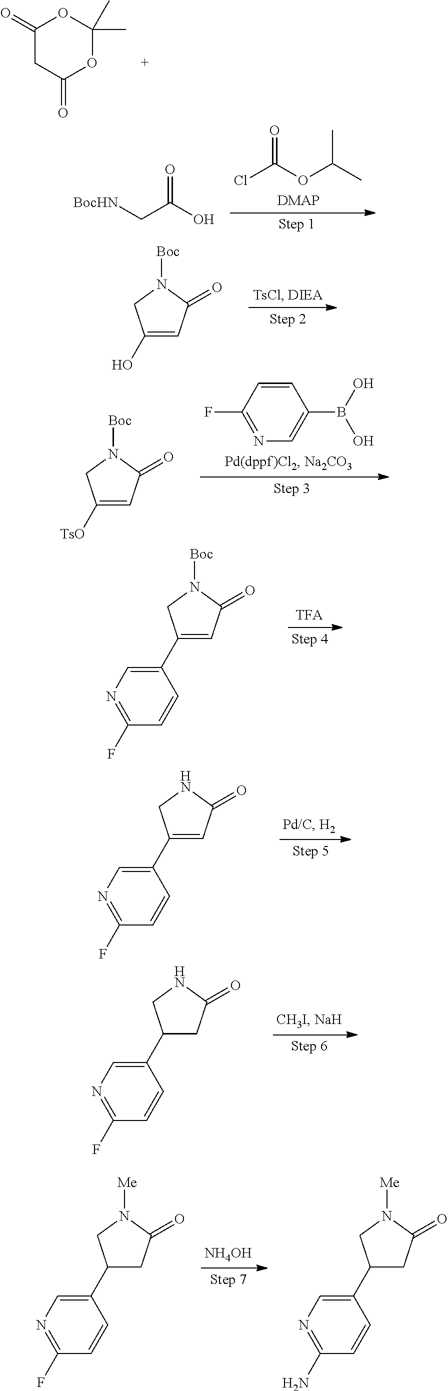

Method 17 is a 7-step protocol, used for the preparation of 4-(6-aminopyridin-3-yl)-1-methylpyrrolidin-2-ones starting from 2,2-dimethyl-1,3-dioxane-4,6-dione that is useful for the synthesis of intermediates en route to the compounds described herein.

### Method 18

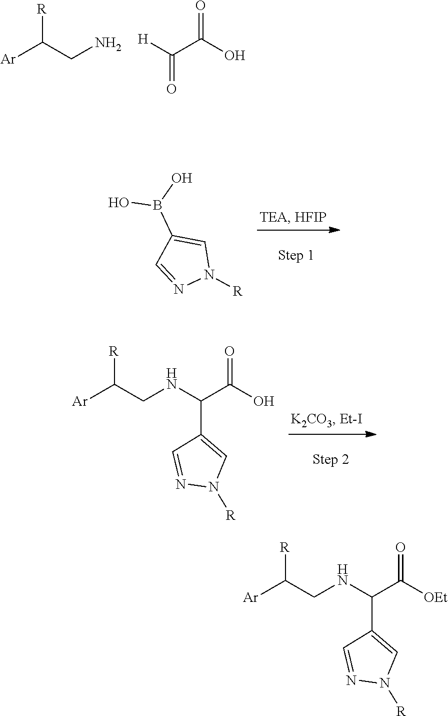

Method 18 is a 2-step protocol, used for the preparation of substituted ethyl 2-(arylethylamino)-2-(1-substituted-1H-pyrazol-4-yl)acetates starting from arylethylamines and substituted boronate (or boronic acid)pyrazoles that is useful for the synthesis of intermediates en route to the compounds described herein.

### Method 19

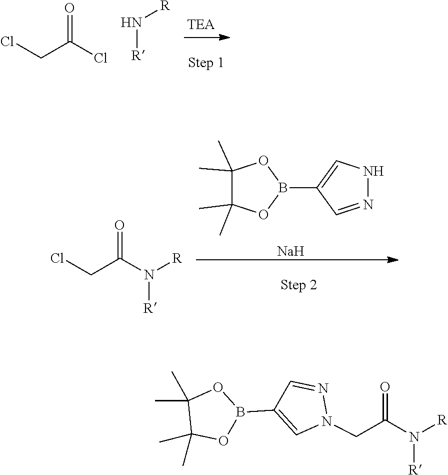

Method 19 is a 2-step protocol, used for the preparation of substituted 1-(amino)-2-(4-(4,4,5,5-tetramethyl-1,3,2-dioxaborolan-2-yl)-1H-pyrazol-1-yl)ethan-1-ones starting from amines that is useful for the synthesis of intermediates en route to the compounds described herein.

### Method 20

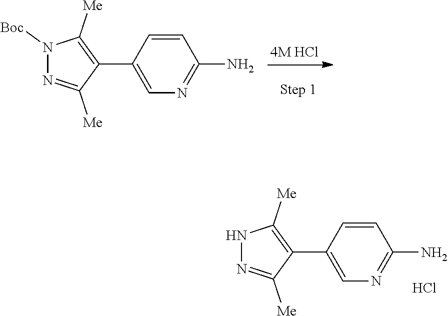 [SMILES: Cc1nn(C(=O)OC(C)(C)C)c(C)c1-c1ccc(N)nc1 ; Cc1n[nH]c(C)c1-c1ccc(N)nc1 ; Cl]

Method 20 is a protocol, used for the preparation of 5-(3,5-dimethyl-1H-pyrazol-4-yl)pyridin-2-amine starting from tert-butyl 4-(6-aminopyridin-3-yl)-3,5-dimethyl-1H-pyrazole-1-carboxylate that is useful for the synthesis of intermediates en route to the compounds described herein.

### Method 21

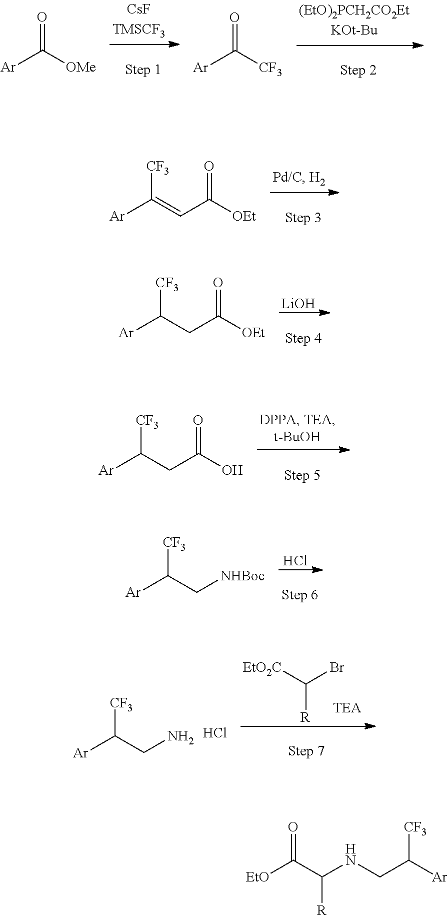

Method 21 is seven-step protocol for the preparation of ethyl trifluoromethyl phenethylalanine derivatives from methyl benzoate derivatives that is useful for the synthesis of intermediates en route to the compounds described herein.

### Method 22

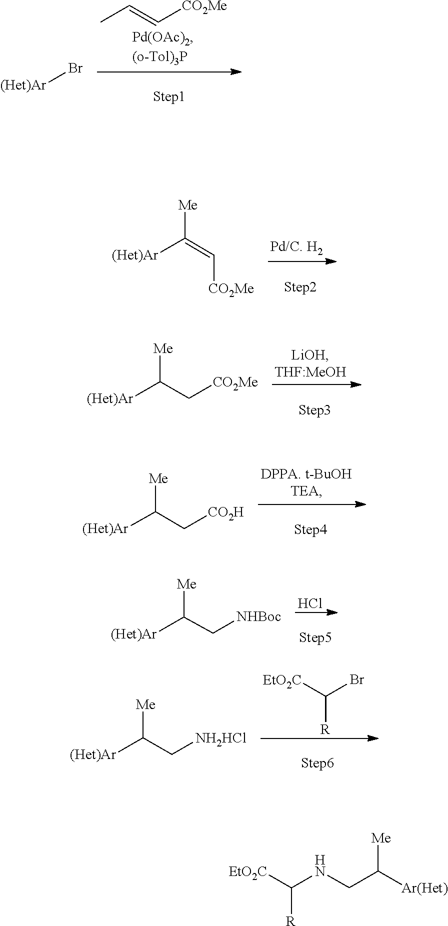

Method 22 is a six-step protocol for the synthesis of ethyl aryl(heteroaryl)propyl alanine derivatives from aryl- and heteroarylbromides that is useful for the synthesis of intermediates en route to the compounds described herein.

### Method 23

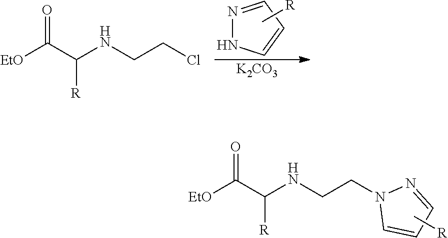

Method 23 is a protocol for the synthesis of ethyl 2-((2-(1H-pyrazol-1-yl)ethyl)amino)-2-acetate derivatives from ethyl 2-((2-chloroethyl)amino)-acetates that is useful for the synthesis of intermediates en route to the compounds described herein.

### Method 24

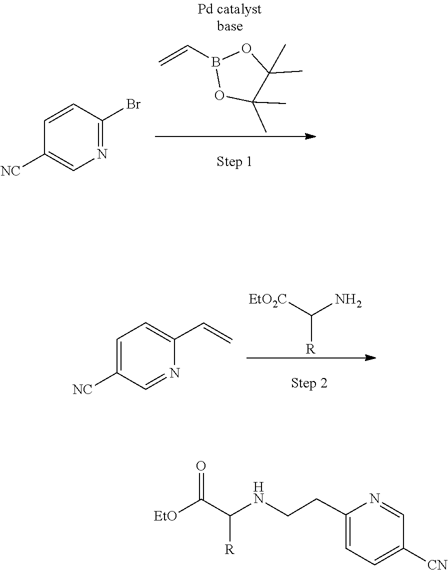

Method 24 is two-step protocol for the synthesis of ethyl 2-((2-(5-cyanopyridin-2-yl)ethyl)amino)-2-acetate derivatives from 2-bromo-5-cyanopyridines that is useful for the synthesis of intermediates en route to the compounds described herein.

### Method 25

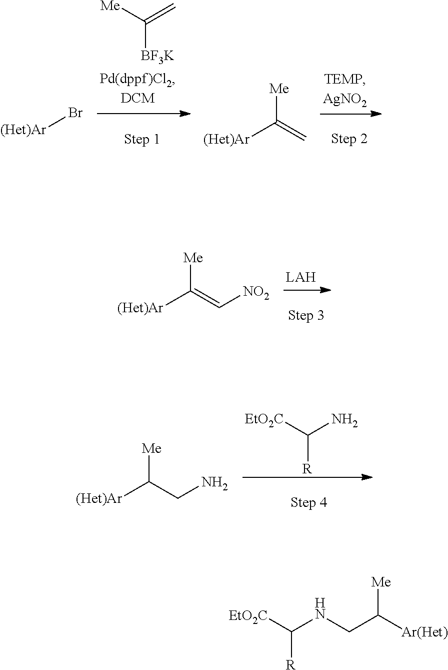

Method 25 is a four-step protocol for the synthesis of ethyl aryl(heteroaryl)propyl alanine derivatives from aryl- or heteroarylbromides that is useful for the synthesis of intermediates en route to the compounds described herein.

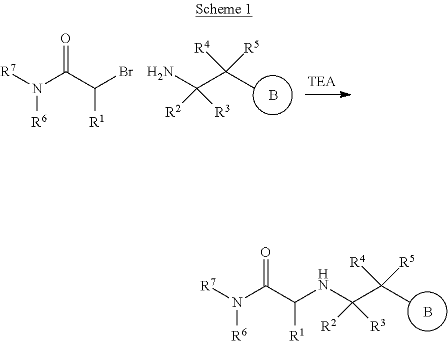

Scheme 1 illustrates a general method for the synthesis of the compounds of this invention via alkylation of amine with an α-bromoketone or α-bromoamide where B, R¹, R², R³, R⁴, R⁵, R⁶, and R⁷ are as described herein.

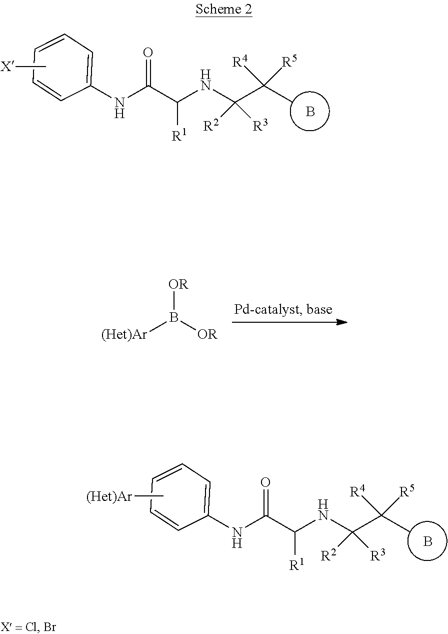

Scheme 2 illustrates a general method for the synthesis of a subset of the compounds described herein via a Suzuki reaction of a variety aryl- or heteroarylboronic esters and acids with a subset substituted compounds of Formula I where B, R¹, R², R³, R⁴, and R⁵ are as described herein.

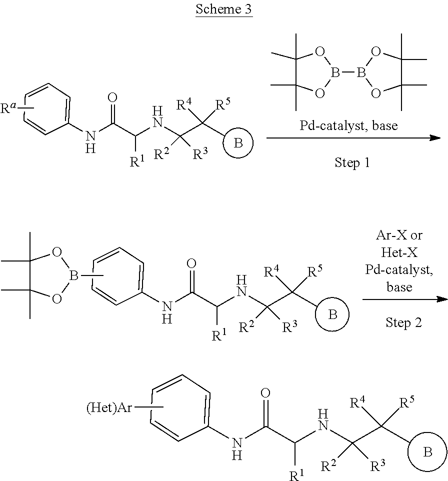

Scheme 3 illustrates a two-step sequence, useful for the synthesis of a subset of the compounds described herein that consists of a palladium-catalyzed borylation reaction of compounds of Formula I where B, R¹, R², R³, R⁴, and R⁵ are as described herein.

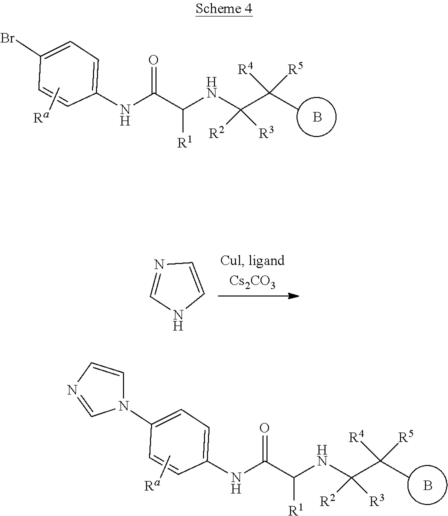

Scheme 4 illustrates a general method for the synthesis of a subset of the compounds described herein via a copper-catalyzed coupling reaction of a variety azoles with a family of substituted compounds of Formula I where B, R^{a}, R¹, R², R³, R⁴, and R⁵ are as described herein.

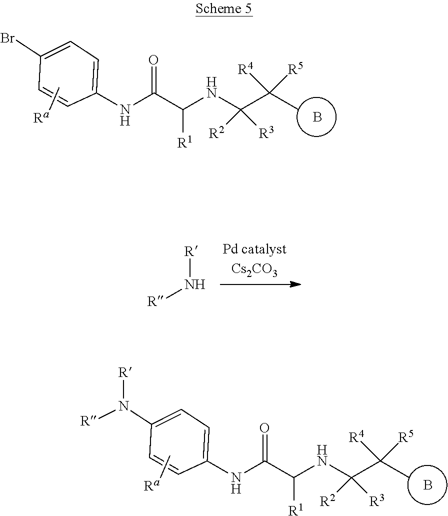

Scheme 5 illustrates a method for the synthesis of a subset of the compounds of this invention via a palladium-catalyzed C—N coupling reaction of amines with a family of substituted compounds of Formula I where B, R^{a}, R¹, R², R³, R⁴, and R⁵ are as described herein.

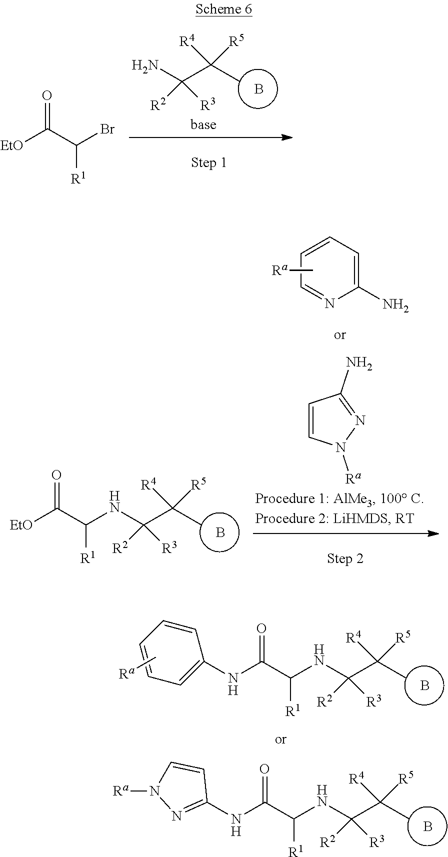

Scheme 6 illustrates a 2-step synthetic sequence for the conversion of an α-bromoester to N-aryl-2-(alkylamino)acetamide. The method is useful for the synthesis of a subset of the compounds of Formula I where R¹ is a substituted phenyl and B, R^{a}, R², R³, R⁴, and R⁵ are as described herein.

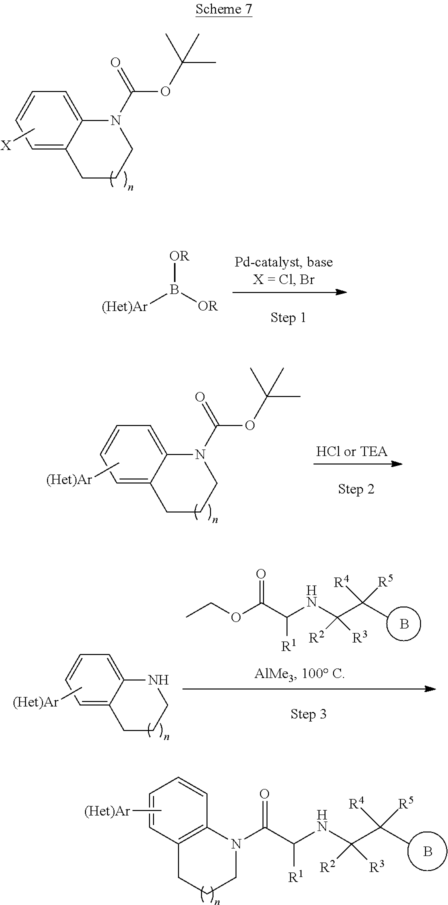

Scheme 7 illustrates a synthetic sequence used for conversion of a halogenated amine, such as a bromotetrahydroquinoline (n=1) or bromoindoline (n=0), into a subset of compounds of Formula I where R¹ is a substituted phenyl and B, R², R³, R⁴, and R⁵ are as described herein.

### Method 1

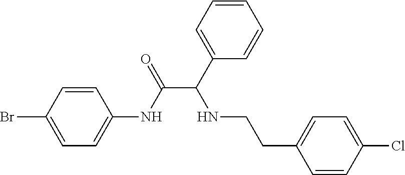 [SMILES: O=C(Nc1ccc(Br)cc1)C(NCCc1ccc(Cl)cc1)c1ccccc1]

### N-(4-Bromophenyl)-2-((4-chlorophenethyl)amino)-2-phenylacetamide

### Method 1, Step 1. 2-Bromo-N-(4-bromophenyl)-2-phenylacetamide

To a stirred solution of 2-bromo-2-phenylacetic acid (1 g, 2.32 mmol) in dry DCM (10 ml) was added thionyl chloride (1.1 ml, 3.95 mmol) dropwise at 0° C. and reaction mixture was stirred at 40° C. overnight. After completion of the reaction, excess of thionyl chloride and DCM were evaporated under reduced pressure. Then to this, THE (10 ml) and 4-bromo aniline (0.79 g, 4.64 mmol) were added and resulting reaction mixture was stirred for 4 hours at room temperature. After completion of the reaction, 1 N aqueous HCl solution was added slowly and the DCM layer was separated. The aqueous layer was extracted with DCM (2×30 ml) and the combined organic layers were washed with 2 N aqueous NaOH solution, dried over anhydrous Na₂SO₄ and concentrated under reduced pressure to afford the title compound (1 g, 65%). LCMS: m/z=367.98 [M+1].

### Method 1, Step 2. N-(4-Bromophenyl)-2-((4-chlorophenethyl)amino)-2-phenylacetamide

A mixture of 2-bromo-N-(4-bromophenyl)-2-phenylacetamide (0.8 g, 2.17 mmol), 2-(4-chlorophenyl)ethan-1-amine (0.680 g, 4.35 mmol) and TEA (0.7 ml, 4.35 mmol) in DMF (15 ml) were heated for 2 hours at 60° C. After completion of the reaction, the reaction mixture was poured into ice cold water (10 ml) and extracted with ethyl acetate (2×30 ml). The combined organic layers were washed with brine (10 ml), dried over anhydrous Na₂SO₄ and concentrated under reduced pressure. The resulting residue was purified by silica gel chromatography to afford the title compound (0.7 g, 67%) as off-white solid. LCMS: m/z=443.5 [M+1] and 445.5 [M+2].

### Method 2

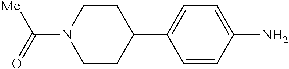 [SMILES: CC(=O)N1CCC(c2ccc(N)cc2)CC1]

### 1-(4-(4-Aminophenyl)piperidin-1-yl)ethan-1-one

### Method 2, Step 1. 1-(4-(4-Aminophenyl)-3,6-dihydropyridin-1(2H)-yl)ethan-1-one

A mixture of 4-bromoaniline (0.3 g, 1.74 mmol), 1-(4-(4,4,5,5-tetramethyl-1,3,2-dioxaborolan-2-yl)-3,6-dihydropyridin-1(2H)-yl)ethan-1-one (0.525 g, 2.09 mmol) and cesium carbonate (1.70 g, 5.23 mmol) in 4:1 dioxane:water (15 ml) was purged for 20 minutes with argon. Then S-Phos Pd-precatalyst G3 (0.066 g, 0.087 mmol) was added and purging with argon was continue for another 10 minutes. The reaction mixture was heated at 90° C. overnight. After completion of reaction (monitored by TLC), the reaction mixture was treated with water (6 ml) and extracted with ethyl acetate (2×15 ml). The combined organic layers were washed with brine (10 ml), dried over anhydrous Na₂SO₄ and concentrated under reduced pressure. The resulting residue was purified by silica gel chromatography to afford the title compound as a solid (0.35 g, 92%). LCMS: m/z=217.32 [M+1].

### Method 2, Step 2. 1-(4-(4-Aminophenyl)piperidin-1-yl)ethan-1-one

1-(4-(4-aminophenyl)-3,6-dihydropyridin-1(2H)-yl)ethan-1-one (350 mg, 1.62 mmol) was dissolved in 1:1 MeOH:ethyl acetate (3.5 ml) in an autoclave and 10% Pd/C (35 mg, 50% moisture) was added. The reaction was heated at 50° C. for 2 hours under 100 PSI of hydrogen gas pressure. After completion of reaction (monitored by TLC), the reaction mixture was filtered through a pad of celite and the filtrate was concentrated to afford the title compound (300 mg, 85%). LCMS: m/z=219.3 [M+1].

### Method 3

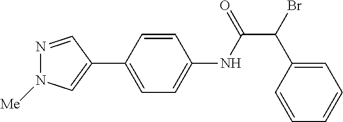 [SMILES: Cn1cc(-c2ccc(NC(=O)C(Br)c3ccccc3)cc2)cn1]

### 2-Bromo-N-(4-(1-methyl-1H-pyrazol-4-yl)phenyl)-2-phenylacetamide

### Method 3, Step 1. 4-(1-Methyl-1H-pyrazol-4-yl)aniline

A mixture of 4-bromoaniline (1.0 g, 5.81 mmol), 1-methyl-4-(4,4,5,5-tetramethyl-1,3,2-dioxaborolan-2-yl)-1H-pyrazole (1.3 g, 6.39 mmol) and cesium carbonate (5.68 g, 17.43 mmol) in 4:1 dioxane:water (20 ml) was purged for 20 minutes with argon. S-Phos Pd-precatalyst G3 (0.213 g, 0.29 mmol) was added and purging was continued for another 10 minutes. The reaction mixture was heated at 100° C. for 2 hours. The reaction mixture was poured into water (15 ml) and extracted with ethyl acetate (2×20 ml). The combined organic layers were washed with brine (10 ml), dried over anhydrous Na₂SO₄ and concentrated under reduced pressure. The resulting residue was purified by silica gel chromatography to afford the title compound (0.965 g, 95%) as solid. ¹H NMR (400 MHz, DMSO-d6): 3.81 (s, 3H), 5.01 (s, 2H, —NH₂), 6.54 (d, J=8.0 Hz, 2H), 7.20 (d, J=8.4 Hz, 2H), 7.63 (s, 1H), 7.86 (s, 1H). LCMS: m/z=174.2 [M+1].

### Method 3, Step 2. 2-Bromo-N-(4-(1-methyl-1H-pyrazol-4-yl)phenyl)-2-phenylacetamide

To a stirred solution of 4-(1-methyl-1H-pyrazol-4-yl) aniline (0.95 g, 5.48 mmol) and 2-bromo-2-phenylacetic acid (1.3 g, 6.03 mmol) in ethyl acetate (10 ml) was added T3P (5.22 g, 8.22 mmol; 50% in ethyl acetate). The reaction mixture was stirred for 30 minutes at room temperature. After 30 minutes DIPEA (1.41 g, 10.96 mmol) was added and the reaction mixture was heated at 60° C. for 3 hours. The reaction mixture was poured into water (15 ml) and extracted with ethyl acetate (2×10 ml). The combined organic layers were washed with brine (10 ml), dried over anhydrous Na₂SO₄ and concentrated under reduced pressure. The resulting residue was purified by silica gel chromatography to afford the title compound (1.2 g, 59%) as a solid. ¹H NMR (400 MHz, DMSO-d6): 3.85 (s, 3H), 5.79 (s, 1H), 7.38-7.44 (m, 3H), 7.52-7.59 (m, 4H), 7.65 (d, J=6.8 Hz, 2H), 7.82 (s, 1H), 8.09 (s, 1H), 10.54 (s, 1H, —NH). LCMS: m/z=370.1 [M+1] and 372.4 [M+2].

### Method 4

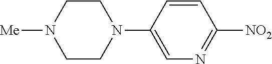 [SMILES: CN1CCN(c2ccc([N+](=O)[O-])nc2)CC1]

### 1-Methyl-4-(6-nitropyridin-3-yl)piperazine

### Method 4, Step 1. 1-Methyl-4-(6-nitropyridin-3-yl)piperazine

To a stirred solution of 5-bromo-2-nitropyridine (0.5 g, 2.46 mmol) in DMSO (5 ml) was added 1-methylpiperazine (0.369 g, 3.69 mmol), K₂CO₃ (0.679 g, 4.92 mmol) and TBAB (0.079 g, 0.0246 mmol) at room temperature. The reaction mixture was stirred at 100° C. for 6 hours. After completion of the reaction (monitored by TLC), the reaction was quenched with 1N HCl (15 ml) and extracted with ethyl acetate (2×15 ml). The aqueous layer was treated with 1N NaOH solution and extracted with ethyl acetate (2×25 ml). The combined organic layers were dried over anhydrous Na₂SO₄ and concentrated under reduced pressure to afford title compound (0.5 g, 91%). ¹H NMR (400 MHz, DMSO-d6): 2.39 (s, 3H), 2.61 (t, J=5.2 Hz, 4H), 3.50 (t, J=5.2 Hz, 4H), 7.22 (dd, J=8.8 Hz, 2.8 Hz, 1H), 8.15-8.20 (m, 2H).

### Method 5

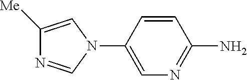 [SMILES: Cc1cn(-c2ccc(N)nc2)cn1]

### 5-(4-Methyl-1H-imidazol-1-yl)pyridin-2-amine

### Method 5, Step 1. 5-(4-Methyl-1H-imidazol-1-yl)pyridin-2-amine

To a stirred solution of 5-bromopyridin-2-amine (0.5 g, 2.89 mmol) in DMF (10 ml) was added 4-methyl-1H-imidazole (1.19 g, 14.45 mmol), Cs₂CO₃ (0.94 g, 2.89 mmol), CuI (0.276 g, 1.45 mmol) and 1-(5,6,7,8-Tetrahydroquinoline-8-yl)ethanone (0.11 g, 0.58 mmol) at room temperature. The reaction mixture was purged with argon gas for 30 minutes and it was heated at 135° C. overnight. After completion of the reaction, water (15 ml) was added and the mixture was extracted with ethyl acetate (2×25 ml). The combined organic layers were dried over anhydrous Na₂SO₄ and concentrated under reduced pressure. The resulting residue was purified by silica gel chromatography to afford the title compound (0.23 g, 46%). LCMS: m/z=175.3 [M+1].

### Method 6

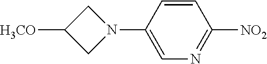 [SMILES: COC1CN(c2ccc([N+](=O)[O-])nc2)C1]

### 5-(3-Methoxyazetidin-1-yl)-2-nitropyridine

### Method 6, Step 1. 5-(3-Methoxyazetidin-1-yl)-2-nitropyridine

To a stirred solution of 5-bromo-2-nitropyridine (0.55 g, 4.44 mmol) in 1,4-dioxane (2.5 ml) was added 3-methoxyazetidine (1.08 g, 5.33 mmol), Cs₂CO₃ (4.38 g, 13.49 mmol), Pd₂(dba)₃ (0.162 g, 0.17 mmol) and Xantphos (0.257 g, 0.44 mmol) at room temperature. The reaction mixture was purged with argon gas for 30 minutes and it was heated at 100° C. for 3 hours. After completion of the reaction, water was added (15 ml) and the aqueous layer was extracted with ethyl acetate (2×25 ml). The combined organic layers were dried over anhydrous Na₂SO₄ and concentrated under reduced pressure. The resulting residue was purified by silica gel chromatography to afford the title compound (0.77 g, 74%). LCMS: m/z=210.1 [M+1].

### Method 7

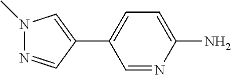

### 5-(1-Methyl-1H-pyrazol-4-yl)pyridin-2-amine

### Method 7. 5-(1-Methyl-1H-pyrazol-4-yl)pyridin-2-amine

A mixture of 5-bromopyridin-2-amine (18.0 g, 104.04 mmol), 1-methyl-4-(4,4,5,5-tetramethyl-1,3,2-dioxaborolan-2-yl)-1H-pyrazole (32.47 g, 156.06 mmol) and cesium carbonate (101.75 g, 312.12 mmol) in dioxane:water (4:1, 360 ml) were purged for 20 minutes with argon gas. To this mixture, Pd(dppf)Cl₂ (7.61 g, 10.40 mmol) was added and purging was continued for another 10 minutes. The reaction mixture was heated at 80° C. for 1.5 hours. The reaction mixture was poured into water (200 ml) and extracted with ethyl acetate (2×200 ml). The combined organic layers were washed with brine (150 ml), dried over anhydrous Na₂SO₄ and concentrated under reduced pressure. The crude product was purified by column chromatography to afford the title compound (15 g, 82%) as a solid. ¹H NMR (400 MHz, DMSO-d6): 3.83 (s, 3H), 5.86 (s, 2H, —NH₂), 6.44 (d, J=8.4 Hz, 1H), 7.20 (dd, J=8.4 Hz, 2.4 Hz, 1H), 7.70 (s, 1H), 7.95 (s, 1H), 8.14 (d, J=2.09 Hz, 1H). LCMS: m/z=175.1 [M+1].

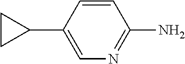

### 5-Cyclopropylpyridin-2-amine

### Method 7. 5-Cyclopropylpyridin-2-amine

5-bromopyridin-2-amine (0.5 g, 2.89 mmol), cyclopropylboronic acid (0.49 g, 5.78 mmol) and K₃PO₄ (1.84 g, 8.67 mmol) were combined in a mixture of toluene:water (4:1, 10 ml) and the mixture was degassed for 20 minutes with argon gas. To the reaction mixture, palladium acetate (0.032 g, 0.144 mmol) and tricyclohexyl-phosphine (0.081 g, 0.289 mmol) were added and degassing was continued for another 10 minutes. The reaction mixture was heated in a sealed tube at 100° C. for 16 hours. The reaction mixture was diluted with water (15 ml) and extracted with ethyl acetate (2×15 ml). The combined organic layers were washed with brine (15 ml), dried over anhydrous Na₂SO₄ and concentrated under reduced pressure. The residue was purified by silica gel chromatography to afford the title compound (0.3 g, 77%) as a solid. ¹H NMR (400 MHz, DMSO-d6): δ 0.49-0.56 (m, 2H), 0.75-0.83 (m, 2H), 1.70-1.77 (m, 1H), 5.65 (s, 2H, —NH₂), 6.36 (d, J=8.4 Hz, 1H), 7.04 (dd, J=8.4 Hz, 2.0 Hz, 1H), 7.74 (d, J=1.6 Hz, 1H). LCMS: m/z=135.2 [M+1].

### Method 8

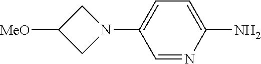 [SMILES: COC1CN(c2ccc(N)nc2)C1]

### Method 8. 5-(3-Methoxyazetidin-1-yl)pyridin-2-amine

To a stirred solution of 5-((1-methylpiperidin-4-yl)oxy)-2-nitropyridine (1.0 g, 4.78 mmol) in a mixture of Methanol (10 ml) was added 10% Pd/C (0.10 g, 10% w/w, 50% moisture). Then reaction mixture was stirred at room temperature under H2 gas atmosphere for 3 hours. After completion of reaction (monitored by TLC), the reaction mixture was diluted with Ethyl acetate and filtered through celite pad. The celite pad was washed with Ethyl acetate (2×25 ml). The combined filtrate was concentrated under reduced pressure to afford the title compound (0.22 g, 44%) as a solid. LCMS: m/z=180.3 [M+1].

### Method 9

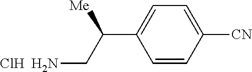

### (S)-4-(1-Aminopropan-2-yl)benzonitrile hydrochloride

### Method 9, Step 1. Ethyl (E,Z)-3-(4-cyanophenyl)but-2-enoate

To a stirred solution of potassium tert-butoxide (10.09 g, 89.7 mmol) in dry THE (90 ml) was added triethyl phosphonoacetate (20.08 g, 89.7 mmol) at 0° C. under an atmosphere of nitrogen. Then the reaction mixture was stirred for 15 minutes at the same temperature. The reaction was then warmed to room temperature and stirred for 1 hour. Then 4-acetylbenzonitrile (10.0 g, 69.0 mmol) was added as a solution in THE (50 ml) and the reaction was heated to 70° C. for 3 hours. After completion of reaction (monitored by TLC), the pH of the reaction mixture was adjusted to 3-4 with 1N HCl. The THF was removed under reduced pressure and the aqueous layer was extracted with ethyl acetate (2×50 ml). The combined organic layers were washed with brine (50 ml), dried over anhydrous Na₂SO₄ and concentrated under reduced pressure. The residue was purified by silica gel chromatography to afford the title compound (8.5 g, 58%). ¹H NMR (400 MHz, DMSO-d6): 1.15 (t, J=6.8 Hz, 1.5H), 1.36 (t, J=6.8 Hz, 3H), 2.21 (s, 1.5H), 2.60 (s, 3H), 4.05 (q, J=7.1 Hz, 1H), 4.27 (q, J=7.2 Hz, 2H), 6.01 (S, 0.5H), 6.19 (S, 1H), 7.30-7.71 (m, 6H).

### Method 9, Step 2. Ethyl 3-(4-cyanophenyl)butanoate

To a stirred solution of ethyl (E, Z) 3-(4-cyanophenyl)but-2-enoate (8.0 g, 37.2 mmol) in methanol:ethyl acetate (1:4, 140 ml) was added Pd/C (0.8 g, 10% w/w, 50% moisture). The reaction was stirred at room temperature under an atmosphere of hydrogen gas for 3 hours. The reaction mixture was diluted with ethyl acetate and filtered through a pad of celite. The combined organic layers were concentrated under reduced pressure to afford the title compound (4.5 g, 56%). ¹H NMR (400 MHz, CDCl₃): 1.23 (t, J=7.2 Hz, 3H), 1.33 (d, J=6.8 Hz, 3H), 2.62 (dd, J=7.6 Hz, 1.2 Hz, 2H), 3.70 (q, J=7.2 Hz, 1H), 4.07-4.15 (m, 2H), 7.37 (d, J=8.0 Hz, 2H), 7.37 (d, J=8.4 Hz, 2H).

### Method 9, Step 3. 3-(4-Cyanophenyl)butanoic acid

To a stirred solution of ethyl 3-(4-cyanophenyl)butanoate (4.5 g, 20.71 mmol) in a mixture of MeOH:THF:H₂O (4:2:1, 100 ml) was added LiOH (3.48 g, 82.95 mmol) at 5° C. to 10° C. The resulting reaction mixture was stirred at room temperature for 1.5 hours. After completion of reaction (monitored by TLC), the reaction solvent was evaporated. The residue was dissolved in water (10 ml) and extracted with ethyl acetate (2×15 ml). The pH of the aqueous layer adjusted to 3-4 with concentrated HCl. The precipitate that formed was filtered off to afford title compound (3.8 g, 97%) as a white solid. ¹H NMR (400 MHz, DMSO-d6): 1.23 (d, J=6.8, 3H), 2.58 (d, J=7.6 Hz, 2H), 3.24 (q, J=7.2, 1H), 7.49 (d, J=8.4 Hz, 2H), 7.77 (d, J=8.4 Hz, 2H), 12.15 (s, 1H).

### Method 9, Step 4. tert-Butyl (2-(4-cyanophenyl)propyl)carbamate

To a stirred solution of 3-(4-cyanophenyl)butanoic acid (5.0 g, 26.45 mmol) in tert-butanol (65 ml) was added triethylamine (11.0 ml, 79.36 mmol) at room temperature. Then the reaction mixture was cooled to 5-10° C. and was added DPPA (12.30 g, 44.97 mmol) drop wise. After formation of acylazide, the reaction was stirred at 90° C. overnight. The reaction mixture was diluted with water (40 ml) and extracted with ethyl acetate (2×40 ml). The combined organic layers were washed with brine (25 ml), dried over anhydrous Na₂SO₄ and concentrated under reduced pressure. The resulting residue was purified by silica gel chromatography to afford the title compound (4.5 g, 66%) as a solid. ¹H NMR (400 MHz, DMSO-d₆): 1.17 (d, J=6.8 Hz, 2H), 1.33 (s, 9H), 2.90-3.00 (m, 1H), 3.04-3.15 (m, 2H), 6.91 (t, J=5.2 Hz, 1H, —NH), 7.42 (d, J=8.4 Hz, 2H), 7.77 (d, J=7.2 Hz, 2H).

### Method 9, Step 5. 4-(1-Aminopropan-2-yl)benzonitrile hydrochloride

To a stirred solution of tert-butyl-(2-(4-cyanophenyl)propyl)carbamate (4.5 g, 17.29 mmol) in methanol (9 ml) was added a solution of 4M HCl in dioxane (10.8 ml, 2.4 vol.) drop wise at 0° C. The resulting mixture was stirred at room temperature for 2 hours. The reaction mixture was concentrated under reduced pressure to afford the title compound (2.81 g, 83%) as a solid. ¹H NMR (400 MHz, DMSO-d₆): 1.28 (d, J=6.8 Hz, 2H), 3.03 (d, J=5.6 Hz, 2H), 3.15-3.26 (m, 1H), 7.55 (d, J=8.0 Hz, 2H), 7.83 (d, J=8.0 Hz, 2H), 8.21 (s, 3H). LCMS: m/z=161.6 [M+1].

### Method 9, Step 6. 4-(1-Aminopropan-2-yl)benzonitrile

4-(1-aminopropan-2-yl)benzonitrile hydrochloride was treated with an aqueous solution of saturated sodium bicarbonate and extracted with ethyl acetate (3×30 ml) to obtained the crude compound as liquid which was further purified by silica gel chromatography (DCM:MeOH=90:10) to afford the racemic title compound a thick oil (2.29 g, 83%). ¹H NMR (400 MHz, CDCl₃): 1.28 (d, J=6.8 Hz, 3H), 2.85 (d, J=5.6 Hz, 3H), 7.34 (d, J=7.2 Hz, 2H), 7.63 (d, J=7.2 Hz, 2H). LCMS: m/z=161.5 [M+1]. The racemic amine may be resolved in the enantiopure title compound by preparative chiral SFC using a CHIRALPAK AD-H column (250 mm, 50 mm, 5 microns; mobile phase 25% Acetonitrile:Methanol:Dimethylamine (80:20:0.1) in 75% CO₂). The early eluting isomer has been unambiguously assigned as (S)-4-(1-aminopropan-2-yl)benzonitrile by obtaining an x-ray co-crystal structures of a truncated form of p300 with both example 22 (isomer 1; (S)-2-((4-cyanophenethyl)amino)-N-(5-(1-methyl-1H-pyrazol-4-yl)pyridin-2-yl)-2-phenylacetamide) and example 33 (isomer 4; (R, S)-2-((2-(4-cyanophenyl)-propyl)amino)-N-(5-(1-methyl-1H-pyrazol-4-yl)pyridin-2-yl)-2-phenylacetamide).

### Method 10

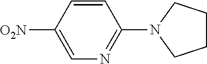 [SMILES: O=[N+]([O-])c1ccc(N2CCCC2)nc1]

### 5-Nitro-2-(pyrrolidin-1-yl)pyridine

### Method 10. 5-Nitro-2-(pyrrolidin-1-yl)pyridine

To a stirred solution of 2-bromo-5-nitro pyridine (0.5 g, 2.46 mmol) in DMSO (2 ml) was added pyrrolidine (0.262 g, 3.69 mmol) at room temperature. The reaction mixture was heated to 120° C. for 1 hour in the microwave. After completion of the reaction, ice cold water was added (15 ml) and the resulting precipitate was filtered through Buchner funnel to obtain the crude compound. The resulting crude compound was purified by trituration using n-hexanes (10 ml) to afford the title compound (0.370 g, 77%). LCMS: m/z=194.01 [M+1].

### Method 11

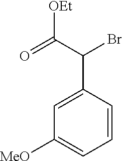 [SMILES: CCOC(=O)C(Br)c1cccc(OC)c1]

### Ethyl 2-bromo-2-(3-methoxyphenyl)acetate

### Method 11, Step 1. Ethyl 2-(3-methoxyphenyl)acetate

To a stirred solution of 3-methoxy-2-phenylacetic acid (5 g, 30 mmol) in absolute ethanol (50 ml), sulfuric acid (0.3 ml) was added at 0° C. and reaction mixture was refluxed at 70° C. for 2 hours. Reaction progress was monitored by TLC. After completion of the reaction, ethanol was removed by evaporation under reduced pressure. Then reaction mixture was neutralized with saturated solution of sodium bicarbonate and extracted with DCM (2×15 ml), dried over anhydrous Na₂SO₄ and concentrated under reduced pressure to afford the title compound (3.82 g, 81%) as colorless liquid. LCMS: m/z=195.26 [M+1].

### Method 11, Step 2. Ethyl 2-bromo-2-(3-methoxyphenyl)acetate

A mixture of ethyl 2-(3-methoxyphenyl)acetate (0.5 g, 2.5 mmol), N-bromosuccinamide (0.50 g, 2.80 mmol) and 2,2′-azobis(2-methylpropionitrile) (0.02, 0.12 mmol) in CCl₄ (10 ml) was refluxed for 2 hours. After completion of the reaction (monitored by TLC), the reaction mixture was diluted with n-hexanes and filtered through a pad of celite. The filtrate was dried over anhydrous Na₂SO₄ and concentrated under reduced pressure. The resulting compound was purified by silica gel chromatography to afford the title compound (0.8 g, 99%) as a yellowish liquid. LCMS: m/z=273.2 [M+1].

### Method 12

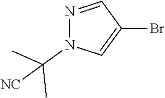

### 2-(4-Bromo-1H-pyrazol-1-yl)-2-methylpropanenitrile

### Method 12, Step 1. Methyl 2-(4-bromo-1H-pyrazol-1-yl)-2-methylpropanoate

To a stirred solution of 4-bromo-1H-pyrazole (3.0 g, 20.41 mmol) in dry DMF (30 ml) was added Cs₂CO₃ (19.95 g, 61.23 mmol) and methyl 2-bromo-2-methylpropanoate (3.96 ml, 30.61 mmol) at room temperature under an atmosphere of nitrogen. Then the reaction mixture was stirred at 80° C. for 18 hours. After completion of reaction (monitored by TLC), the reaction mixture was diluted with ice cold water (30 ml) and extracted with ethyl acetate (2×50 ml). The combined organic layers were washed with brine (50 ml), dried over anhydrous Na₂SO₄ and concentrated under reduced pressure. The resulting residue was purified by silica gel chromatography to afford the title compound (3.0 g, 60%). ¹H NMR (400 MHz, DMSO-d6): 1.76 (s, 6H), 4.63 (s, 3H), 7.61 (s, 1H), 8.21 (s, 1H).

### Method 12, Step 2. 2-(4-Bromo-1H-pyrazol-1-yl)-2-methylpropanamide

An oven dried autoclave was charged with methyl 2-(4-bromo-1H-pyrazol-1-yl)-2-methylpropanoate (1.0 g, 4.05 mmol) and CaCl₂) (0.5 g, 4.46 mmol) in methanol (10 ml). The reaction mixture was cooled −78° C. and NH₃ gas was purged in to it. Then the reaction was stirred for 20 hours at room temperature. After completion of reaction (monitored by TLC), the reaction mixture was diluted with water and extracted with ethyl acetate (2×30 ml). The combined organic layers were concentrated under reduced pressure and purified by silica gel chromatography to afford the title compound (0.55 g, 59%). ¹H NMR (400 MHz, DMSO-d₆): 1.70 (s, 6H), 6.96 (s, NH, 1H), 7.22 (s, NH, 1H), 7.60 (s, 1H), 8.09 (s, 1H).

### Method 12, Step 3. 2-(4-Bromo-1H-pyrazol-1-yl)-2-methylpropanenitrile

A solution of 2-(4-bromo-1H-pyrazol-1-yl)-2-methylpropanamide (0.5 g, 2.16 mmol) in POCl₃ (5 ml) was heated to 90° C. for 1.5 hours. After completion of reaction (confirmed by the TLC), reaction was quenched with saturated aqueous NaHCO₃ solution. The resulting mixture was extracted with ethyl acetate (2×20 ml). The combined organic layers were washed with water (2×20 ml), washed with brine (20 ml), dried over anhydrous Na₂SO₄ and concentrated under reduced pressure to afford the title compound (0.35 g, 75%) as a solid. ¹H NMR (400 MHz, DMSO-d6): 1.98 (s, 6H), 7.78 (s, 1H), 8.34 (s, 1H).

### Method 13

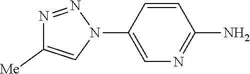 [SMILES: Cc1cn(-c2ccc(N)nc2)nn1]

### 5-(4-Methyl-1H-1, 2, 3-triazol-1-yl)pyridin2-amine

### Method 13. 5-(4-Methyl-1H-1, 2, 3-triazol-1-yl)pyridin2-amine

5-Iodo-2-aminopyridine (0.5 g, 2.28 mmol), NaN₃ (0.22 g, 3.41 mmol), K₂CO₃ (0.38 g, 2.76 mmol), CuSO₄.5H₂O (0.06 g, 0.23 mmol), sodium ascorbate (0.09 g, 0.46 mmol), L-Proline (0.06 g, 0.46 mmol) and 2-butynoic acid (0.28 g, 3.41 mmol) were combined in DMSO (6 ml) at room temperature. Then reaction mixture was heated at 65° C. for 6 hours. After completion of the reaction (monitored by TLC), the reaction was diluted with water (20 ml) and extracted with ethyl acetate (4×25 ml). The combined organic layers were dried over anhydrous Na₂SO₄ and concentrated under reduced pressure to give a residue that was was purified by silica gel chromatography to afford the title compound (0.25 g, 71%). LCMS: m/z 176.1 [M+1].

### Method 14

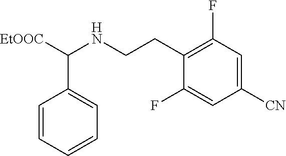 [SMILES: CCOC(=O)C(NCCc1c(F)cc(C#N)cc1F)c1ccccc1]

### Ethyl 2-((4-cyano-2,6-difluorophenEthyl)amino)-2-phenylacetate

### Method 14, Step 1. (E)-3,5-Difluoro-4-(2-methoxyvinyl)benzonitrile

To a stirred solution of methoxymethyltriphenylphosphonium chloride (1.47 g, 4.31 mmol) in THE (6 ml), potassium carbonate (0.594 g, 4.31 mmol) was added at 0 deg and stirred for 30 minutes at room temperature. To this 3,5-difluoro-4-formylbenzonitrile (0.6 g, 3.59 mmol) was added at room temperature and heated to reflux at 60 deg for 16 hours. The reaction mixture was quenched with water (30 ml) and extracted with ethyl acetate (2×30 ml). The organic layer was dried over sodium sulphate and concentrated under reduced pressure. The crude product was purified by silica gel chromatography to afford the title compound (0.24 g, 34%). ¹H NMR (400 MHz, DMSO-d6): δ 7.72-7.72 (m, 2H), 6.65 (d, J=6.4 Hz, 1H), 5.20 (d, J=6.4 Hz, 1H), 3.74 (s, 3H).

### Method 14, Step 2. 3,5-Difluoro-4-(2-oxoethyl)benzonitrile

(E)-3,5-difluoro-4-(2-methoxyvinyl)benzonitrile (0.120 g, 0.614 mmol) was dissolved in THE (3 ml) and 6N HCl (0.6 ml) was added into it. The reaction mixture was heated at 60° C. for 2 hours. The reaction mixture was neutralized with saturated sodium bicarbonate solution (10 ml) and extracted with ethyl acetate (2×20 ml). The organic layer was dried over sodium sulphate and concentrated under reduced pressure to afford the crude title compound which was used in next step without further purification (0.120 g). ¹H NMR (400 MHz, DMSO-d6): δ 9.71 (s, 1H), 7.84-7.86 (m, 2H), 4.10 (s, 2H).

### Method 14, Step 3. Ethyl 2-((4-cyano-2,6-difluorophenethyl)amino)-2-phenylacetate

To a solution of 3,5-difluoro-4-(2-oxoethyl)benzonitrile (0.120 g, 0.66 mmol) and ethyl 2-amino-2-phenylacetate (0.171 g, 0.79 mmol) in a mixture of 1:1 methanol:DCE (4 ml), acetic acid (4 drops) was added followed by powdered molecular sieves (0.1 g). The reaction mixture was stirred at room temperature for 1 hour. To this sodium cyanoborohydride (0.061 g, 0.99 mmol) was added and the reaction mixture was stirred at room temperature for 16 hours. The reaction mixture was quenched with sodium bicarbonate solution (5 ml) and extracted with ethyl acetate (3×10 ml). The organic layer was dried over sodium sulphate and concentrated under reduced pressure. The residue was purified by silica gel chromatography to afford the title compound (0.1 g, 44%). LCMS: m/z=345.5 [M+1].

### Method 15

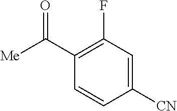 [SMILES: CC(=O)c1ccc(C#N)cc1F]

### 4-Acetyl-3-fluorobenzonitrile

### Method 15, Step 1. 4-Cyano-2-fluoro-N-methoxy-N-methylbenzamide

To a stirred solution of 4-cyano-2-fluorobenzoic acid (15 g, 90.84 mmol) in DMF (100 ml), HATU (51.81 g, 136.36 mmol) and DIPEA (58.70 g, 454.21 mmol) were added and the reaction mixture was stirred at room temperature for 1 hour. To this N, O-dimethyl hydroxylamine hydrochloride (26.60 g, 272.7 mmol) was added at 0 deg and the reaction mixture was stirred at room temperature for 5 hours. The reaction mixture was diluted with cold water (200 ml) and extracted with ethyl acetate (2×250 ml). The combined organic layers were washed with brine (100 ml), dried over anhydrous sodium sulfate, and concentrated under reduced pressure. The residue was purified by silica gel chromatography to afford the title compound (12.5 g, 66%). LCMS: m/z=209.1 [M+1].

### Method 15, Step 2. 4-Acetyl-3-fluorobenzonitrile

To a stirred solution of 4-cyano-2-fluoro-N-methoxy-N-methylbenzamide (15 g, 72.11 mmol) in THE (150 ml), methylmagnesium bromide (154.53 ml, 1.4M in 3:1 THF:Toluene, 216.34 mmol) was added drop wise at 0 deg and stirred for 30 minutes. The reaction mixture was quenched with ice cold water (150 ml) and extracted with ethyl acetate (2×250 ml). The combined organic layers were washed with brine (100 ml), dried over anhydrous sodium sulfate and concentrated under reduced pressure. The residue was purified by silica gel chromatography to afford the title compound (8.1 g, 69%). ¹H NMR (400 MHz, DMSO-d₆): δ 8.05-8.07 (m, 1H), 7.94-7.96 (m, 1H), 7.82-7.84 (m, 1H), 2.62 (s, 3H).

### Method 16

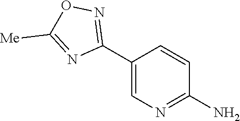 [SMILES: Cc1nc(-c2ccc(N)nc2)no1]

### 5-(5-Methyl-1,2,4-oxadiazol-3-yl)pyridin-2-amine

### Method 16, Step 1. N-(5-Cyanopyridin-2-yl)-4-methylbenzenesulfonamide

To a stirred solution of 6-aminonicotinonitrile (1 g, 8.39 mmol) in dry pyridine (30 ml) was added para-tosylchloride (3.2 g, 16.7 mmol) at 0° C. The reaction mixture was stirred at room temperature for 30 minutes. After 30 minutes, the reaction mixture was heated to 90° C. overnight. The solvent was removed and the residue was treated with water (25 ml). The obtained precipitates were collected by filtration and washed with water (25 ml) to afford the pure title compound (1.1 g, 50%). ¹H NMR (400 MHz, DMSO-d₆): δ 2.36 (s, 3H), 7.11 (d, J=8.8 Hz, 1H), 7.39 (d, J=8 Hz, 2H), 7.78-7.88 (m, 2H), 8.10 (dd, J=8.8 Hz, J=6.8 Hz, 1H), 8.62 (s, 1H), 11.89 (s, 1H). LCMS: m/z=274.26 [M+1].

### Method 16, Step 2. (Z)—N′-hydroxy-6-((4-methylphenyl)sulfonamido)-nicotinimidamide

A mixture of hydroxylamine hydrochloride (0.106 g, 1.53 mmol) and potassium carbonate (0.11 g, 0.80 mmol) in water (2 ml) was added to a solution of N-(5-cyanopyridin-2-yl)-4-methylbenzenesulfonamide (0.2 g, 0.732 mmol) in ethanol (8 ml). The reaction mixture was heated to reflux overnight. The reaction mixture was concentrated and the residue was treated with water (10 ml). The precipitated solid was collected by filtration and washed with water to obtain pure title compound (0.14 g, 62%). LCMS: m/z=307.61 [M+1].

### Method 16, Step 3. 4-Methyl-N-(5-(5-methyl-1,2,4-oxadiazol-3-yl)pyridin-2-yl)benzene-sulfonamide

To a stirred solution of (Z)—N-hydroxy-6-((4-methylphenyl)sulfonamido)-nicotinimidamide (0.72 g, 2.35 mmol) in DMSO (15 ml) was added ethyl acetate (0.35 ml, 3.52 mmol) and the reaction mixture was stirred for 15 minutes. To this, NaOH (0.141 g, 3.52 mmol) powder was added in one portion. After completion of reaction, the reaction was quenched with ice cold water (20 ml) and the aqueous layer was extracted with ethyl acetate (2×50 ml). The combined organic layers were washed with brine (25 ml), dried over anhydrous sodium sulfate and concentrated under reduced pressure to afford the title compound (0.25 g, 33%). ¹H NMR (400 MHz, DMSO-d₆): δ 2.36 (s, 3H), 2.65 (s, 3H), 7.24 (d, J=8.8 Hz, 1H), 7.39 (d, J=8 Hz, 2H), 7.85 (d, J=8 Hz, 2H), 8.21 (dd, J=8.8 Hz, J=6.4 Hz, 1H), 8.67 (s, 1H), 11.74 (s, 1H). LCMS: m/z=331.66 [M+1].

### Method 16, Step 4. 5-(5-Methyl-1,2,4-oxadiazol-3-yl)pyridin-2-amine

4-methyl-N-(5-(5-methyl-1,2,4-oxadiazol-3-yl)pyridin-2-yl)benzenesulfonamide (0.25 g, 0.75 mmol) was taken in vial and conc. H₂SO₄ (2.5 ml) was added at 0° C. After completion of reaction, the reaction was poured into a cooled solution of 50% NaOH (aq.). The obtained precipitate was filtered and washed with cooled water (20 ml). The solid was dried over high vacuum to afford the title compound (0.12 g, 90%). ¹H NMR (400 MHz, DMSO-d6): δ 2.60 (s, 3H), 6.53 (d, J=8.8 Hz, 1H), 6.62 (s, 2H), 7.86 (dd, J=8.4 Hz, 6.4 Hz, 1H), 8.50 (d, J=2 Hz, 1H). LCMS: m/z=177.51 [M+1].

### Method 17

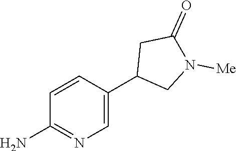 [SMILES: CN1CC(c2ccc(N)nc2)CC1=O]

### 4-(6-Fluoropyridin-3-yl)-1-methylpyrrolidin-2-one

### Method 17, Step 1. tert-Butyl 4-hydroxy-2-oxo-2,5-dihydro-1H-pyrrole-1-carboxylate

To a stirred solution of (tert-butoxycarbonyl)glycine (0.5 g, 2.85 mmol) in DCM was added 2,2-dimethyl-1,3-dioxane-4,6-dione (0.62 g, 4.28 mmol) and DMAP (0.52 g, 4.28 mmol) at room temperature. The reaction mixture was stirred for 15 minutes and EDC HCl (0.82 g, 4.28 mmol) was added at 0° C. The reaction mixture was further stirred at room temperature for 5 hours. After completion of reaction, the reaction mixture was diluted with ethyl acetate (100 ml) and the organic layer was washed with brine (50 ml), 20% aqueous citric acid solution (50 ml), and brine (50 ml). The organic layer was dried over sodium sulphate and evaporated to obtain the crude product. The obtained crude product was refluxed in ethyl acetate (50 ml) for 1 hour. After 1 hour, reaction mixture was concentrated to get pure desired compound (0.5 g, 88%). ¹H NMR (400 MHz, DMSO-d₆): δ 1.45-1.47 (m, 9H), 4.16 (s, 2H), 4.89 (s, 1H), 12.17 (s, 1H). LCMS: m/z=144.25 [M−56].

### Method 17, Step 2. tert-Butyl 2-oxo-4-(tosyloxy)-2,5-dihydro-1H-pyrrole-1-carboxylate

To a stirred solution of tert-butyl 4-hydroxy-2-oxo-2,5-dihydro-1H-pyrrole-1-carboxylate (0.5 g, 2.51 mmol) in DCM (25 ml) was added DIPEA (0.86 ml, 5.02 mmol) at room temperature. The reaction mixture was stirred for 15 minutes and cooled to 0° C. Then para-tosylchloride (0.47 g, 2.51 mmol) was added portion wise to the reaction mixture and the mixture was stirred at room temperature overnight. After completion of the reaction, the reaction mixture was diluted with saturated sodium bicarbonate (50 ml) and the product was extracted with ethyl acetate (2×50 ml). The combined organic layers were washed with brine (50 ml), dried over anhydrous sodium sulfate and concentrated under reduced pressure. The residue was purified by silica gel chromatography to afford the title compound (0.42 g, 48%). ¹H NMR (400 MHz, DMSO-d6): δ 1.44 (s, 9H), 2.46 (s, 3H), 4.36 (s, 2H), 5.80 (s, 1H), 7.56-7.58 (m, 2H), 8.01-8.03 (m, 2H). LCMS: m/z=298.36 [M−56].

### Method 17, Step 3. tert-Butyl 4-(6-fluoropyridin-3-yl)-2-oxo-2,5-dihydro-1H-pyrrole-1-carboxylate

To a mixture of tert-butyl 2-oxo-4-(tosyloxy)-2,5-dihydro-1H-pyrrole-1-carboxylate (1.0 g, 2.83 mmol) and (6-fluoropyridin-3-yl)boronic acid (0.598 g, 4.24 mmol) in 1,2-dimethoxyethane (30 ml) was added Pd(dppf)Cl₂-DCM complex (0.41 g, 0.56 mmol) at room temperature. To the reaction mixture 2M Sodium carbonate solution (10 ml) was added and the reaction mixture was purged with argon gas for 30 minutes. The reaction mixture was heated to 90° C. and stirred for 3 hours. The reaction mixture was diluted with water (50 ml) and extracted with ethyl acetate (2×50 ml). The combined organic layers were washed with brine (50 ml), dried over anhydrous sodium sulfate, and concentrated under reduced pressure. The residue was purified by column chromatography to afford the title compound (0.36 g, 57%). ¹H NMR (400 MHz, DMSO-d₆): δ 1.52 (s, 9H), 4.84 (s, 2H), 6.82 (s, 1H), 7.37 (dd, J=8.4 Hz, 6.0 Hz, 1H), 8.37-8.42 (m, 1H), 8.67 (s, 1H). LCMS: m/z=223.07 [M−56].

### Method 17, Step 4. 4-(6-Fluoropyridin-3-yl)-1,5-dihydro-2H-pyrrol-2-one

To a stirred solution of tert-butyl 4-(6-fluoropyridin-3-yl)-2-oxo-2,5-dihydro-1H-pyrrole-1-carboxylate (1.5 g, 5.39 mmol) in DCM (25 ml) was added TFA (5 ml) drop wise at room temperature. The reaction mixture was further stirred at room temperature for 3 hours. The mixture was concentrated and the residue was co-distilled with toluene twice to afford the title compound, which was directly used in the next step without further purification. LCMS: m/z=179.16 [M+1].

### Method 17, Step 5. 4-(6-Fluoropyridin-3-yl)pyrrolidin-2-one

To a stirred solution of 4-(6-fluoropyridin-3-yl)-1,5-dihydro-2H-pyrrol-2-one (0.25 g, 1.40 mmol) in methanol (2.5 ml) was added 10% Pd/C (0.25 g, 50% moisture). Then the reaction was stirred at room temperature under an atmosphere of hydrogen gas for 3 hours. The reaction mixture was diluted with methanol and filtered through a pad of celite. The eluent was concentrated under reduced pressure to afford the title compound (0.16 g, 44%). ¹H NMR (400 MHz, DMSO-d6): 2.33-2.40 (m, 1H), 3.18-3.27 (m, 2H), 3.60-3.74 (m, 2H), 7.17 (dd, J=8.4 Hz, J=6.0 Hz, 1H), 7.78 (s, 1H), 7.98-8.02 (m, 1H), 8.19 (s, 1H). LCMS: m/z=181.16 [M+1].

### Method 17, Step 6. 4-(6-Fluoropyridin-3-yl)-1-methylpyrrolidin-2-one

To a stirred solution of 4-(6-fluoropyridin-3-yl)pyrrolidin-2-one (0.16 g, 0.88 mmol) in DMF (3 ml) was added 60% NaH (0.053 g, 1.32 mmol) at 0° C. The reaction mixture was stirred at same temperature for 30 minutes and iodomethane (0.25 g, 1.77 mmol) was added. The reaction mixture was stirred at room temperature for another 2 hours. The reaction was quenched with cold water (30 ml) and extracted with ethyl acetate (2×50 ml). The combined organic layers were washed with brine (30 ml), dried over sodium sulphate, and concentrated. The residue was purified by silica gel chromatography to afford the title compound (0.12 g, 70%). ¹H NMR (400 MHz, DMSO-d6): δ 2.38-2.44 (m, 1H), 2.63-2.70 (m, 1H), 2.78 (s, 3H), 3.33-3.37 (m, 1H), 3.61-3.74 (m, 2H), 7.17-7.19 (m, 1H), 7.96-8.01 (m, 1H), 8.19 (s, 1H). LCMS: m/z=195.56 [M++1].

### Method 17, Step 7. 4-(6-Aminopyridin-3-yl)-1-methylpyrrolidin-2-one

A solution of 4-(6-fluoropyridin-3-yl)-1-methylpyrrolidin-2-one (0.30 g, 1.54 mmol) in ammonium hydroxide solution (3 ml) was stirred at 140° C. for 48 hours. The reaction mixture was concentrated under reduced pressure and the residue was purified by reverse phase HPLC to afford the title compound (0.10 g, 34%). ¹H NMR (400 MHz, DMSO-d6): δ 2.23-2.29 (m, 1H), 2.54-2.56 (m, 1H), 2.74 (s, 3H), 3.19-3.24 (m, 1H), 3.36-3.41 (m, 1H), 3.58-3.62 (m, 1H), 5.79 (s, 2H, —NH₂), 6.40 (d, J=8.4 Hz, 1H), 7.33 (dd, J=8.8 Hz, J=2.4 Hz, 1H), 7.79 (d, J=2.4 Hz, 1H). LCMS: m/z=192.20 [M+1].

### Method 18

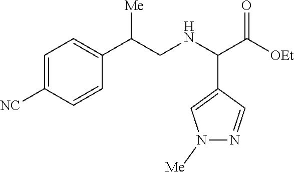 [SMILES: CCOC(=O)C(NCC(C)c1ccc(C#N)cc1)c1cnn(C)c1]

### Ethyl 2-((2-(4-cyanophenyl)propyl)amino)-2-(1-methyl-1H-pyrazol-4-yl)acetate

### Method 18, Step 1. 2-((2-(4-Cyanophenyl)propyl)amino)-2-(1-methyl-1H-pyrazol-4-yl)acetic acid

To a stirred solution of 4-(1-aminopropan-2-yl)benzonitrile hydrochloride (5 g, 30.86 mmol) in DCM (75 ml) were added TEA (3.12 g, 30.86 mmol), 2-oxoacetic acid (2.28 g, 30.86 mmol) and (1-methyl-1H-pyrazol-4-yl)boronic acid (3.80 g, 30.86 mmol) at room temperature. The reaction mixture was stirred at the same temperature for 15 minutes. After that HFIP (13.48 g, 80.24 mmol) was added and the reaction mixture was stirred for 16 hours at room temperature. The reaction was concentrated and the residue was stirred with DCM:pentane (3:7; 150 ml) for 30 minutes. A solid precipitated which was filtered on Buchner funnel and washed with n-pentane to afford title compound (5.5 g, 59%). LCMS: m/z=299 [M+1].

### Method 18, Step 2. Ethyl 2-((2-(4-cyanophenyl)propyl)amino)-2-(1-methyl-1H-pyrazol-4-yl)acetate

A mixture of 2-((2-(4-cyanophenyl)propyl)amino)-2-(1-methyl-1H-pyrazol-4-yl)acetic acid (5 g, 16.77 mmol) in DMF (100 ml) was heated at 80° C. until the reaction mixture became a clear solution. K₂CO₃ (5.79 g, 41.94 mmol) and ethyl iodide (2.61 g, 16.77 mmol) were added at the same temperature and the mixture was stirred for 30 minutes. The reaction mixture was then stirred at room temperature for 16 hours. The reaction was quenched with ice cold water (200 ml) and extracted with ethyl acetate (2×75 ml). The combined organic layers were washed with brine (100 ml), dried over anhydrous sodium sulfate, and concentrated under reduced pressure. The residue was purified by silica gel chromatography to afford the title compound (2.5 g, 45%) as a thick liquid. LCMS: m/z=327.7 [M+1].

### Method 19

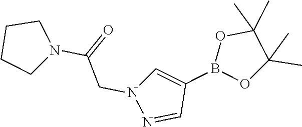 [SMILES: CC1(C)OB(c2cnn(CC(=O)N3CCCC3)c2)OC1(C)C]

### 1-(Pyrrolidin-1-yl)-2-(4-(4,4,5,5-tetramethyl-1,3,2-dioxaborolan-2-yl)-1H-pyrazol-1-yl)ethan-1-one

### Method 19, Step 1. 2-Chloro-1-(pyrrolidin-1-yl)ethan-1-one

Chloroacetyl chloride (3.4 ml, 42.18 mmol) was added dropwise to a stirred solution of pyrrolidine (2 g, 28.12 mmol) and triethylamine (11.7 ml, 84.36 mmol) in DCM (20 ml) cooled to 0° C. The reaction mixture was stirred at room temperature for 2 hours. The reaction mixture was poured into cold 1N HCl solution (20 ml) and extracted with DCM (2×30 ml). The combined organic layers were washed with brine (20 ml), dried over anhydrous sodium sulfate and concentrated under reduced pressure. The residue was purified by silica gel chromatography to afford the title compound (1.1 g, 26%). ¹H NMR (400 MHz, DMSO-d6): δ 4.30 (s, 2H), 3.44-3.47 (m, 2H), 3.30-3.35 (m, 2H), 1.86-1.93 (m, 2H), 1.77-1.82 (m, 2H). LCMS: m/z=148.05 [M+1].

### Method 19, Step 2. 1-(Pyrrolidin-1-yl)-2-(4-(4,4,5,5-tetramethyl-1,3,2-dioxaborolan-2-yl)-1H-pyrazol-1-yl)ethan-1-one

To a stirred solution of 4-(4,4,5,5-tetramethyl-1,3,2-dioxaborolan-2-yl)-1H-pyrazole (0.943 g, 4.86 mmol) in dry DMF (10 ml) was added NaH (0.213 g, 60%, 5.34 mmol) at 0° C. The reaction mixture was stirred at room temperature for 15 minutes. To this 2-chloro-1-(pyrrolidin-1-yl)ethan-1-one (1.0 g, 7.29 mmol) was added at 0° C. and stirred for 30 minutes at same temperature. The reaction mixture was then stirred at room temperature for another 1 hour. The reaction mixture was poured into ice cold water (20 ml) and extracted with DCM (2×30 ml). The combined organic layers were washed with brine (20 ml), dried over anhydrous sodium sulfate and concentrated under reduced pressure. The crude product was purified by silica gel chromatography to afford the title compound (0.81 g, 58%). LCMS: m/z=306.28 [M+1].

### Method 20

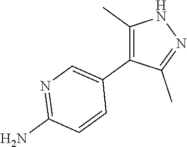

### 5-(3,5-Dimethyl-1H-pyrazol-4-yl)pyridin-2-amine

### Method 24, Step 1. 5-(3,5-Dimethyl-1H-pyrazol-4-yl)pyridin-2-amine

A stirred solution of tert-butyl 4-(6-aminopyridin-3-yl)-3,5-dimethyl-1H-pyrazole-1-carboxylate (0.3 g, 1.04 mmol) in DCM (3 ml) was cooled to 0° C. and HCl (2.2 ml, 8.79 mmol; 4M in 1,4-dioxane) was added drop wise. The reaction mixture was allowed to warm to room temperature and was stirred for 3.5 hours. The reaction mixture was concentrated under reduced pressure to obtain the crude product. The crude product was triturated using n-pentane (3×5 ml) and the solid was filtered off to afford the title compound (175 mg, 75%). LCMS: m/z=189.21 [M+1].

### Method 21

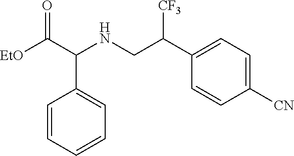 [SMILES: CCOC(=O)C(NCC(c1ccc(C#N)cc1)C(F)(F)F)c1ccccc1]

### Ethyl 2-((2-(4-cyanophenyl)-3,3,3-trifluoropropyl)amino)-2-phenylacetate

### Method 21, Step 1. 4-(2,2,2-Trifluoroacetyl)benzonitrile

To a stirred solution of methyl 4-cyanobenzoate (1.5 g, 9.31 mmol) in dry THE (30 ml) was added trifluoromethyltrimethylsilane (1.98 g, 13.97 mmol) and cesium fluoride (0.14 g, 0.93 mmol) at room temperature and the reaction mixture was stirred for one hour. The pH of the reaction mixture was adjusted to 5-6 with 1N HCl and the aqueous layer was extracted with ethyl acetate (2×50 ml). The combined organic layers were washed with brine (50 ml), dried over anhydrous sodium sulfate and concentrated under reduced pressure. To the resulting residue, TBAF (9.31 ml, 1M in THF, 9.31 mmol) and water (10 ml) was added at room temperature. The reaction mixture was stirred for one hour. Water (50 ml) was added and it the mixture was extracted with ethyl acetate (2×50 ml). The combined organic layers were washed with brine (50 ml), dried over anhydrous sodium sulfate and concentrated under reduced pressure. The residue was purified by silica gel chromatography to afford the title compound (1 g, 51%). ¹H NMR (400 MHz, DMSO-d6): δ 7.77 (d, J=8.4 Hz, 2H), 7.90 (d, J=8.0 Hz, 2H).

### Method 21, Step 2. Ethyl (Z)-3-(4-cyanophenyl)-4,4,4-trifluorobut-2-enoate

To a stirred solution of potassium tert-butoxide (0.12 g, 1.1 mmol) in dry THE (4 ml) was added triethyl phosphonoacetate (0.27 g, 1.1 mmol) at −5 to 0° C. under an atmosphere of nitrogen. Then reaction mixture was stirred for 15 minutes at the same temperature. Then the reaction was warmed to room temperature and stirred for an additional hour. 4-(2,2,2-trifluoroacetyl)benzonitrile (0.2 g, 0.92 mmol) in THE (2 ml) was added to the reaction mixture and reaction was heated to 70° C. for 2 hours. The pH of the reaction mixture was adjusted to 3 to 4 with 1N HCl and THE was then removed under reduced pressure. The aqueous layer was extracted with ethyl acetate (2×50 ml) and the combined organic layers were washed with brine (50 ml), dried over anhydrous sodium sulfate and concentrated under reduced pressure. The resulting residue was purified by silica gel chromatography to afford the title compound (0.1 g, 37%). ¹H NMR (400 MHz, CDCl₃): δ 1.16 (t, J=7.2 Hz, 3H), 4.08-4.14 (m, 2H), 6.71 (s, 1H), 7.45 (d, J=7.6 Hz, 2H), 7.76 (d, J=8.0 Hz, 2H).

### Method 21, Step 3. Ethyl 3-(4-cyanophenyl)-4,4,4-trifluorobutanoate

To a stirred solution of ethyl (Z)-3-(4-cyanophenyl)-4,4,4-trifluorobut-2-enoate (0.1 g, 0.37 mmol) in 1:4 methanol:ethyl acetate (1.5 ml) was carefully added Pd/C (0.02 g, 20% w/w, 50% moisture). The reaction mixture was stirred at room temperature under an atmosphere of hydrogen for 3 hours. Then the reaction mixture was diluted with ethyl acetate (5 ml) and filtered through a pad of celite. The celite pad was washed with ethyl acetate (2×10 ml). The combined filtrate was concentrated under reduced pressure to afford the title compound (0.1 g, Quantitative). ¹H NMR (400 MHz, CDCl₃): δ 1.07 (t, J=6.8 Hz, 3H), 3.13-3.15 (m, 2H), 3.95-4.04 (m, 2H), 4.27-4.33 (m, 1H), 7.71 (d, J=8.0 Hz, 2H), 7.89 (d, J=8.4 Hz, 2H).

### Method 21, Step 4. 3-(4-Cyanophenyl)-4,4,4-trifluorobutanoic acid

To a stirred solution of ethyl 3-(4-cyanophenyl)-4,4,4-trifluorobutanoate (0.28 g, 1.03 mmol) in a mixture of MeOH:THF:H₂O (4:2:1, 10 ml) was added LiOH H₂O (0.08 g, 1.93 mmol) at 5 to 10° C. The resulting reaction mixture was stirred at room temperature for 1.5 hours. Then the organic solvents were removed by evaporation. The crude material was dissolved in water (10 ml) and extracted with ethyl acetate (2×15 ml). The pH of the aqueous layer was adjusted to 3-4 with concentrated HCl. The desired compound precipitated during this process and the solid product was filtered off to afford title compound (0.2 g, 74%) as a white solid. ¹H NMR (400 MHz, DMSO-d₆): δ 3.02 (d, J=7.6 Hz, 2H), 4.21-4.25 (m, 1H), 7.68 (d, J=8.4, 2H), 7.87 (d, J=8.0 Hz, 2H), 12.52 (s, 1H).

### Method 21, Step 5. tert-Butyl (2-(4-cyanophenyl)-3,3,3-trifluoropropyl)carbamate

To a stirred solution of 3-(4-cyanophenyl)-4,4,4-trifluorobutanoic acid (0.5 g, 2.05 mmol) in tert-butanol (5 ml) was added triethylamine (0.86 ml, 5.96 mmol) at room temperature. Then the reaction mixture was cooled to 5-10° C. and DPPA (0.96 g, 3.49 mmol) was added drop wise. After formation of the acylazide as confirmed by TLC (after 1 hour), the reaction was stirred at 90° C. overnight. Then the reaction mixture was diluted with water (30 ml) and extracted with ethyl acetate (2×30 ml). The combined organic layers were washed with brine (25 ml), dried over anhydrous sodium sulphate and concentrated under reduced pressure. The resulting residue was purified by silica gel chromatography to afford the title compound (0.3 g, 46%) as solid. ¹H NMR (400 MHz, DMSO-d₆): δ 1.33 (s, 9H), 2.90-3.00 (m, 1H), 3.04-3.15 (m, 2H), 6.91 (t, J=5.2 Hz, 1H, —NH), 7.42 (d, J=8.4 Hz, 2H), 7.77 (d, J=7.2 Hz, 2H).

### Method 21, Step 6. 4-(3-Amino-1,1,1-trifluoropropan-2-yl)benzonitrile hydrochloride

To a stirred solution of tert-butyl (2-(4-cyanophenyl)-3,3,3-trifluoropropyl)carbamate (0.1 g, 0.31 mmol) in methanol (1 ml) was added a solution of 4M HCl in dioxane (0.24 ml, 2.4 vol.) drop wise at 0° C. The resulting mixture was stirred at room temperature for 2 hours. The reaction mixture was concentrated under reduced pressure to afford the title compound (0.05 g, 63%) as solid. ¹H NMR (400 MHz, DMSO-d₆): δ 3.52-3.57 (m, 2H), 4.33-4.41 (m, 1H), 7.73 (d, J=8.0 Hz, 2H), 7.97 (d, J=8.4 Hz, 2H), 8.36 (s, 3H, —HCl). LCMS: m/z=215.1 [M+1].

### Method 21, Step 7. Ethyl 2-((2-(4-cyanophenyl)-3,3,3-trifluoropropyl)amino)-2-phenylacetate

A mixture of ethyl 2-bromo-2-phenylacetate (0.40 g, 1.44 mmol), 4-(3-amino-1,1,1-trifluoropropan-2-yl)benzonitrile hydrochloride (0.3 g, 1.20 mmol) and triethylamine (0.58 ml, 4.20 mmol) in DMF (3 ml) was heated at 60° C. for 3 hours. The reaction mixture was poured into ice cold water (50 ml) and extracted with ethyl acetate (2×50 ml). The combined organic layers were washed with brine (25 ml), dried over anhydrous sodium sulphate and concentrated under reduced pressure. The resulting residue was purified by silica gel chromatography to afford the title compound (0.40 g, 76%) as a thick yellow oil. ¹H NMR (400 MHz, DMSO-d6): δ 1.08-1.11 (m, 3H), 2.59-2.61 (m, 2H), 2.99-3.10 (m, 3H), 4.01-4.11 (m, 3H), 4.40-4.47 (m, 1H), 7.23-7.36 (m, 5H), 7.55-7.61 (m, 2H), 7.88 (t, J=8 Hz, 2H). LCMS: m/z=377.62 [M+1].

### Method 22

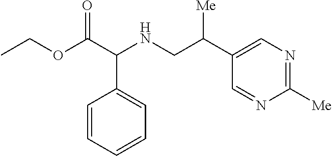 [SMILES: CCOC(=O)C(NCC(C)c1cnc(C)nc1)c1ccccc1]

### Ethyl 2-((2-(2-methylpyrimidin-5-yl)propyl)amino)-2-phenylacetate

### Method 22, Step 1. Methyl (Z)-3-(2-methylpyrimidin-5-yl)but-2-enoate

To a stirred solution of 5-bromo-2-methylpyrimidine (5 g, 28.9 mmol) in dry DMF (3 ml) was added methyl crotonate (3.75 g, 37.57 mmol) at room temperature. To this mixture, Pd(OAc)₂ (0.64 g, 2.89 mmol), tri(o-tolyl)phosphine (0.88 g, 2.89 mmol) and triethylamine (4.80 ml, 34.68 mmol) were added at room temperature. The reaction mixture was then purged with argon for 20 minutes. Then the mixture was heated to 100° C. overnight. Then the reaction mixture was poured into ice cold water (50 ml) and extracted with ethyl acetate (3×50 ml). The combined organic layers were washed with brine (50 ml), dried over anhydrous sodium sulphate and concentrated under reduced pressure. The resulting residue was purified by silica gel chromatography to afford the title compound (1.8 g, 32%). ¹H NMR (400 MHz, DMSO-d₆): δ 2.61 (s, 3H), 2.80 (s, 3H), 3.82 (s, 3H), 6.20 (s, 1H), 8.77 (s, 2H). LCMS: m/z=193.3 [M+1].

### Method 22, Step 2. Methyl 3-(2-methylpyrimidin-5-yl)butanoate

A stirred solution of methyl (Z)-3-(2-methylpyrimidin-5-yl)but-2-enoate (1.8 g, 9.37 mmol) in 1:1 methanol:Ethyl acetate (20 ml) was added 10% Pd/C (0.18 g, 10% w/w, 50% moisture) at room temperature. The reaction mixture was stirred overnight under hydrogen gas pressure (100 psi). The reaction mixture was then filtered through a pad of celite and washed with 1:1 methanol:ethyl acetate (50 ml). The filtrate was concentrated under reduced pressure to afford the title compound (1.2 g, 66%). LCMS: m/z=195.5 [M+1].

### Method 22, Step 3. 3-(2-Methylpyrimidin-5-yl)butanoic acid

To a stirred solution of methyl 3-(2-methylpyrimidin-5-yl)butanoate (1.2 g, 6.18 mmol) in a mixture of MeOH:THF:H₂O (4:2:1, 10 ml) was added LiOH H₂O (0.38 g, 9.23 mmol) at 5 to 10° C. The resulting reaction mixture was stirred at room temperature for 2 hours. Then the reaction solvent was evaporated and the resulting residue was dissolved in water (10 ml) and extracted with ethyl acetate (2×15 ml). The pH of the aqueous layer was adjusted to 3-4 with concentrated HCl. The desired compound precipitated during this processs and the solid filtered off to afford title compound (0.6 g, 54%) as a white solid. LCMS: m/z=181.2 [M+1].

Method 22, Step 4. tert-Butyl (2-(2-methylpyrimidin-5-yl)propyl)carbamate:

To a stirred solution of 3-(2-methylpyrimidin-5-yl)butanoic acid (0.6 g, 3.33 mmol) in tert-butanol (6 ml) was added triethylamine (1.37 ml, 9.99 mmol) at room temperature. Then the reaction mixture was cooled to 5-10° C. and DPPA (1.5 g, 5.45 mmol) was added drop wise. After formation of acylazide as confirmed by the TLC, the reaction mixture was stirred at 90° C. overnight. Then the reaction mixture was diluted with water (30 ml) and extracted with ethyl acetate (2×30 ml). The combined organic layers were washed with brine (25 ml), dried over anhydrous sodium sulphate and concentrated under reduced pressure. The resulting residue was purified by silica gel chromatography to afford the title compound (0.4 g, 47%) as thick oil. LCMS: m/z=252.2 [M+1].

### Method 22, Step 5. 2-(2-Methylpyrimidin-5-yl)propan-1-amine hydrochloride

To a stirred solution of tert-butyl (2-(2-methylpyrimidin-5-yl)propyl)carbamate (0.4 g, 1.59 mmol) in methanol (4 ml) was added a solution of 4M HCl in dioxane (0.96 ml, 2.4 vol.) drop wise at 0° C. The resulting mixture was stirred at room temperature for 2 hours. The reaction mixture was concentrated under reduced pressure to afford the title compound (0.26 g) as solid. LCMS: m/z=152.1 [M+1].

### Method 22, Step 6. Ethyl 2-((2-(2-methylpyrimidin-5-yl)propyl)amino)-2-phenylacetate

A mixture of ethyl 2-bromo-2-phenylacetate (0.37 g, 1.52 mmol), 2-(2-methylpyrimidin-5-yl)propan-1-amine hydrochloride (0.26 g, 1.38 mmol) and triethylamine (0.41 ml, 3.04 mmol) in DMF (3 ml) was heated at 60° C. for 3 hours. The reaction mixture was poured into ice cold water (50 ml) and extracted with ethyl acetate (2×50 ml). The combined organic layers were washed with brine (25 ml), dried over anhydrous sodium sulphate and concentrated under reduced pressure to obtain crude product (0.32 g). LCMS: m/z=314.6 [M+1].

### Method 23

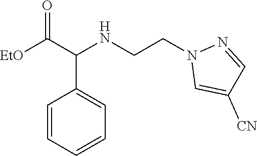 [SMILES: CCOC(=O)C(NCCn1cc(C#N)cn1)c1ccccc1]

### Ethyl 2-((2-(4-cyano-1H-pyrazol-1-yl)ethyl)amino)-2-phenylacetate

### Method 23. Ethyl 2-((2-(4-cyano-1H-pyrazol-1-yl)ethyl)amino)-2-phenylacetate

To a stirred solution of ethyl 2-((2-chloroethyl) amino)-2-phenylacetate (0.10 g, 0.413 mmol) in DMF (1 ml) was added K₂CO₃ (0.114 g, 0.827 mmol) at 25° C. After stirring for 15 minutes, 1H-pyrazole-4-carbonitrile (0.046 g, 0.496 mmol) was added at 25° C. The reaction mixture was heated at 60° C. for 3 hours. Then the reaction mixture was poured into ice water (15 ml) and the product was extracted with ethyl acetate (2×30 ml). The combined organic layers were washed with brine (20 ml), dried over anhydrous sodium sulphate and concentrated under reduced pressure. The residue was purified by silica gel chromatography to afford the title compound (0.050 g, 40%). LCMS: m/z=299.76 [M+1].

### Method 24

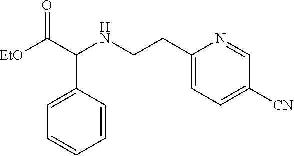 [SMILES: CCOC(=O)C(NCCc1ccc(C#N)cn1)c1ccccc1]

### Ethyl 2-((2-(5-cyanopyridin-2-yl)ethyl)amino)-2-phenylacetate

### Method 24, Step 1. 6-Vinylnicotinonitrile

A mixture of 6-bromonicotinonitrile (2.0 g, 10.92 mmol), 4,4,5,5-tetramethyl-2-vinyl-1,3,2-dioxaborolane (2.5 g, 16.39 mmol) and sodium carbonate (1.4 g, 13.50 mmol) in 4:1 dioxane:water (25 ml) were purged for 20 minutes with argon. To the reaction mixture, Pd(PPh₃)₄ (0.63 g, 0.54 mmol) was added and purging with argon was continued for another 10 minutes. The reaction mixture was heated at 90° C. for 12 hours. The reaction mixture was poured into water (50 ml) and extracted with ethyl acetate (2×100 ml). The combined organic layers were washed with brine (50 ml), dried over anhydrous sodium sulphate and concentrated under reduced pressure. The crude product was purified by silica gel chromatography to afford the title compound (0.7 g, 49%) as solid. ¹H NMR (400 MHz, DMSO-d6): δ 5.72 (d, J=10.8 Hz, 1H), 6.42 (d, J=17.2 Hz, 1H), 6.82-6.89 (m, 1H), 7.44 (d, J=8.0 Hz, 1H), 7.93 (dd, J=8.0 Hz, 2.0 Hz, 1H), 8.85 (s, 1H). LCMS: m/z=131.3 [M+1].

### Method 24, Step 2. Ethyl 2-((2-(5-cyanopyridin-2-yl)ethyl)amino)-2-phenylacetate

To a stirred solution of 6-vinylnicotinonitrile (0.20 g, 15.0 mmol) in ethanol (2 ml) was added triethylamine (2.0 ml, 15.0 mmol) and ethyl 2-amino-2-phenylacetate (0.30 g, 16.7 mmol) at 25° C. The reaction mixture was heated at 90° C. for 5 hours. The reaction mixture was concentrated under reduced pressure and to the residue, water (25 ml) was added. The aqueous layer was extracted with ethyl acetate (2×30 ml). The combined organic layers were washed with brine (20 ml), dried over anhydrous sodium sulphate and concentrated under reduced pressure. The crude product was purified by silica gel chromatography to afford the title compound (0.3 g, 40%). ¹H NMR (400 MHz, DMSO-d6): δ 1.23 (t, J=7.2 Hz, 3H), 2.93-2.98 (m, 1H), 3.03-3.11 (m, 3H), 4.12-4.26 (m, 2H), 4.40 (s, 1H), 7.30-7.43 (m, 7H), 7.89 (dd, J=8.0 Hz, 2.0 Hz, 1H), LCMS: m/z=310.36 [M+1].

### Method 25

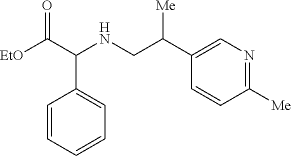 [SMILES: CCOC(=O)C(NCC(C)c1ccc(C)nc1)c1ccccc1]

### Ethyl 2-((2-(6-methylpyridin-3-yl)propyl)amino)-2-phenylacetate

### Method 25, Step 1. 2-Methyl-5-(prop-1-en-2-yl)pyridine

A mixture of 5-bromo-2-methylpyridine (2.0 g, 11.62 mmol), potassium isopropenyl trifluoroborate (2.5 g, 17.43 mmol) and cesium carbonate (11.3 g, 34.88 mmol) in 4:1 isopropanol:water (50 ml) were purged for 20 minutes with argon. To the reaction mixture, Pd(dppf)Cl₂.DCM (0.84 g, 1.16 mmol) was added and purging with argon was continued for another 10 minutes. The reaction mixture was heated at 100° C. for 2-3 hours. The reaction mixture was poured into water (50 ml) and extracted with ethyl acetate (2×100 ml). The combined organic layers were washed with brine (50 ml), dried over anhydrous sodium sulphate and concentrated under reduced pressure. The residue was purified by silica gel chromatography to afford the title compound (1.03 g, 49%) as solid. LCMS: m/z=134.5 [M+1].

### Method 25, Step 2. (E)-2-Methyl-5-(1-nitroprop-1-en-2-yl)pyridine

To a stirred solution of 1-chloro-4-(prop-1-en-2-yl)benzene (0.5 g, 3.73 mmol) in dry DCE (5.0 ml) under an atmosphere of nitrogen was added AgNO₂ (1.72 g, 11.19 mmol), TEMPO (0.23 g, 1.49 mmol), 4 A molecular sieves (1.5 g) at room temperature. The resulting reaction mixture was stirred for 10 minutes at room temperature and then it was heated to 70° C. overnight. The reaction mixture was cooled to room temperature and it was diluted with dichloromethane (50 ml). The mixture was then filtered through a pad of celite pad and the pad was washed with dichloromethane (50 ml). The eluent was then concentrated under reduced pressure. The residue was purified by silica gel column chromatography to afford the title compound (0.4 g, 30%) as light yellow solid. LCMS: m/z=179.5 [M+1].

### Method 25, Step 3. 2-(6-Methylpyridin-3-yl)propan-1-amine

To a solution of (E)-1-chloro-4-(1-nitroprop-1-en-2-yl)benzene (0.45 g, 2.52 mmol) in dry THE (5 ml) under an atmosphere of nitrogen atmosphere was added LAH (5.0 ml, 1M in THF, 5.05 mmol) drop wise at 0° C. The reaction mixture was stirred overnight at room temperature. Saturated aqueous sodium bicarbonate solution (50 ml) was added and reaction mixture was extracted with ethyl acetate (3×50 ml). The combined organic layers were washed with brine, dried over anhydrous sodium sulphate and concentrated under reduced pressure to afford the title compound (0.35 g) as yellow oil. This was used in the next step without further purification.

### Method 25, Step 4. Ethyl 2-((2-(6-methylpyridin-3-yl)propyl)amino)-2-phenylacetate

A mixture of ethyl 2-bromo-2-phenylacetate (0.37 g, 1.55 mmol), 2-(6-methylpyridin-3-yl)propan-1-amine (0.35 g, 2.32 mmol) and triethylamine (0.58 ml, 3.10 mmol) in DMF (5 ml) was heated at 60° C. for 3 hours. The reaction mixture was poured into ice cold water (50 ml) and extracted with ethyl acetate (2×50 ml). The combined organic layers were washed with brine (25 ml), dried over anhydrous sodium sulphate and concentrated under reduced pressure to give the crude product which was purified by silica gel column chromatography to afford the title compound (0.15 g, 20%). LCMS: m/z=313.1 [M+1].

### Scheme 1

The starting materials required for the synthesis of examples prepared using Scheme 1 were either commercially available or prepared using methods 1 through 3.

### Example 1

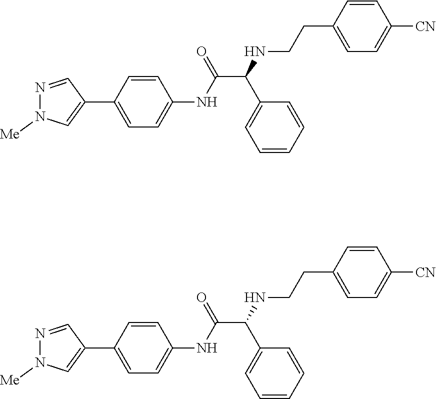 [SMILES: Cn1cc(-c2ccc(NC(=O)[C@@H](NCCc3ccc(C#N)cc3)c3ccccc3)cc2)cn1 ; Cn1cc(-c2ccc(NC(=O)[C@H](NCCc3ccc(C#N)cc3)c3ccccc3)cc2)cn1]

### (S)- and (R)-2-((4-Cyanophenethyl)amino)-N-(4-(1-methyl-1H-pyrazol-4-yl)phenyl)-2-phenylacetamide

### Scheme 1. (S)- and (R)-2-((4-Cyanophenethyl)amino)-N-(4-(1-methyl-1H-pyrazol-4-yl)phenyl)-2-phenylacetamide

A mixture of 2-bromo-N-(4-(1-methyl-1H-pyrazol-4-yl)phenyl)-2-phenylacetamide (0.5 g, 1.35 mmol), 4-(2-aminoethyl)benzonitrile hydrochloride (0.296 g, 2.7 mmol) and TEA (0.6 ml, 4.05 mmol) in DMF (5 ml) was heated for 2 hours at 60° C. After completion of the reaction, the reaction mixture was poured into ice cold water (15 ml) and extracted with ethyl acetate (2×30 ml). The combined organic layers were washed with brine (15 ml), dried over anhydrous Na₂SO₄ and concentrated under reduced pressure. The resulting residue was purified by silica gel chromatography to afford the title compound as solid (0.35 g, 59%) in racemic form.

The racemic title compound was resolved by chiral HPLC (CHIRALPAK AD-H; 30% (50:50 ACN:IPA in liquid CO₂+0.1% DEA) to furnish the enantiopure compounds. The faster-eluting enantiomer of the title compound was obtained as a solid (Isomer 1): ¹H NMR (400 MHz, DMSO-d6): δ 2.76-2.78 (m, 2H), 2.86-2.88 (m, 2H), 3.85 (s, 3H), 4.38 (s, 1H), 7.28-7.37 (m, 3H), 7.45-7.49 (m, 6H), 7.53 (d, J=8.8 Hz, 2H), 7.75 (d, J=8.4 Hz, 2H), 7.79 (s, 1H), 8.06 (s, 1H), 10.04 (s, 1H). LCMS: m/z=436.5 [M+1]. The slower-eluting enantiomer of the title compound was obtained as a solid (Isomer 2: ¹H NMR (400 MHz, DMSO-d6): δ 2.76-2.79 (m, 2H), 2.86-2.89 (m, 2H), 3.85 (s, 3H), 4.38 (s, 1H), 7.28-7.37 (m, 3H), 7.44-7.49 (m, 6H), 7.53 (d, J=8.8 Hz, 2H), 7.75 (d, J=8.0 Hz, 2H), 7.79 (s, 1H), 8.06 (s, 1H), 10.03 (s, 1H). LCMS: m/z=436.5 [M+1].

The following compounds were prepared using similar procedures to those described for Example 1 using the appropriate starting materials. The separated isomers for each compound are listed in the order to which they elute. For example, in instances where there are two isomers, isomer 1 is the faster eluting isomer and isomer 2 is the slower-eluting isomer. In instances where there are four isomers, isomer 1 is the fastest eluting isomer followed by isomer 2, then isomer 3, and then isomer 4. Additionally, when more than one chiral column is listed the columns are used in sequential order as listed. For example, if three columns are listed for the purification of a compound with 2 stereocenters, the first was used to separate the mixture into two mixtures, of stereoisomers 1 and 2 and stereoisomers 3 and 4. The mixture of stereoisomers 1 and 2 were then further separated into the pure stereoisomer by the second column listed and the mixture of stereoisomers 3 and 4 were separated into the pure stereoisomers by the third column listed. In some instances, a single chiral column may resolve all four stereoisomers. Additionally, one column may resolve the mixture into pure stereoisomer 1, pure stereoisomer 2, and a mixture of stereoisomers 3 and 4 and a second chiral column is used to resolve the mixture. The stereochemical representation (i.e., R or S) of each isomer of a compound is not drawn in the table and rather named to make clear that support for both is intended. Chiral carbon atom(s) are designated by the asterisk (*). In some instances, chiral building blocks are used to prepare compounds with multiple stereocenters and certain stereoisomers have not been prepared. In these instances where the stereochemistry of one stereocenter is known it will be drawn as such and the other stereocenters that have not been unambiguously assigned will be designated by an asterisk (*). In instances where a compound is racemic, it has been noted as such. In one aspect, the present disclosure relates to the racemic form of any compound described herein. These conventions are followed throughout the entirety of the application.

TABLE 1

| | | | | Chiral |
|---|---|---|---|---|
| | | | | Column; |
| Example | Structure | | Exact Mass | Mobile |
| Nos. | (Methods and Schemes for Preparation) | IUPAC Name | [M + 1] | Phase |
| 2 Isomer 1 Isomer 2 Isomer 3 Isomer 4 | 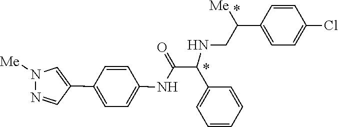 [SMILES: CC(CNC(C(=O)Nc1ccc(-c2cnn(C)c2)cc1)c1ccccc1)c1ccc(Cl)cc1] | (S, S)-, (S, R)-, (R, R)-, and (R, S)-2-((2-(4- chlorophenyl)propyl) amino)-N-(4-(1- methyl-1H-pyrazol-4- yl)phenyl)-2- phenylacetamide | Calc’d 459.2, Found 459.4, 459.2, 4 59.5 and 459.5 | CHIRALPAK AD-H; 30% (50:50 MeOH:IPA) in hexanes + 0.1% DEA |
| 3 Isomer 1 Isomer 2 Isomer 3 Isomer 4 | 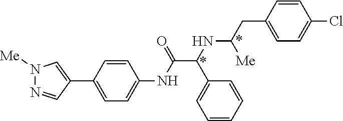 [SMILES: CC(Cc1ccc(Cl)cc1)NC(C(=O)Nc1ccc(-c2cnn(C)c2)cc1)c1ccccc1] | (S, S)-, (S, R)-, (R, R)-, and (R, S)-2-((1-(4- chlorophenyl)propan- 2-yl)amino)-N-(4-(1- methyl-1H-pyrazol-4- yl)phenyl)-2- phenylacetamide | Calc’d 459.2, Found 459.5, 459.5, 459.5 and 459.5 | 1- CHIRALCEL IC; 25% Me0H in Liquid CO₂ + 0.1% DEA 2- CHIRALPAK AD-H; 40% (50:50 MeOH:IPA) in hexanes + 0.1% DEA |
| 4 Isomer 1 Isomer 2 | 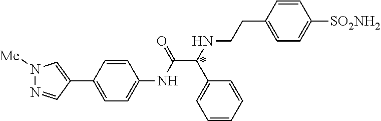 [SMILES: Cn1cc(-c2ccc(NC(=O)C(NCCc3ccc(S(N)(=O)=O)cc3)c3ccccc3)cc2)cn1] | (S)- and (R)-N-(4-(1- methyl-1H-pyrazol-4- yl)phenyl)-2-phenyl- 2-((4- sulfamoylphenethyl) amino)acetamide | Calc’d 490.6, Found 490.5 and 490.5 | CHIRALPAK AD-H; 50% (50:50 MeOH:IPA) in hexanes + 0.1% DEA |

### Scheme 2

The starting materials required for the synthesis of examples prepared using Scheme 2 were either commercially available or prepared using methods 1 through 3.

### Example 5

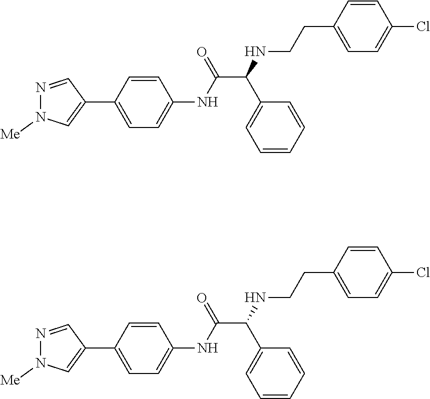 [SMILES: Cn1cc(-c2ccc(NC(=O)[C@@H](NCCc3ccc(Cl)cc3)c3ccccc3)cc2)cn1 ; Cn1cc(-c2ccc(NC(=O)[C@H](NCCc3ccc(Cl)cc3)c3ccccc3)cc2)cn1]

### (S)- and (R)-2-((4-Chlorophenethyl)amino)-N-(4-(1-methyl-1H-pyrazol-4-yl)phenyl)-2-phenylacetamide

### Scheme 2. (S)- and (R)-2-((4-Chlorophenethyl)amino)-N-(4-(1-methyl-1H-pyrazol-4-yl)phenyl)-2-phenylacetamide

A mixture of N-(4-bromophenyl)-2-((4-chlorophenethyl)amino)-2-phenylacetamide (0.2 g, 0.90 mmol), 1-methyl-4-(4,4,5,5-tetramethyl-1,3,2-dioxaborolan-2-yl)-1H-pyrazole (0.205 g, 0.99 mmol) and cesium carbonate (0.9 g, 2.69 mmol) in 4:1 dioxane:water (5 ml) was purged for 20 minutes with argon. S-Phos Pd-precatalyst G3 (0.070 g, 0.089 mmol) was added and purging was continued for another 10 minutes. The reaction mixture was heated in a sealed tube at 100° C. for 2 hours. After completion of reaction (monitored by TLC), the reaction mixture was treated with water (10 ml) and extracted with ethyl acetate (2×15 ml). The combined organic layers were washed with brine (20 ml), dried over anhydrous Na₂SO₄ and concentrated under reduced pressure. The resulting residue was purified by silica gel chromatography to afford the title compound as solid (0.090 g, 55%) in racemic form.

The racemic title compound was resolved by chiral HPLC (CHIRALPAK IB; 30% (50:50 MeOH:IPA) in hexanes+0.1% DEA) to furnish the enantiopure compounds. The faster-eluting enantiomer of the title compound was obtained as a solid (Isomer 1): ¹H NMR (400 MHz, DMSO-d6): δ 2.68-2.78 (m, 4H), 3.85 (s, 3H), 4.37 (s, 1H), 7.25-7.30 (m, 3H), 7.33-7.37 (m, 4H), 7.44-7.49 (m, 4H), 7.53 (d, J=8.4 Hz, 2H), 7.80 (s, 1H), 8.06 (s, 1H), 10.01 (s, 1H). LCMS: m/z=445.57 [M+1]. The slower-eluting enantiomer of the title compound was obtained as a solid (Isomer 2): ¹H NMR (400 MHz, DMSO-d₆): δ 2.72-2.78 (m, 4H), 3.85 (s, 3H), 4.37 (s, 1H), 7.25-7.30 (m, 3H), 7.33-7.37 (m, 4H), 7.44-7.54 (m, 6H), 7.80 (s, 1H), 8.06 (s, 1H), 10.01 (s, 1H). LCMS: m/z=445.62 [M+1].

### Example 6

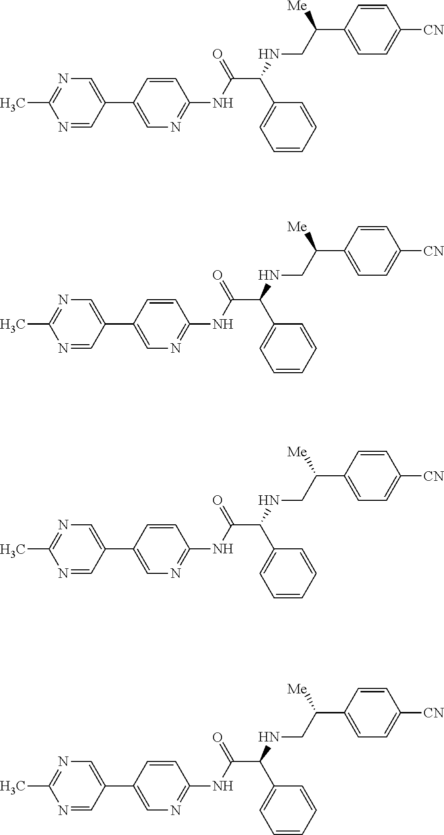 [SMILES: Cc1ncc(-c2ccc(NC(=O)[C@H](NC[C@@H](C)c3ccc(C#N)cc3)c3ccccc3)nc2)cn1 ; Cc1ncc(-c2ccc(NC(=O)[C@@H](NC[C@@H](C)c3ccc(C#N)cc3)c3ccccc3)nc2)cn1 ; Cc1ncc(-c2ccc(NC(=O)[C@H](NC[C@H](C)c3ccc(C#N)cc3)c3ccccc3)nc2)cn1 ; Cc1ncc(-c2ccc(NC(=O)[C@@H](NC[C@H](C)c3ccc(C#N)cc3)c3ccccc3)nc2)cn1]

### (S, S)-, (R, R)-, (S, R)- and (R, S)-2-((2-(4-Cyanophenyl)propyl)amino)-N-(5-(2-methylpyrimidin-5-yl)pyridin-2-yl)-2-phenylacetamide

### Scheme 3, Step 1. 2-((2-(4-Cyanophenyl)propyl)amino)-N-(5-(2-methylpyrimidin-5-yl)pyridin-2-yl)-2-phenylacetamide

A mixture of N-(5-bromopyridin-2-yl)-2-((2-(4-cyanophenyl)propyl)amino)-2-phenylacetamide (0.300 g, 0.66 mmol), 2-methyl-5-(4,4,5,5-tetramethyl-1,3,2-dioxaborolan-2-yl)pyrimidine (0.293 g, 1.33 mmol) and cesium carbonate (0.650 g, 2.0 mmol) in dioxane:water (4:1, 7.5 ml) was degassed with argon gas for 20 minutes. PdCl₂(dppf) (0.049 g, 0.066 mmol) was added and degassing was continued for another 10 minutes. The reaction mixture was heated at 100° C. for 1 hour. After completion of reaction (monitored by TLC), the reaction mixture was diluted with water (20 ml) and extracted with ethyl acetate (2×30 ml). The combined organic layers were washed with brine, dried over anhydrous Na₂SO₄ and concentrated under reduced pressure. The crude product was purified by silica gel chromatography to give title compound (0.270 g, 78%) as off-white solid in racemic form.

The racemic title compound was resolved by chiral HPLC (CHIRALCEL OX—H; 30% (30:70 ACN:IPA) in hexanes+0.1% DEA) then (CHIRALCEL OJ-H; 25% (MeOH) in liquid CO₂+0.1% DEA) to furnish the enantiopure compounds. The first-eluting enantiomer of the title compound was obtained as a solid (Isomer 1): ¹H NMR (400 MHz, DMSO-d₆) δ 1.21 (d, J=7.2 Hz, 3H), 2.66 (s, 3H), 2.69-2.71 (m, 3H), 3.03-3.05 (m, 1H), 4.50 (d, J=7.6 Hz, 1H), 7.25-7.41 (m, 7H), 7.76 (d, J=8 Hz, 2H), 8.12-8.30 (m, 2H), 8.74 (d, J=2 Hz 1H), 9.05 (s, 2H), 10.52 (s, 1H). LCMS: m/z=463.4 [M+1]; The second-eluting enantiomer of the title compound was obtained as a solid (Isomer 2): ¹H NMR (400 MHz, DMSO-d₆) δ 1.22 (d, J=6.8 Hz, 3H), 2.68 (s, 3H), 2.69-2.73 (m, 3H), 3.04-3.06 (m, 1H), 4.52 (d, J=6.0 Hz, 1H), 7.25-7.49 (m, 7H), 7.77 (d, J=8 Hz, 2H), 8.12-8.30 (m, 2H), 8.74 (s, 1H), 9.06 (s, 2H), 10.54 (s, 1H). LCMS: m/z=463.6 [M+1]. The third-eluting enantiomer of the title compound was obtained as a solid (Isomer 3): ¹H NMR (400 Mhz, DMSO-d6) δ 1.23 (d, J=6.8 Hz, 3H), 2.68 (s, 3H), 2.70-2.72 (m, 2H), 3.04-3.06 (m, 1H), 4.51 (d, J=6.0 Hz, 1H), 7.25-7.49 (m, 7H), 7.77 (d, J=8 Hz, 2H), 8.12-8.30 (m, 2H), 8.74 (s, 1H), 9.06 (s, 2H), 10.55 (s, 1H). LCMS: m/z=463.5 [M+1]; The forth-eluting enantiomer of the title compound was obtained as a solid (Isomer 4): ¹H NMR (400 MHz, DMSO-d6) δ 1.23 (d, J=6.8 Hz, 3H), 2.68 (s, 3H), 2.70-2.72 (i, 2H), 3.04-3.06 (m, 1H), 4.51 (d, J=6.4 Hz, 1H), 7.25-7.49 (i, 7H), 7.77 (d, J 8.4 Hz, 2H), 8.14-8.24 (m, 2H), 8.76 (s, 1H), 9.07 (s, 2H), 10.55 (s, 1H) LCMS m/z=463.4 [M+1].

The compounds in Table 2 were prepared using similar procedures to those described for Examples 5 and 6 using the appropriate starting materials.

TABLE 2

| | | | Exact | Chiral Column |
|---|---|---|---|---|
| Example | Structure | | Mass | and |
| No. | (Methods and Schemes for Preparation) | IUPAC Name | [M + 1] | Mobile Phase |
| 7 Isomer 1 Isomer 2 | 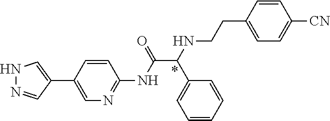 [SMILES: N#Cc1ccc(CCNC(C(=O)Nc2ccc(-c3cn[nH]c3)cn2)c2ccccc2)cc1] | (S)- and (R)-N-(5-(1H- pyrazol-4-yl)pyridin-2-yl)- 2-((4- cyanophenethyl)amino)-2- phenylacetamide | Calc’d 423.5, Found 423.6 and 423.5 | CHIRALCEL OJ-H; 15% MeOH in Liquid CO₂ + 0.1% DEA |
| 8 Isomer 1 Isomer 2 Isomer 3 Isomer 4 | 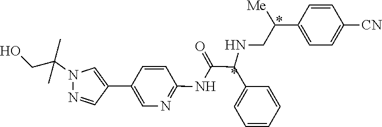 [SMILES: CC(CNC(C(=O)Nc1ccc(-c2cnn(C(C)(C)CO)c2)cn1)c1ccccc1)c1ccc(C#N)cc1] | (S, S), (S, R), (R, R), and (R, S)-2-((2-(4- cyanophenyl)propyl)amino)- N-(5-(1-(1-hydroxy-2- methylpropan-2-yl)-1H- pyrazol-4-yl)pyridin-2-yl)- 2-phenylacetamide | Calc’d 509.6, Found 509.5, 509.5, 509.9 and 509.9 | 1-CHIRALPAK IC; 30% (50:50 MeOH:IPA) in hexanes + 0.1% DEA 2-CHIRALPAK IC; 20% (30:70 ACN:IPA) in hexanes + 0.1% DEA |

### Scheme 3

The starting materials required for the synthesis of examples prepared using Scheme 3 were either commercially available or prepared using methods 1 through 3.

### Example 9

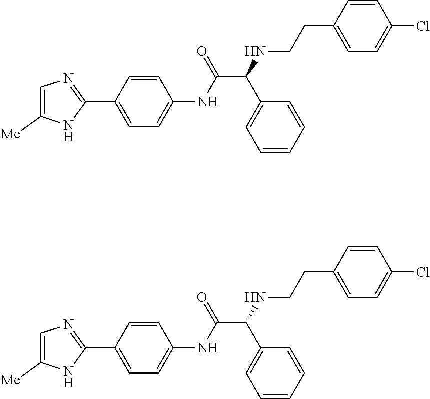 [SMILES: Cc1cnc(-c2ccc(NC(=O)[C@@H](NCCc3ccc(Cl)cc3)c3ccccc3)cc2)[nH]1 ; Cc1cnc(-c2ccc(NC(=O)[C@H](NCCc3ccc(Cl)cc3)c3ccccc3)cc2)[nH]1]

### (S)- and (R)-2-((4-Chlorophenethyl)amino)-N-(4-(5-methyl-1H-imidazol-2-yl)phenyl)-2-phenylacetamide

### Scheme 3, Step 1. 2-((4-Chlorophenethyl)amino)-2-phenyl-N-(4-(4,4,5,5-tetramethyl-1,3,2-dioxaborolan-2-yl)phenyl)acetamide

A mixture of N-(4-bromophenyl)-2-((4-chlorophenethyl)amino)-2-phenyl acetamide (1.5 g, 3.39 mmol), bis(pinacolato)diboran (1.2 g, 5.09 mmol) and KOAc (0.83 g, 8.47 mmol) in 1,4-dioxane (30 ml) was purged for 20 minutes with argon. To this mixture, 1, 1′-bis(diphenylphosphino)ferrocene-palladium(II)dichloride (0.248 g, 0.33 mmol) was added and purging was continued for another 10 minutes. The reaction mixture was heated in a sealed tube at 90° C. for 6 hours. After completion of the reaction (monitored by TLC), the reaction mixture was treated with water (20 ml) and extracted with ethyl acetate (3×20 ml). The combined organic layers were washed with brine (20 ml), dried over anhydrous Na₂SO₄ and concentrated under reduced pressure. The crude residue was purified by silica gel chromatography to afford the title compound as a solid (1.2 g, 72%). ¹H NMR (400 MHz, DMSO-d₆): δ 1.28 (s, 12H), 2.69-2.76 (m, 4H), 4.38 (s, 1H), 7.25-7.30 (m, 3H), 7.33-7.36 (m, 4H), 7.44 (d, J=6.8 Hz, 2H), 7.54 (d, J=8.4 Hz, 2H), 7.60 (d, J=8.4 Hz, 2H), 10.09 (s, —NH). LCMS: m/z=491.5 [M+1].

### Scheme 3, Step 2. (S)- and (R)-2-((4-chlorophenethyl)amino)-N-(4-(5-methyl-1H-imidazol-2-yl)phenyl)-2-phenylacetamide

A mixture of 2-((4-chlorophenethyl)amino)-2-phenyl-N-(4-(4,4,5,5-tetramethyl-1,3,2-dioxaborolan-2-yl)phenyl)acetamide (0.2 g, 0.40 mmol), 2-bromo-5-methyl-1H-imidazole (0.131 g, 0.81 mmol) and cesium carbonate (0.332 g, 1.02 mmol) in 4:1 dioxane:water (10 ml) was purged with argon for 20 minutes. 1,1′-bis(diphenylphosphino)-ferrocene-palladium(II)dichloride (0.029 g, 0.04 mmol) was added and purging was continued for another 10 minutes. The reaction mixture was heated in a sealed tube with microwave irradiation at 135° C. for 2 hours. After completion of the reaction (monitored by TLC), the reaction mixture was treated with water (20 ml) and extracted with ethyl acetate (2×20 ml). The combined organic layers were washed with brine (20 ml), dried over anhydrous Na₂SO₄ and concentrated under reduced pressure. The crude residue was purified by silica gel chromatography to afford the title compound as solid (0.080 g, 44%) in racemic form.

The racemic title compound was resolved by chiral xPLC (CHIRALPAK AD-H; (50:50 ACN:NPA) in liquid CO₂+0.1% DEA) to furnish the enantiopure compounds. The faster-eluting enantiomer of the title compound was obtained as a solid (Isomer 1): ¹H NM/R (400 MHz, DMSO-d6): δ 2.13-2.21 (m, 3H), 2.67-2.77 (m, 4H), 4.37 (s, 1H), 6.64-6.88 (m, 1H), 7.25-7.29 (m, 3H), 7.33-7.36 (m, 4H), 7.44 (d, J=7.6 Hz, 2H), 7.55 (d, J=8.4 Hz, 2H), 7.78 (t, J=5.6 Hz, 2H), 10.08 (s, 1H, —NH), 12.06-12.12 (in, 1H, —NH). LCMS: m/z=445.4 [M+1]. The slower-eluting enantiomer of the title compound was obtained as a solid (Isomer 2): ¹H NMR (400 MHz, DMSO-d6): δ 2.13-2.21 (m, 3H), 2.73-2.77 (m, 4H), 4.37 (s, 1H), 6.64-6.88 (m, 1H), 7.25-7.29 (m, 3H), 7.33-7.36 (m, 4H), 7.44 (d, J=7.2 Hz, 2H), 7.55 (d, J=8.4 Hz, 2H), 7.78-7.80 (i, 2H), 10.08 (s, 1H, —N), 12.06-12.13 (in, 1H, —N). LCMS: m/z=445.5 [M+1].

The following compounds were prepared using similar procedures to those described for Example 9 using the appropriate starting materials.

TABLE 3

| | | | | Chiral Column |
|---|---|---|---|---|
| Example | Structure | | Exact Mass | and |
| No. | (Methods and Schemes for Preparation) | IUPAC Name | [M + 1] | Mobile Phase |
| 10 Isomer 1 Isomer 2 | 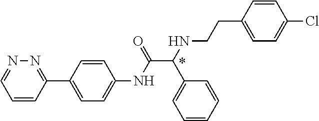 [SMILES: O=C(Nc1ccc(-c2cccnn2)cc1)C(NCCc1ccc(Cl)cc1)c1ccccc1] | (S)- and (R)-2-((4- chlorophenethyl) amino)-2-phenyl-N- (4-(pyridazin-3- yl)phenyl)acetamide | Calc’d 443.2, Found 443.3 and 443.4 | CHIRALPAK IC; 45% (50:50 MeOH:IPA) in hexanes + 0.1% DEA |
| 11 Isomer 1 Isomer2 | 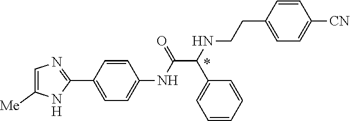 [SMILES: Cc1cnc(-c2ccc(NC(=O)C(NCCc3ccc(C#N)cc3)c3ccccc3)cc2)[nH]1] | (S)- and (R)-2-((4- cyanophenethyl) amino)-N-(4-(5- methyl-1H- imidazol-2- yl)phenyl)-2- phenylacetamide | Calc’d 436.3, Found 436.3 and 436.2 | CHIRALPAK AD- H; 35% (50:50 ACN:IPA) in Liquid CO₂ + 0.1% DEA |
| 12 Isomer 1 Isomer 2 | 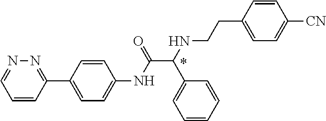 [SMILES: N#Cc1ccc(CCNC(C(=O)Nc2ccc(-c3cccnn3)cc2)c2ccccc2)cc1] | (S)- and (R)-2-((4- cyanophenethyl) amino)-2-phenyl-N- (4-(pyridazin-3- yl)phenyl)acetamide | Calc’d 434.2, Found 434.3 and 434.3 | CHIRALPAK IC; 40% (70:30 MeOH:IPA) in hexanes + 0.1% DEA |
| 13 Isomer 1 Isomer 2 | 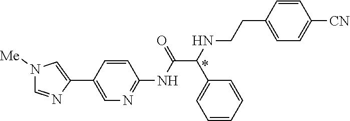 [SMILES: Cn1cnc(-c2ccc(NC(=O)C(NCCc3ccc(C#N)cc3)c3ccccc3)nc2)c1] | (S)- and (R)-2-((4- cyanophenethyl) amino)-N-(5-(1- methyl-1H- imidazol-4- yl)pyridin-2-yl)-2- phenylacetamide | Calc’d 437.2 Found 437.6 and 437.6 | CHIRALPAK IB; 35% (25:75 MeOH:IPA) in hexanes + 0.1% DEA |
| 14 Isomer 1 Isomer 2 | 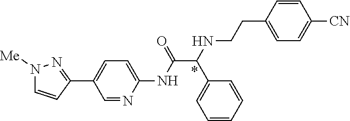 [SMILES: Cn1ccc(-c2ccc(NC(=O)C(NCCc3ccc(C#N)cc3)c3ccccc3)nc2)n1] | (S)- and (R)-2-((4- cyanophenethyl) amino)-N-(5-(1- methyl-1H- pyrazol-3- yl)pyridin-2-yl)-2- phenylacetamide | Calc’d 437.2, Found 437.5 and 437.4 | CHIRALPAK IB; 15% (50:50 MeOH:IPA) in Liquid CO₂ + 0.1% DEA |
| 15 Isomer 1 Isomer 2 Isomer 3 Isomer 4 | 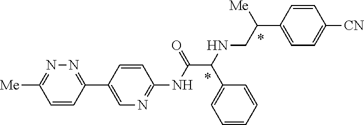 [SMILES: Cc1ccc(-c2ccc(NC(=O)C(NCC(C)c3ccc(C#N)cc3)c3ccccc3)nc2)nn1] | (S, S)-, (S, R)-, (R, R)- and (R, S)-2- ((2-(4- cyanophenyl)propyl) amino)-N-(5-(6- methylpyridazin-3- yl)pyridin-2-yl)-2- phenylacetamide | Calc’d 463.6, Found 463.8, 463.8, 463.8 and 463.8 | 1-CHIRALCEL OX-H; 20% (70:30 IPA:ACN) in hexanes + 0.1% DEA 2-CHIRALPAK IC; 37% MeOH in Liquid CO₂ + 0.1% DEA |

### Scheme 4

The starting materials required for the synthesis of examples prepared using Scheme 4 were either commercially available or prepared using methods 1 through 3.

### Example 16

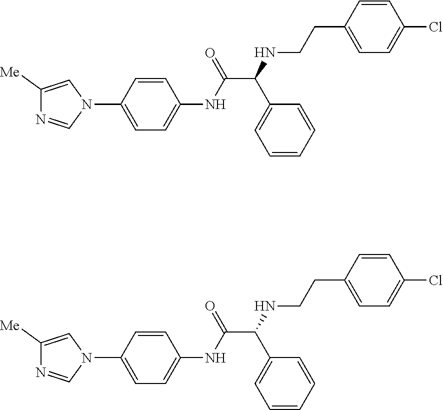 [SMILES: Cc1cn(-c2ccc(NC(=O)[C@@H](NCCc3ccc(Cl)cc3)c3ccccc3)cc2)cn1 ; Cc1cn(-c2ccc(NC(=O)[C@H](NCCc3ccc(Cl)cc3)c3ccccc3)cc2)cn1]

### (S)- and (R)-2-((4-Chlorophenethyl)amino)-N-(4-(4-methyl-1H-imidazol-1-yl)phenyl)-2-phenylacetamide

### Scheme 4. (S)- and (R)-2-((4-Chlorophenethyl)amino)-N-(4-(4-methyl-1H-imidazol-1-yl)phenyl)-2-phenylacetamide

A mixture of N-(4-bromophenyl)-2-((4-chlorophenethyl)amino)-2-phenylacetamide (0.15 g, 0.33 mmol), 4-methyl-1H-imidazole (0.14 g, 1.69 mmol), CuI (0.032 g, 0.16 mmol) and cesium carbonate (0.11 g, 0.33 mmol) in DMF (3 ml) was purged for 20 minutes with argon. 1-(5,6,7,8-tetra hydroquinolin-8-yl)ethanone (0.011 g, 0.06 mmol) was added and purging was continued for another 10 minutes. The reaction mixture was heated in a sealed tube at 135° C. for 16 hours. After completion of the reaction (monitored by TLC), the mixture was treated with water (10 ml) and extracted with ethyl acetate (2×10 ml). The combined organic layers were washed with brine (10 ml), dried over anhydrous Na₂SO₄ and concentrated under reduced pressure. The resulting residue was purified by silica gel chromatography to afford the title compound as solid (0.1 g, 66%) in racemic form.

The racemic title compound was resolved by chiral HPLC (CHIRALPAK OJ-H; 20% MeOH in Liquid CO₂+0.1% DEA) to furnish the enantiopure compounds. The faster-eluting enantiomer of the title compound was obtained as a solid (Isomer 1): ¹H NMR (400 MHz, DMSO-d6): δ 2.15 (s, 3H), 2.68-2.78 (m, 4H), 4.39 (d, J=7.6 Hz, 1H), 7.25-7.31 (m, 3H), 7.33-7.37 (m, 4H), 7.45-7.47 (m, 2H), 7.52 (d, J=8.0 Hz, 2H), 7.66-7.77 (m, 3H), 8.05 (s, 1H), 10.21 (s, —NH). LCMS: m/z=445.4 [M+1]. The slower-eluting enantiomer of the title compound was obtained as a solid (Isomer 2): ¹H NMR (400 MHz, DMSO-d₆): 2.15 (s, 3H), 2.72-2.82 (m, 4H), 4.40 (s, 1H), 7.25-7.30 (m, 3H), 7.33-7.37 (m, 4H), 7.46 (d, J=7.2 Hz, 2H), 7.52 (d, J=8.8 Hz, 2H), 7.66-7.70 (m, 3H), 8.05 (s, 1H), 10.26 (s, —NH). LCMS: m/z=445.4 [M+1].

### Scheme 5

The starting materials required for the synthesis of examples prepared using Scheme 5 were either commercially available or prepared using methods 1 through 3.

### Example 17

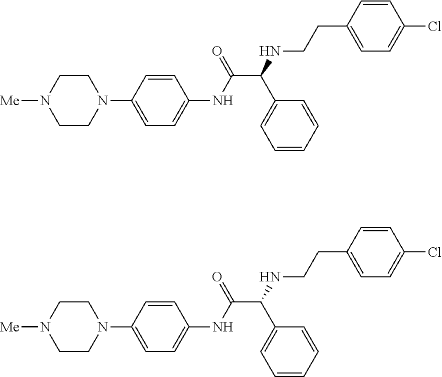 [SMILES: CN1CCN(c2ccc(NC(=O)[C@@H](NCCc3ccc(Cl)cc3)c3ccccc3)cc2)CC1 ; CN1CCN(c2ccc(NC(=O)[C@H](NCCc3ccc(Cl)cc3)c3ccccc3)cc2)CC1]

### (S)- and (R)-2-((4-Chlorophenethyl)amino)-N-(4-(4-methylpiperazin-1-yl)phenyl)-2-phenylacetamide

### Scheme 6. (S)- and (R)-2-((4-Chlorophenethyl)amino)-N-(4-(4-methylpiperazin-1-yl)phenyl)-2-phenylacetamide

A mixture of N-(4-bromophenyl)-2-((4-chlorophenethyl)amino)-2-phenylacetamide (0.4 g, 0.79 mmol), 1-methylpiprazine (0.1 g, 1.01 mmol) and cesium carbonate (0.55 g, 1.69 mmol) in dioxane (4 ml) was purged for 20 minutes with argon. Brett-Phos Pd-precatalyst G3 (0.061 g, 0.067 mmol) was added and purging was continued for another 10 minutes. The reaction mixture was heated in a sealed tube with microwave irradiation at 135° C. for 2 hours. After completion of the reaction (monitored by TLC), the mixture was treated with water (15 ml) and extracted with ethyl acetate (2×15 ml). The combined organic layers were washed with brine (15 ml), dried over anhydrous Na₂SO₄ and concentrated under reduced pressure. The resulting residue was purified by silica gel chromatography to give afford the title compound as solid (0.08 g, 25%) in racemic form.

The racemic title compound was resolved by chiral HPLC (CHIRALCEL OX—H; 35% (50:50 MeOH:IPA) in hexanes+0.1% DEA) to furnish the enantiopure compounds. The faster-eluting enantiomer of the title compound was obtained as a solid (Isomer 1): ¹H NMR (400 MHz, DMSO-d₆): δ 2.21 (s, 3H), 2.42-2.45 (m, 4H), 2.68-2.77 (m, 5H), 3.04-3.06 (m, 4H), 4.32 (d, J=7.2 Hz, 1H), 6.86 (d, J=8.8 Hz, 2H), 7.24-7.44 (m, 11H), 9.83 (s, 1H). LCMS: m/z=463.1 [M+1]. The slower-eluting enantiomer of the title compound was obtained as a solid (Isomer 2): ¹H NMR (400 MHz, DMSO-d₆): δ 2.23 (s, 3H), 2.46-2.51 (m, 4H), 2.69-2.77 (m, 5H), 3.04-3.06 (m, 4H), 4.33 (s, 1H), 6.86 (d, J=8.8 Hz, 2H), 7.24-7.44 (m, 11H), 9.83 (s, 1H). LCMS: m/z=463.5 [M+1].

The following compounds were prepared using similar procedures to those described for Example 17 using the appropriate starting materials.

TABLE 4

| | | | | Chiral |
|---|---|---|---|---|
| | | | Exact | Column |
| Example | Structure | | Mass | and |
| No. | (Methods and Schemes for Preparation) | IUPAC Name | [M + 1] | Mobile Phase |
| 18 Isomer 1 Isomer 2 | 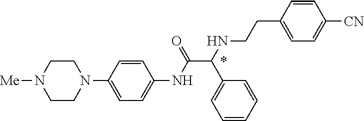 [SMILES: CN1CCN(c2ccc(NC(=O)C(NCCc3ccc(C#N)cc3)c3ccccc3)cc2)CC1] | (S)- and (R)-2-((4- cyanophenethyl)amino)- N-(4-(4-methylpiperazin- 1-yl)phenyl)-2- phenylacetamide | Calc’d 454.3, Found 454.5 and 454.5 | CHIRALCEL OX-H; MeOH in Liquid CO₂ + 0.1% DEA |

### Scheme 6

The starting materials required for the synthesis of examples prepared using Scheme 7 were generally prepared using methods 1 through 3 or were commercially available.

### Example 19

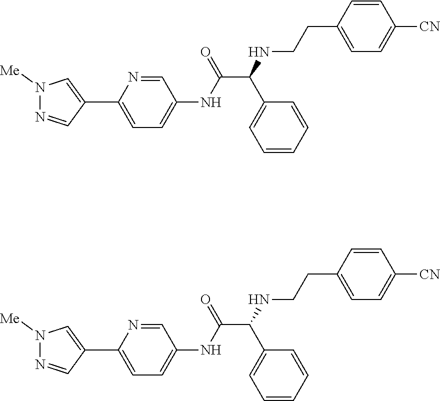 [SMILES: Cn1cc(-c2ccc(NC(=O)[C@@H](NCCc3ccc(C#N)cc3)c3ccccc3)cn2)cn1 ; Cn1cc(-c2ccc(NC(=O)[C@H](NCCc3ccc(C#N)cc3)c3ccccc3)cn2)cn1]

### (S)- and (R)-2-((4-cyanophenethyl)amino)-N-(6-(1-methyl-1H-pyrazol-4-yl)pyridin-3-yl)-2-phenylacetamide

### Scheme 6, Step 1. Ethyl 2-((4-cyanophenethyl)amino)-2-phenylacetate

A mixture of ethyl 2-bromo-2-phenylacetate (2.0 g, 8.22 mmol), 4-(2-aminoethyl)benzonitrile hydrochloride (2.25 g, 12.33 mmol) and TEA (2.50 g, 24.66 mmol) in DMF (20 ml) was heated for 3 hours at 60° C. The reaction mixture was poured into ice cold water (50 ml) and extracted with ethyl acetate (2×50 ml). The combined organic layers were washed with brine (25 ml), dried over anhydrous Na₂SO₄ and concentrated under reduced pressure. The resulting residue was purified by silica gel chromatography to afford the title compound (2.2 g, 86%) as a thick liquid. ¹H NMR (400 MHz, DMSO-d6): δ 1.10 (t, J=7.2 Hz, 3H), 2.62-2.82 (m, 4H), 4.02-4.09 (m, 2H), 4.39 (d, J=8.4 Hz, 1H), 7.28-7.35 (m, 5H), 7.40 (d, J=8.4 Hz, 2H), 7.72 (d, J=8.4 Hz, 2H). LCMS: m/z=309.28 [M+1].

### Scheme 6, Step 2, procedure 1. (S)- and (R)-2-((4-Cyanophenethyl)amino)-N-(6-(1-methyl-1H-pyrazol-4-yl)pyridin-3-yl)-2-phenylacetamide

To a mixture of 6-(1-methyl-1H-pyrazol-4-yl)pyridin-3-amine (250 mg, 1.44 mmol) and ethyl 2-((4-cyanophen ethyl)amino)-2-phenylacetate (531 mg, 1.72 mmol) in toluene, trimethyl aluminium (2.9 ml, 2.870 mmol; 1 M in toluene) was added under an atmosphere of nitrogen at room temperature. The resulting reaction mixture was heated to 100° C. for 2 hours. After completion of the reaction (monitored by TLC), the mixture was diluted with ethyl acetate (20 ml) and slowly quenched with water (20 ml) at room temperature. The aqueous layer was extracted with ethyl acetate (2×20 ml). The combined organic layers were washed with brine (20 ml), dried over anhydrous Na₂SO₄ and concentrated under reduced pressure. The resulting residue was purified by silica gel chromatography to afford the title compound (150 mg, 30%) in racemic form.

The racemic title compound was resolved by chiral HPLC (CHIRALPAK IB; 55% (50:50 MeOH:IPA) in hexanes+0.1% DEA) to furnish the enantiopure compounds. The faster-eluting enantiomer of the title compound was obtained as a solid (Isomer 1): ¹H NMR (400 MHz, DMSO-d6): δ 2.61-2.82 (m, 2H), 2.87-2.89 (m, 2H), 3.87 (s, 3H), 4.43 (s, 1H), 7.27-7.31 (m, 1H), 7.34-7.39 (m, 2H), 7.44-7.48 (m, 4H), 7.57-7.59 (d, J=8.8 Hz, 1H), 7.74-7.76 (d, J=8.0 Hz, 2H), 7.92 (s, 1H), 8.01 (dd, J=8.8 Hz, 2.4 Hz, 1H), 8.20 (s, 1H), 8.66 (d, J=2.4 Hz, 1H), 10.34 (s, 1H, —NH). LCMS: m/z=437.24 [M+1]. The slower-eluting enantiomer of the title compound was obtained as a solid (Isomer 2): ¹H NMR (400 MHz, DMSO-d6): δ 2.77-2.78 (m, 2H), 2.87-2.89 (m, 2H), 3.87 (s, 3H), 4.42 (s, 1H), 7.27-7.31 (m, 1H), 7.34-7.39 (m, 2H), 7.44-7.48 (m, 4H), 7.59 (d, J=8.4 Hz, 1H), 7.75 (d, J=8.0 Hz, 2H), 7.92 (s, 1H), 8.01 (dd, J=8.4 Hz, 2.4 Hz, 1H), 8.20 (s, 1H), 8.66 (d, J=2.0 Hz, 1H), 10.34 (s, 1H, —NH). LCMS: m/z=437.24 [M+1].

### Example 22

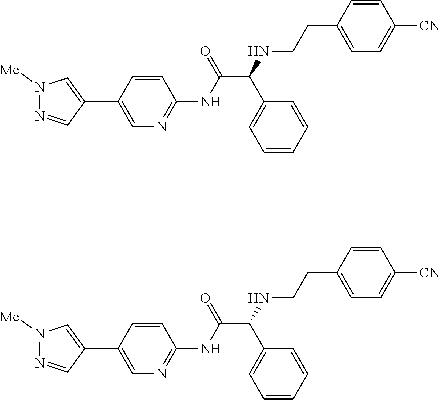 [SMILES: Cn1cc(-c2ccc(NC(=O)[C@@H](NCCc3ccc(C#N)cc3)c3ccccc3)nc2)cn1 ; Cn1cc(-c2ccc(NC(=O)[C@H](NCCc3ccc(C#N)cc3)c3ccccc3)nc2)cn1]

### (S)- and (R)-2-((4-cyanophenethyl)amino)-N-(5-(1-methyl-1H-pyrazol-4-yl)pyridin-2-yl)-2-phenylacetamide

### Scheme 6, Step 2, procedure 1. (S)- and (R)-2-((4-cyanophenethyl)amino)-N-(5-(1-methyl-1H-pyrazol-4-yl)pyridin-2-yl)-2-phenylacetamide

To a stirred solution of 5-(1-methyl-1H-pyrazol-4-yl)pyridin-2-amine (1.0 g, 5.74 mmol), ethyl 2-((4-cyanophenethyl)amino)-2-phenylacetate (2.12 g, 6.88 mmol) in dry toluene (10 ml) was added trimethylaluminium (5.8 ml, 2M in toluene, 11.48 mmol) at 0° C. The reaction mixture was stirred at 100° C. for 2 hours. After completion of the reaction, the reaction mixture was poured into ice cold water (50 ml) and extracted with ethyl acetate (2×100 ml). The combined organic layers were washed with brine (50 ml), dried over anhydrous sodium sulphate and concentrated under reduced pressure. The resulting residue was purified by silica gel chromatography to afford the title compound (0.30 g, 12%) as racemic mixture.

The racemic title compound was resolved by chiral HPLC (CHIRALCEL OJ-H; 14% MeOH in liquid CO₂+0.1% DEA) to furnish the enantiopure compounds. The faster-eluting enantiomer (example 22, isomer 1 in tables 5 and 8 below) of the title compound was obtained as a solid. ¹H NMR (400 MHz, DMSO-d₆): δ 2.73-2.80 (m, 2H), 2.85-2.88 (m, 3H), 3.86 (s, 3H), 4.53 (d, J=8.8 Hz, 1H), 7.25-7.29 (m, 1H), 7.32-7.35 (m, 2H), 7.44 (d, J=8.0 Hz, 4H), 7.73 (d, J=8.0 Hz, 2H), 7.89 (s, 1H), 7.92-7.95 (m, 1H), 8.02 (d, J=8.4 Hz, 1H), 8.17 (s, 1H), 8.55 (d, J=1.6 Hz, 1H), 10.48 (s, 1H). LCMS: m/z=437.22 [M+1].

### Example 100

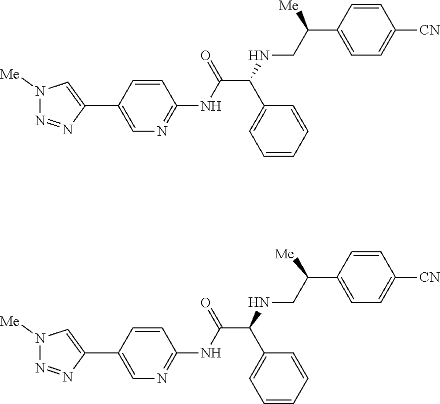 [SMILES: C[C@H](CN[C@@H](C(=O)Nc1ccc(-c2cn(C)nn2)cn1)c1ccccc1)c1ccc(C#N)cc1 ; C[C@H](CN[C@H](C(=O)Nc1ccc(-c2cn(C)nn2)cn1)c1ccccc1)c1ccc(C#N)cc1]

### Scheme 6, Step 2, procedure 1. (R, S)-, (S, S)-2-((2-(4-cyanophenyl)propyl)amino)-N-(5-(1-methyl-1H-1,2,3-triazol-4-yl)pyridin-2-yl)-2-phenylacetamide

To a stirred solution of 5-(1-methyl-1H-1,2,3-triazol-4-yl)pyridin-2-amine (0.1 g, 0.56 mmol), a 1:1 mixture of (S, R)- and (S, S)-ethyl 2-((4-cyanophenethyl)amino)-2-phenylacetate (0.27 g, 0.85 mmol) in dry toluene (2 ml) was added trimethylaluminium (0.6 ml, 2M in toluene, 1.13 mmol) at 0° C. The reaction mixture was stirred at 100° C. for 2 hours. After completion of the reaction, the reaction mixture was poured into ice cold water (25 ml) and extracted with ethyl acetate (2×50 ml). The combined organic layers were washed with brine (25 ml), dried over anhydrous sodium sulphate and concentrated under reduced pressure. The resulting residue was purified by silica gel chromatography to afford the title compounds (0.078 g, 31%) as mixture.

The title compounds were resolved by chiral HPLC (CHIRALPAK IC; 10% (70:30 IPA:ACN) in n-hexanes+0.1% DEA). The slower-eluting enantiomer (example 100, isomer 2 in tables 5 and 8 below) of the title compound was obtained as a solid. ¹H NMR (400 MHz, DMSO-d6): δ 1.24 (d, J=5.6 Hz, 1H), 2.51-2.66 (m, 3H), 3.04 (d, J=7.2 Hz, 1H), 4.10 (s, 3H), 4.50 (d, J=7.6 Hz, 1H), 7.28-7.47 (m, 7H), 7.76 (d, J=7.2 Hz, 2H), 8.12-8.18 (m, 2H), 8.57 (s, 1H), 8.79 (s, 1H), 10.64 (s, 1H). LCMS: m/z=452.52=[M+1].

### Example 20

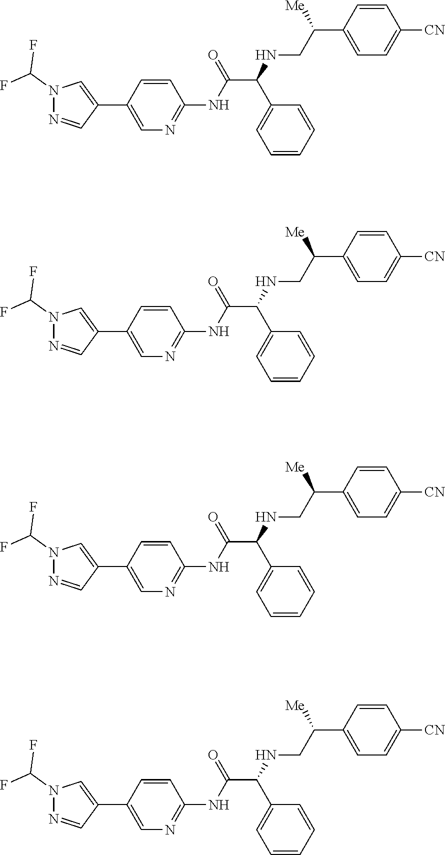 [SMILES: C[C@@H](CN[C@H](C(=O)Nc1ccc(-c2cnn(C(F)F)c2)cn1)c1ccccc1)c1ccc(C#N)cc1 ; C[C@H](CN[C@@H](C(=O)Nc1ccc(-c2cnn(C(F)F)c2)cn1)c1ccccc1)c1ccc(C#N)cc1 ; C[C@H](CN[C@H](C(=O)Nc1ccc(-c2cnn(C(F)F)c2)cn1)c1ccccc1)c1ccc(C#N)cc1 ; C[C@@H](CN[C@@H](C(=O)Nc1ccc(-c2cnn(C(F)F)c2)cn1)c1ccccc1)c1ccc(C#N)cc1]

### (S, R)-, (R, S)-, (S, S)-, (R, R)-(2-((2-(4-cyanophenyl)propyl)amino)-N-(5-(1-(difluoromethyl)-1H-pyrazol-4-yl)pyridin-2-yl)-2-phenylacetamide

### Scheme 6, Step 2, procedure 2. (S, R)-, (R, S)-, (S, S)-, (R, R)-(2-((2-(4-Cyanophenyl)propyl)amino)-N-(5-(1-(difluoromethyl)-1H-pyrazol-4-yl)pyridin-2-yl)-2-phenylacetamide

To a stirred solution of 5-(1-(difluoromethyl)-1H-pyrazol-4-yl)pyridin-2-amine (0.35 g, 1.67 mmol), ethyl 2-((4-cyanophenethyl)amino)-2-phenylacetate (0.59 g, 1.83 mmol) in dry THE (4 ml) was added LiHMDS (2 ml, 1M in THF, 3.34 mmol) at 0° C. The reaction mixture was stirred at room temperature for 1 hour. After completion of the reaction (monitored by TLC), the reaction mixture was poured into ice cold water (15 ml) and extracted with ethyl acetate (2×25 ml). The combined organic layers were washed with brine (15 ml), dried over anhydrous Na₂SO₄ and concentrated under reduced pressure. The resulting residue was purified by silica gel chromatography to afford a mixture of the title compounds (0.5 g, 61%).

The mixture was resolved by chiral HPLC (CHIRALCEL OX—H; 45% (50:50 MeOH:IPA) in hexanes+0.1% DEA) then (CHIRALPAK IC; 30% (50:50 MeOH:IPA) in hexanes+0.1% DEA) to furnish the enantiopure compounds. The first-eluting enantiomer of the title compound was obtained as a solid (Isomer 1): ¹H NMR (400 MHz, DMSO-d6) δ 1.22 (d, J=6.8 Hz, 3H), 2.69-2.71 (m, 3H), 3.03-3.07 (m, 1H), 4.49 (d, J=6.8 Hz, 1H), 7.26-7.49 (m, 7H), 7.72-7.78 (m, 2H), 7.87 (s, 1H), 8.02-8.12 (m, 2H), 8.33 (s, 1H), 8.71 (d, J=1.2 Hz, 1H), 8.79 (s, 1H), 10.46 (s, 1H). LCMS: m/z=487.7 [M+1]; The second-eluting enantiomer of the title compound was obtained as a solid (Isomer 2): ¹H NMR (400 MHz, DMSO-d6) δ 1.24 (d, J=6.8 Hz, 3H), 2.60-2.71 (m, 3H), 3.01-3.07 (m, 1H), 4.50 (d, J=8.4 Hz, 1H), 7.21-7.49 (m, 7H), 7.72-7.80 (m, 2H), 7.87 (s, 1H), 8.02-8.12 (m, 2H), 8.33 (s, 1H), 8.72 (s, 1H), 8.79 (s, 1H), 10.61 (s, 1H). LCMS: m/z=487.7 [M+1]; The third-eluting enantiomer of the title compound was obtained as a solid (Isomer 3): ¹H NMR (400 MHz, DMSO-d6) δ 1.22 (d, J=6.8 Hz, 3H), 2.69-2.71 (m, 3H), 3.04-3.07 (m, 1H), 4.49 (d, J=7.6 Hz, 1H), 7.26-7.49 (m, 7H), 7.72-7.78 (m, 2H), 7.87 (s, 1H), 8.02-8.12 (m, 2H), 8.33 (s, 1H), 8.71 (s, 1H), 8.79 (s, 1H), 10.46 (s, 1H). LCMS: m/z=487.7 [M+1]; The forth-eluting enantiomer of the title compound was obtained as a solid (Isomer 4): ¹H NMR (400 MHz, DMSO-d6) δ 1.24 (d, J=6.4 Hz, 3H), 2.62-2.70 (m, 3H), 3.01-3.07 (m, 1H), 4.50 (d, J=8.8 Hz, 1H), 7.22-7.49 (m, 7H), 7.72-7.78 (m, 2H), 7.87 (s, 1H), 8.02-8.13 (m, 2H), 8.33 (s, 1H), 8.72 (s, 1H), 8.79 (s, 1H), 10.61 (s, 1H). LCMS: m/z=487.7 [M+1].

### Example 33

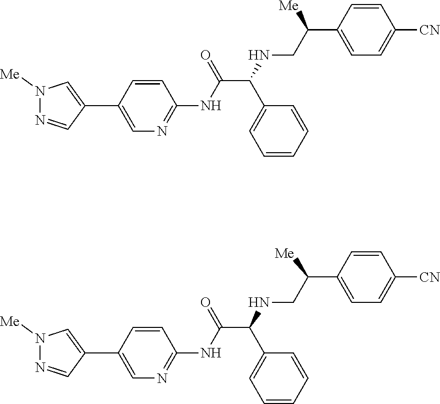 [SMILES: C[C@H](CN[C@@H](C(=O)Nc1ccc(-c2cnn(C)c2)cn1)c1ccccc1)c1ccc(C#N)cc1 ; C[C@H](CN[C@H](C(=O)Nc1ccc(-c2cnn(C)c2)cn1)c1ccccc1)c1ccc(C#N)cc1]

### (R, S)-, (S, S)-2-(4-cyanophenyl)propyl)amino)-N-(5-(1-methyl-1H-pyrazol-4-yl)pyridin-2-yl)-2-phenylacetamide

### Scheme 6, Step 1. (R, S)-, (S, S)-ethyl 2-((2-(4-cyanophenyl)propyl)amino)-2-phenylacetate

A mixture of ethyl 2-bromo-2-phenylacetate (9.11 g, 37.5 mmol), (S)-4-(1-aminopropan-2-yl)benzonitrile (5.0 g, 31.2 mmol) and TEA (13.1 ml, 93.7 mmol) in DMF (50 ml) was heated at 60° C. for 3 hours. The reaction mixture was poured into ice cold water (150 ml) and extracted with ethyl acetate (2×150 ml). The combined organic layers were washed with brine (150 ml), dried over anhydrous Na₂SO₄ and concentrated under reduced pressure. The resulting residue was purified by silica gel chromatography to afford a mixture of the title compounds (7.0 g, 70%) as a thick liquid. ¹H NMR (400 MHz, DMSO-d₆): 1.08 (t, J=6.8 Hz, 3H), 1.16 (d, J=6.8 Hz, 3H), 2.35-2.44 (m, 1H), 2.49-2.66 (m, 1H), 2.96 (q, J=6.8 Hz, 1H), 3.96-4.06 (m, 2H), 4.32 (s, 1H), 7.26-7.42 (m, 7H), 7.74 (t, J=7.6 Hz, 2H). LCMS: m/z=323.6 [M+1].

### Scheme 6, Step 2, procedure 2. (R, S)-, (S, S)-2-(4-cyanophenyl)propyl)amino)-N-(5-(1-methyl-1H-pyrazol-4-yl)pyridin-2-yl)-2-phenylacetamide

To a stirred solution of 5-(1-methyl-1H-pyrazol-4-yl)pyridin-2-amine (2.5 g, 14.4 mmol), a 1:1 mixture of (S, R)- and (S, S)-ethyl 2-((2-(4-cyanophenyl)propyl)amino)-2-phenylacetate (7.0 g, 21.7 mmol) in dry THE (50 ml) was added LiHMDS (37 ml, 1M in THF, 36.2 mmol) at 0° C. The reaction mixture was stirred at room temperature for 1 hour. After completion of the reaction, the reaction mixture was poured into ice cold water (100 ml) and extracted with ethyl acetate (2×75 ml). The combined organic layer were washed with brine (100 ml), dried over anhydrous Na₂SO₄ and concentrated under reduced pressure. The resulting residue was purified by silica gel chromatography to afford a mixture of the title compounds (5.0 g, 51%).

The title compounds were resolved by chiral HPLC (CHIRALCEL OJ-H; 15% MeOH in liquid CO₂+0.1% DEA) to obtain the slower-eluting isomer (example 33, isomer 4 in tables 5 and 8 below). ¹H NMR (400 MHz, DMSO-d6): 1.23 (d, J=6.8 Hz, 3H), 2.64-2.69 (m, 3H), 3.02 (q, J=6.8 Hz, 1H), 3.86 (s, 3H), 4.47 (d, J=7.6 Hz, 1H), 7.24-7.45 (m, 7H), 7.75 (d, J=8.4 Hz, 2H); 7.90 (s, 1H), 7.92-8.03 (m, 2H), 8.18 (s, 1H), 8.56 (d, J=1.6 Hz, 1H), 10.52 (s, —NH, 1H). LCMS: m/z=451.5 [M+1].

### Example 84

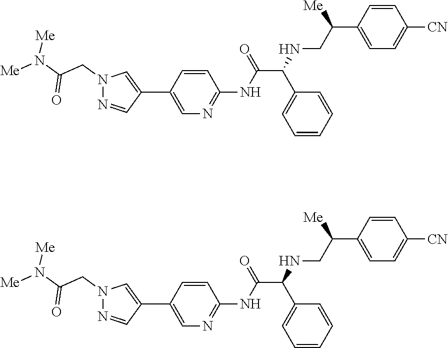 [SMILES: C[C@H](CN[C@@H](C(=O)Nc1ccc(-c2cnn(CC(=O)N(C)C)c2)cn1)c1ccccc1)c1ccc(C#N)cc1 ; C[C@H](CN[C@H](C(=O)Nc1ccc(-c2cnn(CC(=O)N(C)C)c2)cn1)c1ccccc1)c1ccc(C#N)cc1]

### (S, S)-,(R, S)-2-((2-(4-cyanophenyl)propyl)amino)-N-(5-(1-(2-(dimethylamino)-2-oxoethyl)-1H-pyrazol-4-yl)pyridin-2-yl)-2-phenylacetamide

### Scheme 6, Step 2, procedure 2. (S, S)-, (R, S)-2-((2-(4-cyanophenyl)propyl)amino)-N-(5-(1-(2-(dimethylamino)-2-oxoethyl)-1H-pyrazol-4-yl)pyridin-2-yl)-2-phenylacetamide

To a stirred solution of 2-(4-(6-aminopyridin-3-yl)-1H-pyrazol-1-yl)-N,N-dimethylacetamide (2.0 g, 8.15 mmol), a 1:1 mixture of (S, R)- and (S, S)-ethyl 2-((2-(4-cyanophenyl)propyl)amino)-2-phenylacetate (3.94 g, 12.23 mmol) in dry THE (30 ml) was added LiHMDS (16.3 ml, 1M in THF, 16.30 mmol) at 0° C. The reaction mixture was stirred at room temperature for 1 hour. After completion of the reaction, the reaction mixture was poured into ice cold water (50 ml) and extracted with ethyl acetate (2×100 ml). The combined organic layers were washed with brine (50 ml), dried over anhydrous sodium sulphate and concentrated under reduced pressure. The resulting residue was purified by silica gel chromatography to afford a mixture of the title compounds (2.5 g, 59%).

The mixture was resolved by Chiral HPLC (CHIRALCEL OJ-H; 15% MeOH in Liquid CO₂+0.1% DEA) to furnish the enantiopure compounds. The slower-eluting enantiomer (example 84, isomer 2 in tables 5 and 8 below) of the title compound was obtained as a solid. ¹H NMR (400 MHz, DMSO-d6) δ 1.24 (d, J=6.8 Hz, 3H), 2.67 (d, J=6.4 Hz, 3H), 2.87 (s, 3H), 3.01-3.05 (m, 4H), 4.48 (d, J=7.2 Hz, 1H), 5.14 (s, 2H), 7.25-7.46 (m, 7H), 7.76 (d, J=8.0 Hz, 2H), 7.93-8.05 (m, 3H), 8.12 (s, 1H), 8.59 (d, J=1.6 Hz, 1H), 10.50 (s, 1H). LCMS: m/z=522.61 [M+1].

### Example 104

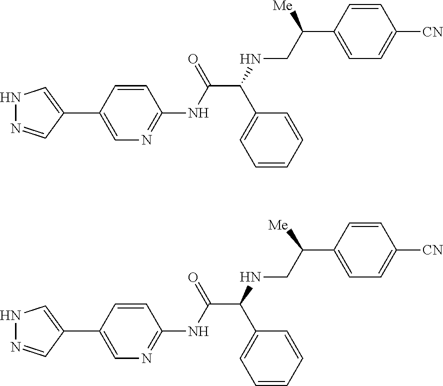 [SMILES: C[C@H](CN[C@@H](C(=O)Nc1ccc(-c2cn[nH]c2)cn1)c1ccccc1)c1ccc(C#N)cc1 ; C[C@H](CN[C@H](C(=O)Nc1ccc(-c2cn[nH]c2)cn1)c1ccccc1)c1ccc(C#N)cc1]

### (R, S)-, (S, S)—N-(5-(1H-pyrazol-4-yl)pyridin-2-yl)-2-((2-(4-cyanophenyl)propyl)amino)-2-phenylacetamide

### Scheme 6, Step 2, procedure 2. (R, S)-, (S, S)—N-(5-(1H-pyrazol-4-yl)pyridin-2-yl)-2-((2-(4-cyanophenyl)propyl)amino)-2-phenylacetamide

To a stirred solution of tert-butyl 4-(6-aminopyridin-3-yl)-1H-pyrazole-1-carboxylate (0.8 g, 3.07 mmol), a 1:1 mixture of (S, R)- and (S, S)-ethyl 2-((4-cyanophenethyl)amino)-2-phenylacetate (1.38 g, 4.30 mmol) in dry THE (20 ml) was added LiHMDS (7.6 ml, 1M in THF, 7.69 mmol) at 0° C. The reaction mixture was stirred at room temperature for 1 hour. After completion of the reaction, the reaction mixture was poured into ice cold water (50 ml) and extracted with ethyl acetate (2×50 ml). The combined organic layers were washed with brine (50 ml), dried over anhydrous sodium sulphate and concentrated under reduced pressure. The resulting residue was purified by silica gel chromatography to afford a mixture of the title compounds (0.52 g, 40%).

The mixture was resolved by Chiral HPLC (CHIRALPAK IG; 100% (70:30 MeOH:ACN) to furnish the enantiopure compounds. The slower-eluting enantiomer (example 104, isomer 2 in tables 5 and 8 below) of the title compound was obtained as a solid. ¹H NMR (400 MHz, DMSO-d6) δ 1.24 (d, J=8.0 Hz, 3H), 2.66 (s, 3H), 3.01-3.06 (s, 1H), 4.49 (s, 1H), 7.25-7.46 (m, 7H), 7.76 (d, J=8.0 Hz, 2H), 7.97-8.04 (m, 3H), 8.25 (s, 1H), 8.62 (s, 1H), 10.51 (s, 1H), 13.02 (s, 1H). LCMS: m/z=437.46 [M+1].

### Example 127

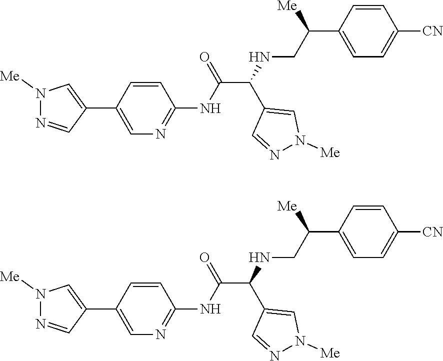 [SMILES: C[C@H](CN[C@@H](C(=O)Nc1ccc(-c2cnn(C)c2)cn1)c1cnn(C)c1)c1ccc(C#N)cc1 ; C[C@H](CN[C@H](C(=O)Nc1ccc(-c2cnn(C)c2)cn1)c1cnn(C)c1)c1ccc(C#N)cc1]

### (R, S)-, (S, S)-2-((2-(4-cyanophenyl)propyl)amino)-2-(1-methyl-1H-pyrazol-4-yl)-N-(5-(1-methyl-1H-pyrazol-4-yl)pyridin-2-yl)acetamide

### Scheme 6, Step 2, procedure 2. (R, S)-, (S, S)-((2-(4-cyanophenyl)propyl)amino)-2-(1-methyl-1H-pyrazol-4-yl)-N-(5-(1-methyl-H-pyrazol-4-yl)pyridin-2-yl)acetamide

To a stirred solution of 5-(1-methyl-1H-pyrazol-4-yl)pyridin-2-amine (1.5 g, 8.61 mmol), a 1:1 mixture of (S, R)- and (S, S)-ethyl 2-((2-(4-cyanophenyl)propyl)amino)-2-(1-methyl-1H-pyrazol-4-yl)acetate (3.37 g, 10.33 mmol) in dry THE (30 ml) was added LiHMDS (22.0 ml, 1M in THF, 21.52 mmol) at 0° C. The reaction mixture was stirred at room temperature for 1 hour. After completion of the reaction, the reaction mixture was poured into ice cold water (50 ml) and extracted with ethyl acetate (2×100 ml). The combined organic layers were washed with brine (50 ml), dried over anhydrous sodium sulphate and concentrated under reduced pressure. The resulting residue was purified by silica gel chromatography to afford a mixture of the title compounds (2.9 g, 74%).

The mixture was resolved by Chiral HPLC (CHIRALCEL OJ-H; 10% MeOH in Liquid CO₂+0.1% DEA) to furnish the enantiopure compounds. The slower-eluting enantiomer (example 127, isomer 4 in tables 5 and 8 below) of the title compound was obtained as a solid. ¹H NMR (400 MHz, DMSO-d6) δ 1.24 (d, J=6.8 Hz, 3H), 2.68 (s, 2H), 2.97-3.03 (s, 1H), 3.57 (s, 1H), 3.77 (s, 3H), 3.87 (s, 3H), 4.36 (s, 1H), 7.34 (s, 1H), 7.45 (d, J=8.4 Hz, 2H), 7.59 (s, 1H), 7.75 (d, J=8.4 Hz, 2H), 7.91 (s, 1H), 7.95 (dd, J=2.0 Hz, 8.4 Hz, 2H), 8.18 (s, 1H), 8.57 (d, J=1.6 Hz, 1H), 10.36 (s, 1H). LCMS: m/z=455.51 [M+1].

The following compounds were prepared using similar procedures to those described for Examples 19, 22, 100, 20, 33, 84, 104, and 127 using the appropriate starting materials.

TABLE 5

| | | | Exact | Chiral Column |
|---|---|---|---|---|
| Example | | | Mass | and |
| No. | Structure (Methods and Schemes for Preparation) | IUPAC Name | [M + 1] | Mobile Phase |
| 21 Isomer 1 Isomer 2 | 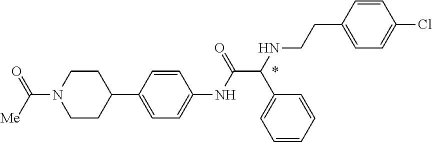 [SMILES: CC(=O)N1CCC(c2ccc(NC(=O)C(NCCc3ccc(Cl)cc3)c3ccccc3)cc2)CC1] | (S)- and (R)-N- (4-(1-acetylpi- peridin-4-yl)- phenyl)-2-((4- chlorophenyl- ethyl)amino)- 2-phenylacet- amide | Calc'd 490.2, Found 490.5 and 490.5 | CHIRALCEL OJ-H; 18% MeOH in Liquid CO₂ + 0.1% DEA |
| 22 Isomer 1 Isomer 2 | 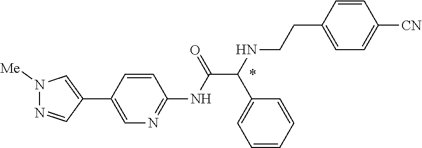 [SMILES: Cn1cc(-c2ccc(NC(=O)C(NCCc3ccc(C#N)cc3)c3ccccc3)nc2)cn1] | (S)- and (R)-2- ((4-cyanophen- ethyl)amino)- N-(5-(1-methyl- 1H-pyrazol-4- yl)pyridin-2- yl)-2-phenyl- acetamide | Calc'd 437.2, Found 437.3 and 437.3 | CHIRALCEL OJ-H; 15% (50:50 ACN:IPA) in Liquid CO₂ + 0.1% DEA |
| 23 Isomer 1 Isomer 2 | 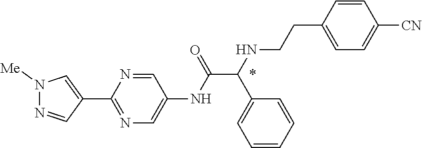 [SMILES: Cn1cc(-c2ncc(NC(=O)C(NCCc3ccc(C#N)cc3)c3ccccc3)cn2)cn1] | (S)- and (R)- 2- ((4-cyanophen- ethyl)amino)- N-(2-(1-methyl- 1H-pyrazol-4- yl)pyrimidin- 5-yl)-2-phenyl- acetamide | Calc'd 438.2, Found 438.3 and 438.3 | CHIRALCEL OJ-H; 20% MeOH in Liquid CO₂ + 0.1% DEA |
| 24 Isomer 1 Isomer 2 | 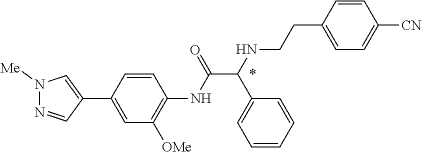 [SMILES: COc1cc(-c2cnn(C)c2)ccc1NC(=O)C(NCCc1ccc(C#N)cc1)c1ccccc1] | (S)- and (R)-2- ((4-cyanophen- ethyl)amino)-N- (2-methoxy-4- (1-methyl-1H- pyrazol-4-yl)- phenyl)-2-phen- ylacetamide | Calc'd 466.2, Found 466.2 and 466.2 | CHIRALPAK AD-H; 35% MeOH in Liquid CO₂ + 0.1% DEA |
| 25 Isomer 1 Isomer 2 | 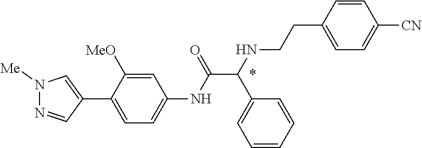 [SMILES: COc1cc(NC(=O)C(NCCc2ccc(C#N)cc2)c2ccccc2)ccc1-c1cnn(C)c1] | (S)- and (R)- 2- ((4-cyanophen- ethyl)amino)-N- (3-methoxy-4- (1-methyl-1H- pyrazol-4-yl)- phenyl)-2-phen- ylacetamide | Calc'd 466.2, Found 466.5 and 466.5 | CHIRALPAK AD-H; 25% MeOH in Liquid CO₂ + 0.1% DEA |
| 26 Isomer 1 Isomer 2 | 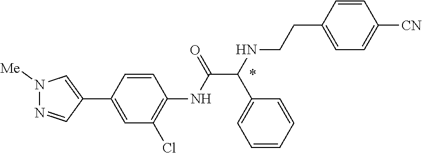 [SMILES: Cn1cc(-c2ccc(NC(=O)C(NCCc3ccc(C#N)cc3)c3ccccc3)c(Cl)c2)cn1] | (S)- and (R)-N- (2-chloro-4-(1- methyl-1H- pyrazol-4-yl)- phenyl)-2-((4- cyanophen- ethyl)amino)- 2-phenylacet- amide | Calc'd 470.2, Found 470.2 and 470.2 | CHIRALPAK IB; 20% MeOH in Liquid CO₂ + 0.1% DEA |
| 27 Isomer 1 Isomer 2 | 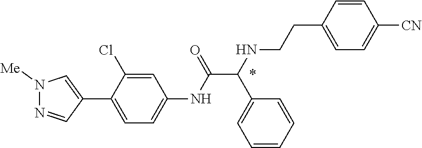 [SMILES: Cn1cc(-c2ccc(NC(=O)C(NCCc3ccc(C#N)cc3)c3ccccc3)cc2Cl)cn1] | (S)- and (R)-N- (3-chloro-4-(1- methyl-1H- pyrazol-4-yl)- phenyl)-2-((4- cyanophen- ethyl)amino)- 2-phenylacet- amide | Calc'd 470.2, Found 470.5 and 470.5 | CHIRALPAK AD-H; 38% MeOH in Liquid CO₂ + 0.1% DEA |
| 28 Isomer 1 Isomer 2 | 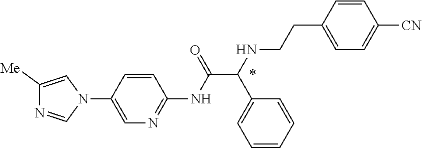 [SMILES: Cc1cn(-c2ccc(NC(=O)C(NCCc3ccc(C#N)cc3)c3ccccc3)nc2)cn1] | (S)- and (R)-2- ((4-cyanophen- ethyl)amino)- N-(5-(4-methyl- 1H-imidazol-1- yl)pyridin-2-yl)- 2-phenylacet- amide | Calc'd 437.2, Found 437.6 and 437.6 | CHIRALCEL OJ-H; 20% MeOH in liquid CO₂ + 0.1% DEA |
| 29 Isomer 1 Isomer 2 | 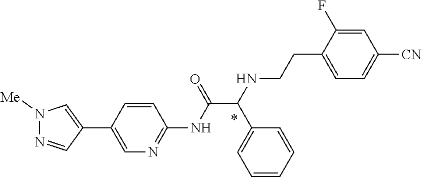 [SMILES: Cn1cc(-c2ccc(NC(=O)C(NCCc3ccc(C#N)cc3F)c3ccccc3)nc2)cn1] | (S)- and (R)-2- ((4-cyano-2- fluorophen- ethyl)amino)- N-(5-(1-methyl- 1H-pyrazol-4- yl)pyridin-2-yl)- 2-phenylacet- amide | Calc'd 455.2, Found 455.4 and 455.4 | CHIRALCEL OX-H; 40% (50:50 MeOH:IPA) in hexanes + 0.1% DEA |
| 30 Isomer 1 Isomer 2 | 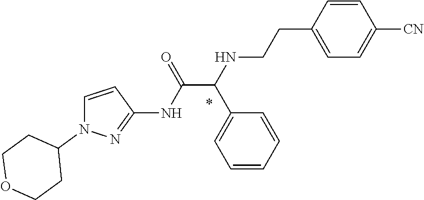 [SMILES: N#Cc1ccc(CCNC(C(=O)Nc2ccn(C3CCOCC3)n2)c2ccccc2)cc1] | (S)- and (R)-2- ((4-cyanophen- ethyl)amino)-2- phenyl-N-(1- (tetrahydro-2H- pyran-4-yl)-1H- pyrazol-3-yl)- acetamide | Calc'd 430.2, Found 430.3 and 430.2 | CHIRALCEL OX-H; 18% (50:50 ACN:MeOH) in Liquid CO₂ + 0.1% DEA |
| 31 Isomer 1 Isomer 2 | 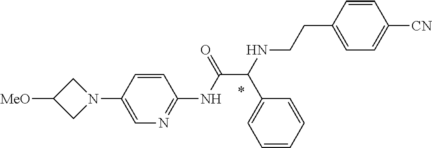 [SMILES: COC1CN(c2ccc(NC(=O)C(NCCc3ccc(C#N)cc3)c3ccccc3)nc2)C1] | (S)- and (R)-2- ((4-cyanophen- ethyl)amino)-N- (5-(3-methoxy- azetidin-1-yl)- pyridin-2-yl)-2- phenylacetamide | Calc'd 442.5, Found 442.4 and 442.4 | CHIRALCEL OX-H; 60% (50:50 MeOH:IPA) in hexanes + 0.1% DEA |
| 32 Isomer 1 Isomer 2 Isomer 3 Isomer 4 | 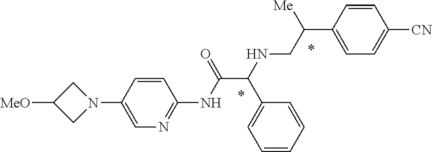 [SMILES: COC1CN(c2ccc(NC(=O)C(NCC(C)c3ccc(C#N)cc3)c3ccccc3)nc2)C1] | (S, S)-, (S, R)-, (R, R)- and (R, S)-2-((2-(4- cyanophenyl)- propyl)amino)- N-(5-(3-meth- oxyazetidin-1- yl)pyridin-2-yl)- 2-phenylacet- amide | Calc'd 456.3, Found 456.3, 456.3, 456.7 and 456.7 | 1-CHIRAL- CEL OX-H; 25% (70:30 IPA:ACN) in hexanes + 0.1% DEA 2-CHIRAL- CEL OX-H; 50% (50:50 MeOH:IPA) |
| | | | | in hexanes + |
| | | | | 0.1% DEA |
| 33 Isomer 1 Isomer 2 Isomer 3 Isomer 4 | 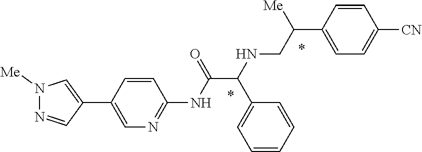 [SMILES: CC(CNC(C(=O)Nc1ccc(-c2cnn(C)c2)cn1)c1ccccc1)c1ccc(C#N)cc1] | (S, S)-, (S, R)-, (R, R)- and (R, S)-2-((2-(4- cyanphenyl)- propyl)amino)- N-(5-(1-methyl- 1H-pyrazol-4- yl)pyridin-2-yl)- 2-phenylacet- amide | Calc'd 451.2, Found 451.3, 451.3, 449.1 and 451.2 | 1-CHIRAL- PAK IC; 40% (50:50 IPA:MeOH) in hexanes + 0.1% DEA 2-CHIRAL- CEL OJ-H; 15% MeOH in Liquid CO₂ + |
| | | | | 0.1% DEA |
| 34 Isomer 1 Isomer 2 | 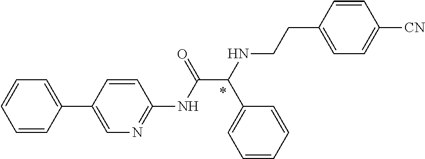 [SMILES: N#Cc1ccc(CCNC(C(=O)Nc2ccc(-c3ccccc3)cn2)c2ccccc2)cc1] | (S)- and (R)-2- ((4-cyanophen- ethyl)amino)-2- phenyl-N-(5- phenylpyridin- 2-yl)acetamide | Calc'd 433.2, Found 433.2 and 433.2 | CHIRALPAK IC; 25% MeOH in Liquid CO₂ + 0.1% DEA |
| 35 Isomer 1 Isomer 2 | 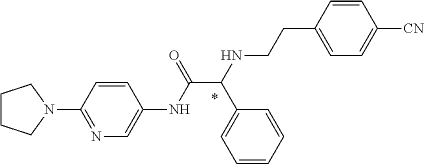 [SMILES: N#Cc1ccc(CCNC(C(=O)Nc2ccc(N3CCCC3)nc2)c2ccccc2)cc1] | (S)- and (R)-2- ((4-cyanophen- ethyl)amino)-2- phenyl-N-(6- (pyrrolidin-1- yl)-pyridin-3- yl)acetamide | Calc'd 426.2, Found 426.2 and 426.2 | CHIRALPAK IB: 30% MeOH in Liquid CO₂ + 0.1% DEA |
| 36 Isomer 1 Isomer 2 | 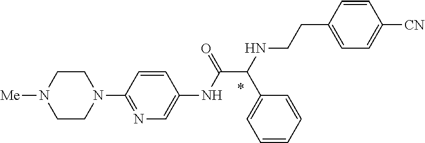 [SMILES: CN1CCN(c2ccc(NC(=O)C(NCCc3ccc(C#N)cc3)c3ccccc3)cn2)CC1] | (S)- and (R)-2- ((4-cyanophen- ethyl)amino)- N-(6-(4-methyl- piperazin-1-yl)- pyridin-3-yl)-2- phenylacetamide | Calc'd 455.3, Found 455.5 and 455.5 | CHIRALCEL OX-H; 20% (50:50 ACN:IPA) in Liquid CO₂ + 0.1% DEA |
| 37 Isomer 1 Isomer 2 | 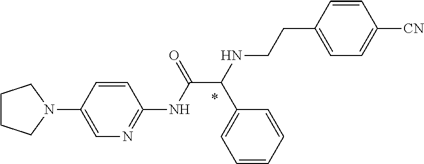 [SMILES: N#Cc1ccc(CCNC(C(=O)Nc2ccc(N3CCCC3)cn2)c2ccccc2)cc1] | (S)- and (R)-2- ((4-cyanophen- ethyl)amino)-2- phenyl-N-(5- (pyrrolidin-1- yl)pyridin-2-yl)- acetainide | Calc'd 426.2, Found 426.3 and 426.3 | CHIRALCEL OJ-H; 15% MeOH in Liquid CO₂ + 0.1% DEA |
| 38 Isomer 1 Isomer 2 | 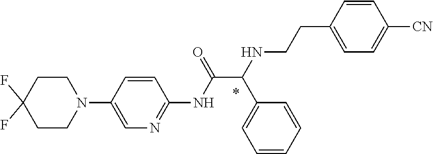 [SMILES: N#Cc1ccc(CCNC(C(=O)Nc2ccc(N3CCC(F)(F)CC3)cn2)c2ccccc2)cc1] | (S)- and (R)-2- ((4-cyanophen- ethyl)amino)-N- (5-(4,4-difluoro- piperidin-1-yl)- pyridin-2-yl)-2- phenylacetamide | Calc'd 476.2, Found 476.5 and 476.3 | CHIRALCEL OX-H; 25% (30:70 ACN:IPA) in hexanes + 0.1% DEA |
| 39 Isomer 1 Isomer 2 Isomer 3 Isomer 4 | 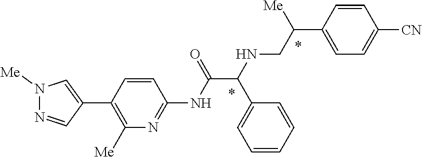 [SMILES: Cc1nc(NC(=O)C(NCC(C)c2ccc(C#N)cc2)c2ccccc2)ccc1-c1cnn(C)c1] | (S, S)-, (S, R)-, (R, R)- and (R, S)-2-((2-(4- cyanophenyl)- propyl)amino)- N-(6-methyl-5- (1-methyl-1H- pyrazol-4-yl)- pyridin-2-yl)-2- phenylacetamide | Calc'd 465.3, Found 465.4, 465.4, 465.6 and 465.7 | 1-CHIRAL- PAK IC; 40% (50:50 MeOH:IPA) in hexanes + 0.1% DEA 2-CHIRAL- CEL OX-H; 30% (30:70 ACN:IPA) |
| | | | | in hexanes + |
| | | | | 0.1% DEA |
| | | | | 3-CHIRAL- |
| | | | | PAK AD-H; |
| | | | | 20% MeOH in |
| | | | | Liquid CO₂ + |
| | | | | 0.1 % DEA |
| 40 Isomer 1 Isomer 2 | 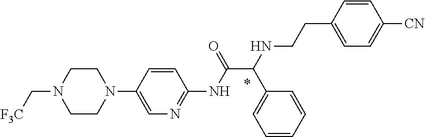 [SMILES: N#Cc1ccc(CCNC(C(=O)Nc2ccc(N3CCN(CC(F)(F)F)CC3)cn2)c2ccccc2)cc1] | (S)- and (R)-2- ((4-cyanophen- ethyl)amino)-2- phenyl-N-(5-(4- (2,2,2-trifluoro- ethyl)piperazin- 1-yl)pyridin-2- yl)acetamide | Calc'd 523.2, Found 523.4 and 523.4 | CHIRALCEL OX-H; 15% (50:50 ACN:EPA) in Liquid CO₂ + 0.1% DEA |
| 41 Isomer 1 Isomer 2 | 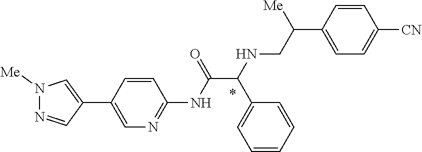 [SMILES: CC(CNC(C(=O)Nc1ccc(-c2cnn(C)c2)cn1)c1ccccc1)c1ccc(C#N)cc1] | (S)- and (R)-2- ((4-cyano-2- methylphen- ethyl)amino)- N-(5-(1-methyl- 1H-pyrazol-4- yl)pyridin-2-yl)- 2-phenylacet- amide | Calc'd 451.2, Found 451.5 and 451.4 | CHERALCEL OX-H; 55% (50:50 MeOH:IPA) in hexanes + 0.1% DEA |
| 42 Isomer 1 Isomer 2 | 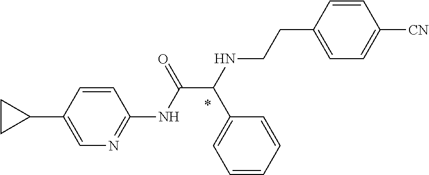 [SMILES: N#Cc1ccc(CCNC(C(=O)Nc2ccc(C3CC3)cn2)c2ccccc2)cc1] | (S)- and (R)-2- ((4-cyanophen- ethyl)amino)-N- (5-cyclopropyl- pyridin-2-yl)-2- phenylacetamide | Calc'd 397.2, Found 397.4 and 397.4 | CHIRALCEL OJ-H; 10% (50:50 ACN:IPA) in Liquid CO₂ + 0.1% DEA |
| 43 Isomer 1 Isomer 2 | 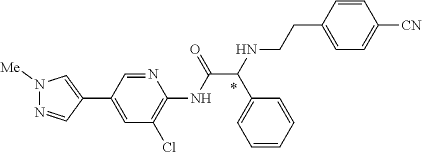 [SMILES: Cn1cc(-c2cnc(NC(=O)C(NCCc3ccc(C#N)cc3)c3ccccc3)c(Cl)c2)cn1] | (S)- and (R)-N- (3-chloro-5-(1- methyl-1H- pyrazol-4-yl)- pyridin-2-yl)-2- ((4-cyanophen- ethyl)amino)-2- phenylacetamide | Calc'd 471.2, Found 471.5 and 471.5 | CHIRALCEL OX-H; 70% (25:75 MeOH:IPA) in hexanes + 0.1% DEA |
| 44 Isomer 1 Isomer 2 | 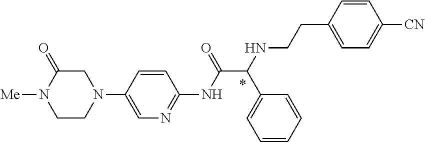 [SMILES: CN1CCN(c2ccc(NC(=O)C(NCCc3ccc(C#N)cc3)c3ccccc3)nc2)CC1=O] | (S)- and (R)-2- ((4-cyanophen- ethyl)amino)-N- (5-(4-methyl-3- oxopiperazin-1- yl)pyridin-2-yl)- 2-phenylacet- amide | Calc'd 469.6, Found 469.7 and 469.8 | CHIRALPAK IC; 45% (30:70 ACN:EPA) in hexanes + 0.1% DEA |
| 45 Isomer 1 Isomer 2 | 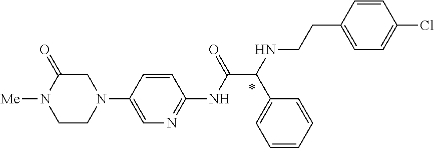 [SMILES: CN1CCN(c2ccc(NC(=O)C(NCCc3ccc(Cl)cc3)c3ccccc3)nc2)CC1=O] | (S)- and (R)-2- ((4-chlorophen- ethyl)amino)-N- (5-(4-methyl-3- oxopiperazin-1- yl)pyridin-2-yl)- 2-phenylacet- amide | Calc'd 479.2, Found 478.8 and 478.7 | CHIRALCEL OX-H; 60% (50:50 MeOH:IPA) in hexanes + 0.1% DEA |
| 46 Isomer 1 Isomer 2 | 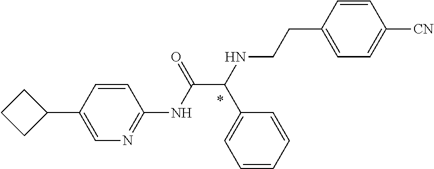 [SMILES: N#Cc1ccc(CCNC(C(=O)Nc2ccc(C3CCC3)cn2)c2ccccc2)cc1] | (S)- and (R)-2- ((4-cyanophen- ethyl)amino)-N- (5-cyclobutyl- pyridin-2-yl)-2- phenylacetamide | Calc'd 411.2, Found 411.3 and 411.3 | CHIRALPAK IB; 10% MeOH in Liquid CO₂ + 0.1% DEA |
| 47 Isomer 1 Isomer 2 Isomer 3 Isomer 4 | 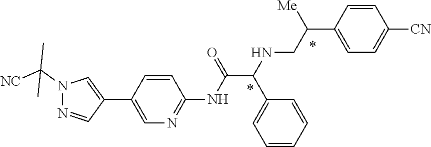 [SMILES: CC(CNC(C(=O)Nc1ccc(-c2cnn(C(C)(C)C#N)c2)cn1)c1ccccc1)c1ccc(C#N)cc1] | (S, S)-, (S, R)-, (R, R)- and (R, S)- 2-((2-(4-cyano- phenyl)propyl)- amino)-N-(5-(1-(2- cyanopropan-2-yl)- 1H-pyrazol-4-yl)- pyridin-2-yl)-2- phenylacetamide | Calc'd 504.2, Found 504.4, 504.7, 504.7 and 504.6 | 1-CHIRAL- PAK IC; 20% MeOH in Liquid CO₂ + 0.1% DEA 2-CHIRAL- PAK AD-H; 35% MeOH in Liquid CO₂ + |
| | | | | 0.1% DEA |
| 48 Isomer 1 Isomer 2 | 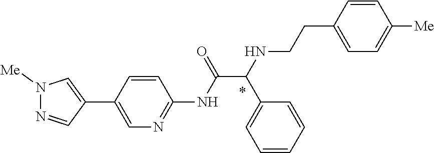 [SMILES: Cc1ccc(CCNC(C(=O)Nc2ccc(-c3cnn(C)c3)cn2)c2ccccc2)cc1] | (S)- and (R)-N- (5-(1-methyl- 1H-pyrazol-4- yl)pyridin-2-yl)- 2-((4-methyl- phenethyl)- amino)-2-phen- ylacetamide | Calc'd 426.2, Found 426.4 and 426.4 | CHIRALCEL OJ-H; 30% MeOH in Liquid CO₂ + 0.1% DEA |
| 49 Isomer 1 Isomer 2 | 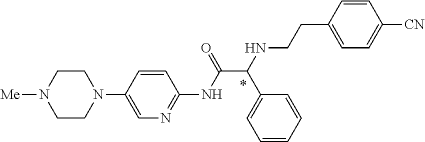 [SMILES: CN1CCN(c2ccc(NC(=O)C(NCCc3ccc(C#N)cc3)c3ccccc3)nc2)CC1] | (S)- and (R)-2- ((4-cyanophen- ethyl)amino)- N-(5-(4-methyl- piperazin-1-yl)- pyridin-2-yl)-2- phenylacetamide | Calc'd 455.3, Found 455.6 and 455.5 | CHIRALCEL OJ-H; 18% MeOH in Liquid CO₂ + 0.1% DEA |
| 50 Isomer 1 Isomer 2 | 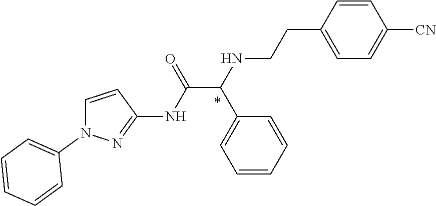 [SMILES: N#Cc1ccc(CCNC(C(=O)Nc2ccn(-c3ccccc3)n2)c2ccccc2)cc1] | (S)- and (R)-2- ((4-cyanophen- ethyl)amino)-2- phenyl-N-(1- phenyl-1H- pyrazol-3-yl)- acetamide | Calc'd 422.3, Found 422.6 and 422.3 | CHIRALCEL OJ-H; 30% MeOH in Liquid CO₂ + 0.1% DEA |
| 51 Isomer 1 Isomer 2 | 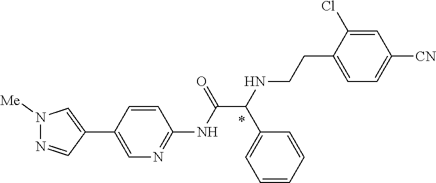 [SMILES: Cn1cc(-c2ccc(NC(=O)C(NCCc3ccc(C#N)cc3Cl)c3ccccc3)nc2)cn1] | (S)- and (R)-2- ((2-chloro-4- cyanophen- ethyl)amino)-N- (5-(1-methyl- 1H-pyrazol-4- yl)pyridin-2-yl)- 2-phenylacet- amide | Calc'd 471.2, Found 471.3 and 471.3 | CHIRALCEL OX-H; 50% (50:50 IPA:MeOH) in hexanes + 0.1% DEA |
| 52 Isomer 1 Isomer 2 Isomer 3 Isomer 4 | 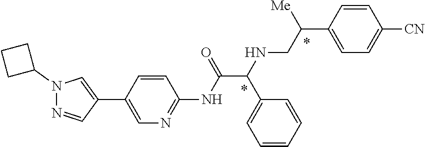 [SMILES: CC(CNC(C(=O)Nc1ccc(-c2cnn(C3CCC3)c2)cn1)c1ccccc1)c1ccc(C#N)cc1] | (S, S)-,(S, R)-, (R, R)-and (R, S)- 2-((2-(4-cyano- phenyl)propyl)- amino)-N-(5-(1- cyclobutyl-1H- pyrazol-4-yl)-pyri- din-2-yl)-2-phenyl- acetamide | Calc'd 491.3, Found 491.6, 491.5, 491.6 and 491.6 | 1-CHIRAL- CEL OX-H; 20% (50:50; ACN:IPA) in Liquid CO₂ + 0.1% DEA 2-CHIRAL- CEL OX-H; 45% (50:50 |
| | | | | MeOH:IPA) |
| | | | | in hexanes + |
| | | | | 0.1% DEA |
| 53 Isomer 1 Isomer 2 | 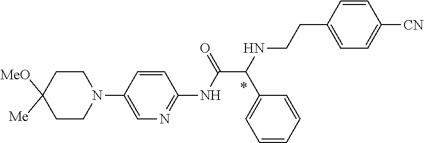 [SMILES: COC1(C)CCN(c2ccc(NC(=O)C(NCCc3ccc(C#N)cc3)c3ccccc3)nc2)CC1] | (S)- and (R)-2- ((4-cyanophen- ethyl)amino)-N- (5-(4-methoxy- 4-methylpiperi- din-1-yl)pyridin- 2-yl)-2-phenyl- acetamide | Calc'd 484.3, Found 484.5 and 484.6 | CHIRALCEL OX-H; 55% (50:50 MeOH:IPA) in hexanes + 0.1% DEA |
| 54 Isomer 1 Isomer 2 Isomer 3 Isomer 4 | 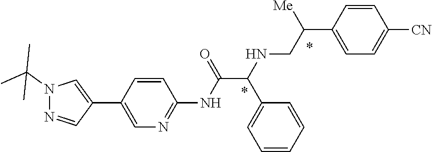 [SMILES: CC(CNC(C(=O)Nc1ccc(-c2cnn(C(C)(C)C)c2)cn1)c1ccccc1)c1ccc(C#N)cc1] | (S,S)-, (S, R)-, (R, R)-and (R, S)-N-(5-(1- (tert-butyl)-1H- pyrazol-4-yl)- pyridin-2-yl)-2- ((2-(4-cyano- phenyl)propyl)- amino)-2-phen- ylacetamide | Calc'd 493.3, Found 493.6, 493.4, 493.7 and 493.7 | 1-CHIRAL- CEL OX-H; 35% (50:50 IPA:MeOH) in hexanes + 0.1% DEA 2-CHIRAL- PAK IC; 30% (50:50 IPA:MeOH) |
| | | | | in hexanes + |
| | | | | 0.1% DEA |
| 55 Isomer 1 Isomer 2 | 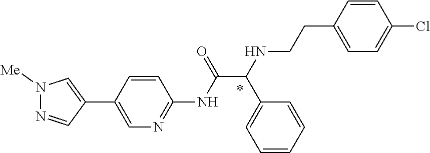 [SMILES: Cn1cc(-c2ccc(NC(=O)C(NCCc3ccc(Cl)cc3)c3ccccc3)nc2)cn1] | (S)- and (R)-2- ((4-chlorophen- ethyl)amino)- N-(5-(1-methyl- 1H-pyrazol-4- yl)-pyridin-2- yl)-2-phenyl- acetamide | Calc'd 446.2, Found 446.5 and 446.5 | CHIRALCEL OJ-H; 30% MeOH in Liquid CO₂ + 0.1% DEA |
| 56 Isomer 1 Isomer 2 | 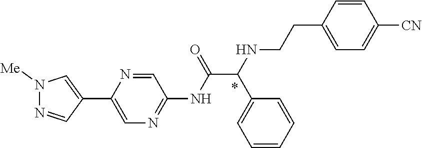 [SMILES: Cn1cc(-c2cnc(NC(=O)C(NCCc3ccc(C#N)cc3)c3ccccc3)cn2)cn1] | (S)- and (R)-2- ((4-cyanophen- ethyl)amino)- N-(5-(1-methyl- 1H-pyrazol-4- yl)pyrazin-2- yl)-2-phenylacet- amide | Calc'd 438.2, Found 438.5 and 438.5 | CHIRALPAK IB; 45% (50:50 MeOH:EPA) in hexanes + 0.1% DEA |
| 57 Isomer 1 Isomer 2 | 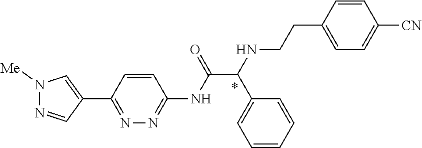 [SMILES: Cn1cc(-c2ccc(NC(=O)C(NCCc3ccc(C#N)cc3)c3ccccc3)nn2)cn1] | (S)- and (R)-2- ((4-cyanophen- ethyl)amino)-N- (6-(1-methyl- 1H-pyrazol-4- yl)pyridazin-3- yl)-2-phenyl- acetamide | Calc'd 438.2, Found 438.3 and 438.3 | CHIRALPAK IC; 35% (50:50 ACN:IPA) in hexanes + 0.1% DEA |
| 58 Isomer 1 Isomer 2 | 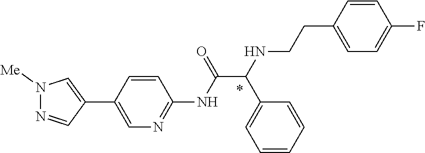 [SMILES: Cn1cc(-c2ccc(NC(=O)C(NCCc3ccc(F)cc3)c3ccccc3)nc2)cn1] | (S)- and (R)-2- ((4-fluorophen- ethyl)amino)-N- (5-(1-methyl- 1H-pyrazol-4- yl)pyridin-2-yl)- 2-phenylacet- amide | Calc'd 430.2, Found 430.3 and 430.5 | CHIRALCEL OJ-H; 20% MeOH in Liquid CO₂ + 0.1% DEA |
| 59 Isomer 1 Isomer 2 Isomer 3 Isomer 4 | 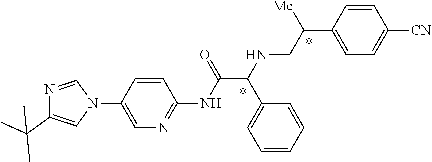 [SMILES: CC(CNC(C(=O)Nc1ccc(-n2cnc(C(C)(C)C)c2)cn1)c1ccccc1)c1ccc(C#N)cc1] | (S, S)-,(S, R)-, (R, R)- and (R, S)-N-(5-(4- (tert-butyl)-1H- imidazol-1-yl)- pyridin-2-yl)-2- ((2-(4-cyano- phenyl)propyl)- amino)-2-phenyl- acetamide | Calc'd 493.3, Found 493.7, 493.6, 493.7 and 493.8 | 1-CHIRAL- CEL OJ-H; 10% MeOH in Liquid CO₂ + 0.1% DEA 2-CHIRAL- CEL OX-H; 25% (70:30 IPA:ACN) in hexanes + |
| | | | | 0.1% DEA |
| 60 Isomer 1 Isomer 2 | 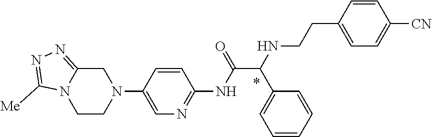 [SMILES: Cc1nnc2n1CCN(c1ccc(NC(=O)C(NCCc3ccc(C#N)cc3)c3ccccc3)nc1)C2] | (S)- and (R)-2- ((4-cyanophen- ethyl)amino)-N- (5-(3-methyl- 5,6-dihydro- [1,2,4]triazolo- [4,3-a]pyrazin- 7(8H)-yl)pyridin- 2-yl)-2-phenyl- | Calc'd 493.2, Found 493.2 and 493.4 | CHIRALCEL OX-H; 100% (50:50 MeOH:IPA) + 0.1% DEA |
| | | acetamide | | |
| 61 Isomer 1 Isomer 2 Isomer 3 Isomer 4 | 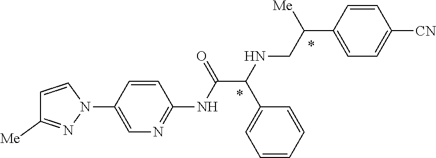 [SMILES: Cc1ccn(-c2ccc(NC(=O)C(NCC(C)c3ccc(C#N)cc3)c3ccccc3)nc2)n1] | (S, S)-, (S, R)-, (R, R)-and (R, S)-2-((2-(4- cyanophenyl)- propyl)amino)- N-(5-(3-methyl- 1H-pyrazol-1- yl)pyridin-2-yl)- 2-phenylacet- amide | Calc'd 451.2, Found 451.2, 451.2, 451.2 and 451.2 | 1-CHIRAL- CEL OX-H; 35% (50:50 MeOH:IPA) in hexanes + 0.1% DEA 2-CHIRAL- CEL OX-H; 15% (50:50 MeOH:IPA) |
| | | | | in hexanes + |
| | | | | 0.1% DEA |
| 62 Isomer 1 Isomer 2 Isomer 3 Isomer 4 | 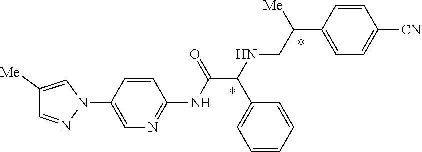 [SMILES: Cc1cnn(-c2ccc(NC(=O)C(NCC(C)c3ccc(C#N)cc3)c3ccccc3)nc2)c1] | (S, S)-,(S, R)-, (R, R)- and (R, S)-2-((2-(4- cyanophenyl)- propyl)amino)- N-(5-(4-methyl- 1H-pyrazol-1- yl)pyridin-2-yl)- 2-phenylacet- amide | Calc'd 451.2, Found 451.4, 451.2, 451.3 and 451.3 | 1-CHIRAL- CEL OX-H; 20% MeOH in Liquid CO₂ + 0.1% DEA 2-CHIRAL- PAK IC; 15% (30:70 ACN:IPA) in hexanes + |
| | | | | 0.1% DEA |
| 63 Isomer 1 Isomer 2 | 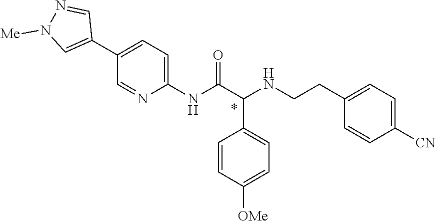 [SMILES: COc1ccc(C(NCCc2ccc(C#N)cc2)C(=O)Nc2ccc(-c3cnn(C)c3)cn2)cc1] | (S)- and (R)-2- ((4-cyanophen- ethyl)amino)- 2-(4-methoxy- phenyl)-N-(5- (1-methyl-1H- pyrazol-4-yl)- pyridin-2-yl)- acetamide | Calc'd 467.2, Found 467.5 and 467.6 | CHIRALPAK IC; 37% MeOH in Liquid CO₂ + 0.1% DEA |
| 64 Isomer 1 Isomer 2 | 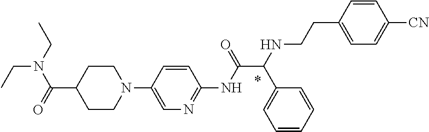 [SMILES: CCN(CC)C(=O)C1CCN(c2ccc(NC(=O)C(NCCc3ccc(C#N)cc3)c3ccccc3)nc2)CC1] | (S)- and (R)-1- (6-(2-((4-cyano- phenethyl)amino)- 2-phenylacet- amido)pyridin-3- yl)-N,N-diethyl- piperidine-4- carboxamide | Calc'd 539.3, Found 539.9 and 539.9 | CHIRALPAK AD-H; 35% (50:50 ACN:IPA) in Liquid CO₂ + 0.3% DEA |
| 65 Isomer 1 Isomer 2 | 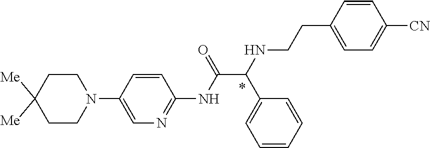 [SMILES: CC1(C)CCN(c2ccc(NC(=O)C(NCCc3ccc(C#N)cc3)c3ccccc3)nc2)CC1] | (S)- and (R)-2- ((4-cyanophen- ethyl)amino)- N-(5-(4,4- dimethylpiperi- din-1-yl)pyridin- 2-yl)-2-phenyl- acetamide | Calc'd 468.3, Found 468.5 and 468.6 | CHIRALCEL OX-H; 35% (50:50 MeOH:IPA) in hexanes + 0.1% DEA |
| 66 Isomer 1 Isomer 2 | 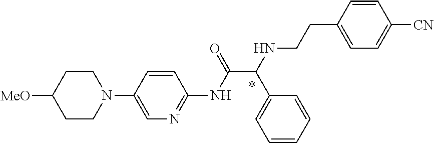 [SMILES: COC1CCN(c2ccc(NC(=O)C(NCCc3ccc(C#N)cc3)c3ccccc3)nc2)CC1] | (S)- and (R)-2- ((4-cyanophen- ethyl)amino)-N- (5-(4-methoxy- piperidin-1-yl)- pyridin-2-yl)-2- phenylacetamide | Calc'd 470.3, Found 470.5 and 470.5 | CHIRALCEL OX-H; 45% (50:50 MeOH:TPA) in hexanes + 0.1% DEA |
| 67 Isomer 1 Isomer 2 | 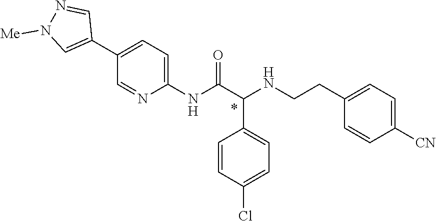 [SMILES: Cn1cc(-c2ccc(NC(=O)C(NCCc3ccc(C#N)cc3)c3ccc(Cl)cc3)nc2)cn1] | (S)- and (R)-2- (4-chlorophenyl)- 2-((4-cyanophen- ethyl)amino)-N- (5-(1-methyl- 1H-pyrazol-4- yl)pyridin-2-yl)- acetamide | Calc'd 471.2, Found 471.5 and 471.3 | CHIRALCEL OX-H; 35% (30:70 ACN:IPA) in hexanes + 0.1% DEA |
| 68 Isomer 1 Isomer 2 | 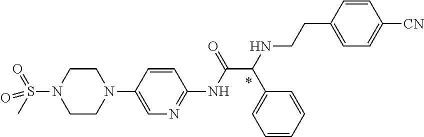 [SMILES: CS(=O)(=O)N1CCN(c2ccc(NC(=O)C(NCCc3ccc(C#N)cc3)c3ccccc3)nc2)CC1] | (S)- and (R)-2- ((4-cyanophen- ethyl)amino)-N- (5-(4-(methyl- sulfonyl)pipera- zin-1-yl)-pyridin- 2-yl)-2-phenyl- acetamide | Calc'd 519.2, Found 519.5 and 519.6 | CIIIRALPAK IC; 35% (70:30 IPA:ACN) in hexanes + 0.1% DEA |
| 69 Isomer 1 Isomer 2 | 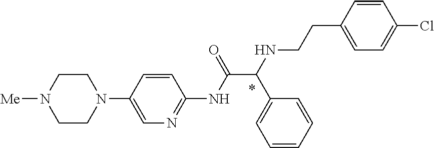 [SMILES: CN1CCN(c2ccc(NC(=O)C(NCCc3ccc(Cl)cc3)c3ccccc3)nc2)CC1] | (S)- and (R)-2- ((4-chlorophen- ethyl)amino)- N-(5-(4-methyl- piperazin-1-yl)- pyridin-2-yl)-2- phenylacetamide | Calc'd 464.2, Found 464.5 and 464.5 | CIIIRALCEL OX-H; 35% (50:50 MeOH:IPA) in hexanes + 0.1% DEA |
| 70 Isomer 1 Isomer 2 | 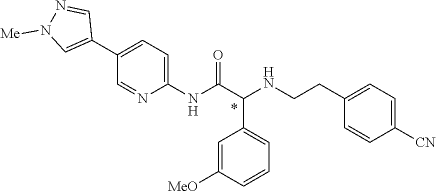 [SMILES: COc1cccc(C(NCCc2ccc(C#N)cc2)C(=O)Nc2ccc(-c3cnn(C)c3)cn2)c1] | (S)- and (R)-2- ((4-cyanophen- ethyl)amino)- 2-(3-methoxy- phenyl)-N-(5- (1-methyl-1H- pyrazol-4-yl)- pyridin-2-yl)- acetamide | Calc'd 467.2, Found 467.3 and 467.3 | CHIRALCEL OJ-H; 20% MeOH in Liquid CO₂ + 0.1% DEA |
| 71 Isomer 1 Isomer 2 | 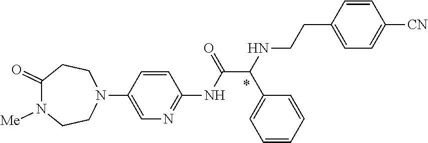 [SMILES: CN1CCN(c2ccc(NC(=O)C(NCCc3ccc(C#N)cc3)c3ccccc3)nc2)CCC1=O] | (S)- and (R)-2- ((4-cyanophen- ethyl)amino)-N- (5-(4-methyl-5- oxo-1,4-diaze- pan-1-yl)pyridin- 2-yl)-2-phenyl- acetamide | Calc'd 483.2, Found 483.6 and 483.6 | CHIRALCEL OJ-H; 35% MeOH in Liquid CO₂ + 0.1% DEA |
| 72 Isomer 1 Isomer 2 | 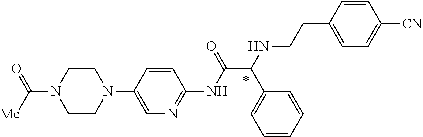 [SMILES: CC(=O)N1CCN(c2ccc(NC(=O)C(NCCc3ccc(C#N)cc3)c3ccccc3)nc2)CC1] | (S)- and (R)-N- (5-(4-acetyl- piperazin-1-yl)- pyridin-2-yl)-2- ((4-cyanophen- ethyl)amino)- 2-phenylacet- amide | Calc'd 483.2, Found 483.4 and 483.6 | CHIRALCEL OX-H; 55% (50:50 MeOH:IPA) in hexanes + 0.1% DEA |
| 73 Isomer 1 Isomer 2 | 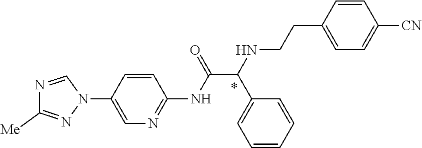 [SMILES: Cc1ncn(-c2ccc(NC(=O)C(NCCc3ccc(C#N)cc3)c3ccccc3)nc2)n1] | (S)- and (R)- 2-((4-cyano- phenethyl)- amino)-N-(5- (2-methoxy- ethoxy)pyridin- 2-yl)-2-phenyl- acetamide | Calc'd 438.2, Found 438.4 and 438.5 | CHIRALCEL OX-H; 25% MeOH in Liquid CO₂ + 0.1% DEA |
| 74 Isomer 1 Isomer 2 | 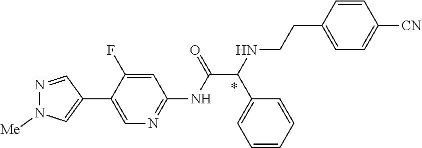 [SMILES: Cn1cc(-c2cnc(NC(=O)C(NCCc3ccc(C#N)cc3)c3ccccc3)cc2F)cn1] | (S)- and (R)-2- ((4-cyanophen- ethyl)amino)-N- (4-fluoro-5-(1- methyl-1H- pyrazol-4-yl)- pyridin-2-yl)-2- phenylacetamide | Calc'd 455.2, Found 455.7 and 455.3 | CHIRALCEL OJ-H; 10% (50:50 MeOH:IPA) in Liquid CO₂ + 0.1% DEA |
| 75 Isomer 1 Isomer 2 | 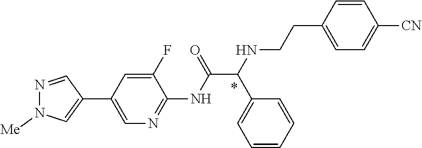 [SMILES: Cn1cc(-c2cnc(NC(=O)C(NCCc3ccc(C#N)cc3)c3ccccc3)c(F)c2)cn1] | (S)- and (R)-2- ((4-cyanophen- ethyl)amino)- N-(3-fluoro-5- (1-methyl-1H- pyrazol-4-yl)- pyridin-2-yl)-2- phenylacetamide | Calc'd 455.2, Found 455.7 and 455.3 | CHIRALCEL OX-H; 60% (30:70 ACN:IPA) in hexanes + 0.1% DEA |
| 76 Isomer 1 Isomer 2 | 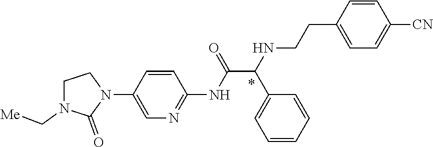 [SMILES: CCN1CCN(c2ccc(NC(=O)C(NCCc3ccc(C#N)cc3)c3ccccc3)nc2)C1=O] | (S)- and (R)-2- ((4-cyanophen- ethyl)amino)-N- (5-(3-ethyl-2- oxoimidazolidin- 1-yl)pyridin-2- yl)-2-phenyl- acetamide | Calc'd 469.2, Found 469.6 and 469.6 | CHIRALCEL OJ-H; 20% MeOH in Liquid CO₂ + 0.1% DEA |
| 77 Isomer 1 Isomer 2 | 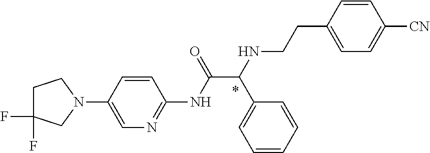 [SMILES: N#Cc1ccc(CCNC(C(=O)Nc2ccc(N3CCC(F)(F)C3)cn2)c2ccccc2)cc1] | (S)- and (R)-2- ((4-cyanophen- ethyl)amino)-N- (5-(3,3-difluoro- pyrrolidin-1-yl)- pyridin-2-yl)-2- phenylacetamide | Calc'd 462.2, Found 462.7 and 462.7 | CHIRALCEL OX-H; 25% (70:30 IPA:ACN) in hexanes + 0.1% DEA |
| 78 Isomer 1 Isomer 2 | 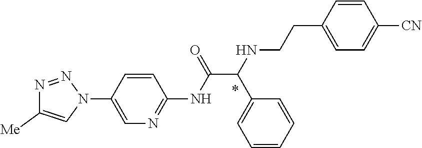 [SMILES: Cc1cn(-c2ccc(NC(=O)C(NCCc3ccc(C#N)cc3)c3ccccc3)nc2)nn1] | (S)- and (R)-2- ((4-cyanophen- ethyl)amino)-N- (5-(4-methyl- 1H-1,2,3-triazol- 1-yl)pyridin-2- yl)-2-phenyl- acetamide | Calc'd 438.2, Found 438.5 and 438.4 | CHIRALCEL OX-H; 15% MeOH in Liquid CO₂ + 0.1% DEA |
| 79 Isomer 1 Isomer 2 | 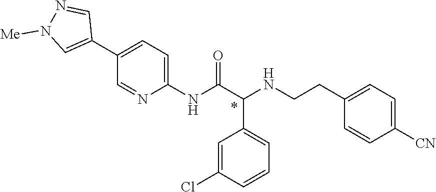 [SMILES: Cn1cc(-c2ccc(NC(=O)C(NCCc3ccc(C#N)cc3)c3cccc(Cl)c3)nc2)cn1] | (S)- and (R)-2- (3-chlorophenyl)- 2-((4-cyanophen- ethyl)amino)-N- 5-(1-methyl-1H- pyrazol-4-yl)- pyridin-2-yl)- acetamide | Calc'd 471.2, Found 471.4 and 471.5 | CHIRALCEL OX-H; 20% (70:30 IPA:ACN) in hexanes + 0.1% DEA |
| 80 Isomer 1 Isomer 2 | 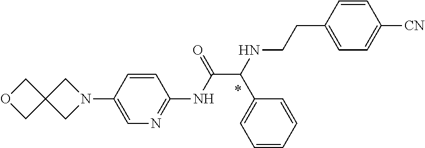 [SMILES: N#Cc1ccc(CCNC(C(=O)Nc2ccc(N3CC4(COC4)C3)cn2)c2ccccc2)cc1] | (S)- and (R)-N- (5-(2-oxa-6- azaspiro[3.3]- heptan-6-yl)- pyridin-2-yl)-2- ((4-cyanophen- ethyl)amino)-2- phenylacetamide | Calc'd 454.2, Found 454.7 and 454.7 | CHIRALCEL OX-H; 28% MeOH in Liquid CO₂ + 0.1% DEA |
| 81 Isomer 1 Isomer 2 | 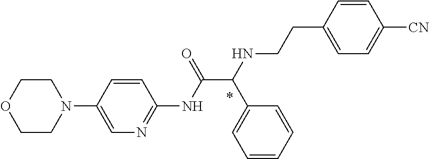 [SMILES: N#Cc1ccc(CCNC(C(=O)Nc2ccc(N3CCOCC3)cn2)c2ccccc2)cc1] | (S)- and (R)-2- ((4-cyanophen- ethyl)amino)-N- (5-morpholino- pyridin-2-yl)-2- phenylacetamide | Calc'd 442.2, Found 442.6 and 442.6 | CIIIRALCEL OJ-H; 13% MeOH in Liquid CO₂ + 0.1% DEA |
| 82 Isomer 1 Isomer 2 Isomer 3 Isomer 4 | 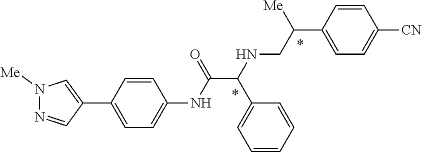 [SMILES: CC(CNC(C(=O)Nc1ccc(-c2cnn(C)c2)cc1)c1ccccc1)c1ccc(C#N)cc1] | (S, S)-, (S, R)-, (R, R)- and (R, S)-2-((2-(4- cyanophenyl)- propyl)amino)- N-(4-(1-methyl- 1H-pyrazol-4- yl)phenyl)-2- phenylacetamide | Calc'd 450.2, Found 450.5, 450.3, 450.3 and 450.4 | 1-CHIRAL- PAK IC; 20% (30:70 ACN:IPA) in hexanes + 0.1% DEA 2-CHIRAL- CEL OJ-H; 15% (50:50 IPA:ACN) in |
| | | | | Liquid CO₂ + |
| | | | | 0.1% DEA |
| 83 Isomer 1 Isomer 2 Isomer 3 Isomer 4 | 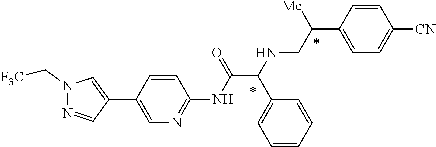 [SMILES: CC(CNC(C(=O)Nc1ccc(-c2cnn(CC(F)(F)F)c2)cn1)c1ccccc1)c1ccc(C#N)cc1] | (S, S)-,(S, R)-, (R,R)- and (R, S)-2-((2-(4- cyanophenyl)- propyl)amino)- 2-phenyl-N-(5- (1-(2,2,2-tri- fluoroEthyl)- 1H-pyrazol-4- | Calc'd 519.2, Found 519.4, 519.4, 519.3 and 519.4 | 1-CHIRAL- CEL OX-H; 8% MeOH in Liquid CO₂ + 0.1% DEA 2-CHIRAL- CEL OX-H; 40% (70:30 IPA:ACN) |
| | | yl)pyridin-2- | | in hexanes + |
| | | yl)acetamide | | 0.1% DEA |
| 84 Isomer 1 Isomer 2 | 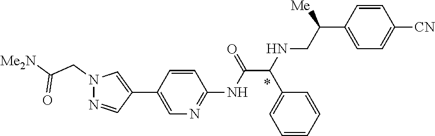 [SMILES: C[C@H](CNC(C(=O)Nc1ccc(-c2cnn(CC(=O)N(C)C)c2)cn1)c1ccccc1)c1ccc(C#N)cc1] | (R, S)- and (S, S)-((2-(4-cyano- phenyl)propyl)- amino)-N-(5-(1- (2-(dimethyl- amino)-2-oxo- ethyl)-1H-pyra- zol-4-yl)pyridin- 2-yl)-2-phenyl- | Calc'd 522.3, Found 522.8 and 522.6 | CHIRALCEL OJ-H; 15% MeOH in Liquid CO₂ + 0.1% DEA |
| | | acetamide | | |
| 85 Isomer 1 Isomer 2 | 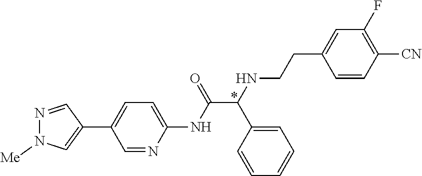 [SMILES: Cn1cc(-c2ccc(NC(=O)C(NCCc3ccc(C#N)c(F)c3)c3ccccc3)nc2)cn1] | (S)- and (R)-2- ((4-cyano-3- fluorophen- Ethyl)amino)- N-(5-(1-methyl- 1H-pyrazol-4- yl)pyridin-2-yl)- 2-phenylacet- amide | Calc'd 455.2, Found 455.3 and 455.3 | CHIRALCEL OX-H; 30% (70:30 IPA:ACN) in hexanes + 0.1% DEA |
| 86 Racemic | 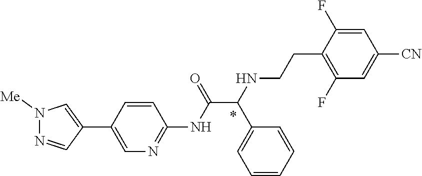 [SMILES: Cn1cc(-c2ccc(NC(=O)C(NCCc3c(F)cc(C#N)cc3F)c3ccccc3)nc2)cn1] | (S)- and (R)- 2- ((4-cyano-2,6- difluorophen- Ethyl)amino)- N-(5-(1-methyl- 1H-pyrazol-4- yl)pyridin-2-yl)- 2-phenylacet- amide | Calc'd 473.2, Found 473.3 | Racemic |
| 87 Isomer 1 Isomer 2 | 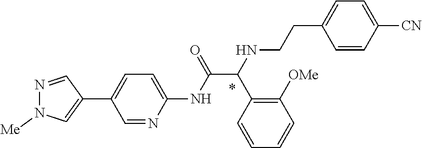 [SMILES: COc1ccccc1C(NCCc1ccc(C#N)cc1)C(=O)Nc1ccc(-c2cnn(C)c2)cn1] | (S)-and (R)- 2- ((4-cyanophen- Ethyl)amino)- 2-(2-methoxy- phenyl)-N-(5- (1-methyl-1H- pyrazol-4-yl)- pyridin-2-yl)- acetamide | Calc'd 467.2, Found 467.4 and 467.4 | CHIRALCEL OX-H; 20% (50:50 IPA:MeOH) in hexanes + 0.1% DEA |
| 88 Isomer 1 Isomer 2 | 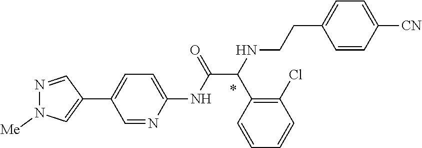 [SMILES: Cn1cc(-c2ccc(NC(=O)C(NCCc3ccc(C#N)cc3)c3ccccc3Cl)nc2)cn1] | (S)- and (R)- 2-(2-chloro- phenyl)-2-((4- cyanophen- Ethyl)amino)- N-(5-(1-methyl- 1H-pyrazol-4- yl)-pyridin-2-yl)- acetamide | Calc'd 471.2, Found 471.3 and 471.3 | CHIRALCEL OJ-H; 20% MeOH in Liquid CO₂ + 0.1% DEA |
| 89 Isomer 1 Isomer 2 | 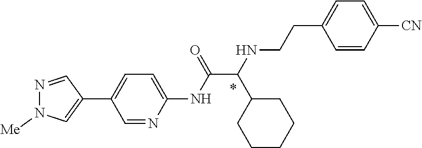 [SMILES: Cn1cc(-c2ccc(NC(=O)C(NCCc3ccc(C#N)cc3)C3CCCCC3)nc2)cn1] | (S)- and (R)- 2- ((4-cyanophen- Ethyl)amino)- 2-cyclohexyl-N- (S-(1-methyl- 1H-pyrazol-4- yl)pyridin-2-yl)- acetamide | Calc'd 443.3, Found 443.6 and 443.5 | CHIRALCEL OX-H; 25% (70:30 IPA:ACN) in hexanes + 0.1% DEA |
| 90 Isomer 1 Isomer 2 Isomer 3 Isomer 4 | 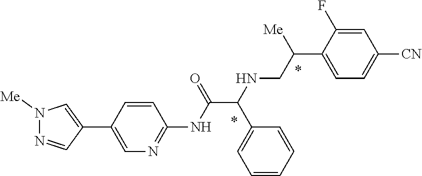 [SMILES: CC(CNC(C(=O)Nc1ccc(-c2cnn(C)c2)cn1)c1ccccc1)c1ccc(C#N)cc1F] | (S, S)-, (S, R)-, (R,R)- and (R, 5)-2-((2-(4- cyano-2-fluoro- phenyl)propyl)- amino)-N-(5- (1-methyl-1H- pyrazol-4-yl)- pyridin-2-yl)-2- phenylacetamide | Calc'd 469.2, Found 469.6, 469.7, 469.7 and 469.7 | 1-CHIRAL- CEL OX-H; 40% (50:50 IPA:MeOH) in hexanes + 0.1% DEA 2-CHIRAL- CEL OJ-H; 18% MeOH in Liquid CO₂ + 0.1% DEA |
| 91 Isomer 1 Isomer 2 Isomer 3 Isomer 4 | 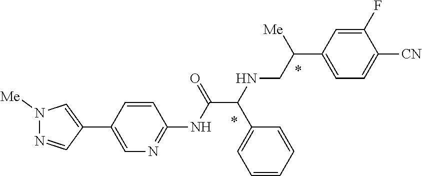 [SMILES: CC(CNC(C(=O)Nc1ccc(-c2cnn(C)c2)cn1)c1ccccc1)c1ccc(C#N)c(F)c1] | (S, S)-, (S, R)-, (R, R)- and (R, S)-2-((2-(4- cyano-3-fluoro- phenyl)propyl)- amino)-N-(5-(1- methyl-1H- pyrazol-4-yl)- pyridin-2-yl)-2- phenylacetamide | Calc'd 469.2, Found 469.5, 469.5, 469.5 and 469.4 | 1-CHIRAL- CEL OX-H; 35% (50:50 IPA:MeOH) in hexanes + 0.1% DEA 2-CHIRAL- CEL OX-H; 45% (50:50 IPA:MeOH) in hexanes + |
| | | | | 0.1% DEA |
| 92 Isomer 1 Isomer 2 Isomer 3 Isomer 4 | 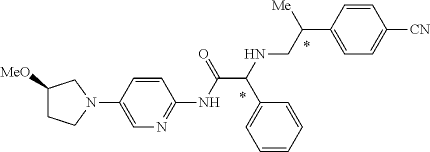 [SMILES: CO[C@@H]1CCN(c2ccc(NC(=O)C(NCC(C)c3ccc(C#N)cc3)c3ccccc3)nc2)C1] | (S, S, R)-, (S, R, R)-, (R, R, R)- and (R, S, R)-2- ((2-(4-cyano- phenyl)propyl)- amino)-N-(5- ((R)-3-methoxy- pyrrolidin-1-yl)- pyridin-2-yl)-2- phenylacetamide | Calc'd 470.3, Found 470.3, 470.4, 470.8 and 470.8 | 1-CHIRAL- CEL OX-H; 28% MeOH in Liquid CO₂ + 0.1% DEA 2-CHIRAL- PAK AD-H; 100% (80:20 ACN:MeOH) + 0.1% DEA |
| 93 Isomer 1 Isomer 2 Isomer 3 Isomer 4 | 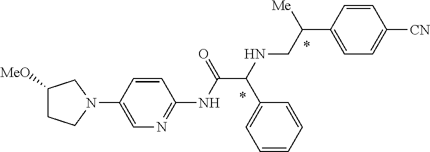 [SMILES: CO[C@H]1CCN(c2ccc(NC(=O)C(NCC(C)c3ccc(C#N)cc3)c3ccccc3)nc2)C1] | (S, S, S)-, (S, R, S)-, (R, R, S)- and (R, S, S)-2- ((2-(4-cyano- phenyl)propyl)- amino)-N-(5- ((S)-3-methoxy- pyrrolidin-1-yl)- pyridin-2-yl)-2- phenylacetamide | Calc'd 470.3, Found 470.7, 470.7, 470.7 and 470.7 | 1-CHIRAL- CEL OX-H; 35% (50:50 MeOH:IPA) in Liquid CO₂ + 0.1% DEA 2-CHIRAL- CEL OS-H; 35% (50:50 MeOH:IPA) in |
| | | | | Liquid CO₂ + |
| | | | | 0.1% DEA |
| 94 Isomer 1 Isomer 2 | 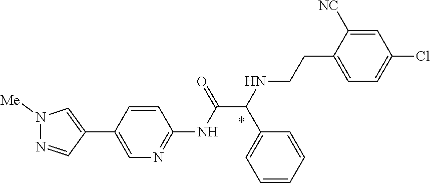 [SMILES: Cn1cc(-c2ccc(NC(=O)C(NCCc3ccc(Cl)cc3C#N)c3ccccc3)nc2)cn1] | (S)- and (R)- 2- ((4-chloro-2- cyanophen- Ethyl)amino)- N-(5-(1-methyl- 1H-pyrazol-4- yl)pyridin-2-yl)- 2-phenylacet- amide | Calc'd 471.2, Found 471.4 and 471.4 | CHIRALCEL OX-H; 45% (50:50 MeOH:IPA) in hexanes + 0.1% DEA |
| 95 Isomer 1 Isomer 2 | 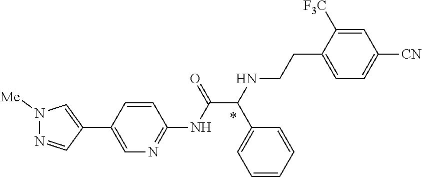 [SMILES: Cn1cc(-c2ccc(NC(=O)C(NCCc3ccc(C#N)cc3C(F)(F)F)c3ccccc3)nc2)cn1] | (S)- and (R)- 2- ((4-cyano-2- (trifluorometh- yl)phenethyl)- amino)-N-(5- (1-methyl-1H- pyrazol-4-yl)- pyridin-2-yl)-2- phenylacetamide | Calc'd 505.2, Found 505.7 and 505.7 | CHIRALCEL OX-H; 25% (70:30 IPA:ACN) in hexanes + 0.1% DEA |
| 96 Isomer 1 Isomer 2 | 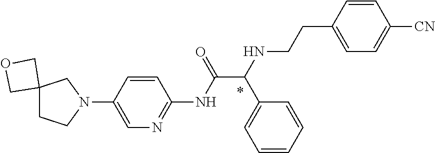 [SMILES: N#Cc1ccc(CCNC(C(=O)Nc2ccc(N3CCC4(COC4)C3)cn2)c2ccccc2)cc1] | (S)- and (R)- N- (5-(2-oxa-6- azaspiro[3.4]- octan-6-yl)pyri- din-2-yl)-2-((4- cyanophen- Ethyl)amino)- 2-phenylacet- amide | Calc'd 468.2, Found 468.5 and 468.4 | CHIRALCEL OJ-H; 35% MeOH in Liquid CO₂ + 0.1% DEA |
| 97 Isomer 1 Isomer 2 | 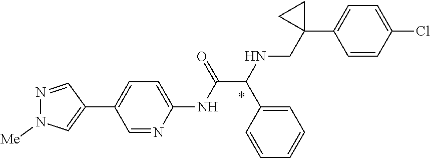 [SMILES: Cn1cc(-c2ccc(NC(=O)C(NCC3(c4ccc(Cl)cc4)CC3)c3ccccc3)nc2)cn1] | (S)- and (R)- 2-(((1-(4- chlorophenyl)- cyclopropyl)- methyl)amino)- N-(5-(1-methyl- 1H-pyrazol-4- yl)pyridin-2-yl)- 2-phenylacet- amide | Calc'd 472.2, Found 472.3 and 472.3 | CHIRALCEL OX-H; 65% (50:50 EPA:MeOH) in hexanes + 0.1% DEA |
| 98 Isomer 1 Isomer 2 Isomer 3 Isomer 4 | 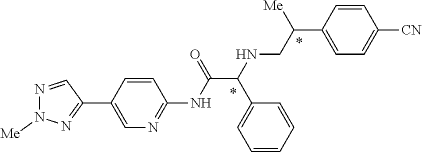 [SMILES: CC(CNC(C(=O)Nc1ccc(-c2cnn(C)n2)cn1)c1ccccc1)c1ccc(C#N)cc1] | (S, S)-, (S, R)-, (R, R)- and (R, S)-2-((2-(4- cyanophenyl)- propyl)amino)- N-(5-(2-methyl- 2H-1,2,3-triazol- 4-yl)pyridin-2- yl)-2-phenylacet- amide | Calc'd 452.2, Found 452.3, 452.3, 452.6 and 452.6 | 1-CHIRAL- CEL OJ-H; 18% MeOH in Liquid CO₂ + 0.1% DEA 2-CHIRAL- PAK IC; 35% (50:50 IPA:MeOH) in hexanes + |
| | | | | 0.1% DEA |
| 99 Isomer 1 Isomer 2 Isomer 3 Isomer 4 | 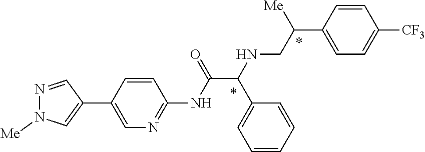 [SMILES: CC(CNC(C(=O)Nc1ccc(-c2cnn(C)c2)cn1)c1ccccc1)c1ccc(C(F)(F)F)cc1] | (S, S)-,(S, R)-, (R, R)- and (R, S)-N-(5-(1- methyl-1H- pyrazol-4-yl)- pyridin-2-yl)- 2-phenyl-2- ((2-(4-trifluoro- methyl)phenyl)- propyl)amino)- | Calc'd 494.2, Found 494.4, 494.3, 494.5 and 494.5 | 1-CHIRAL- CEL OX-H; 35% (50:50 IPA:MeOH) in hexanes + 0.1% DEA 2-CHIRAL- CEL OJ-H; 15% MeOH in Liquid CO₂ + |
| | | acetamide | | 0.1% DEA |
| 100 Isomer 1 Isomer 2 Isomer 3 Isomer 4 | 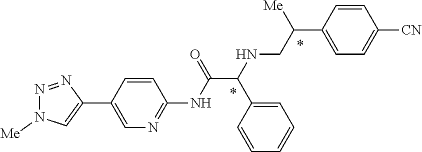 [SMILES: CC(CNC(C(=O)Nc1ccc(-c2cn(C)nn2)cn1)c1ccccc1)c1ccc(C#N)cc1] | (S, S)-, (S, R)-, (R, R)- and (R, S)-2-((2-(4- cyanophenyl)- propyl)amino)- N-(5-(1-methyl- 1H-1,2,3-triazol- 4-yl)pyridin-2- yl)-2-phenyl- acetamide | Calc'd 452.2, Found 452.5, 452.5, 452.5 and 452.5 | 1-CHIRAL- PAK IC; 10% (70:30 IPA:ACN) in hexanes + 0.1% DEA 2-CHIRAL- PAK IC; 28% MeOH in Liquid CO₂ + |
| | | | | 0.1% DEA |
| 101 Isomer 1 Isomer 2 Isomer 3 Isomer 4 | 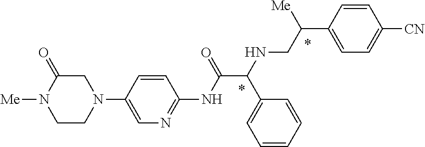 [SMILES: CC(CNC(C(=O)Nc1ccc(N2CCN(C)C(=O)C2)cn1)c1ccccc1)c1ccc(C#N)cc1] | (S, S)-, (S, R)-, (R, R)- and (R, S)-2-((2-(4- cyanophenyl)- propyl)amino)- N-(5-(4-methyl- 3-oxopiperazin- 1-yl)pyridin-2- yl)-2-phenylacet- | Calc'd 483.2, Found 483.5, 483.4, 483.8 and 483.8 | 1-CHIRAL- CEL OJ-H; 20% MeOH in Liquid CO₂ + 0.1% DEA 2-CHIRAL- CEL OJ-H; 20% (50:50 IPA:MeOH) in |
| | | amide | | Liquid CO₂ + |
| | | | | 0.1% DEA |
| 102 Isomer 1 Isomer 2 | 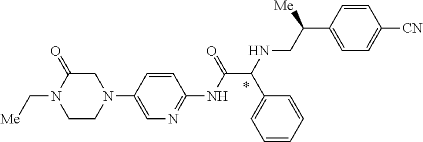 [SMILES: CCN1CCN(c2ccc(NC(=O)C(NC[C@@H](C)c3ccc(C#N)cc3)c3ccccc3)nc2)CC1=O] | (S, S)- and (R, S)- 2-((2-(4-cyano- phenyl)propyl)- amino)-N-(5-(4- Ethyl-3-oxopi- perazin-1-yl)- pyridin-2-yl)-2- phenylacetamide | Calc'd 497.3, Found 497.5 and 497.9 | CHIRALCEL OJ-H; 20% (50:50 IPA:MeOH) in Liquid CO₂ + 0.1% DEA |
| 103 Isomer 1 Isomer 2 Isomer 3 Isomer 4 | 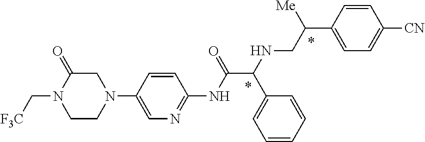 [SMILES: CC(CNC(C(=O)Nc1ccc(N2CCN(CC(F)(F)F)C(=O)C2)cn1)c1ccccc1)c1ccc(C#N)cc1] | (S, S)-, (S, R)- (R, R)- and (R, S)-2-((2-(4-cyano- phenyl)propyl)- amino)-N-(5-(3- oxo-4-(2,2,2- trifluoroEthyl)- piperazin-1-yl)- pyridin-2-yl)-2- | Calc'd 551.2, Found 551.9, 551.6, 551.8 and 551.7 | 1-CHIRAL- PAK IB; 40% (50:50 IPA:MeOH) in hexanes + 0.1% DEA 2-CHIRAL- CEL OX-H; 80% (50:50 |
| | | phenylacetamide | | IPA:MeOH) |
| | | | | in hexanes + |
| | | | | 0.1% DEA |
| 104 Isomer 1 Isomer 2 Isomer 3 Isomer 4 | 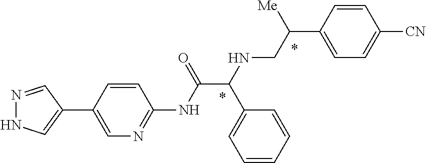 [SMILES: CC(CNC(C(=O)Nc1ccc(-c2cn[nH]c2)cn1)c1ccccc1)c1ccc(C#N)cc1] | (S, S)-,(S, R)-, (R, R)- and (R, S)-N-(5-(1H- pyrazol-4-yl)- pyridin-2-yl)- 2-((2-(4-cyano- phenyl)propyl)- amino)-2-phenyl- acetamide | Calc'd 437.2, Found 437.6, 437.6, 437.4 and 437.4 | 1-CHIRAL- PAK AD-H; 25% (70:30 IPA:ACN) in hexanes + 0.1% DEA 2-CHIRAL- CEL OX-H; 25% (70:30 IPA:ACN) |
| | | | | in hexanes + |
| | | | | 0.1% DEA |
| 105 Isomer 1 Isomer 2 | 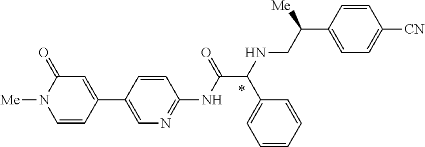 | (R, S)- and (S, S)- 2-((2-(4-cyano- phenyl)propyl)- amino)-N-(1′- methyl-2′-oxo- 1′,2′-dihydro- [3,4′-bipyridin]-6- yl)-2-phenylacet- amide | Calc'd 478.2, Found 478.8 and 478.8 | CHIRALCEL OX-H; 50% (70:30 IPA:ACN) in hexanes + 0.1% DEA |
| 106 Isomer 1 Isomer 2 | 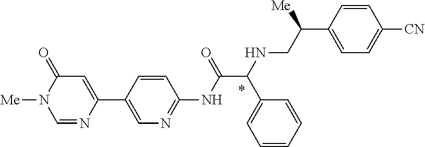 | (R, S)- and (S, S)- 2-((2-(4-cyano- phenyl)propyl)- amino)-N-(5-(1- methyl-6-oxo- 1,6-dihydropyrim- idin-4-yl)pyridin- 2-yl)-2-phenyl- acetamide | Calc'd 479.2, Found 479.7 and 479.7 | CHIRALPAK IC; 35% (70:30 TPA:ACN) in hexanes + 0.1% DEA |
| 107 Isomer 1 Isomer 2 | 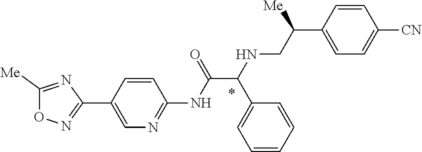 | (R, S)- and (S, S)- 2-((2-(4-cyano- phenyl)propyl)- amino)-N-(5-(5- methyl-1,2,4- oxadiazol-3-yl)- pyridin-2-yl)-2- phenylacetamide | Calc'd 453.2, Found 453.7 and 453.4 | CHIRALCEL OJ-H; 35% (50:50 IPA:MeOH) in hexanes + 0.1% DEA |
| 108 Isomer 1 Isomer 2 Isomer 3 Isomer 4 | 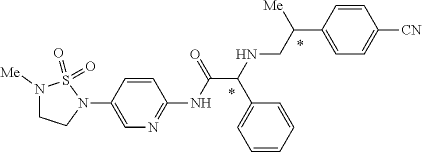 [SMILES: CC(CNC(C(=O)Nc1ccc(N2CCN(C)S2(=O)=O)cn1)c1ccccc1)c1ccc(C#N)cc1] | (S, S)-, (S, R)-, (R, R)- and (R, S)-2-((2-(4- cyanophenyl)- propyl)amino)- N-(5-(5-methyl- 1,1-dioxido- 1,2,5-thiadiazol- idin-2-yl)pyridin- 2-yl)-2-phenyl- | Calc'd 505.2, Found 505.5, 505.5 506.2 and 505.5 | 1-CHIRAL- CEL OJ-H; 20% MeOH in Liquid CO₂ + 0.1% DEA 2-CHIRAL- CEL OJ-H; 15% MeOH in Liquid CO₂ + 0.1% DEA |
| | | acetamide | | |
| 109 Isomer 1 Isomer 2 Isomer 3 Isomer 4 | 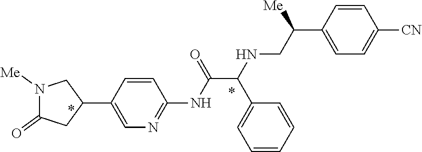 | (R, S, S )-, (R, S, R), (S, S, S)- and (S, S, R)-2-((2- (4-cyanophenyl)- propyl)amino)-N- (5-(1-methyl-5- oxopyrrolidin-3- yl)pyridin-2-yl)-2- phenylacetamide | Calc'd 468.3, Found 468.9, 468.9, 468.8 and 468.8 | CHIRALPAK IC; 35% (70:30 IPA:ACN) in hexanes + 0.1% DEA |
| 110 Isomer 1 Isomer 2 | 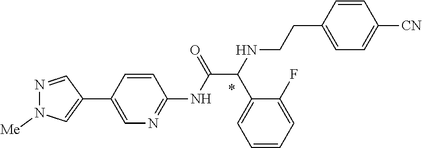 [SMILES: Cn1cc(-c2ccc(NC(=O)C(NCCc3ccc(C#N)cc3)c3ccccc3F)nc2)cn1] | (S)- and (R)- 2- ((4-cyanophen- Ethyl)amino)-2- (2-fluorophenyl)- N-(5-(1-methyl- 1H-pyrazol-4-yl)- pyridin-2-yl)- acetamide | Calc'd 455.2, Found 455.4 and 455.5 | CHIRALCEL OX-H; 40% (70:30 EPA:ACN) in hexanes + 0.1% DEA |
| 111 Isomer 1 Isomer 2 | 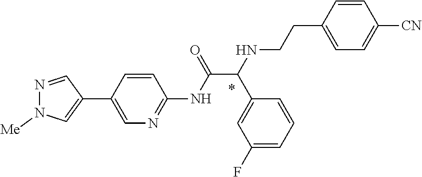 [SMILES: Cn1cc(-c2ccc(NC(=O)C(NCCc3ccc(C#N)cc3)c3cccc(F)c3)nc2)cn1] | (S)- and (R)- 2- ((4-cyanophen- Ethyl)amino)-2- (3-fluorophenyl)- N-(5-(1-methyl- 1H-pyrazol-4-yl)- pyridin-2-yl)acet- amide | Calc'd 455.2, Found 455.4 and 455.7 | CHIRALCEL OX-H; 25% (70:30 IPA:ACN) in hexanes + 0.1% DEA |
| 112 Isomer 1 Isomer 2 | 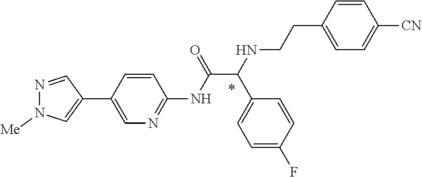 [SMILES: Cn1cc(-c2ccc(NC(=O)C(NCCc3ccc(C#N)cc3)c3ccc(F)cc3)nc2)cn1] | (S)- and (R)- 2- ((4-cyanophen- Ethyl)amino)- 2-(4-fluoro- phenyl)-N-(5- (1-methyl-1H- pyrazol-4-yl)- pyridin-2-yl)- acetamide | Calc'd 455.2, Found 455.8 and 455.4 | CHIRALCEL OJ-H; 50% (50:50 IPA:MeOH) in hexanes + 0.1% DEA |
| 113 Isomer 1 Isomer 2 | 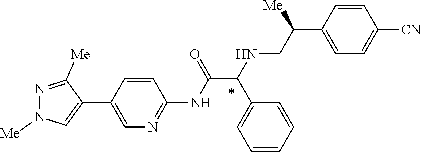 | (R, S)- and (S, S)- 2-((2-(4-cyano- phenyl)propyl)- amino)-N-(5-(1,3- dimethyl-1H- pyrazol-4-yl)- pyridin-2-yl)-2- phenylacetamide | Calc'd 465.2, Found 465.6 and 465.9 | CHIRALCEL OJ-H; 18% MeOH in Liquid CO₂+ 0.1% DEA |
| 114 Isomer 1 Isomer 2 | 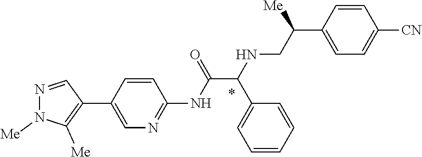 | (R, S)- and (S, S)- 2-((2-(4-cyano- phenyl)propyl)- amino)-N-(5- (1,5-dimethyl- 1H-pyrazol-4-yl)- pyridin-2-yl)-2- phenylacetamide | Calc'd 465.2, Found 465.8 and 465.6 | CHIRALPAK IB; 18% (50:50 EPA:MeOH) in hexanes + 0.1% DEA |
| 115 Isomer 1 Isomer 2 | 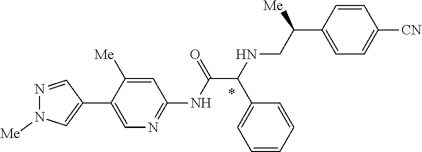 | (R, S)- and (S, S)- 2-((2-(4-cyano- phenyl)propyl)- amino)-N-(4- methyl-5-(1-meth- yl-1H-pyrazol-4- yl)pyridin-2-yl)-2- phenylacetamide | Calc'd 465.2, Found 465.4 and 465.4 | CHIRALCEL OJ-H; 35% MeOH in Liquid CO₂ + 0.1% DEA |
| 116 Isomer 1 Isomer 2 | 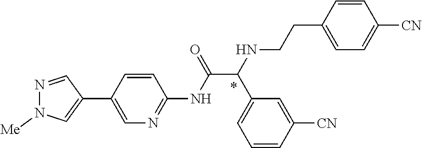 [SMILES: Cn1cc(-c2ccc(NC(=O)C(NCCc3ccc(C#N)cc3)c3cccc(C#N)c3)nc2)cn1] | (S)- and (R)-2- ((4-cyanophen- Ethyl)amino)-2- (3-cyanophenyl)- N-(5-(1-methyl- 1H-pyrazol-4-yl)- pyridin-2-yl)acet- amide | Calc'd 462.2, Found 462.7 and 462.7 | CHIRALCEL OX-H; 60% (50:50 IPA:MeOH) in hexanes + 0.1% DEA |
| 117 Isomer 1 Isomer 2 | 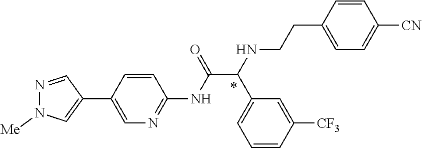 [SMILES: Cn1cc(-c2ccc(NC(=O)C(NCCc3ccc(C#N)cc3)c3cccc(C(F)(F)F)c3)nc2)cn1] | (S)- and (R)-2- ((4-cyanophen- Ethyl)amino)- N-(5-(1-methyl- 1H-pyrazol-4- yl)pyridin-2-yl)- 2-(3-(trifluoro- methyl)phenyl)- acetamide | Calc'd 505.2, Found 505.7 and 505.2 | CHIRALCEL OX-H; 15% MeOH in Liquid CO₂ + 0.1% DEA |
| 118 Isomer 1 Isomer 2 | 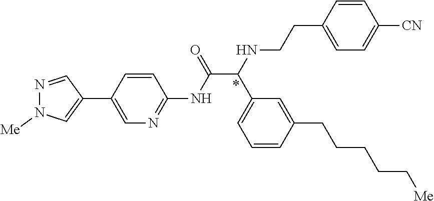 [SMILES: CCCCCCc1cccc(C(NCCc2ccc(C#N)cc2)C(=O)Nc2ccc(-c3cnn(C)c3)cn2)c1] | (S)- and (R)-2- ((4-cyanophen- Ethyl)amino)-2- (3-hexylphenyl)- N-(5-(1-methyl- 1H-pyrazol-4- yl)pyridin-2-yl)- acetamide | Calc'd 521.3, Found 521.8 and 521.8 | CHIRALCEL OX-H; 20% (70:30 IPA:ACN) in hexanes + 0.1% DEA |
| 119 Isomer 1 Isomer 2 | 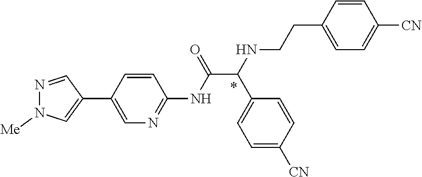 [SMILES: Cn1cc(-c2ccc(NC(=O)C(NCCc3ccc(C#N)cc3)c3ccc(C#N)cc3)nc2)cn1] | (S)- and (R)-2- ((4-cyanophen- Ethyl)amino)- 2-(4-cyanophen- yl)-N-(5-(1-meth- yl-1H-pyrazol-4- yl)pyridin-2-yl)- acetamide | Calc'd 462.2, Found 462.7 and 462.6 | CHIRALCEL OX-H; 55% (50:50 EPA:MeOH) in hexanes + 0.1% DEA |
| 120 Isomer 1 Isomer 2 | 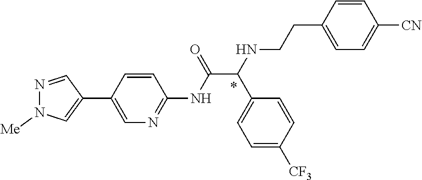 [SMILES: Cn1cc(-c2ccc(NC(=O)C(NCCc3ccc(C#N)cc3)c3ccc(C(F)(F)F)cc3)nc2)cn1] | (S)- and (R)-2- ((4-cyanophen- Ethyl)amino)- N-(5-(1-methyl- 1H-pyrazol-4- yl)pyridin-2-yl)- 2-(4-(trifluoro- methyl)phenyl)- acetamide | Calc'd 505.2, Found 505.2 and 505.2 | CHIRALCEL OX-H; 20% (50:50 IPA:ACN) in Liquid CO₂ + 0.1% DEA |
| 121 Isomer 1 Isomer 2 | 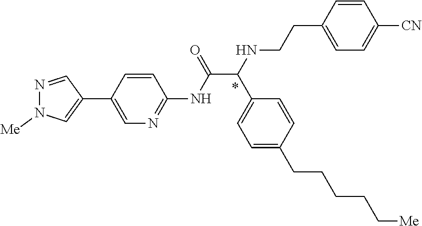 [SMILES: CCCCCCc1ccc(C(NCCc2ccc(C#N)cc2)C(=O)Nc2ccc(-c3cnn(C)c3)cn2)cc1] | (S)- and (R)-2- ((4-cyanophen- Ethyl)amino)-2- (4-hexylphenyl)- N-(5-(1-methyl- 1H-pyrazol-4- yl)pyridin-2-yl)- acetamide | Calc'd 521.3, Found 521.7 and 522.0 | CHIRALPAK IC; 28% (50:50 MeOH:ACN) in Liquid CO₂ + 0.1% DEA |
| 122 Isomer 1 Isomer 2 | 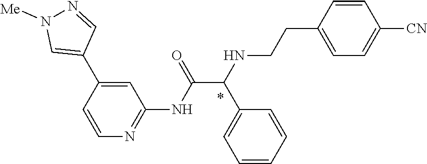 [SMILES: Cn1cc(-c2ccnc(NC(=O)C(NCCc3ccc(C#N)cc3)c3ccccc3)c2)cn1] | (S)- and (R)-2- ((4-cyanophen- Ethyl)amino)-N- (4-(1-methyl-1H- pyrazol-4-yl)- pyridin-2-yl)-2- phenylacetamide | Calc'd 437.2 Found 437.7 and 437.7 | CHIRALCEL OX-H; 20% (70:30 IPA:ACN) in hexanes + 0.1% DEA |
| 123 Isomer 1 Isomer 2 | 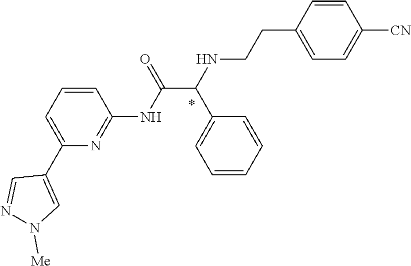 [SMILES: Cn1cc(-c2cccc(NC(=O)C(NCCc3ccc(C#N)cc3)c3ccccc3)n2)cn1] | (S)- and (R)-2- ((4-cyanophen- Ethyl)amino)-N- (6-(1-methyl-1H- pyrazol-4-yl)- pyridin-2-yl)-2- phenylacetamide | Calc'd 437.2, Found 437.6 and 437.5 | CHIRALCEL OJ-H; 15% MeOH in Liquid CO₂ + 0.1% DEA |
| 124 Isomer 1 Isomer 2 | 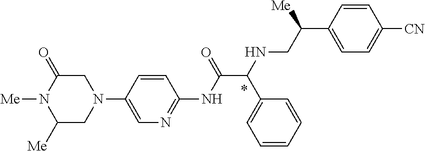 [SMILES: CC1CN(c2ccc(NC(=O)C(NC[C@@H](C)c3ccc(C#N)cc3)c3ccccc3)nc2)CC(=O)N1C] | (R, S, rac)- and (S, S, rac)-2-((2- (4-cyanophenyl)- propyl)amino)-N- (5-(3,4-dimethyl- 5-oxopiperazin-1- yl)pyridin-2-yl)- 2-phenylacet- amide | Calc'd 497.3, Found 497.7 and 497.7 | CHIRALCEL OJ-H; 25% MeOH in Liquid CO₂ + 0.1% DEA |
| 125 Isomer 1 Isomer 2 Isomer 3 Isomer 4 | 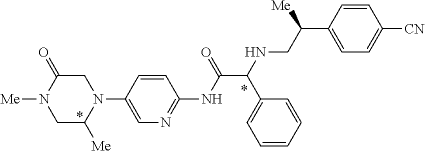 [SMILES: CC1CN(C)C(=O)CN1c1ccc(NC(=O)C(NC[C@@H](C)c2ccc(C#N)cc2)c2ccccc2)nc1] | (R, S, S)-, (R, S, R), (S, S, S)- and (S, S, S)-2-((2- (4-cyanophenyl)- propyl)amino)-N- (5-(2,4-dimethyl- 5-oxopiperazin-1- yl)pyridin-2-yl)-2- phenylacetamide | Calc'd 497.3, Found 497.7, 497.7, 497.7 and 497.5 | 1-CHIRAL- CEL OX-H; 40% (70:30 EPA:ACN) in hexanes + 0.1% DEA 2-CHIRAL- CEL OJ-H; 20% MeOH in Liquid CO₂ + |
| | | | | 0.1% DEA |
| 126 Isomer 1 Isomer 2 Isomer 3 Isomer 4 | 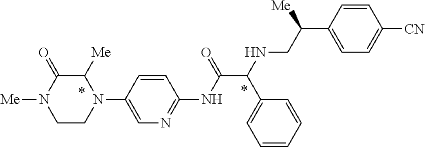 [SMILES: CC1C(=O)N(C)CCN1c1ccc(NC(=O)C(NC[C@@H](C)c2ccc(C#N)cc2)c2ccccc2)nc1] | (R, S, S)-,(R, S, R), (S, S, S)- and (S, S, R)-2-((2- (4-cyanophenyl)- propyl)amino)-N- (5-(2,4-dimethyl- 3-oxopiperazin-1- yl)pyridin-2-yl)-2- phenylacetamide | Calc'd 497.3, Found 497.2, 497.2, 497.2 and 497.2 | 1-CHIRAL CEL OJ-H; 30% MeOH in Liquid CO₂ + 0.1% DEA 2-CHIRAL- CEL OX-H; 35% (70:30 IPA:ACN) |
| | | | | in hexanes + |
| | | | | 0.1% DEA |
| 127 Isomer 1 Isomer 2 Isomer 3 Isomer 4 | 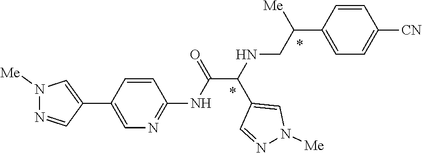 [SMILES: CC(CNC(C(=O)Nc1ccc(-c2cnn(C)c2)cn1)c1cnn(C)c1)c1ccc(C#N)cc1] | (S, S)-,(S, R)-,(R, R)- and (R, S)-2- ((2-(4-cyanophen- yl)propyl)amino)- 2-(1-methyl-1H- pyrazol-4-yl)-N- (5-(1-methyl- 1H-pyrazol-4-yl)- pyridin-2-yl)acet- amide | Calc'd 455.2, Found 455.5, 455.5, 455.7 and 455.7 | 1-CHIRAL- CEL OJ-H; 15% MeOH in Liquid CO₂ + 0.1% DEA 2-CHIRAL- PAK IC; 32% (50:50 MeOH:ACN) in Liquid CO₂ + |
| | | | | 0.1% DEA |
| 128 Isomer 1 Isomer 2 Isomer 3 Isomer 4 | 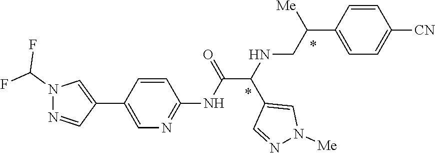 [SMILES: CC(CNC(C(=O)Nc1ccc(-c2cnn(C(F)F)c2)cn1)c1cnn(C)c1)c1ccc(C#N)cc1] | (S, S)-,(S, R)-,(R, R)- and (R, S)-2- ((2-(4-cyanophen- yl)propyl)amino)- N-(5-(1-(difluoro- methyl)-1H-pyra- zol-4-yl)pyridin- 2-yl)-2-(1-methyl- 1H-pyrazol-4-yl)- acetamide | Calc'd 491.2, Found 491.6, 491.7, 491.7 and 491.8 | 1-CHIRAL- CEL OJ-H; 15% MeOH in Liquid CO₂ + 0.1% DEA 2-CHIRAL- PAK AD-H; 22% (50:50 MeOH:ACN) in Liquid CO₂ + |
| | | | | 0.1% DEA |
| 129 Isomer 1 Isomer 2 | 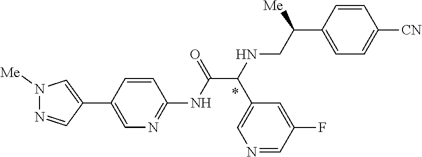 [SMILES: C[C@H](CNC(C(=O)Nc1ccc(-c2cnn(C)c2)cn1)c1cncc(F)c1)c1ccc(C#N)cc1] | (R, S)- and (S, S)- 2-((2-(4-cyano- phenyl)propyl)- amino)-2-(5- fluoropyridin-3- yl)-N-(5-(1-meth- yl-1H-pyrazol-4- yl)pyridin-2-yl)- acetamide | Calc'd 470.2, Found 470.5 and 470.5 | CHIRALCEL OX-H; 45% (70:30 IPA:ACN) in hexanes + 0.1% DEA |
| 130 Isomer 1 Isomer 2 | 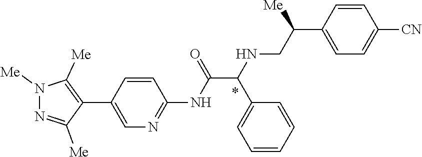 [SMILES: Cc1nn(C)c(C)c1-c1ccc(NC(=O)C(NC[C@@H](C)c2ccc(C#N)cc2)c2ccccc2)nc1] | (R, S)- and (S, S)- 2-((2-(4-cyano- phenyl)propyl)- amino)-2-phenyl- N-(5-(1,3,5- trimethyl-1H- pyrazol-4-yl)- pyridin-2-yl)- acetamide | Calc'd 479.3, Found 479.9 and 479.9 | CHIRALPAK IC; 24% (50:50 MeOH:ACN) in Liquid CO₂ + 0.1% DEA |
| 131 Isomer 1 Isomer 2 | 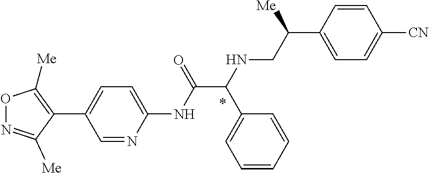 [SMILES: Cc1noc(C)c1-c1ccc(NC(=O)C(NC[C@@H](C)c2ccc(C#N)cc2)c2ccccc2)nc1] | (R, S)- and (S, S)- 2-((2-(4-cyano- phenyl)propyl)- amino)-N-(5-(3,5- dimethylisoxazol- 4-yl)pyridin-2-yl)- 2-phenylacetamide | Calc'd 466.2, Found 466.6 and 466.6 | CHIRALPAK AD-H; 100% MeOH + 0.1% DEA |
| 132 Isomer 1 Isomer 2 | 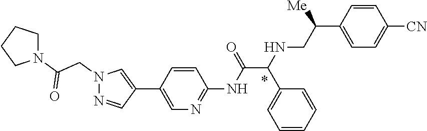 [SMILES: C[C@H](CNC(C(=O)Nc1ccc(-c2cnn(CC(=O)N3CCCC3)c2)cn1)c1ccccc1)c1ccc(C#N)cc1] | (R, S)- and (S, S)- 2-((2-(4-cyano- phenyl)propyl)- amino)-N-(5-(1- (2-oxo-2-(pyrrol- idin-1-yl)Ethyl)- 1H-pyrazol-4-yl)- pyridin-2-yl)-2- phenylacetamide | Calc'd 548.3, Found 549.0 and 548.9 | CHIRALCEL OJ-H; 15% MeOH in Liquid CO₂ + 0.1% DEA |
| 133 Isomer 1 Isomer 2 | 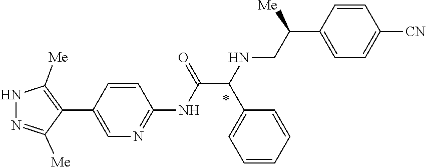 [SMILES: Cc1n[nH]c(C)c1-c1ccc(NC(=O)C(NC[C@@H](C)c2ccc(C#N)cc2)c2ccccc2)nc1] | (R, S)- and (S, S)- 2-((2-(4-cyano- phenyl)propyl)- amino)-N-(5-(3,5- dimethyl-1H- pyrazol-4-yl)- pyridin-2-yl)-2- phenylacetamide | Calc'd 465.2, Found 465.7 and 465.8 | CHIRALCEL OJ-H; 15% MeOH in Liquid CO₂ + 0.1% DEA |
| 134 Isomer 1 Isomer 2 | 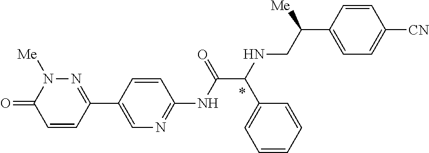 [SMILES: C[C@H](CNC(C(=O)Nc1ccc(-c2ccc(=O)n(C)n2)cn1)c1ccccc1)c1ccc(C#N)cc1] | (R, S)- and (S, S)- 2-((2-(4-cyano- phenyl)propyl)- amino)-N-(5-(1- methyl-6-oxo- 1,6-dihydropyrid- azin-3-yl)pyridin- 2-yl)-2-phenyl- acetainide | Calc'd 479.2, Found 479.7 and 479.6 | CHIRALCEL OJ-H; 18% MeOH in Liquid CO₂ + 0.1% DEA |
| 135 Isomer 1 Isomer 2 | 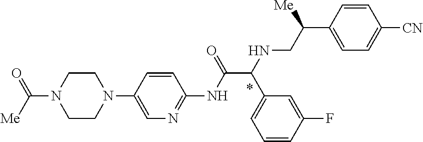 [SMILES: CC(=O)N1CCN(c2ccc(NC(=O)C(NC[C@@H](C)c3ccc(C#N)cc3)c3cccc(F)c3)nc2)CC1] | (R, S)- and (S, S)- N-(5-(4-acetylpi- perazin-1-yl)pyr- idin-2-yl)-2-((2- (4-cyanophenyl)- propyl)amino)-2- (3-fluorophenyl)- acetamide | Calc'd 515.3, Found 515.6 and 515.6 | CHIRALCEL OJ-H; 20% MeOH in Liquid CO₂ + 0.1% DEA |
| 136 Isomer 1 Isomer 2 Isomer 3 Isomer 4 | 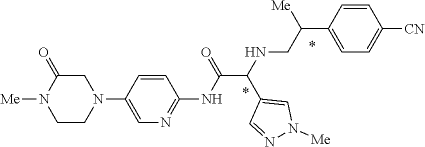 [SMILES: CC(CNC(C(=O)Nc1ccc(N2CCN(C)C(=O)C2)cn1)c1cnn(C)c1)c1ccc(C#N)cc1] | (S, S)-, (S, R)-, (R, R)- and (R, S)-2-((2-(4- cyanophenyl)- propyl)amino)-2- (1-methyl-1H- pyrazol-4-yl)-N- (5-(4-methyl-3- oxopiperazin-1- | Calc'd 487.3, Found 487.6, 487.7, 487.6 and 487.6 | 1-CHIRAL- PAK IG; 100% (30:70 MeOH:ACN) + 0.1% DEA 2-CHIRAL- PAK AD-H; 35% (50:50 MeOH:ACN) in |
| | | yl)pyridin-2-yl)- | | Liquid CO₂ + |
| | | acetamide | | 0.1% DEA |
| 137 Isomer 1 Isomer 2 | 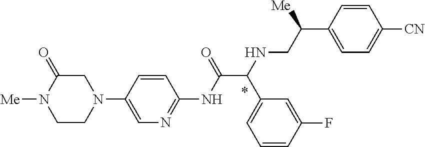 [SMILES: C[C@H](CNC(C(=O)Nc1ccc(N2CCN(C)C(=O)C2)cn1)c1cccc(F)c1)c1ccc(C#N)cc1] | (R, S)- and (S, S)- 2-((2-(4-cyano- phenyl)propyl)- amino)-2-(3- fluorophenyl)-N- (5-(4-methyl-3- oxopiperazin-1- yl)pyridin-2-yl)- acetamide | Calc'd 501.3, Found 501.7 and 501.7 | CHIRALCEL OJ-H; 15% MeOH in Liquid CO₂ + 0.1% DEA |
| 138 Isomer 1 Isomer 2 | 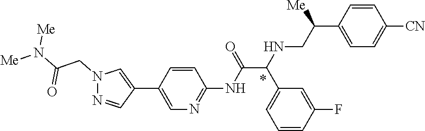 [SMILES: C[C@H](CNC(C(=O)Nc1ccc(-c2cnn(CC(=O)N(C)C)c2)cn1)c1cccc(F)c1)c1ccc(C#N)cc1] | (R, S)- and (S, S)- 2-((2-(4-cyano- phenyl)propyl)- amino)-N-(5-(1- (2-(dimethyl- amino)-2-oxo- Ethyl)-1H-pyra- zol-4-yl)pyridin- | Calc'd 540.2, Found 540.8 and 540.8 | CHIRALCEL OJ-H; 20% MeOH in Liquid CO₂ + 0.1% DEA |
| | | 2-yl)-2-(3-fluoro- | | |
| | | phenyl)acetamide | | |
| 139 Isomer 1 Isomer 2 | 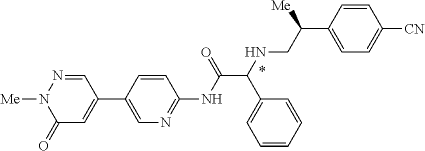 [SMILES: C[C@H](CNC(C(=O)Nc1ccc(-c2cnn(C)c(=O)c2)cn1)c1ccccc1)c1ccc(C#N)cc1] | (R, S)- and (S, S)- 2-(2-(4-cyano- phenyl)propyl)- amino)-N-(5-(1- methyl-6-oxo- 1,6-dihydropyrid- azin-4-yl)pyridin- 2-yl)-2-phenyl- acetamide | Calc'd 479.2, Found 479.5 and 479.5 | CHIRALCEL OJ-H; 50:50 ACN:EPA in Liquid CO₂ + 0.1% DEA |
| 140 Isomer 1 Isomer 2 | 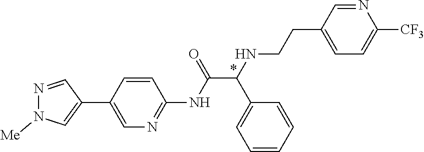 [SMILES: Cn1cc(-c2ccc(NC(=O)C(NCCc3ccc(C(F)(F)F)nc3)c3ccccc3)nc2)cn1] | (S)- and (R)-N- (5-(1-methyl-1H- pyrazol-4-yl)- pyridin-2-yl)-2- phenyl-2-((2-(6- (trifluorometh- yl)pyridin-3-yl)- ethyl)amino)- acetamide | Calc'd 481.2, Found 481.8 and 481.8 | CHIRALCEL OJ-H; MeOH in Liquid CO₂ + 0.1% DEA |
| 141 Isomer 1 Isomer 2 | 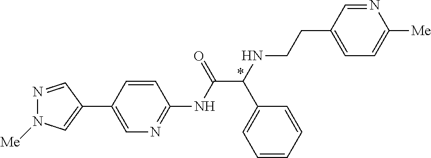 [SMILES: Cc1ccc(CCNC(C(=O)Nc2ccc(-c3cnn(C)c3)cn2)c2ccccc2)cn1] | (S)- and (R)-(5- (1-methyl-1H- pyrazol-4-yl)- pyridin-2-yl)-2- ((2-(6-methyl- pyridin-3-yl)- ethyl)amino)-2- phenylacetamide | Calc'd 427.2, Found 427.8 and 427.6 | CHIRALCEL OJ-H; 50:50 MeOH:EPA in Liquid CO₂ + 0.1% DEA |
| 142 Isomer 1 Isomer 2 | 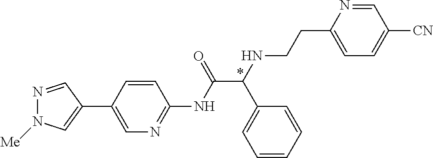 [SMILES: Cn1cc(-c2ccc(NC(=O)C(NCCc3ccc(C#N)cn3)c3ccccc3)nc2)cn1] | (S)- and (R)-2-((2- (5-cyanopyridin- 2-yl)ethyl)amino)- N-(5-(1-methyl- 1H-pyrazol-4-yl)- pyridin-2-yl)-2- phenylacetamide | Calc'd 438.2, Found 438.8 and 438.7 | CHIRALCEL OX-H; 35% (70:30 IPA:ACN) in Hexanes + 0.1% DEA |
| 143 Isomer 1 Isomer 2 Isomer 3 Isomer 4 | 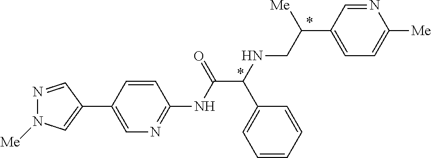 [SMILES: Cc1ccc(C(C)CNC(C(=O)Nc2ccc(-c3cnn(C)c3)cn2)c2ccccc2)cn1] | (S, S)-, (S, R)-, (R, R)- and (R, S)- N-(5-(1-methyl- 1H-pyrazol-4-yl)- pyridin-2-yl)-2- ((2-(6-methylpyr- idin-3-yl)propyl)- amino)-2-phenyl- acetamide | Calc'd 441.2, Found 441.8, 441.8, 441.9, and 441.9 | 1-CHIRAL- CEL OX-H; 40% (70:30 IPA:ACN) in hexanes + 0.1% DEA then 2-CHIRAL- CEL AD-H; 35% IPA in Liquid CO₂ + |
| | | | | 0.1% DEA |
| 144 Isomer 1 Isomer 2 | 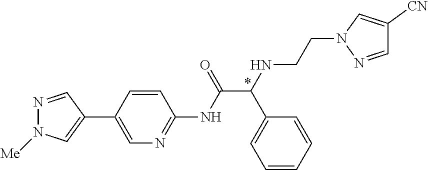 [SMILES: Cn1cc(-c2ccc(NC(=O)C(NCCn3cc(C#N)cn3)c3ccccc3)nc2)cn1] | (S)- and (R)-2-((2- (4-cyano-1H-pyr- azol-1-yl)ethyl)- amino)-N-(5-(1- methyl-1H-pyr- azol-4-yl)pyridin- 2-yl)-2-phenyl- acetamide | Calc'd 427.2, Found 427.8 and 427.8 | CHIRALPAK IG; 90:10 ACN:MeOH in Liquid CO₂ + 0.1% DEA |
| 145 Isomer 1 Isomer 2 | 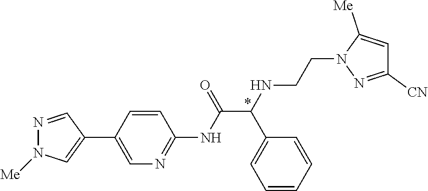 [SMILES: Cc1cc(C#N)nn1CCNC(C(=O)Nc1ccc(-c2cnn(C)c2)cn1)c1ccccc1] | (S)- and (R)-2-((2- (3-cyano-5-meth- yl-1H-pyrazol-1- yl)ethyl)amino)- N-(5-(1-methyl- 1H-pyrazol-4-yl)- pyridin-2-yl)-2- phenylacetamide | Calc'd 441.2, Found 441.7 and 441.7 | CHIRALCEL OX-H; 50:50 MeOH:EPA in Hexane + 0.1% DEA |
| 146 Isomer 1 Isomer 2 | 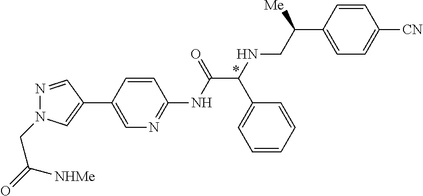 [SMILES: CNC(=O)Cn1cc(-c2ccc(NC(=O)C(NC[C@@H](C)c3ccc(C#N)cc3)c3ccccc3)nc2)cn1] | (R, S)- and (S, S)- 2-((2-(4-cyano- phenyl)propyl)- amino)-N-(5-(1- (2-(methylamino)- 2-oxoethyl)-1H- pyrazol-4-yl)pyri- din-2-yl)-2-phen- ylacetamide | Calc'd 508.2, Found 508.4 and 508.4 | CHIRALCEL OJ-H; MEOH in Liquid CO₂ + 0.1% DEA |
| 147 Isomer 1 Isomer 2 | 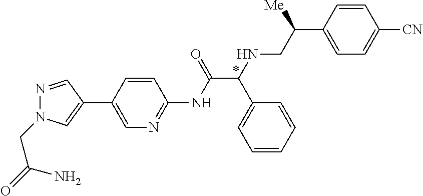 [SMILES: C[C@H](CNC(C(=O)Nc1ccc(-c2cnn(CC(N)=O)c2)cn1)c1ccccc1)c1ccc(C#N)cc1] | (R, S)- and (S, S)- N-(5-(1-(2-amino- 2-oxoethyl)-1H- pyrazol-4-yl)pyri- din-2-yl)-2-((2-(4- cyanophenyl)- propyl)amino)-2- phenylacetamide | Calc'd 494.2, Found 494.2 and 494.2 | CHIRALCEL OJ-H; MeOH in Liquid CO₂ + 0.1% DEA |
| 148 Isomer 1 Isomer 2 | 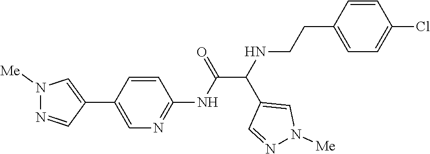 [SMILES: Cn1cc(-c2ccc(NC(=O)C(NCCc3ccc(Cl)cc3)c3cnn(C)c3)nc2)cn1] | (S)- and (R)-2- ((4-chlorophen- ethyl)amino)-2- (1-methyl-1H- pyrazol-4-yl)-N- (5-(1-methyl-1H- pyrazol-4-yl)- pyridin-2-yl)- acetamide | Calc'd 450.2, Found 450.2 and 450.2 | Regis (S,S)- Whelk O-1; 35% MeOH in LIQUID CO₂ + 0.1% isopropylamine |
| 149 Isomer 1 Isomer 2 Isomer 3 Isomer 4 | 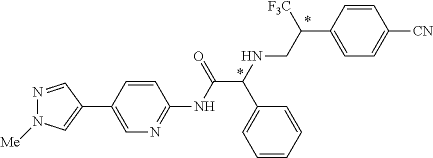 [SMILES: Cn1cc(-c2ccc(NC(=O)C(NCC(c3ccc(C#N)cc3)C(F)(F)F)c3ccccc3)nc2)cn1] | (S, S)-, (S, R)-, (R, R)- and (R, S)-2-((2-(4- cyanophenyl)- 3,3,3-trifluoro- propyl)amino)- N-(5-(1-methyl- 1H-pyrazol-4-yl)- pyridin-2-yl)-2- phenylacetamide | Calc'd 505.5, Found 505.7, 505.7, 505.7, and 505.7 | 1-CHIRAL- CEL OX-H; 33% (50:50 MeOH:IPA) in hexanes + 0.1% DEA then 2-CHIRAL- CEL OJ-H; 10% MeOH in LIQUID CO₂ + |
| | | | | 0.1% DEA) |
| 150 Isomer 1 Isomer 2 Isomer 3 Isomer 4 | 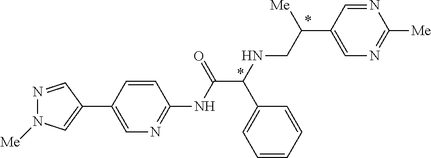 [SMILES: Cc1ncc(C(C)CNC(C(=O)Nc2ccc(-c3cnn(C)c3)cn2)c2ccccc2)cn1] | (S, S)-, (S, R)-, (R, R)- and (R, S)-N-(5-(1-meth- yl-1H-pyrazol- 4-yl)pyridin-2-yl)- 2-((2-(2-methyl- pyrimidin-5-yl)- propyl)amino)-2- phenylacetamide | Calc'd 442.5, Found 442.8, 442.8, 442.8, and 442.8 | CHIRALPAK IG; 100% (70:30 MeOH: ACN) + 0.1% Diethylamine |

### Scheme 7

The starting materials required for the synthesis of examples prepared using Scheme 8. The examples were generally prepared using methods 1 through 16 or were commercially available.

### Example 151

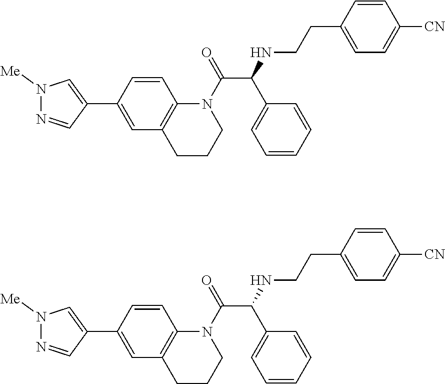 [SMILES: Cn1cc(-c2ccc3c(c2)CCCN3C(=O)[C@@H](NCCc2ccc(C#N)cc2)c2ccccc2)cn1 ; Cn1cc(-c2ccc3c(c2)CCCN3C(=O)[C@H](NCCc2ccc(C#N)cc2)c2ccccc2)cn1]

### (S)- and (R)-4-(2-((2-(6-(1-Methyl-1H-pyrazol-4-yl)-3,4-dihydroquinolin-1(2H)-yl)-2-oxo-1-phenyl ethyl)amino)ethyl)benzonitrile

### Scheme 7, Step 1. tert-Butyl 6-(1-methyl-1H-pyrazol-4-yl)-3,4-dihydroquinoline-1(2H)-carboxylate

A mixture of tert-butyl 6-bromo-3,4-dihydroquinoline-1(2H)-carboxylate (0.55 g, 1.76 mmol), 1-methyl-4-(4,4,5,5-tetramethyl-1,3,2-dioxaborolan-2-yl)-1H-pyrazole (0.439 g, 2.11 mmol) and cesium carbonate (1.43 g, 4.40 mmol) in mixture of 4:1 Dioxane:water (10 ml) was purged for 20 minutes with argon gas. S-Phos Pd-G3-precatalyst (0.066 g, 0.08 mmol) was added and purging was continued for another 10 minutes. The reaction mixture was heated at 100° C. for 2 hours. The reaction mixture was poured into water (25 ml) and extracted with ethyl acetate (2×30 ml). The combined organic layers were washed with brine (20 ml), dried over anhydrous Na₂SO₄ and concentrated under reduced pressure. The resulting residue was purified by silica gel chromatography to afford the title compound (0.55 g, 99%) as a solid. ¹H NMR (400 MHz, DMSO-d₆): 1.08 (s, 9H), 1.81-1.87 (m, 2H), 2.74 (t, J=6.4 Hz, 2H), 3.63 (t, J=6.0 Hz, 2H), 3.85 (s, 3H), 7.29-7.31 (m, 2H), 7.54 (d, J=9.2 Hz, 1H), 7.80 (s, 1H), 8.07 (s, 1H); LCMS: m/z=314.2 [M+1].

### Scheme 7, Step 2. 6-(1-Methyl-1H-pyrazol-4-yl)-1,2,3,4-tetrahydroquinoline

To a stirred solution of tert-butyl 6-(1-methyl-1H-pyrazol-4-yl)-3,4-dihydroquinoline-1(2H)-carboxylate (0.1 g, 0.31 mmol) in dry 1,4-dioxane (1 ml) was added 4M HCl in dioxane (1 ml) dropwise at 0° C. The reaction mixture was stirred at room temperature for 2 hours. The reaction mixture was concentrated and neutralized with saturated sodium bicarbonate and extracted with ethyl acetate (3×10 ml). The combined organic layer was washed with brine (10 ml), dried over anhydrous Na₂SO₄ and concentrated under reduced pressure to afford the title compound (0.050 g, 73%). LCMS: m/z=214.2 [M+1].

### Method 7, Step 3. (S)-and (R)-4-(2-((2-(6-(1-Methyl-1H-pyrazol-4-yl)-3,4-dihydroquinolin-1(2H)-yl)-2-oxo-1-phenylethyl)amino)ethyl)benzonitrile

To a mixture of 6-(1-methyl-1H-pyrazol-4-yl)-1,2,3,4-tetrahydroquinoline (0.05 g, 0.23 mmol) and ethyl 2-((4-cyanophenethyl)amino)-2-phenylacetate (0.060 g, 0.19 mmol) in toluene (0.6 ml) was added TMA (0.19 ml, 2M in Toluene, 0.39 mmol) under an atmosphere of nitrogen at 0° C. The resulting reaction mixture was heated at 100° C. for 2 hours. After completion of the reaction (monitored by TLC), the mixture was slowly quenched with saturated sodium bicarbonate (10 ml) and aqueous layer was extracted with ethyl acetate (2×10 ml). The combined organic layers were washed with brine (10 ml), dried over anhydrous Na₂SO₄ and concentrated under reduced pressure. The resulting residue was purified by silica gel chromatography to afford the title compound (0.03 g, 32%) as a racemic mixture. The racemic compound was resolved by chiral HPLC (CHIRALCEL OJ-H; 15% (50:50 ACN:IPA ) in Liquid CO₂+0.1% DEA) to furnish the enantiopure compounds. The faster-eluting enantiomer of the title compound was obtained as a solid (Isomer 1): ¹H NMR (400 MHz, DMSO-d6): δ 1.71-1.77 (m, 2H), 2.58-2.79 (m, 6H), 3.36-3.52 (m, 1H), 3.79-3.85 (m, 1H), 3.86 (s, 3H), 4.86 (s, 1H), 6.85-7.19 (m, 2H), 7.25-7.35 (m, 6H), 7.41 (d, J=7.6 Hz, 2H), 7.75 (d, J=8.0 Hz, 2H), 7.85 (s, 1H), 8.12 (s, 1H). LCMS: m/z=476.3 [M+1]. The slower-eluting enantiomer of the title compound was obtained as a solid (Isomer 2): ¹H NMR (400 MHz, DMSO-d6): δ 1.71-1.77 (m, 2H), 2.65-2.79 (m, 6H), 3.36-3.46 (m, 1H), 3.79-3.81 (m, 1H), 3.86 (s, 3H), 4.86 (s, 1H), 6.85-7.15 (m, 2H), 7.25-7.35 (m, 6H), 7.41 (d, J=8.0 Hz, 2H), 7.75 (d, J=8.0 Hz, 2H), 7.85 (s, 1H), 8.12 (s, 1H). LCMS: m/z=476.3 [M+1].

The following compounds were prepared using similar procedures to those described for Example 151 using the appropriate starting materials.

TABLE 6

| | | | Exact | Chiral Column |
|---|---|---|---|---|
| Example | | | Mass | and |
| No. | Structure (Methods and Schemes for Preparation) | IUPAC Name | [M + 1] | Mobile Phase |
| 152 Isomer 1 Isomer 2 | 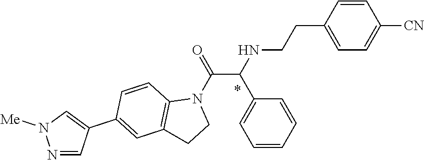 [SMILES: Cn1cc(-c2ccc3c(c2)CCN3C(=O)C(NCCc2ccc(C#N)cc2)c2ccccc2)cn1] | (S)- and (R)-4-(2- ((2-(5-(1-methyl- 1H-pyrazol-4-yl)- indolin-1-yl)-2- oxo-1-phenyl- ethyl)amino)- ethyl)benzonitrile | Calc'd 462.2, Found 462.4 and 462.4 | CHIRALPAK IB; 40% (50:50 IPA: MeOH) in hexanes + 0.1% DEA |

### Example 153

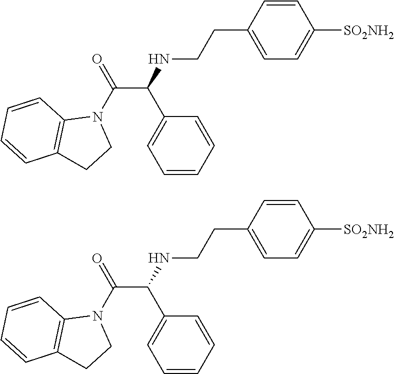 [SMILES: NS(=O)(=O)c1ccc(CCN[C@H](C(=O)N2CCc3ccccc32)c2ccccc2)cc1 ; NS(=O)(=O)c1ccc(CCN[C@@H](C(=O)N2CCc3ccccc32)c2ccccc2)cc1]

### (S)- and (R)-4-(2-((2-(indolin-1-yl)-2-oxo-1-phenylethyl)amino)ethyl)benzenesulfonamide

To a solution of indoline (0.5 g, 4.19 mmol) and TEA (0.849 g, 8.39 mmol) in DMF (10 ml) was added 2-chloro-2-phenylacetyl chloride (0.79 g, 4.19 mmol) drop wise at 0° C. and the reaction mixture was stirred at room temperature for 2 hours. The above reaction mixture was added dropwise to a stirred solution of 4-(2-aminoethyl)benzenesulfonamide (1.67 g, 8.38 mmol) in DMF (5 ml) at room temperature over a period of 10 minutes. The resulting reaction mixture was stirred at room temperature for 2 hours. The reaction mixture was poured into ice cold water (15 ml) and extracted with ethyl acetate (2×15 ml). The combined organic layers were washed with brine (15 ml), dried over anhydrous Na₂SO₄ and concentrated under reduced pressure. The resulting reside was purified by silica gel chromatography to afford the title compound as solid (0.25 g, 14%) in racemic form.

The racemic title compound was resolved by chiral HPLC (CHIRALPAK IB; 40%(50:50 MeOH:IPA) in hexanes+0.1% DEA) to furnish the enantiopure compounds. The faster-eluting enantiomer of the title compound was obtained as a solid (Isomer 1): ¹H NMR (400 MHz, DMSO-d6): δ 2.61-2.84 (m, 4H), 2.99-3.15 (m, 2H), 3.68-3.75 (m, 1H), 4.31-4.38 (m, 1H), 4.69 (s, 1H), 7.01 (t, J=8.0 Hz, 1H), 7.12-7.25 (m, 2H), 7.28-7.44 (m, 8H), 7.73 (d, J=8.0 Hz, 2H), 8.15 (d, J=8.0 Hz, 1H). LCMS: (Method C-3): RT 1.54 min; m z 436.5 [M+1]. The slower-eluting enantiomer of the title compound was obtained as a solid (Isomer 2): ¹H NMR (400 MHz, DMSO-d6): δ 2.64-2.83 (m, 4H), 2.99-3.18 (m, 2H), 3.68-3.75 (m, 1H), 4.31-4.38 (m, 1H), 4.69 (s, 1H), 7.01 (t, J=8.0 Hz, 1H), 7.14-7.22 (m, 2H), 7.28-7.41 (m, 8H), 7.73 (d, J=8.0 Hz, 2H), 8.15 (d, J=8.0 Hz, 1H). LCMS: m/z=436.5 [M+1].

The following compounds were prepared using similar procedures to those described for Example 153 using the appropriate starting materials.

TABLE 7

| | | | Exact | Chiral Column |
|---|---|---|---|---|
| Example | | | Mass | and |
| No. | Structure (Methods and Schemes for Preparation) | IUPAC Name | [M + 1] | Mobile Phase |
| 154 | 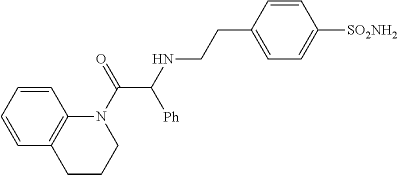 [SMILES: NS(=O)(=O)c1ccc(CCNC(C(=O)N2CCCc3ccccc32)c2ccccc2)cc1] | 4-(2-((2-(3,4- dihydroquinolin- 1(2H)-yl)-2-oxo- 1-phenylethyl)- amino)ethyl)- benzenesulfon- amide | Calc'd 450.6, Found 450.3 | racemic |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating a neurological disorder in a subject in need thereof comprising administering to the subject an effective amount of a compound having the Formula I: 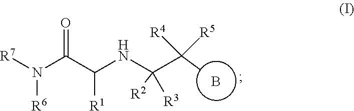
or a pharmaceutically acceptable salt thereof, wherein
Ring B is aryl, heterocyclyl, or heteroaryl each of which may be optionally substituted with 1 to 4 groups selected from R^{b};
R⁶ is a hydrogen or C₁₋₆alkyl;
R⁷ is aryl or heteroaryl, each of which is substituted with one group selected from R^{f}, and wherein said aryl and heteroaryl for R⁷ may also be optionally substituted with 1 to 4 groups selected from R^{a}; or R⁶ and R⁷ taken together with the nitrogen ring to which they are attached form a fused bicyclic heterocyclyl optionally substituted with 1 to 4 groups selected from R^{a};
R¹ is C₁₋₆alkyl, C₁₋₆haloalkyl, C₂₋₆alkenyl, —C₁₋₆alkylOR^{c}, —C₁₋₆alkylN(R^{d})₂, —C₁₋₆alkylC(O)OR^{d}, —C₁₋₆alkylOC₁₋₆alkylN(R^{d})₂, —C₁₋₆alkylSOR^{d}, —C₁₋₆alkylS(O)₂R^{d}, —C₁₋₆alkylSON(R^{d})₂, —C₁₋₆alkylSO₂N(R^{d})₂, —C₁₋₆alkylcycloalkyl, —C₁₋₆alkylheterocyclyl, —C₁₋₆alkylheteroaryl, —C₁₋₆alkylaryl, cycloalkyl, aryl, heteroaryl, or heterocyclyl, wherein each of said cycloalkyl, heterocyclyl, aryl, and heteroaryl alone and in connection with —C₁₋₆alkylcycloalkyl, —C₁₋₆alkylaryl, —C₁₋₆alkylheteroaryl, and —C₁₋₆alkylheterocyclyl are optionally substituted with 1 to 3 groups selected from R^{c};
each of R², R³, R⁴, and R⁵ are independently hydrogen or C₁₋₆alkyl, wherein said C₁₋₆alkyl is optionally substituted with 1 or 2 groups selected from halo, —C(O)OR^{d}, —OC₁₋₆alkylN(R^{d})₂, —C₁₋₆alkylN(R^{d})₂, —N(R^{d})₂, —NR^{d}C₁₋₆alkylOR^{d}, —SOR^{d}, —S(O)₂R^{d}, —SON(R^{d})₂, —SO₂N(R^{d})₂, C₃₋₁₀cycloalkyl, C₅₋₁₀heterocyclyl, C₅₋₁₀heteroaryl, and C₆₋₁₀aryl;
each of R^{a}, R^{b}, and R^{c} are each independently halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅,—Ocycloalkyl, —O—C₁₋₄alkylaryl, —C₁₋₆alkylcycloalkyl, —C₁₋₆alkylaryl, —C₁₋₆alkylheteroaryl, —C₁₋₆alkylheterocyclyl, cycloalkyl, heterocyclyl, heteroaryl, or aryl, wherein each of said cycloalkyl, heterocyclyl, aryl, and heteroaryl alone and in connection with —Ocycloalkyl, —C₁₋₆alkylcycloalkyl, —C₁₋₆alkylaryl, —C₁₋₆alkylheteroaryl, and —C₁₋₆alkylheterocyclyl are optionally substituted with 1 to 3 groups selected from halo, C₁₋₆alkyl, C₁₋₆haloalkyl, C₁₋₆alkoxy, C₁₋₆haloalkoxy, —N(R^{d})₂, —C(O)R^{d}, and —C₁₋₆alkylOR^{d};
each R^{d} is independently hydrogen, C₁₋₆haloalkyl, or C₁₋₆alkyl; and
each R^{f} is independently cycloalkyl, heterocyclyl, heteroaryl, or aryl, wherein each of said cycloalkyl, heterocyclyl, aryl, and heteroaryl are optionally substituted with 1 to 3 groups selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅,—Ocycloalkyl;
provided the compound is not N-[1,1′-biphenyl]-2-yl-2-[[2-(3,4-dimethoxyphenyl)ethyl]amino]-propanamide, or 2-[(2-phenylpropyl)amino]-N-[4-(1H-1,2,4-triazol-1-yl)phenyl]-propanamide, or a salt thereof.

2. The method of claim 1, wherein the compound is of the Formula II or III: 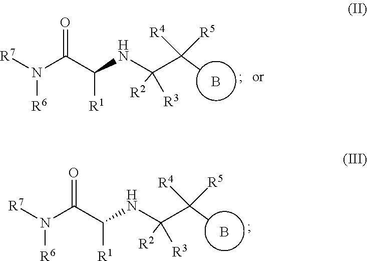
or a pharmaceutically acceptable salt thereof.

3. The method of claim 1 or 2, wherein R⁶ is hydrogen; and R⁷ is aryl or heteroaryl, each of which is substituted with one group selected from R^{f}, and wherein said aryl and heteroaryl for R⁷ may also be optionally substituted with 1 to 4 groups selected from R^{a}; or R⁶ and R⁷ taken together with the nitrogen ring to which they are attached form a fused bicyclic heterocyclyl optionally substituted with 1 to 4 groups selected from R^{a}.

4. The method of any one of claims 1 to 3, wherein R⁶ is hydrogen; and R⁷ is phenyl, pyridyl, pyrimidinyl, or quinolinyl, each of which is substituted with one group selected from R^{f}, and wherein said phenyl, pyridyl, pyrimidinyl, and quinolinyl for R⁷ may also be optionally substituted with 1 to 4 groups selected from R^{a}; or R⁶ and R⁷ taken together with the nitrogen ring to which they are attached form a 5,6- or 6,6-fused bicyclic heterocyclyl optionally substituted with 1 to 4 groups selected from R^{a}.

5. The method of any one of claims 1 to 4, wherein R⁶ is hydrogen; R⁷ is selected from phenyl, 2-pyridinyl, 3-pyridinyl, pyrimidin-5-yl, and quinolin-6-yl, each of which is substituted with one group from R, and wherein said phenyl, 2-pyridinyl, 3-pyridinyl, pyrimidin-5-yl, and quinolin-6-yl for R⁷ may also be optionally substituted with 1 to 4 groups selected from R^{a}; or R⁶ and R⁷ taken together with the nitrogen ring to which they are attached form indolin-1-yl or dihydroquinolin-1(2H)-yl, each of which may be optionally substituted with 1 to 4 groups selected from R^{a}.

6. The method of any one of claims 1 to 5, wherein Ring B is phenyl optionally substituted with 1 to 3 groups selected from R^{b}.

7. The method of any one of claims 1 to 6, wherein R¹ is phenyl optionally substituted with 1 to 3 groups selected from R^{c}.

8. The method of any one of claims 1 to 7, wherein R³ is hydrogen.

9. The method of any one of claims 1 to 8, wherein R⁵ is hydrogen.

10. The method of any one of claims 1 to 9, wherein R² is hydrogen or C₁₋₄alkyl.

11. The method of any one of claims 1 to 10, wherein R² is hydrogen or methyl.

12. The method of any one of claims 1 to 11, wherein R² is hydrogen.

13. The method of any one of claims 1 to 12, wherein R⁴ is hydrogen or C₁₋₄alkyl.

14. The method of any one of claims 1 to 13, wherein R⁴ is hydrogen or methyl.

15. The method of any one of claims 1 to 14, wherein R⁴ is hydrogen.

16. The method of any one of claims 1 to 15, wherein the compound is of the Formula IV or V: 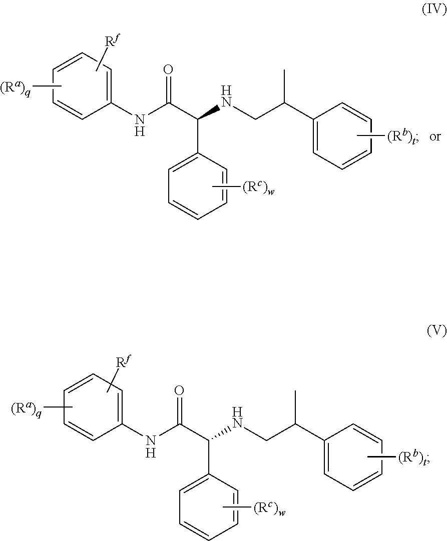
or a pharmaceutically acceptable salt thereof, wherein w, q, and t are each independently 0, 1, or 2.

17. The method of any one of claims 1 to 15, wherein the compound is of the Formula VI or VII: 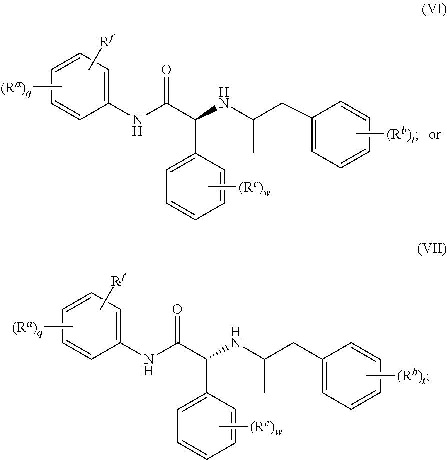
or a pharmaceutically acceptable salt thereof, wherein w, q, and t are each independently 0, 1, or 2.

18. The method of any one of claims 1 to 15, wherein the compound is of the Formula VIII or IX: 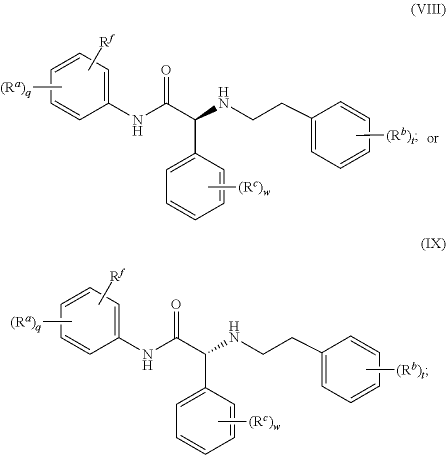
or a pharmaceutically acceptable salt thereof, wherein w, q, and t are each independently 0, 1, or 2.

19. The method of any one of claims 1 to 18, wherein R^{c}, if present, is C₁₋₆alkyl, C₁₋₆alkoxy, C₁₋₆haloalkoxy, or C₁₋₆haloalkyl.

20. The method of any one of claims 1 to 15, wherein the compound is of the Formula X or XI: 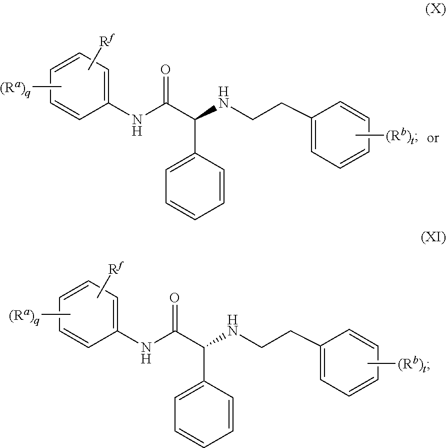
or a pharmaceutically acceptable salt thereof.

21. The method of any one of claims 1 to 20, wherein q is 0 or 1.

22. The method of any one of claims 1 to 21, wherein R^{a} is C₁₋₄alkoxy or halo.

23. The method of any one of claims 1 to 22, wherein R is heteroaryl or heterocyclyl, each of which may be optionally substituted with 1 to 3 groups selected from selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅,—Ocycloalkyl.

24. The method of any one of claims 1 to 23, wherein R is pyrazolyl, imidazolyl, pyridazinyl, piperazinyl, or piperidinyl, each of which may be optionally substituted with 1 to 3 groups selected from selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅,—Ocycloalkyl.

25. The method of any one of claims 1 to 24, wherein R^{f} is pyrazolyl, imidazolyl, pyridazinyl, piperazinyl, or piperidinyl, each of which may be optionally substituted with 1 to 3 groups selected from selected from C₁₋₄alkyl and —C(O)R^{d}, wherein R^{d} is C₁₋₄alkyl.

26. The method of any one of claims 1 to 25, wherein R^{b} is halo, cyano, or —SO₂NH₂.

27. The method of any one of claims 1 to 15, wherein the compound is of the Formula XII or XIII: 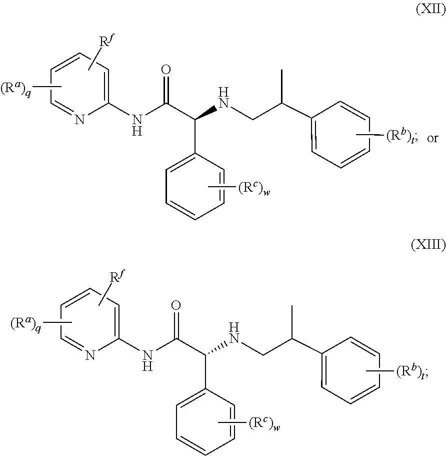
or a pharmaceutically acceptable salt thereof, wherein w, q, and t are each independently 0, 1, or 2.

28. The method of any one of claims 1 to 15, wherein the compound is of the Formula XIV or XV: 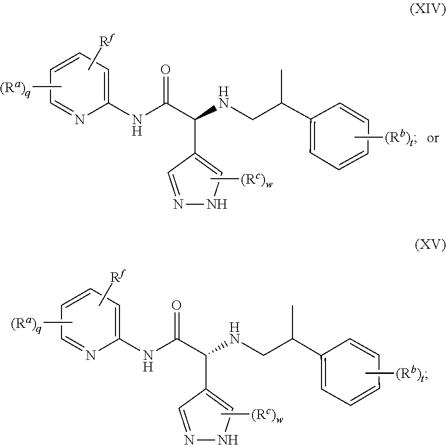
or a pharmaceutically acceptable salt thereof, wherein w, q, and t are each independently 0, 1, or 2.

29. The method of any one of claims 1 to 15, wherein the compound is of the Formula XVI or XVII: 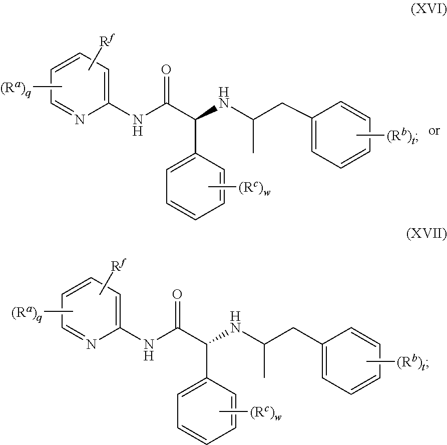
or a pharmaceutically acceptable salt thereof, wherein w, q, and t are each independently 0, 1, or 2.

30. The method of any one of claims 1 to 15, wherein the compound is of the Formula XVIII or XIX: 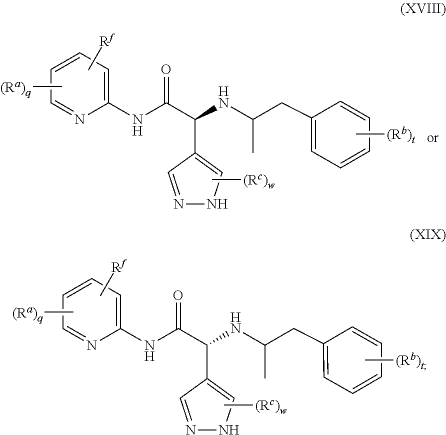
or a pharmaceutically acceptable salt thereof, wherein w, q, and t are each independently 0, 1, or 2.

31. The method of any one of claims 1 to 15, wherein the compound is of the Formula XX or XXI: 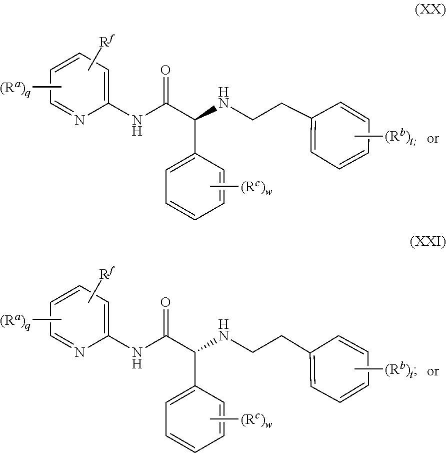
or a pharmaceutically acceptable salt thereof, wherein w, q, and t are each independently 0, 1, or 2.

32. The method of any one of claims 1 to 15, wherein the compound is of the Formula XXII or XXIII: 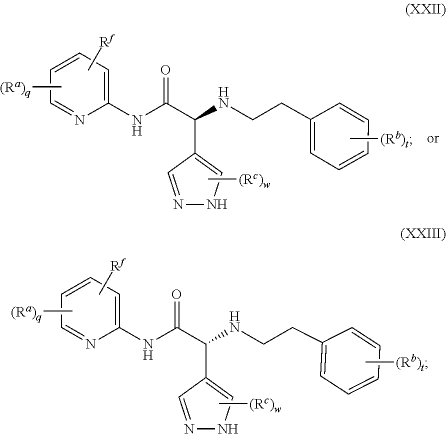
or a pharmaceutically acceptable salt thereof, wherein w, q, and t are each independently 0, 1, or 2.

33. The method of any one of claims 27 to 32, wherein R^{c}, if present, is independently C₁₋₆alkyl, halo, or CN.

34. The method of any one of claims 27 to 33, wherein R^{c}, if present, is C₁₋₄alkyl.

35. The method of any one of claims 27 to 34, wherein w is 0 or 1.

36. The method of any one of claims 27 to 35, wherein R^{b} is halo, cyano, or —SO₂NH₂.

37. The method of any one of claims 27 to 36, wherein R^{b} is cyano.

38. The method of any one of claims 27 to 37, wherein t is 1.

39. The method of any one of claims 27 to 38, wherein q is 1.

40. The method of any one of claims 27 to 39, wherein R is cycloalkyl, phenyl, heteroaryl, or heterocyclyl, each of which may be optionally substituted with 1 to 3 groups selected from halo, CN, oxo, NO₂, C₁₋₆alkyl, C₂₋₆alkenyl, C₁₋₆alkoxy, C₁₋₆haloalkoxy, C₁₋₆haloalkyl, —C₁₋₆alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅,—Ocycloalkyl.

41. The method of any one of claims 27 to 40, wherein R^{f} is pyrimidinyl, phenyl, cyclobutanyl, cyclopropyl, pyrazolyl, imidazolyl, azetidinyl, piperidinyl, pyrrolidinyl, piperazinyl, triazolopyrazinyl, triazolyl, imidazolidinyl, thiadiazolidinyl, morpholinyl, oxaazaspiroheptanyl, oxaazaspirooctanyl, dihydropyrimidinyl, oxadiazolyl, isoxazolyl, or dihydropyridazinyl, each of which may be optionally substituted with 1 to 3 groups selected from halo, CN, oxo, NO₂, C₁₋₆alkyl, C₂₋₆alkenyl, C₁₋₆alkoxy, C₁₋₆haloalkoxy, C₁₋₆haloalkyl, —C_{1 6}alkylOR^{d}, —C(O)R^{d}, —C(O)OR^{d}, —C₁₋₆alkylC(O)OR^{d}, —C(O)N(R^{d})₂, —C(O)NR^{d}C₁₋₆alkylOR^{d}, —OC₁₋₆alkylN(R^{d})₂, —C₁₋₆alkylC(O)N(R^{d})₂, —C₁₋₆alkylN(R^{d})₂, —N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, SF₅,—Ocycloalkyl.

42. The method of any one of claims 27 to 41, wherein R^{f} is pyrimidinyl, phenyl, pyrazolyl, imidazolyl, azetidinyl, piperidinyl, pyrrolidinyl, piperazinyl, triazolopyrazinyl, triazolyl, imidazolidinyl, thiadiazolidinyl, morpholinyl, oxaazaspiroheptanyl, oxaazaspirooctanyl, dihydropyrimidinyl, oxadiazolyl, isoxazolyl, or dihydropyridazinyl, each of which may be optionally substituted with 1 to 3 groups selected from halo, oxo, C₁₋₆alkyl, C₁₋₆alkoxy, C₁₋₆haloalkyl, —C₁₋₆alkylOR^{d}, —C(O)R^{d}, —C(O)N(R^{d})₂, —C₁₋₆alkylC(O)N(R^{d})₂, and —S(O)₂R^{d}.

43. The method of any one of claims 27 to 42, wherein R^{f} is pyrazolyl or triazolyl, each of which may be optionally substituted with C₁₋₃alkyl or —C(O)N(R^{d})₂.

44. The method of any one of claims 27 to 43, wherein R^{d} is hydrogen or C₁₋₃alkyl.

45. The method of any one of claims 27 to 44, wherein R^{d} is C₁₋₃alkyl.

46. The method of any one of claims 1 to 45, wherein the neurological disorder is selected from frontotemporal dementia, Alzheimer's disease, tauopathies, vascular dementia, Parkinson's disease, and dementia with Lewy bodies
